(19) United States
(12) Patent Application Publication
(10) Pub. No.: US 2022/0312817 A1
(43) Pub. Date: Oct. 6, 2022

(54) MICROBIOTA-DIRECTED FOODS TO REPAIR A SUBJECT'S GUT MICROBIOTA

(71) Applicants: Washington University, St. Louis, MO (US); International Centre For Diarrhoeal Disease Research, Bangladesh, Dhaka (BD)

(72) Inventors: ROBERT YUZEN CHEN, St. Louis, MO (US); MATTHEW CHARLES HIBBERD, St. Louis, MO (US); JEFFREY l. GORDON, St. Louis, MO (US); MICHAEL J. BARRATT, St. Louis, MO (US); MD MUNIRUL ISLAM, Dhaka (BD); HAO-WEI CHANG, St. Louis, MO (US); JEANETTE GEHRIG, St. Louis, MO (US); TAHMEED AHMED, Dhaka (BD); SIDDARTH VENKATESH, St. Louis, MO (US)

(21) Appl. No.: 17/618,314

(22) PCT Filed: Jun. 10, 2020
(86) PCT No.: PCT/US20/37085

§ 371 Date: Dec. 10, 2021

Related U.S. Application Data
(60) Provisional application No. 62/859,582, filed on Jun. 10, 2019.

(51) Int. Cl. A23L 33/15; A23L 33/22; A23L 33/115; A23L 33/125; A23L 33/135; A23L 33/16; A23L 33/00; A61K 35/74; A23L 11/00

(52) U.S. Cl. CPC A23L 33/15; A23L 33/22; A23L 33/115; A23L 33/125; A23L 33/135; A23L 33/16; A23L 33/40; A61K 35/74; A23L 11/07

(57) ABSTRACT

The present disclosure provides composition and methods to improve the nutritional status of a subject, as well as aid in the maturation of the gut microbiota of a subject. The disclosure encompasses edible compositions that, when eaten in a manner described herein, impacts the subject's gut microbiota by changing the relative abundances of a plurality of health-discriminatory gut taxa in a statistically significant manner towards chronologically age-matched healthy subjects.

### CROSS-REFERENCE TO RELATED APPLICATION

This application claims priority to U.S. Provisional Application No. 62/859,582, filed Jun. 10, 2019, the disclosures of which are incorporated herein by reference.

### GOVERNMENTAL RIGHTS

This invention was made with government support under DK030292 awarded by the National Institutes of Health. The government has certain rights in the invention.

### FIELD

The present disclosure provides composition and methods to improve the nutritional status of a subject, as well as aid in the repair of a subject's gut microbiota.

### BACKGROUND

Childhood undernutrition is a vexing, pressing, and in many respects overwhelming global health issue. Undernutrition contributes to more than 40% of deaths worldwide among children under 5 years old. Acute undernutrition affects more than 50 million children and is defined by a low weight-for-height Z (WHZ) score [the number of standard deviations from the median value for a reference, multinational World Health Organization (WHO) cohort of children with healthy growth phenotypes]. Preschool children with severe wasting (WHZ <−3) have a 10-fold higher mortality rate than that of their well-nourished counterparts. In 2014, chronic undernutrition, which manifests as stunting [low height-for-age Z score (HAZ)], affected 159 million children, with almost all living in low-income countries. Despite these categorical distinctions, deficits in ponderal and linear growth frequently coexist and increase the risk that children will experience persistent stunting, defective immune responses, and impaired neurocognitive function into adulthood.

Current approaches to treatment have only modest effects in correcting these long-term sequelae, suggesting that certain features of host biology are not being adequately repaired. This has led to the hypothesis that healthy growth is dependent, in part, on normal postnatal development of the gut microbiota and that perturbations in its development are causally related to undernutrition.

### SUMMARY

In an aspect, the present disclosure encompasses a composition comprising chickpea flour, peanut flour, soy flour, green banana, and a micronutrient premix, wherien the micronutrient premix provides at least 60% of the recommended daily allowance of vitamin A, vitamin C, vitamin D, vitamin E, vitamin B, calcium, copper, iron, magnesium, manganese, phosphorus, potassium, and zinc for a child aged 12-18 months; wherein the composition contains no milk, powdered milk or milk product; wherein the composition has about 300 to about 560 kcal per 100 g of the composition, a protein energy ratio (PER) of about 8% to about 20%, and a fat energy ratio (FER) of about 30% to about 60%, and wherein the amount of protein is at least 11 g per 100 g of the composition and the amount of fat is not more than 36 g per 100 g of the composition; and wherein the chickpea flour, the peanut flour, the soy flour, and the green banana, in total, provide at least 9 g of protein per 100 g of the composition.

In another aspect, the present disclosure encompasses a composition comprising chickpea flour, peanut flour, soy flour, green banana, and a micronutrient premix, wherien the micronutrient premix provides at least 60% of the recommended daily allowance of vitamin A, vitamin C, vitamin D, vitamin E, vitamin B, calcium, copper, iron, magnesium, manganese, phosphorus, potassium, and zinc for a child aged 12-18 months; wherein the composition contains no milk, powdered milk or milk product; wherein the composition has about 400 to about 560 kcal per 100 g of the composition, about 20 g to about 36 g of fat per 100 g of the composition, about 11 g to about 16 g of protein per 100 g of the composition, a protein energy ratio (PER) of about 8% to about 12%, and a fat energy ratio (FER) of about 45% to about 60%; and wherein the chickpea flour, the peanut flour, the soy flour, and the green banana, in total, provide at least 9 g of protein per 100 g of the composition.

In another aspect, the present disclosure encompasses a composition comprising chickpea flour, peanut flour, soy flour, green banana, and a micronutrient premix, wherein the micronutrient premix provides at least 60% of the recommended daily allowance of vitamin A, vitamin C, vitamin D, vitamin E, vitamin B, calcium, copper, iron, magnesium, manganese, phosphorus, potassium, and zinc for a child aged 12-18 months; wherein the composition contains no milk, powdered milk or milk product; wherein the composition has about 400 to about 560 kcal per 100 g of the composition, about 20 g to about 36 g of fat per 100 g of the composition, about 11 g to about 16 g of protein per 100 g of the composition, a protein energy ratio (PER) of about 8% to about 12%, and a fat energy ratio (FER) of about 45% to about 60%; wherein some or all the chickpea flour is replaced with a glycan equivalent of chickpea flour, some or all the peanut flour is replaced with a glycan equivalent of peanut flour, some or all the soy flour is replaced with a glycan equivalent of soy flour, or some or all the green banana is replaced with a glycan equivalent of green banana; and wherein the chickpea flour or equivalent, the peanut flour or equivalent, the soy flour or equivalent, and the green banana or equivalent, in total, provide at least 9 g of protein per 100 g of the composition.

In another aspect, the present disclosure encompasses a method for repairing a subject's gut microbiota, improving a subject's growth, or improving the health of subject in need thererof, the method comprising administering to the subject an effective amount of composition of the above paragraphs to the subject.

In another aspect, the present disclosure encompasses a method of treating malnutrition in a subject in need thereof, the method comprising administering an effective amount of a composition of the above paragraphs to the subject.

In another aspect, the present disclosure encompasses a method for increasing the abundance of mediators of bone growth, mediators of neurodevelopment, mediators of inflammation, or any combination thereof, the method comprising administering an effective amount of a composition of the above paragraphs to the subject.

In another aspect, the present disclosure encompasses a method of analyzing the efficacy of a therapeutic intervention on the nutritional status of a subject in need thereof, the method comprising (a) determining the concentration of a plurality of healthy-discriminatory protein in a biological sample obtained from the subject, (b) administering the therapeutic intervention, (c) determining the post-therapeutic intervention concentration of each healthy-discriminatory protein from step (a), (d) determining if the concentration of each healthy-discriminatory protein was modified by the therapeutic intervention, and (e) categorizing the therapeutic intervention as efficacious in improving the nutritional status of the subject when the concentrations of more than 50% of the healthy-discriminatory proteins statistically change in a manner towards those encountered in healthy individuals after administration of the therapeutic intervention.

In another aspect, the present disclosure encompasses a method of analyzing the efficacy of a therapeutic intervention on the physical characteristics of a subject in need thereof, the method comprising (a) determining the concentration of a plurality of LAZ-discriminatory proteins or WHZ-discriminatory proteins in a biological sample from the subject, (b) administering the therapeutic intervention, (c) determining the post-therapeutic intervention concentration of each LAZ-discriminatory proteins or WLZ-discriminatory protein measured in step (a), (d) determining if the concentration of each of the LAZ or WLZ-discriminatory proteins was modified by the therapeutic intervention, and (e) categorizing the therapeutic intervention as efficacious in improving the physical characteristics of the subject when more than 50% of the positively correlated LAZ or WLZ-discriminatory protein concentrations rose after administration of the therapeutic intervention, or when more than 50% of the negatively correlated LAZ-discriminatory protein concentrations fell after administration of the therapeutic intervention.

In another aspect, the present disclosure encompasses a method of analyzing the efficacy of a therapeutic intervention on the maturity of a subject's gut microbiota, the method comprising (a) measuring the subject's gut microbiota health; (b) administering the therapeutic intervention; (c) re-measuring the subject's gut microbiota health by the method used in step (a); and (d) categorizing the therapeutic intervention as efficacious when the subject's gut microbiota health is repaired.

Other aspects and iterations of the invention are described more thoroughly below.

### BRIEF DESCRIPTION OF THE FIGURES

The application file contains at least one photograph executed in color. Copies of this patent application publication with color photographs will be provided by the Office upon request and payment of the necessary fee.

FIG. 1A is an illustration depicting the study design of Example 1.

FIG. 1B and FIG. 1C graphically depict outcomes from a longitudinal study of Bangladeshi children with SAM treated with therapeutic foods. FIG. 1B depicts anthropometry and MAZ scores. Gray bars represent three time points at which blood samples were collected. FIG. 1C is a graphical summary of MAZ score for children with SAM (QHZ<−3; n=96 fecal samples) and subsequently post-SAM MAM WHZ>−3 and <−2; n=151 fecal samples), plus healthy children aged 6-24 months living in the same area where the SAM study was conducted (n=450 fecal samples). Mean values for WHZ, WAZ, HAZ, and MAZ±SEM are plotted in the x-axes of panels B and C. ****, p<0.0001 (one-way ANOVA followed by Tukey's multiple comparisons test).

FIG. 2A, FIG. 2B, FIG. 2C, FIG. 2D, FIG. 2E, FIG. 2F, and FIG. 2G graphically depict metabolic features of children with SAM prior to and following treatment with therapeutic foods. Levels of standard clinical metabolites (FIG. 2A, FIG. 2B), acylcarnitines (FIG. 2C, FIG. 2D), and amino acids and ketoacids (FIG. 2E, FIG. 2F, FIG. 2G) in plasma collected from children at enrollment (B1 blood sample in FIG. 1A), discharge (B2 blood sample in FIG. 1A) and 6-months after discharge (B3 blood sample in FIG. 1A). Abbreviations for branched chain ketoacids in FIG. 2C: KIC, α-ketoisocaproate; KIV, α-ketoisovalerate; and KMV, α-keto-β-methylvalerate. Mean values±SEM are plotted. *, p<0.05; **, p<0.01; ***, p<0.001; ****, p<0.0001 (paired t-test followed by FDR correction). In all figures, blue (far left column) is enrollment (B1), red (middle column) is discharge (B2), and green (far right column) is 6-month post discharge (B3).

FIG. 3 depicts a sparse 30 OTU RF-derived model generated from healthy members of the Mirpur birth cohort (n=25 individuals; 539 fecal samples). OTUs are ranked in descending order of their importance to the accuracy of the model. The x-axis plots the increase in mean-squared error when abundance values from each OTU are randomly permuted. The inset shows the cross-validation curves that result from reducing the number of 97% ID OTUs used for model training.

FIG. 4A, FIG. 4B, FIG. 4C, FIG. 4D, FIG. 4E, FIG. 4F, and FIG. 4G provide comparisons of the fecal microbiomes of children with healthy growth phenotypes and those treated for SAM. FIG. 4A and FIG. 4B depict changes in representation of mcSEED subsystems/pathway modules in the fecal microbiomes of healthy Mirpur children (n=30) sampled during the first three postnatal years (n=30 individuals). (FIG. 4B is a continuation of FIG. 4A.) FIG. 4C shows ranked feature importance of the age-discriminatory mcSEED subsystems/pathway modules comprising the RF-derived model of gut microbiome development in Mirpur infants/children with healthy growth phenotypes. The mcSEED subsystem/pathway module ‘lipoate biosynthesis’ had the highest feature importance score; lipoate/lipoic acid is an essential cofactor of dehydrogenase enzymes including branched-chain ketoacid dehydrogenase, a regulator of branched-chain amino acid catabolism. The representation of subsystems (pathways) involved in amino acid metabolism (including branched-chain amino acids and tryptophan), B vitamin metabolism, plus carbohydrate metabolism and fermentation are important contributors to the accuracy of the model. FIG. 4D is a heatmap of the changes in representation of the top 30 most age-discriminatory mcSEED subsystems/pathway modules in the fecal microbiomes of the healthy Mirpur children. FIG. 4E depicts relative functional maturity of the microbiomes of children hospitalized with SAM at enrollment, just prior to treatment, at discharge, and at 1, 6, 8, and 12 months post-discharge, calculated using the RF-derived model. ns, not significant; *, p<0.05; (one-way ANOVA, Tukey's multiple comparisons test). Note that the statistically significant improvement in functional maturity at 1-month post-discharge compared to that at enrollment is similar to the improvement in MAZ (FIG. 1B). Moreover, there is a significant positive correlation between MAZ and relative functional microbiome maturity (r=0.55, p<0.0001). However, this improvement is not sustained; there is no statistically significant difference in this parameter between initiation of treatment and 8 or 12-months post-discharge (one-way ANOVA followed by Tukey's multiple comparisons test; adjusted p-values=0.077 and 0.083, respectively). FIG. 4F and FIG. 4F depict differences in the representation of mcSEED subsystems/pathway modules in the fecal microbiomes of children prior to, during and after treatment for SAM compared to healthy individuals. Abundances of the 30 most age-discriminatory mcSEED subsystems/pathway modules in 15 individuals treated for SAM compared to age-matched healthy individuals. Subsystems/pathway modules whose representation is significantly different between healthy and SAM children for at least three of the six time points are highlighted in red. Mean values±SD are shown. *, p<0.05; **, p<0.01; ***, p<0.001; ****, p<0.0001 (Mann-Whitney test with FDR correction). In both FIG. 4E and FIG. 4F, SAM subjects are grey and healthy subjects are black.

FIG. 5A, FIG. 5B, FIG. 5C, and FIG. 5D show the results of a diet oscillation study to identify complementary foods that selectively boost the relative abundance of weaning-phase age-discriminatory bacterial strains in gnotobiotic mice. FIG. 5A is a heatmap showing changes in the abundances of OTUs in the developing microbiota of healthy members of the Mirpur birth cohort that correspond to cultured strains used to colonize gnotobiotic mice. Rows are arranged based on unsupervised hierarchical clustering of the strains' temporal abundance profiles. Strains in red font were obtained from a 24-month-old donor with SAM. B. pseudocatenulatum and B. catenulatum have the same V4-16S rDNA sequences as B. breve and B. longum. B. longum subsp. infantis was distinguished from B. longum subsp. longum based on its five genomic loci encoding numerous carbohydrate active enzymes (13). FIG. 5B and FIG. 5C show an experimental design for a diet oscillation study. Fourteen unique complementary food combinations (CFCs) were designed by random sampling of 12 ingredients (FIG. 5B). The composition of these CFCs and their order of administration were based on the following considerations. First, every CFC contained six different complementary food ingredients at six different levels, one of which was dominant (see FIG. 5C; the size of the circle in the bubble plot indicates the relative level of the ingredient, the colors correspond to FIG. 5B). In addition to these six ingredients, each CFC also contained bovine milk powder and soybean oil to reflect the fact that children in Mirpur with MAM or SAM are typically treated with therapeutic foods that contain these ingredients. The Spearman correlation coefficient between the amounts of any two ingredients across all diets was minimized so that the abundances of targeted taxa could be clearly related to a given ingredient (see, Table s8 of Gehrig et al. Science, 2019, 365(6449):eaau4732, which is incorporated by reference in its entirety). Each selected formulation was prepared in ways that reflected common culinary practices in Mirpur, processed as a homogeneous blend, extruded into pellets, and sterilized by irradiation. Second, each group of mice received a different weekly sequence of the different diet formulations in order to identify ingredient-microbe interactions that are robust to order of diet presentation (i.e., to avoid hysteresis effects). Third, no group of mice received a formulation dominated by a particular complementary food ingredient more than once during the course of the experiment, although a given ingredient was represented in multiple formulations at different concentrations. Fourth, while no two groups of mice were fed diets dominated by the same complementary food ingredient within the same week, replication was achieved by administering it as the dominant ingredient to different groups at least twice over the course of an experiment (n =8-12 mice/diet). Shotgun sequencing (COPRO-Seq) of total DNA isolated from fecal samples collected on the last day of each 1-week diet block was used to determine the relative abundance of each community member (see, Table s9 of Gehrig et al. Science, 2019, 365(6449):eaau4732, which is incorporated by reference in its entirety). Fourteen of the 16 cultured isolates successfully colonized all animals. Streptococcus gordonii and Streptococcus salivarius were the exceptions: they were below the limit of detection in fecal samples obtained at the 9 time points surveyed per mouse; their corresponding OTUs have feature importance scores of 30 and 23, respectively, in the sparse 30 OTU Bangladeshi RF-derived model of normal gut community development. FIG. 5D shows hierarchical clustering of Spearman's rank correlation coefficients between the relative abundance of each bacterial strain in the fecal microbiota of recipient mice and levels of the indicated complementary food ingredient. *, p<0.05; **, p<0.01; ***, p<0.001; ****, p<0.0001 (Benjamini-Hochberg-corrected p-values).

FIG. 6A and FIG. 6B depicts graphs showing relative abundance of OTUs corresponding to age-discriminator strains in the fecal microbiota of children living in Mirpur. Abundances of the indicated taxa were determined by V4-16S rDNA sequence using fecal samples collected monthly from healthy subjects in the birth cohort (mean values±SD are shown) and from children with SAM prior to treatment. Note that 29 of these 38 children were enrolled in the present study while 9 were from a prior SAM cohort described in (2). Neither B. breve and B. longum, nor B. pseudocatenulatum and B. catenulatum can be accurately distinguished from one another based on their V4-16S rDNA sequences. Green =healthy subjects; red =SAM subjects.

FIG. 7A, FIG. 7B, FIG. 7C, FIG. 7D, FIG. 7E, FIG. 7F, FIG. 7G, FIG. 7H, and FIG. 7I are graphical comparisons of microbial community and host effects of an initial microbiome-directed complementary food (MDCF) prototype versus Milk Suji/Khichuri-Halwa (MS/KH). Germ-free mice or animals colonized with the defined consortium of 14 bacterial strains were fed two diets, monotonously, for 25 days after which time they were euthanized and cecal contents were analyzed. FIG. 7A depicts the relative abundance of strains in the cecal microbiota of colonized mice. Mean values±SD shown. FIG. 7B, FIG. 7C, FIG. 7D, FIG. 7E depict diet- and colonization-dependent effects on cecal levels of short chain fatty acids (FIG. 7B, FIG. 7C) and essential amino acids plus the tryptophan metabolite, indole 3-lactic acid (FIG. 7D, FIG. 7E). Each dot represents a sample from a mouse in the indicated treatment group. Means values±SD values are shown. ***, p<0.001; ****, p<0.0001 (2-way ANOVA followed by Tukey's multiple comparisons test). FIG. 7F depicts diet- and colonization-dependent effects on serum IGF-1 levels. FIG. 7G depicts effects of diet on levels of liver proteins involved in IGF signaling and IGF-1 production. Levels of phosphorylated proteins were normalized to the total amount of the corresponding non-phosphorylated protein or to GAPDH. FIG. 7H depicts impact of diet and colonization status on cortical thickness of femoral bone. FIG. 71 depicts effects of diet in colonized gnotobiotic mice on branched-chain amino acids in serum and acylcarnitines in muscle and liver. [C3-DC/C5-OH are isobars that are not resolved by flow injection MS/MS. C2-DC (malonyl carnitine) is thought to be a surrogate of malonyl CoA while C5-OH is a mix of 3-hydroxy-2-methylbutryl carnitine (derived from the classical isoleucine catabolic intermediate 3-hydroxy-2-methylbutryl CoA) and 3-hydroxyisovaleryl carnitine (a non-canonical leucine metabolite)]. For FIG. D-G, Mean values±SD are shown. ns, not significant. *, p<0.05; **, p<0.01, ****, p<0.001 (Mann-Whitney test).

FIG. 8A depicts an experimental design. The diagram portrays the three different doses of each complementary food ingredient (colored according to the key) added to the base Mirpur-18 diet (grey) and the different order of presentation of the different diets to different mice. Each diet was monotonously given for 1 week.

FIG. 8B and FIG. 8C graphically depict the effects of supplement the Mirpur-18 diet with 16 plant-derived complementary food ingredients in mice colonized with an 18-member consortium of age-/growth-discriminatory bacterial strains. FIG. 8B and FIG. 8C shows abundances of strains in the fecal microbiota of mice fed three different doses of peanut flour and chickpea flour, respectively (see Table 6C). Mean values±SD are shown. *, p<0.05; **, p<0.01 (Kruskal-Wallis test followed by post-hoc Dunn's test; Benjamin-Hochberg-corrected p-values; n=4 mice/dose/diet). The x-axis of FIG. 8B is the same as labeled in FIG. 8C. In FIG. 8C, SAM-derived strains are B. pseudocatenulaum, E. avlium, E. fergusoni, and S. pasteurianus; milk-adapted strains are B. breve and B. longum subsp. Infantis. The remaining strains are weaning phase strains.

FIG. 9A, FIG. 9B, FIG. 9C, FIG. 9D, and FIG. 9E graphically depict effects of Mirpur-18 diet supplementation on a post-SAM MAM donor microbiota transplanted into gnotobiotic mice. FIG. 9A depicts the experimental design. dpg, days post gavage of the donor microbiota. Mirpur(P), Mirpur-18 supplemented with peanut flour. Mirpur(PCSB), Mirpur-18 supplemented with peanut flour, chickpea flour, soy flour and banana. FIG. 9B depicts expression of microbial mcSEED pathway/modules in the ceca of gnotobiotic mouse recipients of the post-SAM MAM donor gut community, as a function of diet treatment. *, p<0.05; **, p,0.001; ***, p<0.0001 (gene set enrichment analysis; all p-values FDR adjusted). FIG. 9C depicts effects of supplementing Mirpur-18 with one or all four complementary food ingredients on the relative abundances of a weaning-phase and a milk-phase associated taxon in feces obtained at dpg 21 (one-way ANOVA followed by Tukey's multiple comparisons test). FIG. 9D depicts relative abundances of the two-taxa in mucosae harvested by laser capture microdissection (LCM) from the proximal, middle, and distal thirds of the small intestine (Mann-Whitney test); FIG. 9E is a cartoon depicting locations in the small intestine where LCM was performed (see FIG. 9D and FIG. 9E). The same color for diets is used in all figures. *, p<0.05; ** p<0.05; ****p>0.00002.

FIG. 10A, FIG. 10B, FIG. 10C, FIG. 10D, FIG. 10E, FIG. 10F, FIG. 10G, FIG. 10H and FIG. 101 graphically depict the results of targeted mass spectrometry of cecal contents of gnotobiotic mice colonized with a post-SAM MAM donor microbiota and from germ-free controls. FIG. 10A, FIG. 10B, FIG. 10C, FIG. 10D, FIG. 10E, FIG. 10F, and FIG. 10G show levels of amino acids, and FIG. 10H and FIG. 101 show levels of short chain fatty acids, in animals fed unsupplemented or supplemented Mirpur-18 diets. nd., not detected. *, p<0.05; **, p<0.01, ***, p<0.001, ****, p<0.0001 (two-way ANOVA followed by Tukey's multiple comparisons test). Brown =Mirpur-18; orange =Mirpur(P); green=Mirpur(PCSB).

FIG. 11A and FIG. 11B show diet- and colonization-dependent increases in submucosal lymphoid aggregates in the small intestine of gnotobiotic mice. Mice colonized with the post-SAM MAM donor microbiota and control germ-free animals were fed supplemented or supplemented Mirpur-18 diets. Lymphoid aggregates present in hematoxylin- and eosin-stained sections from the proximal third of small intestinal segments SI-1 and SI-2 (see FIG. 9D) were counted for all mice in all six treatment groups (n=5 animals/group), and the results are shown in FIG. 11A. ‘Size’ represents the largest cross-sectional diameter for each aggregate in each section scored from each small intestinal segment from each animal. Mean values±SD are shown. FIG. 11B are histo- and immunohistochemical characterization of a representative jejunal lymphoid aggregate from a representative colonized mouse fed the Mirpur(PCSB) diet. *, p<0.05; **, p<0.01 (Kruskal-Wallis test with Dunn's correction for multiple comparisons).

FIG. 12A, FIG. 12B, FIG. 12C, FIG. 12D, FIG. 12E, and FIG. 12F depict effects of two different MDCF prototypes in gnotobiotic piglets. FIG. 12A depicts an experimental design. FIG. 12B depicts weight gain in piglets weaned onto isocaloric MDCF prototypes containing peanut flour, chickpea flour, soy flour, and banana [MDCF(PCSB), green circles] or chickpea and soy flours [(MDCF(CS), grey circles outlined in black]. FIG. 12C depicts micro-computed tomography data of femoral bone obtained at sacrifice. FIG. 12D depicts effects of the MDCFs on the relative abundances of community members in cecal and distal colonic contents. FIG. 12E depicts non-limiting examples of serum proteins with significantly different post-treatment levels between the two diet groups. FIG. 12F depicts effect of diet on serum C3 acylcarnitive levels. Mean values±SD are plotted. *, p<0.05; **, p<0.01; ***, p<0.005, ****, p<0.001 (Two-way ANOVA in FIG. 12B, unpaired t-test in FIG. 12C, FIG. 12D, and FIG. 12F). The color code (i.e., key) provided in FIG. 12B also applies to FIG. 12C, FIG. 12D, and FIG. 12F.

FIG. 13A is an illustration of a study design comparing the effects of MDCF formulations on health status of Bangladeshi children with MAM, and composition of diets. In this figure, total carbohydrate includes all components except added sugar.

FIG. 13B, FIG. 13C, FIG. 13D, and FIG. 13E depict graphs showing the effects of MDCF formulations on health status of Bangladeshi children with MAM. FIG. 13B and FIG. 13B depict the results of a quantitative proteomic analysis of the average fold-change, per treatment group, in the abundances of the 50 plasma proteins most discriminatory for healthy growth, and the 50 plasma proteins most discriminatory for SAM, respectively (protein abundance is row normalized across treatment groups). FIG. 13D and FIG. 13E show average fold-change in abundances of plasma proteins that significantly positively or negatively correlate with HAZ, respectively (absolute value of Pearson correlation >0.25, FDR-corrected p-value <0.05; abundance is row normalized as in FIG. 13B and FIG. 13C). The color scale shown in FIG. 13E is also used in FIG. 13B-D.

FIG. 14A, FIG. 14B, FIG. 14C, FIG. 14D, and FIG. 14E graphically depict the effects of different MDCF formulations on biomarkers and mediators of bone and CNS development, plus NF-κB signaling. Average fold-change (normalized across treatment groups) in the abundances of plasma proteins belonging to GO categories related to bone and CNS development, and agonists and components of the NF-κB signaling pathway. Proteins in the GO category that were significantly higher in the plasma of healthy compared to SAM children (threshold >30%; FDR adjusted p value <0.05) are labeled ‘healthy growth-discriminatory’ while those higher in SAM compared to healthy children (threshold >30%; FDR adjusted p value <0.05) are labeled SAM discriminatory'. Levels of multiple ‘healthy growth discriminatory’ proteins associated with GO processes ‘osteoblast differentiation’ and ‘ossificiation’ (FIG. 14A and FIG. 14B), the GO process ‘CNS development’ (FIG. 14C and FIG. 14D) are enhanced by MDCF-2 treatment while NF-KB signaling is suppressed (FIG. 14E). Healthy growth discriminatory proteins are in FIG. 14A and FIG. 14C. SAMdiscriminatory proteins are in FIG. 14B, FIG. 14D and FIG. 14E. The color scale shown in FIG. 14A is also used in FIG. 14B-E.

FIG. 15A, FIG. 15B, FIG. 15C, and FIG. 15D depict analysis of the fecal microbiota of children in the MAM trial. FIG. 15A and FIG. 15B show quantification of enteropathogen burden in children in the MAM trial before and after treatment. Results are expressed as log-transformed pg genomic DNA (bacteria and parasites), copy number (RNA viruses), and mass per cell lysate mass (Adenovirus). FIG. 15C shows bacterial OTUs with significant changes in their percent relative abundances in the fecal microbiota of children in the MDCF-2 treatment arm. Mean values±SEM are shown *p<0.05; **, p<0.01 (paired t-test). FIG. 15D shows phylogenetic trees of OTUs that significantly increased or decreased in abundance after MDCF-2 treatment (arrows), and the most abundant and prevalent OTUs in the fecal microbiota of Bangladeshi cohort members studies (minimum relative abundance of 1% in at least 25% of healthy individuals, individuals treated for SAM, or individuals with MAM treated with MDCF/RUSF). The color scale shown in FIG. 15C is also used in FIG. 15A and FIG. 15B.

FIG. 16A is an illustration depicting the study design of Example 7.

FIG. 16B, FIG. 16C, and FIG. 16D graphically depict primary outcomes from a randomized controlled trial of MDCF-2 or RUSF supplementation in children with MAM. WLZ (FIG. 16B) WAZ (FIG. 16C), and MUAC (FIG. 16D) during treatment and one month after completion of nutritional supplementation. Vertical gray lines indicate p-values at day 90 and day 120 after starting supplementation for the interaction term between treatment and time. Best-fit linear regression lines are colored green (MDCF-2) or red (RUSF), and the lighter shaded areas around the lines indicate 95% confidence bands. In all three figures, MDCF-2 regression lines are on top.

FIG. 17A, FIG. 17B, FIG. 17C, FIG. 17D, FIG. 17E, FIG. 17F, FIG. 17G, FIG. 17H, and FIG. 171 show effects of nutritional intervention on ponderal growth-associated proteins. FIG. 17A, FIG. 17B, and FIG. 17C are schematics depicting the calculation of ‘β-WLZ’ for each participant (FIG. 17A), ‘Δprotein abundance’ for each participant (FIG. 17B) and the correlation between these two values (FIG. 17C). FIG. 17D shows a gene set enrichment analysis (GSEA) of proteins whose abundances were correlated with ponderal growth. The vertical gray line indicates q<0.05. FIG. 17E (ossification), FIG. 17F (CNS Development), FIG. 17G (Acute phase response), and FIG. 17H (Response to type I interferon) show representative GO terms enriched in the set of WLZ-associated proteins. Shown are the correlation strengths between proteins belonging to a GO term and ponderal growth. Only proteins whose correlations with β-WLZ reached an unadjusted p<0.01 are shown. Proteins are ordered by correlation strength and colored by their p-value (transformed to a -log10 scale so that decreasing values indicate less statistical significance). FIG. 17I shows differential effects of MDCF-2 and RUSF on WLZ-associated proteins. Proteins are ordered by the log₂(fold-change) of the treatment effect of MDCF-2 over RUSF after three months of supplementation. GSEA was used to calculate the enrichment of proteins whose abundances were increased more by MDCF-2 compared to RUSF for the 70 proteins that are positively correlated with WLZ.

FIG. 18A, FIG. 18B, FIG. 18C, FIG. 18D, and FIG. 18E show response of the gut microbiota to MDCF-2 and RUSF supplementation. FIG. 18A shows an analytical scheme for linear mixed effects modeling of the relationship between WLZ and taxon abundance during supplementation. The coefficient β₁ represents the change in WLZ for a unit change in the variance-stabilizing, transformed abundance of an ASV. FIG. 18B is a volcano plot illustrating taxa whose abundances were significantly associated with WLZ (p_{adj}<0.05) as determined by linear mixed effects modeling. FIG. 18C is a barplot indicating the linear model coefficients±SEM for each taxon that was significantly associated with WLZ. Taxa in bold-face were previously identified as ‘ecogroup’ taxa (Raman et al., 2019), while those with asterisks have previously described associations with weight gain in gnotobiotic mice harboring gut microbial communities obtained from healthy and undernourished children (Blanton et al., 2016). FIG. 18D shows abundance changes of WLZ-associated taxa over 3-month treatment period (‘Δ_{ASV}’) with MDCF-2 (left panel) versus RUSF (right panel). Mean values±SEM are shown. FIG. 18E shows ratio of 3-month DASV between MDCF-2 and RUSF treatment arms. A positive ratio indicates a greater average increase in MDCF-2 treated individuals. Color scheme in panels B-E: red bars/points, ASVs with significant positive associations with WLZ; blue bars/points, ASVs with significant negative associations.

FIG. 19A, FIG. 19B, FIG. 19C, and FIG. 19D shows relationships between features of the plasma proteome to members of the gut microbiota. FIG. 19A and FIG. 19B are schematics summarizing how the negative-binomial cross-association matrix was created (FIG. 19A), and of how negative-binomial singular value decomposition was performed (FIG. 19B). Samples from each participant at baseline, one month, and three months after starting intervention were row-concatenated into bacterial (A^{MxN}) or proteomic (P^{MxP}) abundance matrices. For each ASV-protein pair, Empirical Bayes negative binomial regression using DESeq2 was performed, and the resulting test statistic was stored in a cross-association matrix (C^{NxP}). Singular value decomposition was performed on C^{NxP}; the top most positively and negatively projecting bacterial and proteomic features along each singular vector (SV) represent members of a unique cross-association profile that link the abundances of bacterial taxa to the abundances of plasma proteins. FIG. 19C shows representative GO terms from gene set enrichment analysis performed on the cross-association profile of singular vector 8 (SV8). FIG. 19D is a heatmap of the pair-wise cross-associations (DESeq2 test-statistics) between the top 20 and top 50 most positively projecting ASVs and proteins, respectively, along SV8. ASVs are arranged from left to right while proteins are arranged from top to bottom by decreasing projection values. Positive ‘WLZ-associated’ taxa and proteins are highlighted in red.

FIG. 20A, FIG. 20B, FIG. 20C, FIG. 20D, and FIG. 20E show determinants and predictors of MDCF-2 responsiveness. FIG. 20A shows ponderal growth of participants in the upper- and lower-quartile of b-WLZ responses. Faded lines are the WLZ trajectories of individual participants. Circles and error bars represent the mean and SEM. Statistical significance between children in the upper- and lower-quartiles at each timepoint was calculated using an unpaired two-sided t-test. n.s., not significant. **, p<0.01; ***, p<0.001; ****, p<0.0001. FIG. 20B shows change in WLZ between the end of the 3-month intervention and at 1-month post-intervention timepoint. Lower values indicate regression of ponderal growth after intervention. Statistical significance was calculated using an unpaired two-sided t-test. *, p<0.05. FIG. 20C is a gene set enrichment analysis (GSEA) of plasma proteins that were differentially abundant at baseline, or whose abundances showed differential change after 1-month or 3-months of MDCF-2 supplementation between children in the upper- and lower-quartile of WLZ responses. The color of each circle indicates the direction of enrichment (red, higher in upper-quartile responders; blue means lower in upper-quartile responders). The darkness of each circle represents the normalized enrichment score from GSEA. The size of each circle represents the statistical significance. Circles that are outlined in black reached the statistically significant threshold of q<0.1. FIG. 20D shows the abundance response of WLZ-associated taxa over the 3-month period of treatment with MDCF-2 in those classified as having upper-quartile (left panel) versus lower-quartile (right panel) b-WLZ responses. Mean values±SEM are shown. FIG. 20E shows the durability of microbiota response. Durability is defined by comparing (i) changes in the abundances after 3-months of treatment with MDCF-2 of all 209 ASVs present in at least 5% of all of 939 fecal samples analyzed with (ii) their abundance changes between the 3-month end-of-treatment and 1-month post-treatment time points. ASVs with the top 10 greatest magnitude of positive or negative change are labeled. The inset in the lower left portion of the panel shows the relationship between changes in WLZ from baseline to 3-months and between the 3- and 4-month time points. Color legend for panels D and E: Red bars/points, ASVs with significant positive associations with WLZ; blue bars/points, ASVs with significant negative associations. Black points in panel D denote taxa that do not have significant associations with WLZ.

FIG. 21 is a schematic showing enrollment, randomization and follow-up.

FIG. 22A, FIG. 22B, FIG. 22C, and FIG. 22D show effects of MDCF-2 and RUSF on illness and co-morbidities. Change in the proportion of participants with reported cough (FIG. 22A), runny-nose (FIG. 22B), fever (FIG. 22C), or diarrhea (FIG. 22D) throughout the 3-month supplementation. Each dot represents the mean proportion of participants with the reported co-morbidity. Shaded regions around linear regression lines represent 95% confidence intervals. P-values for the interaction between treatment and time since starting the intervention are reported as insets. The color code (i.e., key) provided in FIG. 22AD also applies to FIG. 22B, FIG. 22C, and FIG. 22D

FIG. 23A and FIG. 23B describe quality control of proteins represented on the SOMAscan platform. FIG. 23A depicts a workflow for quality control (QC) filtering. FIG. 24B depicts the distribution of signal-to-noise ratios for the SOMAmers that passed the first two QC filters. SOMAmers with median abundances across plasma samples greater than 4.9 median average deviances (MAD) from the median of blank, buffer alone samples (indicated by the vertical line), were considered signal above noise.

FIG. 24A and FIG. 24B show effects of MDCF-2 and RUSF on WLZ-associated proteins. Log2(fold-changes) of ‘WLZ-associated’ proteins after 3-month supplementation with MDCF-2 (FIG. 24A) or RUSF (FIG. 24B). Gene set enrichment analysis was used to calculate the enrichment of proteins whose abundances were increased after MDCF-2 or RUSF treatment for the 70 ‘WLZ-associated’ proteins.

FIG. 25 illustrates the determination of the number of singular vectors with cross-association information between plasma proteins and gut bacterial taxa. SVD was performed on the cross-association matrix generated by NB-SVD analysis as well as the same cross-association matrix whose columns were randomly shuffled to remove information regarding the relationships between plasma protein and ASV abundances. The percent variance explained of each singular vector (SV) generated from decomposing the cross-association (blue curve) or shuffled (gray curve) matrix are plotted in descending order. The noise threshold was chosen to be the percent variance explained by the first SV of the shuffled matrix (horizontal line); SV10 (vertical line) from the SVD of the cross-association matrix was the last SV that explained more variance than the noise threshold.

FIG. 26 depicts a complete SV8 cross-association profile identified by NB-SVD analysis. The top 20 most positively and negatively projecting bacterial taxa and the top 50 most positively and negatively projecting plasma proteins were identified in the cross-association matrix produced by NB-SVD analysis and plotted as a heatmap. Each element represents the DESeq2 test-statistic, a measure of association between the abundance of a bacterial taxon and plasma protein. Features are ranked by their projections along SV8. Positively WLZ-associated proteins and taxa are highlighted in red. Bifidobacterium sp. (likely B. longum) is highlighted in blue and was the only WLZ-associated taxon in the top 20 negative projections along SV8.

FIG. 27A, FIG. 27B, and FIG. 27C depict effects of nutritional supplementation on the repertoire of carbohydrate active enzymes in the gut metagenome. FIG. 27A graphically depicts CAZymes that are significantly correlated to WLZ. Red indicates CAZymes that are positively correlated with WLZ while blue indicates CAZymes that are negatively correlated with WLZ. FIG. 27B graphically depicts differential effects of MDCF-2 and RUSF on positive WLZ-correlated CAZymes. Only CAZymes with a log₂(fold-change) of greater than 0.5 in either direction are highlighted. Positive log₂(fold-changes) indicate larger magnitude changes in MDCF-2 compared to RUSF diet. FIG. 27C graphically depicts differential effects of MDCF-2 and RUSF on negative WLZ-correlated CAZymes. Only CAZymes with a log2(fold-change) of greater than 0.25 in either direction are highlighted. Positive log2(fold-changes) indicate larger magnitude changes in the MDCF-2 arm compared to the RUSF arm.

### DETAILED DESCRIPTION

The present disclosure describes an approach for integrating preclinical gnotobiotic animal models with human studies to understand the contributions of impaired gut microbial community development to childhood undernutrition. Combining metabolomic and proteomic analyses of serially collected plasma samples with metagenomic analyses of fecal samples, the biological state of Bangladeshi children with severe acute malnutrition (SAM) was characterized as they transitioned, following standard treatment, to moderate acute malnutrition (MAM) with persistent microbiota immaturity. Gnotobiotic mice were subsequently colonized with a defined consortium of bacterial strains representing different stages of microbiota development in healthy children. Administering different combinations of Bangladeshi complementary food ingredients to colonized and germ-free mice revealed diet-dependent changes in the relative abundance and metabolism of weaning-phase bacterial taxa underrepresented in SAM and MAM microbiota, plus diet- and colonization-dependent effects on host metabolism and growth-associated signaling pathways. Host and microbial effects of microbiota-directed complementary food (MDCF) prototypes were subsequently examined in gnotobiotic mice colonized with post-SAM MAM microbiota and in gnotobiotic piglets colonized with a defined consortium of targeted age- and growth-discriminatory bacteria. A randomized, double-blind study identified a lead MDCF that changes the abundances of targeted bacterial taxa and increases plasma levels of biomarkers and mediators of growth, bone formation, neurodevelopment, and immune function in children with MAM. The beneficial effects of the lead MDCF were confirmed in a subsequent clinical trial.

Accordingly, provided herein are compositions and methods to improve the nutritional status and health of a subject in need thereof, including malnourished children, as well as aid in the maturation of the gut microbiota of these subjects. Various aspects of these compositions and methods are described in more detail below.

As used herein, “about” refers to numeric values, including whole numbers, fractions, percentages, etc., whether or not explicitly indicated. The term “about” generally refers to a range of numerical values, for instance,±0.5-1%,±1-5% or±5-10% of the recited value, that one would consider equivalent to the recited value, for example, having the same function or result.

The term “comprising” means “including, but not necessarily limited to”; it specifically indicates open-ended inclusion or membership in a so-described combination, group, series and the like. The terms “comprising” and “including” as used herein are inclusive and/or open-ended and do not exclude additional, unrecited elements or method processes. The term “consisting essentially of” is more limiting than “comprising” but not as restrictive as “consisting of.” Specifically, the term “consisting essentially of” limits membership to the specified materials or steps and those that do not materially affect the essential characteristics of the claimed invention.

The term “carbohydrate”, as used herein, refers to an organic compound with the formula Cₘ(H₂O)ₙ, where m and n may be the same or different number, provided the number is greater than 3.

The term “glycan” refers to a linear or branched homo- or heteropolymer of two or more monosaccharides linked glycosidically. As such, the term “glycan” includes disaccharides, oligosaccharides and polysaccharides. The term also encompasses a polymer that has been modified, whether naturally or otherwise; non-limiting examples of such modifications include acetylation, alkylation, esterification, etherification, oxidation, phosphorylation, selenization, sulfonation, or any other manipulation.

As used herein, the term “malnutrition” refers to one or more forms of undernutrition—for example, wasting (low weight-for-length), stunting (low length-for-age), underweight (low weight-for age), deficiencies in vitamins and minerals, etc. A subject in need of treatment for malnutrition may also be referred to herein as a malnourished subject.

A length-for-age Z Score (LAZ) refers to the number of standard deviations of the actual length of a child from the median length of the children of his/her age as determined from the standard sample. This is prefixed by a positive sign (+) or a negative sign (−) depending on whether the child's actual length is more than the median length or less than the median length. The terms length and height are used interchangeably herein. Therefore, length-for-age Z Score (LAZ) and height-for-age Z Score (HAZ) refer to the same measurement.

A weight-for-age Z score (WAZ) refers to the number of standard deviations of the actual weight of a child from the median weight of the children of his/her age as determined from the standard sample. This is prefixed by a positive sign (+) or a negative sign (−) depending on whether the child's actual weight is more than the median weight or less than the median weight.

A weight-for-length Z score (WLZ) refers to the number of standard deviations of the actual weight of a child from the median weight of the children of his/her length as determined form the standard sample. This is prefixed by a positive sign (+) or a negative sign (−) depending on whether the child's actual weight is more than the median weight or less than the median weight for the same length. The terms length and height are used interchangeably herein. Therefore, weight-for-height Z score (WHZ) and weight-for-length Z score (WLZ) refer to the same measurement.

A mid-upper-arm-circumference score (MUAC) is an independent anthropometric measurement used to identify malnutrition.

Moderate acute malnutrition (MAM) is defined by a WHZ less than or equal to −2 and greater than or equal to −3.

Severe acute malnutrition (SAM) is defined by a WHZ less than −3 and/or bipedal edema, and/or a mid-upper arm circumference (MUAC) less than 11.5 cm.

As used herein, a “healthy child” has a LAZ and WLZ consistently no more than 1.5 standard deviations below the median calculated from a World Health Organization (WHO) reference healthy growth cohort as described in WHO Multicentre Reference Study (MGRS), 2006 (www.who.int/childgrowth/mgrs/en).

As used herein, “statistically significant” is a p-value <0.05, <0.01, <0.001, <0.0001, or <0.00001.

The terms “treat,” “treating,” or “treatment” as used herein, refer to both therapeutic treatment and prophylactic or preventative measures, wherein the object is to prevent or slow down (lessen) an undesired physiological change or disease/disorder. Beneficial or desired clinical results include, but are not limited to, alleviation of symptoms, diminishment of extent of disease, stabilization (i.e., not worsening) of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented.

As used herein, the term “effective amount” means an amount of a substance (e.g. a composition of the present disclosure) that leads to measurable and beneficial effects for the subject administered the substance, i.e., significant efficacy.

As used herein, the term “raw banana” refers to an unripe, green banana in the genus Musa. “Raw bananas” are also referred to as “green bananas” in the art, and the terms are used interchangeably herein. As is understood in the art, raw bananas are processed (e.g., baked, boiled, steamed, etc.) prior to use.

### I. Compositions

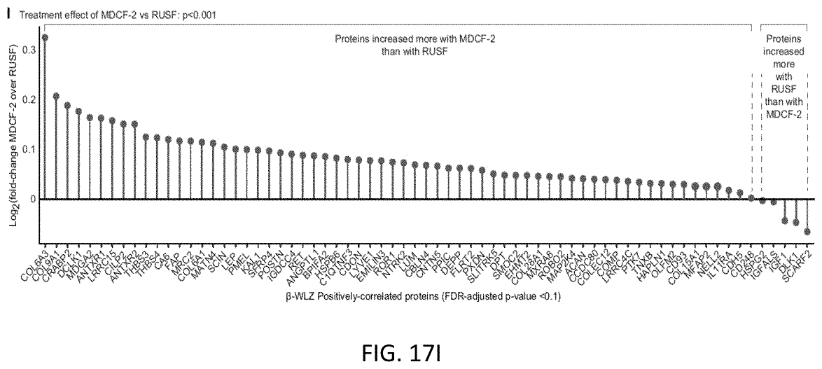

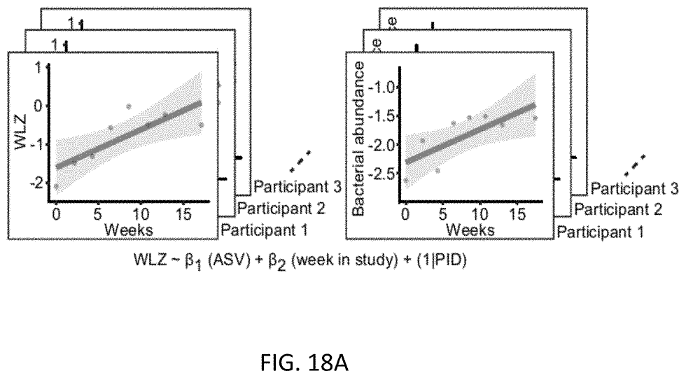

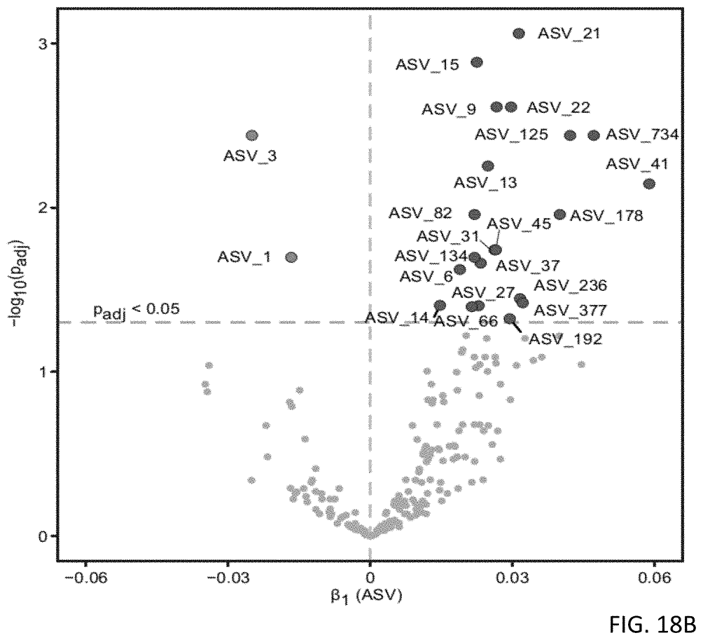

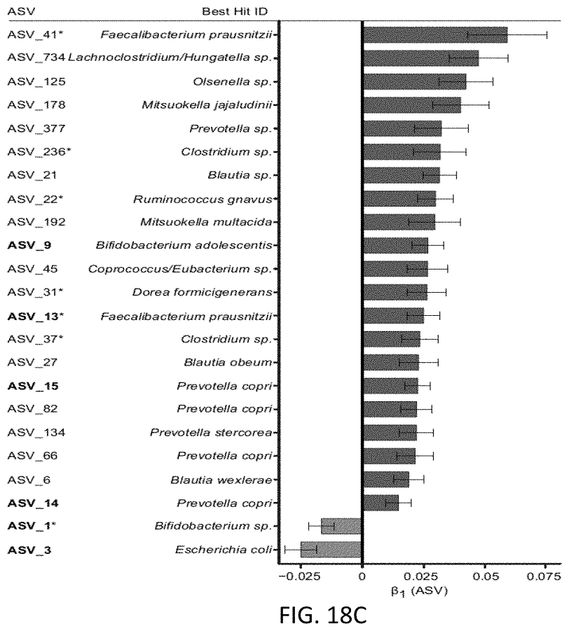

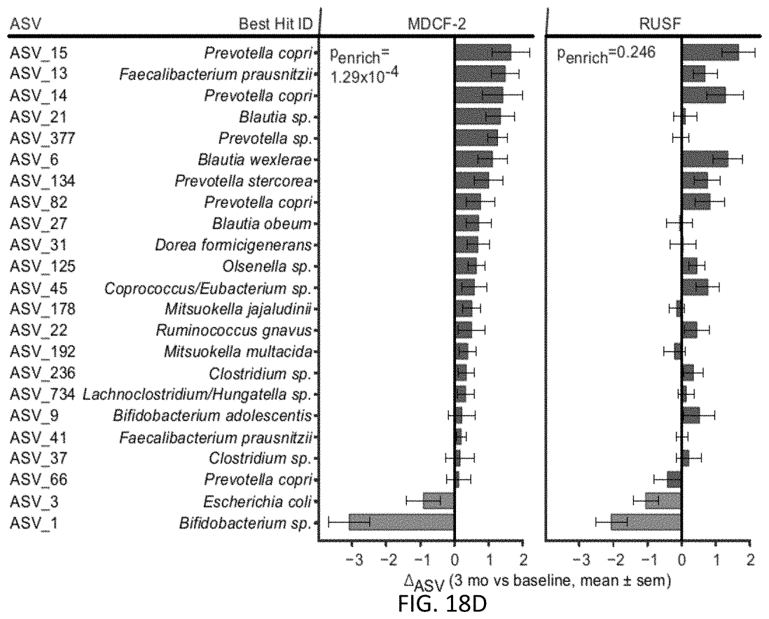

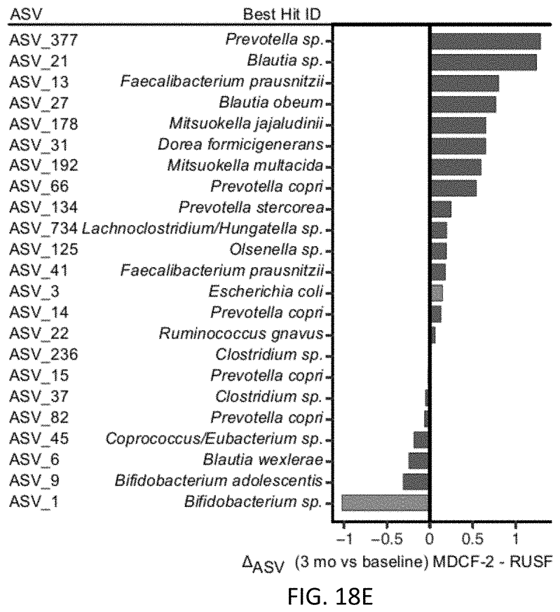

In some embodiments, the present disclosure encompasses an edible composition that, when eaten in a manner described herein, impacts the subject's gut microbiota by changing the relative abundances of a plurality (e.g. 50% or more) of health discriminatory gut taxa in a statistically significant manner towards chronologically age-matched healthy subjects. “Health discriminatory gut taxa” are gut microbial strains significantly associated with a measurable indicator of health (e.g., weight, height, ponderal growth rate, biomarkers, etc.). As a non-limiting example, health discriminatory taxa may be gut microbial strains significantly associated with WLZ (“WLZ-associated taxa”). Methods for identifying WLZ-associated taxa are described in detail in the examples, and WLZ-associated taxa for subjects 6 months to 18 months are identified in FIG. 18C. The same approach, or a substantially similar approach, may be used to identify WLZ-associated taxa for other age groups and to identify other health discriminatory taxa including but not limited to gut microbial strains significantly associated with WAZ (“WAZ-associated taxa”), LAZ (“LAZ-associated taxa”), MUAC (“MUAC-associated taxa”), or any combination thereof.

For instance, the present disclosure encompasses an edible composition comprising carbohydrates that, when eaten, modulates the relative abundances of at least 11 WLZ-associated taxa of FIG. 18C in a statistically significant manner towards chronologically age-matched healthy subjects. In further embodiments, the present disclosure encompasses an edible composition comprising carbohydrates that, when eaten, modulates the relative abundances of at least 11 WLZ-associated taxa of FIG. 18C in a statistically significant manner towards chronologically age-matched healthy subjects, wherein at least six of the taxa are ASV_9, ASV_13, ASV_15, ASV_14, ASV_1, and ASV_3. In still further embodiments, the present disclosure encompasses an edible composition of the present disclosure comprising carbohydrates that, when eaten, modulates the relative abundances of at least 11 WLZ-associated taxa of FIG. 18C in a statistically significant manner towards chronologically age-matched healthy subjects, wherein at least seven of the taxa are ASV_41, ASV_236, ASV_22, ASV_31, ASV_13, ASV_37, and ASV_1. In still further embodiments, the present disclosure encompasses an edible composition comprising carbohydrates that, when eaten, modulates the relative abundances of at least 11 WLZ-associated taxa of FIG. 18C in a statistically significant manner towards chronologically age-matched healthy subjects, wherein at least five of the taxa are ASV_15, ASV_13, ASV_14, ASV_21, and ASV_377. In the above embodiments, the present disclosure encompasses an edible composition comprising carbohydrates that modulates the relative abundances of 11, 12, 13, 14, 15, 16, or 17 WLZ-associated taxa in a statistically significant manner. Alternatively, the present disclosure encompasses an edible composition comprising carbohydrates that modulates the relative abundances of 18, 19, 20, 21, 22, or 23 WLZ-associated taxa in a statistically significant manner. In certain embodiments, an edible composition comprising carbohydrates of the present disclosure is a composition described herein in Section I.

In some embodiments, the present disclosure encompasses an edible composition that impacts the subject's gut microbiota in a manner to modulate abundance of nucleic acids encoding proteins in particular CAZyme families, such that physiological parameters of the subject are improved, e.g., ponderal growth or rate of ponderal growth. For instance, the present disclosure encompasses an edible composition comprising carbohydrates that increases abundance of nucleic acids encoding proteins in about 75%, 80%, 85%, 90%, 95%, 96%, 97%, 98%, 99%, or 100% of the CAZyme families indicated in Table A. The present disclosure also encompasses an edible composition comprising carbohydrates that decreases abundance of nucleic acids encoding proteins in about 75%, 80%, 85%, 90%, 95%, 96%, 97%, 98%, 99%, or 100% of the CAZyme families indicated in Table B. In preferred embodiments, the present disclosure encompasses an edible composition comprising carbohydrates that increases abundance of nucleic acids encoding proteins in about 75%, 80%, 85%, 90%, 95%, 96%, 97%, 98%, 99%, or 100% of the CAZyme families indicated in Table A and decreases abundance of nucleic acids encoding proteins in about 75%, 80%, 85%, 90%, 95%, 96%, 97%, 98%, 99%, or 100% of the CAZyme families indicated in Table B. In a particular preferred embodiment, the present disclosure encompasses an edible composition comprising carbohydrates that increases abundance of nucleic acids encoding proteins in about 95%, 96%, 97%, 98%, 99%, or 100% of the CAZyme families indicated in Table A and decreases abundance of nucleic acids encoding proteins in about 95%, 96%, 97%, 98%, 99%, or 100% of the CAZyme families indicated in Table B. In still another preferred embodiment, the present disclosure encompasses an edible composition comprising carbohydrates that increases abundance of nucleic acids encoding proteins in each of the CAZyme families indicated in Table A and decreases abundance of nucleic acids encoding proteins in each of the CAZyme families indicated in Table B. In each of the above embodiments “increases abundance” or “decreases abundance” refers to a change in abundance compared to the same subject before ingestion of the edible composition.

In certain embodiments, an edible composition comprising carbohydrates of the present disclosure is a composition described herein in Section I.

TABLE A

| ORF ID | logFC | t | p-value | q-value | CAZyme Family |
|---|---|---|---|---|---|
| METADB4286D7_277490 | 1.38 | 2.75 | 6.05E−03 | 1.00E+00 | GH78 |
| METAB633EE12_512080 | 0.77 | 2.10 | 3.60E−02 | 1.00E+00 | GT2 |
| META46898562_766340 | 0.77 | 1.99 | 4.74E−02 | 1.00E+00 | GH32 |
| METACFE9C49C_162320 | 0.75 | 1.85 | 6.45E−02 | 1.00E+00 | GT28 |
| META0D3D3092_18185 | 0.61 | 1.84 | 6.63E−02 | 1.00E+00 | GT2 |
| META6562874C_424995 | 0.62 | 1.81 | 7.03E−02 | 1.00E+00 | GH23 |
| METAA3075C7F_597380 | 0.56 | 1.81 | 7.11E−02 | 1.00E+00 | GT2 |
| METAFC3F3567_35445 | 0.60 | 1.75 | 7.97E−02 | 1.00E+00 | GT2 |
| META6B8C2E08_322695 | 0.87 | 1.64 | 1.00E−01 | 1.00E+00 | CBM48-GH13_9 |
| METADB4286D7_443415 | 0.98 | 1.62 | 1.05E−01 | 1.00E+00 | GT35 |
| META70372ECF_263000 | 1.05 | 1.61 | 1.07E−01 | 1.00E+00 | GH43_34 |
| METACFE9C49C_418055 | 0.61 | 1.61 | 1.09E−01 | 1.00E+00 | GH29 |
| META886A343C_245840 | 1.31 | 1.60 | 1.10E−01 | 1.00E+00 | GH5_21 |
| META37C55EB9_260265 | 0.58 | 1.59 | 1.12E−01 | 1.00E+00 | GH36 |
| META14843150_39125 | 0.87 | 1.57 | 1.16E−01 | 1.00E+00 | GH32 |
| METAB633EE12_28445 | 0.57 | 1.43 | 1.52E−01 | 1.00E+00 | GT4 |
| METAA79B98C3_256500 | 0.59 | 1.43 | 1.53E−01 | 1.00E+00 | GH13_9 |
| META675C6E24_224380 | 0.72 | 1.43 | 1.54E−01 | 1.00E+00 | GH77 |
| META77DD1015_127535 | −1.01 | −1.41 | 1.59E−01 | 1.00E+00 | GT51 |
| META6C7F2A20_354800 | 0.53 | 1.41 | 1.60E−01 | 1.00E+00 | GH29 |
| METAC0E28048_297040 | 0.71 | 1.40 | 1.62E−01 | 1.00E+00 | GT28 |
| METAD29BD862_186400 | 0.54 | 1.38 | 1.67E−01 | 1.00E+00 | GT2 |
| METAEF1EC719_199970 | 0.66 | 1.38 | 1.69E−01 | 1.00E+00 | CBM9 |
| META56CEA8D1_20710 | −0.91 | −1.35 | 1.77E−01 | 1.00E+00 | GH5_4 |
| METADA5F5064_169165 | 0.51 | 1.34 | 1.81E−01 | 1.00E+00 | GH3 |
| META4745DA27_178440 | 0.90 | 1.33 | 1.82E−01 | 1.00E+00 | PL1 |
| META41A0FA1D_59615 | 0.63 | 1.33 | 1.83E−01 | 1.00E+00 | GT2 |
| META1BDE7F7F_06845 | 0.57 | 1.33 | 1.83E−01 | 1.00E+00 | GH13_5 |
| META67F8845E_294345 | 0.78 | 1.31 | 1.90E−01 | 1.00E+00 | GT51 |
| META40DCD9F7_69065 | 0.54 | 1.30 | 1.95E−01 | 1.00E+00 | GT28-GT30 |
| META6B8C2E08_593315 | 0.84 | 1.26 | 2.07E−01 | 1.00E+00 | GH36 |
| META8344E514_231570 | −0.80 | −1.26 | 2.10E−01 | 1.00E+00 | GH66-CBM72 |
| METADB4286D7_59780 | −0.99 | −1.25 | 2.12E−01 | 1.00E+00 | GH43_12 |
| META56CEA8D1_103755 | 0.67 | 1.25 | 2.13E−01 | 1.00E+00 | GH23-CBM50 |
| METAFE63B677_09355 | 0.61 | 1.24 | 2.15E−01 | 1.00E+00 | GT5 |
| META687B4180_102210 | 0.86 | 1.21 | 2.27E−01 | 1.00E+00 | GT8 |
| METAAB2BBC95_260115 | 0.72 | 1.20 | 2.29E−01 | 1.00E+00 | GH105 |
| META976EC559_168565 | 0.85 | 1.20 | 2.31E−01 | 1.00E+00 | GH28 |
| META63788F6C_285115 | 0.60 | 1.20 | 2.32E−01 | 1.00E+00 | GH29 |
| METACB04D34A_181065 | 0.45 | 1.20 | 2.32E−01 | 1.00E+00 | GH13_4 |
| META56CEA8D1_126680 | 0.77 | 1.20 | 2.32E−01 | 1.00E+00 | PL1-CBM77 |
| META2C2D88EE_616895 | 0.74 | 1.17 | 2.43E−01 | 1.00E+00 | CBM32-GH2 |
| METAA323FF6A_133380 | 0.42 | 1.15 | 2.49E−01 | 1.00E+00 | GH32 |
| META7796BCC3_172575 | 0.55 | 1.15 | 2.50E−01 | 1.00E+00 | GH1 |
| METADB4286D7_324850 | 0.63 | 1.14 | 2.56E−01 | 1.00E+00 | GH29 |
| METAB4CA71E7_429085 | 0.45 | 1.14 | 2.56E−01 | 1.00E+00 | GH1 |
| META85128387_139615 | 0.70 | 1.13 | 2.58E−01 | 1.00E+00 | GH43_4 |
| METAF4AB60B2_577410 | 0.61 | 1.10 | 2.70E−01 | 1.00E+00 | GH127 |
| META1DFF011B_561075 | 0.55 | 1.10 | 2.72E−01 | 1.00E+00 | GT2 |
| META9545EEA3_112555 | 0.92 | 1.09 | 2.75E−01 | 1.00E+00 | GH31 |
| META886A343C_147760 | −0.83 | −1.09 | 2.77E−01 | 1.00E+00 | GH13 |
| METAC1677873_189130 | 0.90 | 1.08 | 2.79E−01 | 1.00E+00 | GH53 |
| META976EC559_390510 | −0.82 | −1.06 | 2.88E−01 | 1.00E+00 | GH95 |
| METABFB860FB_107645 | −0.81 | −1.04 | 2.97E−01 | 1.00E+00 | GH2 |
| METABC7545BB_153365 | 0.64 | 1.04 | 3.00E−01 | 1.00E+00 | CBM34-GH13_20 |
| METAD6BA71B8_312260 | 0.62 | 1.03 | 3.02E−01 | 1.00E+00 | GT4 |
| META63788F6C_184225 | −0.72 | −0.99 | 3.21E−01 | 1.00E+00 | GH106 |
| META0A2F4A81_44090 | 0.67 | 0.98 | 3.26E−01 | 1.00E+00 | GH95 |
| META0A69D94D_297535 | 0.37 | 0.97 | 3.32E−01 | 1.00E+00 | GH51 |
| METAE15293D1_178730 | −0.56 | −0.97 | 3.32E−01 | 1.00E+00 | GH10-CBM4-CBM4-GH10 |
| METAB2EF0315_199875 | −0.77 | −0.97 | 3.33E−01 | 1.00E+00 | GH13_14 |
| META79F7DB64_226695 | 0.37 | 0.97 | 3.34E−01 | 1.00E+00 | GH84-CBM32-CBM32 |
| METAB2EF0315_190630 | 0.57 | 0.96 | 3.35E−01 | 1.00E+00 | GH20 |
| METAE07878D2_126780 | 0.54 | 0.95 | 3.41E−01 | 1.00E+00 | GH13 |
| META8344E514_476160 | −0.65 | −0.95 | 3.45E−01 | 1.00E+00 | GH28 |
| META7D10DFE7_133630 | 0.56 | 0.94 | 3.48E−01 | 1.00E+00 | GH94 |
| METABF1B8FA8_490445 | 0.57 | 0.94 | 3.49E−01 | 1.00E+00 | GH133 |
| META6562874C_205190 | −0.56 | −0.93 | 3.52E−01 | 1.00E+00 | GT4 |
| META5FDAC71F_481265 | 0.49 | 0.91 | 3.63E−01 | 1.00E+00 | GT4 |
| META886A343C_512240 | 0.49 | 0.90 | 3.68E−01 | 1.00E+00 | CBM51-CBM51-GH27 |
| METAEB3CE14A_450890 | 0.42 | 0.89 | 3.71E−01 | 1.00E+00 | GH32 |
| META6562874C_199020 | 0.46 | 0.88 | 3.78E−01 | 1.00E+00 | GH84 |
| METADB4286D7_215360 | 0.54 | 0.85 | 3.94E−01 | 1.00E+00 | GT5 |
| METAFE58A94B_49720 | 0.54 | 0.85 | 3.95E−01 | 1.00E+00 | GH95 |
| METAB4EBD9C4_71905 | −0.71 | −0.85 | 3.96E−01 | 1.00E+00 | GH32 |
| METAF0677D79_284170 | −0.63 | −0.85 | 3.96E−01 | 1.00E+00 | GH23 |
| METAF590B30C_601920 | 0.43 | 0.84 | 4.03E−01 | 1.00E+00 | GH43_10 |
| METACBC77C2A_458210 | 0.62 | 0.83 | 4.05E−01 | 1.00E+00 | GH3 |
| META8BC9F76E_755345 | −0.63 | −0.83 | 4.06E−01 | 1.00E+00 | GT30 |
| META6CD5EEA8_618835 | 0.49 | 0.83 | 4.07E−01 | 1.00E+00 | GT51 |
| METABB96A33D_45980 | 0.48 | 0.83 | 4.07E−01 | 1.00E+00 | GT4 |
| METAE5493260_301700 | 0.39 | 0.82 | 4.12E−01 | 1.00E+00 | GH32 |
| META784D3033_127260 | −0.66 | −0.82 | 4.15E−01 | 1.00E+00 | GH20 |
| META25C30777_290180 | 0.43 | 0.82 | 4.15E−01 | 1.00E+00 | GT4 |
| META9AE76C6E_39655 | 0.37 | 0.81 | 4.17E−01 | 1.00E+00 | CBM48-GH13_9 |
| METAE07878D2_183195 | 0.46 | 0.81 | 4.18E−01 | 1.00E+00 | CBM72 |
| META8344E514_402800 | 0.50 | 0.79 | 4.32E−01 | 1.00E+00 | GH13 |
| METAC230BFD3_491230 | 0.42 | 0.77 | 4.39E−01 | 1.00E+00 | GT35 |
| METAF486615E_383670 | 0.55 | 0.77 | 4.40E−01 | 1.00E+00 | GH16_3 |
| METAFC87AF8B_83655 | −0.59 | −0.77 | 4.42E−01 | 1.00E+00 | GH2 |
| METAB2EF0315_28785 | −0.54 | −0.76 | 4.45E−01 | 1.00E+00 | GH2 |
| META6B8C2E08_147430 | 0.37 | 0.75 | 4.53E−01 | 1.00E+00 | GT2 |
| META3AAC7993_676505 | 0.49 | 0.74 | 4.60E−01 | 1.00E+00 | GT23 |
| META14843150_126195 | 0.45 | 0.73 | 4.65E−01 | 1.00E+00 | GH31-CBM32-DOC |
| META67F8845E_51505 | 0.39 | 0.72 | 4.69E−01 | 1.00E+00 | GH31 |
| META67F8845E_345870 | −0.56 | −0.72 | 4.74E−01 | 1.00E+00 | GH51 |
| METAEF21E9EB_327615 | 0.39 | 0.71 | 4.79E−01 | 1.00E+00 | GH25 |
| METAFE58A94B_22945 | 0.45 | 0.70 | 4.83E−01 | 1.00E+00 | GH2-CBM32 |
| META14843150_152995 | 0.32 | 0.70 | 4.84E−01 | 1.00E+00 | GH2 |
| METAE7E80DED_282710 | 0.30 | 0.69 | 4.89E−01 | 1.00E+00 | GT35 |
| META73E4BF65_198935 | 0.30 | 0.69 | 4.89E−01 | 1.00E+00 | GH2 |
| META90554699_84495 | 0.41 | 0.69 | 4.90E−01 | 1.00E+00 | GH13_8 |
| META14843150_163320 | 0.52 | 0.69 | 4.91E−01 | 1.00E+00 | GH73-CBM50 |
| METAFE58A94B_505565 | 0.42 | 0.69 | 4.93E−01 | 1.00E+00 | GT2 |
| METAB2CC138D_712320 | 0.38 | 0.68 | 4.97E−01 | 1.00E+00 | GT2 |
| META76B38A95_311225 | 0.48 | 0.68 | 4.99E−01 | 1.00E+00 | PL1 |
| METADB4286D7_67600 | 0.42 | 0.67 | 5.00E−01 | 1.00E+00 | GH29 |
| METAF4AB60B2_424455 | 0.29 | 0.67 | 5.01E−01 | 1.00E+00 | GT51 |
| METAFAAD88FF_142235 | 0.38 | 0.67 | 5.01E−01 | 1.00E+00 | GH28 |
| META76B38A95_90370 | 0.54 | 0.67 | 5.02E−01 | 1.00E+00 | GH92 |
| METAB677B55C_164800 | 0.39 | 0.67 | 5.05E−01 | 1.00E+00 | GH28 |
| METAF4AB60B2_270595 | 0.46 | 0.66 | 5.07E−01 | 1.00E+00 | GT2 |
| METAF7761834_539770 | 0.31 | 0.66 | 5.09E−01 | 1.00E+00 | GH13_5 |
| METAF564A3A0_97120 | −0.51 | −0.66 | 5.09E−01 | 1.00E+00 | GH97 |
| METAFD6AD8AE_360065 | 0.40 | 0.65 | 5.15E−01 | 1.00E+00 | GH109 |
| METAD6BA71B8_326540 | 0.33 | 0.65 | 5.18E−01 | 1.00E+00 | GH20 |
| METACBC77C2A_477405 | −0.47 | −0.64 | 5.24E−01 | 1.00E+00 | GH35 |
| META7D10DFE7_374100 | 0.44 | 0.64 | 5.25E−01 | 1.00E+00 | GH92 |
| METAD6BA71B8_16145 | 0.38 | 0.63 | 5.27E−01 | 1.00E+00 | GT35 |
| METADB4286D7_262330 | 0.38 | 0.63 | 5.28E−01 | 1.00E+00 | GH57 |
| META7FC3DEDC_479360 | −0.45 | −0.63 | 5.29E−01 | 1.00E+00 | GH23-CBM50 |
| META34DCCD6F_218260 | 0.27 | 0.61 | 5.42E−01 | 1.00E+00 | GH166 |
| METAB88AC952_143510 | 0.46 | 0.60 | 5.49E−01 | 1.00E+00 | GH95 |
| META8344E514_446780 | 0.32 | 0.59 | 5.54E−01 | 1.00E+00 | GH13 |
| METAEB6EC57B_399900 | 0.28 | 0.59 | 5.58E−01 | 1.00E+00 | GH166 |
| METAB2EF0315_413195 | 0.34 | 0.58 | 5.63E−01 | 1.00E+00 | GH95 |
| METAF564A3A0_177455 | 0.34 | 0.58 | 5.63E−01 | 1.00E+00 | GT2 |
| METAAC57F7F7_222660 | 0.27 | 0.57 | 5.68E−01 | 1.00E+00 | GT5 |
| META650EBBEB_314705 | −0.44 | −0.57 | 5.69E−01 | 1.00E+00 | GH29 |
| META8344E514_54330 | 0.31 | 0.57 | 5.70E−01 | 1.00E+00 | GT2 |
| META704F8AAB_250475 | −0.26 | −0.56 | 5.76E−01 | 1.00E+00 | GH112 |
| META95D5664D_723850 | 0.32 | 0.55 | 5.83E−01 | 1.00E+00 | GT2 |
| META2DB76672_284490 | −0.21 | −0.55 | 5.84E−01 | 1.00E+00 | CBM50 |
| METAC4ED4080_141420 | 0.42 | 0.54 | 5.86E−01 | 1.00E+00 | GH144 |
| META56CEA8D1_271275 | 0.26 | 0.54 | 5.86E−01 | 1.00E+00 | GT2 |
| METAAB555260_190575 | −0.40 | −0.54 | 5.90E−01 | 1.00E+00 | GT2 |
| META7D10DFE7_84125 | −0.41 | −0.54 | 5.90E−01 | 1.00E+00 | GH97 |
| META245698C2_216710 | −0.42 | −0.53 | 5.94E−01 | 1.00E+00 | GH3 |
| META6562874C_318070 | −0.34 | −0.53 | 5.95E−01 | 1.00E+00 | GT4 |
| METADCF12AEB_74590 | −0.40 | −0.53 | 5.98E−01 | 1.00E+00 | GH130 |
| METAF486615E_386320 | −0.42 | −0.53 | 5.99E−01 | 1.00E+00 | CBM20-GH77 |
| META1CBC1A82_211505 | 0.40 | 0.52 | 6.02E−01 | 1.00E+00 | CBM72 |
| METADB48B6AC_135885 | 0.25 | 0.52 | 6.05E−01 | 1.00E+00 | GH13_36 |
| METAC1732D1C_187200 | 0.21 | 0.51 | 6.09E−01 | 1.00E+00 | GH1 |
| META56CEA8D1_218385 | −0.35 | −0.50 | 6.16E−01 | 1.00E+00 | GH25 |
| METAC4ED4080_201195 | −0.36 | −0.49 | 6.21E−01 | 1.00E+00 | GT2 |
| META8BC9F76E_314915 | −0.38 | −0.49 | 6.21E−01 | 1.00E+00 | GT2 |
| META6CD5EEA8_352050 | 0.26 | 0.49 | 6.28E−01 | 1.00E+00 | GH85 |
| METAB2EF0315_429785 | −0.36 | −0.48 | 6.30E−01 | 1.00E+00 | GT4 |
| META6C29AB57_464575 | 0.27 | 0.48 | 6.31E−01 | 1.00E+00 | GH13_20 |
| META56CEA8D1_416970 | 0.31 | 0.48 | 6.34E−01 | 1.00E+00 | CBM48-GH13_14 |
| METAD6BA71B8_249960 | −0.34 | −0.47 | 6.35E−01 | 1.00E+00 | GT2 |
| META865DF95A_396200 | 0.20 | 0.47 | 6.40E−01 | 1.00E+00 | GH51 |
| METAEAA5A2EB_104810 | 0.21 | 0.46 | 6.43E−01 | 1.00E+00 | GH4 |
| META56CEA8D1_02445 | 0.26 | 0.46 | 6.43E−01 | 1.00E+00 | GT35 |
| METAF7365A95_110080 | 0.35 | 0.46 | 6.45E−01 | 1.00E+00 | GH28 |
| META720DC391_226045 | 0.21 | 0.46 | 6.48E−01 | 1.00E+00 | GT2 |
| META6562874C_530525 | 0.31 | 0.45 | 6.52E−01 | 1.00E+00 | GH78 |
| META6CF5E124_52630 | 0.19 | 0.45 | 6.53E−01 | 1.00E+00 | GH77 |
| META25907FDA_182185 | 0.26 | 0.45 | 6.53E−01 | 1.00E+00 | GH13_39 |
| META07F61BCF_118205 | 0.36 | 0.45 | 6.55E−01 | 1.00E+00 | GH20 |
| METADD3DA67D_54920 | 0.20 | 0.45 | 6.56E−01 | 1.00E+00 | GH77 |
| METAEB6EC57B_502880 | 0.25 | 0.44 | 6.61E−01 | 1.00E+00 | GH13 |
| META10B1C6A8_123425 | 0.20 | 0.43 | 6.65E−01 | 1.00E+00 | GH1 |
| METABFB860FB_78515 | 0.24 | 0.43 | 6.66E−01 | 1.00E+00 | GT4 |
| META67F8845E_108415 | 0.32 | 0.43 | 6.68E−01 | 1.00E+00 | GT2 |
| META49506969_305445 | −0.31 | −0.42 | 6.72E−01 | 1.00E+00 | GT51 |
| METAB2EF0315_464970 | −0.31 | −0.42 | 6.72E−01 | 1.00E+00 | GH57 |
| META32FFA236_117995 | 0.29 | 0.42 | 6.76E−01 | 1.00E+00 | GT2 |
| META0A69D94D_301885 | −0.30 | −0.41 | 6.82E−01 | 1.00E+00 | GH43_5 |
| META2840A711_465080 | −0.16 | −0.41 | 6.82E−01 | 1.00E+00 | GH20 |
| META6CD5EEA8_611460 | 0.19 | 0.41 | 6.84E−01 | 1.00E+00 | GH1 |
| METACBC77C2A_361320 | −0.22 | −0.41 | 6.85E−01 | 1.00E+00 | GH92 |
| METADF44B175_10130 | 0.22 | 0.40 | 6.88E−01 | 1.00E+00 | GH13_8 |
| METAE3E9F7E9_354350 | −0.14 | −0.39 | 6.98E−01 | 1.00E+00 | GH1 |
| META85417ED2_327095 | 0.29 | 0.39 | 6.98E−01 | 1.00E+00 | GH28 |
| META0C6FF5CF_586565 | −0.28 | −0.38 | 7.06E−01 | 1.00E+00 | GH26 |
| META63788F6C_240860 | 0.20 | 0.37 | 7.10E−01 | 1.00E+00 | GH20 |
| METAC230BFD3_581855 | 0.16 | 0.37 | 7.13E−01 | 1.00E+00 | GH1 |
| METAB2EF0315_181210 | −0.27 | −0.37 | 7.15E−01 | 1.00E+00 | GH26 |
| META6CD5EEA8_319615 | 0.27 | 0.36 | 7.15E−01 | 1.00E+00 | GH32 |
| META7FC3DEDC_423290 | −0.26 | −0.36 | 7.16E−01 | 1.00E+00 | GT2 |
| METADF44B175_399665 | 0.20 | 0.35 | 7.23E−01 | 1.00E+00 | GT2 |
| METAA20921FD_558940 | 0.22 | 0.35 | 7.27E−01 | 1.00E+00 | GT51 |
| META33775318_54655 | −0.19 | −0.35 | 7.29E−01 | 1.00E+00 | GH130 |
| META25907FDA_128905 | 0.17 | 0.34 | 7.35E−01 | 1.00E+00 | GT4 |
| META85C0B70F_147460 | −0.26 | −0.34 | 7.35E−01 | 1.00E+00 | GT2 |
| METAAD2E8EAD_29210 | −0.24 | −0.34 | 7.36E−01 | 1.00E+00 | GH51 |
| META90554699_576465 | −0.24 | −0.33 | 7.39E−01 | 1.00E+00 | GT2 |
| METADB4286D7_482260 | −0.17 | −0.32 | 7.48E−01 | 1.00E+00 | GH25 |
| META6CD5EEA8_833560 | 0.22 | 0.32 | 7.53E−01 | 1.00E+00 | GH29 |
| METAF486615E_193885 | −0.24 | −0.31 | 7.56E−01 | 1.00E+00 | GH127 |
| META6C29AB57_623205 | −0.13 | −0.31 | 7.56E−01 | 1.00E+00 | GH43_22 |
| META44A0D997_39610 | −0.23 | −0.31 | 7.56E−01 | 1.00E+00 | GT2 |
| METABDF80FC2_40200 | −0.23 | −0.30 | 7.64E−01 | 1.00E+00 | CBM50 |
| METAB2CC138D_827030 | 0.16 | 0.30 | 7.64E−01 | 1.00E+00 | GT2 |
| METAD8023847_87595 | 0.24 | 0.30 | 7.64E−01 | 1.00E+00 | GH67 |
| META2E2167AF_284950 | 0.15 | 0.30 | 7.65E−01 | 1.00E+00 | GT5 |
| META56CEA8D1_33630 | 0.17 | 0.30 | 7.66E−01 | 1.00E+00 | GH2 |
| META7D10DFE7_438335 | 0.17 | 0.30 | 7.68E−01 | 1.00E+00 | GH25 |
| META837CC15C_564450 | 0.18 | 0.29 | 7.71E−01 | 1.00E+00 | GT51 |
| META9AFC44FC_752845 | 0.14 | 0.29 | 7.72E−01 | 1.00E+00 | CBM48-GH13_14 |
| METAEF1EC719_131500 | −0.21 | −0.29 | 7.74E−01 | 1.00E+00 | GH57 |
| META7D10DFE7_49655 | 0.13 | 0.29 | 7.74E−01 | 1.00E+00 | GT2 |
| META886A343C_191360 | −0.21 | −0.29 | 7.74E−01 | 1.00E+00 | GT28 |
| METAB2EF0315_264220 | 0.16 | 0.29 | 7.74E−01 | 1.00E+00 | GH23-CBM50 |
| METAF564A3A0_222525 | 0.21 | 0.29 | 7.75E−01 | 1.00E+00 | CBM20-CBM20 |
| METAE5DB790E_192930 | 0.21 | 0.27 | 7.87E−01 | 1.00E+00 | GH109 |
| META4DF6FBFF_51240 | 0.16 | 0.27 | 7.88E−01 | 1.00E+00 | GH2 |
| META8BC9F76E_368610 | −0.18 | −0.27 | 7.91E−01 | 1.00E+00 | GT2 |
| METAD68F6467_201080 | 0.20 | 0.26 | 7.94E−01 | 1.00E+00 | GH25 |
| META0FDDCADE_305335 | −0.19 | −0.26 | 7.97E−01 | 1.00E+00 | GH13_38 |
| META886A343C_146055 | 0.18 | 0.25 | 8.01E−01 | 1.00E+00 | GT51 |
| META518263D0_508105 | 0.21 | 0.25 | 8.04E−01 | 1.00E+00 | GH20 |
| METAE689F1DA_500450 | −0.16 | −0.23 | 8.17E−01 | 1.00E+00 | GT51 |
| META79F7DB64_238250 | 0.09 | 0.22 | 8.23E−01 | 1.00E+00 | GH13_30 |
| META63788F6C_29510 | 0.13 | 0.22 | 8.25E−01 | 1.00E+00 | CBM20-GH77 |
| META16EBD6B2_165315 | 0.16 | 0.22 | 8.25E−01 | 1.00E+00 | GH109 |
| META6CF5E124_387035 | 0.12 | 0.22 | 8.30E−01 | 1.00E+00 | GT28 |
| META111B0EC3_144500 | 0.15 | 0.21 | 8.32E−01 | 1.00E+00 | GH3 |
| META051E86A7_327005 | 0.17 | 0.21 | 8.32E−01 | 1.00E+00 | GH33 |
| META4582DD79_144470 | −0.09 | −0.21 | 8.33E−01 | 1.00E+00 | GT4 |
| META6CD5EEA8_763685 | −0.12 | −0.21 | 8.37E−01 | 1.00E+00 | GH95 |
| META650EBBEB_153155 | 0.15 | 0.20 | 8.39E−01 | 1.00E+00 | GH3 |
| META5D18B996_142460 | 0.12 | 0.20 | 8.39E−01 | 1.00E+00 | GT4 |
| METAC00CDA30_74065 | −0.09 | −0.20 | 8.44E−01 | 1.00E+00 | GH106 |
| META56CEA8D1_94865 | 0.11 | 0.19 | 8.46E−01 | 1.00E+00 | CBM72 |
| METAF4AB60B2_514795 | −0.14 | −0.18 | 8.54E−01 | 1.00E+00 | GH51 |
| META7D10DFE7_186990 | 0.10 | 0.18 | 8.56E−01 | 1.00E+00 | GH29 |
| META968686B5_38270 | 0.07 | 0.17 | 8.66E−01 | 1.00E+00 | GH53 |
| META1D69F13F_356785 | −0.08 | −0.17 | 8.67E−01 | 1.00E+00 | GH1 |
| METABAE494BD_08270 | 0.07 | 0.16 | 8.69E−01 | 1.00E+00 | GH73 |
| METAB143E28D_161985 | 0.07 | 0.16 | 8.69E−01 | 1.00E+00 | GT2 |
| METAB2CC138D_157595 | 0.11 | 0.16 | 8.71E−01 | 1.00E+00 | GH27 |
| META677B5746_77390 | 0.08 | 0.16 | 8.71E−01 | 1.00E+00 | GH1 |
| META49BF5A08_491680 | 0.07 | 0.16 | 8.76E−01 | 1.00E+00 | GT2 |
| META1CBC1A82_67610 | 0.08 | 0.16 | 8.76E−01 | 1.00E+00 | GT2 |
| METAF2D36A90_163550 | 0.07 | 0.15 | 8.78E−01 | 1.00E+00 | GT5 |
| METAB2EF0315_298555 | −0.12 | −0.15 | 8.80E−01 | 1.00E+00 | GH2 |
| META69C30C11_235005 | 0.08 | 0.14 | 8.87E−01 | 1.00E+00 | CBM32 |
| META7D10DFE7_194315 | −0.06 | −0.14 | 8.89E−01 | 1.00E+00 | CBM48-GH13_9 |
| META14843150_190705 | 0.10 | 0.13 | 8.95E−01 | 1.00E+00 | GT28 |
| META69C30C11_338030 | 0.06 | 0.13 | 8.95E−01 | 1.00E+00 | GH3 |
| METAF486615E_240260 | 0.10 | 0.13 | 8.96E−01 | 1.00E+00 | GH29 |
| METAB2EF0315_05565 | −0.07 | −0.13 | 9.00E−01 | 1.00E+00 | GH92 |
| META85128387_119510 | 0.09 | 0.12 | 9.05E−01 | 1.00E+00 | GT2 |
| META5D18B996_319790 | −0.09 | −0.12 | 9.05E−01 | 1.00E+00 | GT2 |
| META63788F6C_188475 | −0.06 | −0.12 | 9.05E−01 | 1.00E+00 | GH13 |
| META6DF66ED4_198800 | 0.05 | 0.12 | 9.07E−01 | 1.00E+00 | GH32 |
| METAEB3CE14A_42225 | −0.05 | −0.11 | 9.09E−01 | 1.00E+00 | GH1 |
| META8858A9DB_596480 | −0.05 | −0.11 | 9.11E−01 | 1.00E+00 | GH13 |
| METAB4E4AFBD_261965 | 0.05 | 0.11 | 9.12E−01 | 1.00E+00 | GH1 |
| META7D10DFE7_187000 | −0.06 | −0.11 | 9.14E−01 | 1.00E+00 | GH16 |
| META25907FDA_456160 | −0.08 | −0.11 | 9.15E−01 | 1.00E+00 | CBM50 |
| METAB2EF0315_345145 | 0.09 | 0.11 | 9.16E−01 | 1.00E+00 | GT26 |
| METADF44B175_79950 | −0.07 | −0.10 | 9.18E−01 | 1.00E+00 | GH2 |
| META6CD5EEA8_826230 | −0.07 | −0.10 | 9.20E−01 | 1.00E+00 | GH133 |
| METAFE58A94B_500310 | 0.05 | 0.10 | 9.21E−01 | 1.00E+00 | GT2 |
| METAC6FCD67E_228670 | 0.05 | 0.10 | 9.24E−01 | 1.00E+00 | GH43_4 |
| META7FC3DEDC_450885 | −0.07 | −0.09 | 9.27E−01 | 1.00E+00 | GH28-GH105 |
| METAA47594ED_44995 | −0.04 | −0.09 | 9.29E−01 | 1.00E+00 | GH1 |
| METAB2EF0315_185045 | −0.07 | −0.09 | 9.29E−01 | 1.00E+00 | GH28 |
| META52203B6D_114575 | 0.04 | 0.09 | 9.30E−01 | 1.00E+00 | GH32 |
| META6562874C_359165 | −0.05 | −0.08 | 9.33E−01 | 1.00E+00 | GH33 |
| METAFE58A94B_289020 | 0.04 | 0.08 | 9.33E−01 | 1.00E+00 | GH32 |
| METAD8929501_313840 | 0.04 | 0.08 | 9.36E−01 | 1.00E+00 | GH1 |
| META56CEA8D1_99770 | −0.04 | −0.08 | 9.37E−01 | 1.00E+00 | GH73-CBM50 |
| META937356F7_143510 | −0.03 | −0.08 | 9.40E−01 | 1.00E+00 | GT28 |
| META820EF1DE_148925 | 0.03 | 0.07 | 9.41E−01 | 1.00E+00 | GH23 |
| METAAB2BBC95_295545 | −0.05 | −0.07 | 9.42E−01 | 1.00E+00 | GT2 |
| META63788F6C_81220 | −0.03 | −0.05 | 9.59E−01 | 1.00E+00 | GH2 |
| METAE5DB790E_285990 | −0.03 | −0.05 | 9.60E−01 | 1.00E+00 | CBM50 |
| META1C72518C_409690 | −0.02 | −0.05 | 9.62E−01 | 1.00E+00 | CBM50 |
| META44A0D997_148050 | −0.03 | −0.05 | 9.63E−01 | 1.00E+00 | GT4 |
| META14843150_131955 | −0.04 | −0.04 | 9.65E−01 | 1.00E+00 | GH29 |
| META56CEA8D1_51165 | −0.02 | −0.04 | 9.66E−01 | 1.00E+00 | PL11 |
| METAD6BA71B8_98765 | 0.02 | 0.04 | 9.71E−01 | 1.00E+00 | GT19 |
| META73E4BF65_220130 | 0.01 | 0.03 | 9.72E−01 | 1.00E+00 | CBM32-GH20-CBM32 |
| META8CE2E347_97180 | −0.01 | −0.03 | 9.74E−01 | 1.00E+00 | GT4 |
| METACBC77C2A_565950 | 0.02 | 0.03 | 9.74E−01 | 1.00E+00 | GT2 |
| METADB4286D7_136735 | −0.02 | −0.03 | 9.76E−01 | 1.00E+00 | GT14 |
| META90554699_361310 | −0.01 | −0.02 | 9.80E−01 | 1.00E+00 | GT2 |
| METAE144DAE7_274600 | 0.01 | 0.02 | 9.84E−01 | 1.00E+00 | GH133 |
| METAD6BA71B8_479530 | 0.01 | 0.02 | 9.84E−01 | 1.00E+00 | GH16_3 |
| METAB2DAECE5_164195 | −0.01 | −0.02 | 9.84E−01 | 1.00E+00 | GH1 |
| METAC2EFD82B_279855 | −0.01 | −0.01 | 9.89E−01 | 1.00E+00 | GH133 |
| META7D10DFE7_393980 | −0.01 | −0.01 | 9.91E−01 | 1.00E+00 | GH43_12 |
| METAB2CC138D_162000 | 0.00 | 0.01 | 9.94E−01 | 1.00E+00 | GH77 |
| META6562874C_118910 | 0.00 | 0.00 | 9.97E−01 | 1.00E+00 | GH13_38 |
| META9035C4E6_148515 | 0.00 | 0.00 | 9.97E−01 | 1.00E+00 | GT2 |
| META46A9B8E2_129450 | 0.00 | 0.00 | 9.99E−01 | 1.00E+00 | GH28 |
| METAA9CC0F58_366930 | 0.00 | 0.00 | 1.00E+00 | 1.00E+00 | GH27 |

TABLE B

| ORF ID | logFC | t | p-value | q-value | CAZyme Family |
|---|---|---|---|---|---|
| METAF92B9794_285455 | 0.91 | 1.87 | 6.24E−02 | 1.00E+00 | GH104 |
| META4E8F9235_37115 | −0.92 | −1.67 | 9.47E−02 | 1.00E+00 | CBM5-CBM5-CBM5-CBM5- |
| | | | | | CBM5-GH18 |
| META28CE6F2A_414590 | −0.96 | −1.66 | 9.65E−02 | 1.00E+00 | GT51 |
| META0E525DAB_09330 | −1.10 | −1.60 | 1.11E−01 | 1.00E+00 | GH125 |
| META81621659_23615 | 0.64 | 1.35 | 1.78E−01 | 1.00E+00 | GH1 |
| META71A94948_27195 | −0.71 | −1.28 | 2.03E−01 | 1.00E+00 | GH25 |
| METAAD53F7F1_309665 | −0.69 | −1.24 | 2.16E−01 | 1.00E+00 | GH4 |
| METAB095DB19_39225 | −0.76 | −1.21 | 2.26E−01 | 1.00E+00 | GT51 |
| META8243BDCF_652510 | −0.48 | −1.14 | 2.54E−01 | 1.00E+00 | GT2 |
| META45D2EA48_243550 | −0.68 | −1.12 | 2.64E−01 | 1.00E+00 | GH5_18 |
| METAAD53F7F1_226785 | 0.46 | 1.06 | 2.91E−01 | 1.00E+00 | GH92 |
| META675C6E24_69355 | −0.63 | −1.00 | 3.17E−01 | 1.00E+00 | GT9 |
| META1271C981_04310 | −0.64 | −0.95 | 3.45E−01 | 1.00E+00 | GH77 |
| META4D033ABD_08455 | −0.63 | −0.94 | 3.48E−01 | 1.00E+00 | GH13 |
| META5FA34F77_143250 | −0.65 | −0.92 | 3.60E−01 | 1.00E+00 | GH112 |
| METAEEAA667F_02025 | −0.52 | −0.91 | 3.61E−01 | 1.00E+00 | GH127 |
| META3F0CAFD1_166080 | −0.50 | −0.90 | 3.70E−01 | 1.00E+00 | GT20 |
| META93970388_05650 | −0.48 | −0.86 | 3.88E−01 | 1.00E+00 | GH42 |
| META99B4D712_01845 | 0.49 | 0.79 | 4.31E−01 | 1.00E+00 | GT2 |
| META37509A1D_197845 | 0.37 | 0.78 | 4.37E−01 | 1.00E+00 | GH112 |
| META7B9A48E1_64050 | −0.47 | −0.76 | 4.46E−01 | 1.00E+00 | GT4 |
| META245698C2_20690 | 0.44 | 0.74 | 4.57E−01 | 1.00E+00 | GT2 |
| METAE9A0B17A_155845 | −0.49 | −0.72 | 4.69E−01 | 1.00E+00 | GH20 |
| META24230478_173415 | −0.26 | −0.71 | 4.77E−01 | 1.00E+00 | CBM48-GH13_9 |
| META684C8266_73285 | −0.41 | −0.71 | 4.78E−01 | 1.00E+00 | CBM50 |
| META25027D66_120560 | 0.39 | 0.69 | 4.93E−01 | 1.00E+00 | GT8 |
| META976EC559_281260 | 0.40 | 0.69 | 4.93E−01 | 1.00E+00 | GT8 |
| METAD29BD862_18190 | −0.45 | −0.68 | 4.95E−01 | 1.00E+00 | GH23 |
| META0A69D94D_139360 | −0.40 | −0.67 | 5.03E−01 | 1.00E+00 | GT35 |
| META6841149F_171740 | 0.44 | 0.64 | 5.23E−01 | 1.00E+00 | GH13_30 |
| META3C0CE0E5_300350 | −0.43 | −0.64 | 5.26E−01 | 1.00E+00 | GT4 |
| META63A86881_32910 | −0.35 | −0.60 | 5.49E−01 | 1.00E+00 | CBM50 |
| META679DF154_125440 | −0.34 | −0.60 | 5.51E−01 | 1.00E+00 | GH1 |
| META2ECE99EF_55435 | −0.32 | −0.59 | 5.57E−01 | 1.00E+00 | GH43_29 |
| META6CE575C5_170840 | −0.34 | −0.58 | 5.63E−01 | 1.00E+00 | GH3 |
| META0FDDCADE_105805 | −0.33 | −0.56 | 5.73E−01 | 1.00E+00 | GH23 |
| META67F8845E_379625 | 0.32 | 0.55 | 5.83E−01 | 1.00E+00 | CBM50 |
| META3B57598A_70465 | −0.33 | −0.53 | 5.99E−01 | 1.00E+00 | GT30 |
| META15B498E8_124525 | 0.30 | 0.50 | 6.17E−01 | 1.00E+00 | GT14 |
| META63788F6C_198365 | 0.30 | 0.50 | 6.19E−01 | 1.00E+00 | GH1 |
| METAEF1EC719_320120 | 0.28 | 0.49 | 6.21E−01 | 1.00E+00 | GH73 |
| META7796BCC3_107370 | 0.28 | 0.48 | 6.32E−01 | 1.00E+00 | GH2 |
| METAAB43407A_151825 | −0.25 | −0.43 | 6.69E−01 | 1.00E+00 | GH8 |
| META00196547_08350 | −0.28 | −0.43 | 6.70E−01 | 1.00E+00 | GH33 |
| META379B2819_72155 | −0.24 | −0.42 | 6.78E−01 | 1.00E+00 | GH37 |
| METAC8F85FF3_267915 | −0.21 | −0.38 | 7.04E−01 | 1.00E+00 | GT4 |
| METAD8543035_10500 | −0.23 | −0.37 | 7.13E−01 | 1.00E+00 | GH18 |
| META4B3CD142_347055 | −0.17 | −0.35 | 7.30E−01 | 1.00E+00 | GT2 |
| METAA7775064_09335 | −0.22 | −0.34 | 7.32E−01 | 1.00E+00 | GH1 |
| METAFCBEFEC7_93550 | 0.20 | 0.34 | 7.36E−01 | 1.00E+00 | GT83 |
| META6ED44E38_09355 | 0.20 | 0.34 | 7.36E−01 | 1.00E+00 | GT8 |
| META36701F4D_333630 | −0.19 | −0.32 | 7.45E−01 | 1.00E+00 | GT19 |
| META0F55B860_164405 | −0.20 | −0.32 | 7.50E−01 | 1.00E+00 | GH73 |
| METACF1C1A6A_38020 | 0.17 | 0.31 | 7.56E−01 | 1.00E+00 | GH13_30 |
| METAD489796F_117085 | −0.18 | −0.31 | 7.60E−01 | 1.00E+00 | GH23 |
| METACEB2A5B8_164245 | −0.20 | −0.31 | 7.60E−01 | 1.00E+00 | CBM50 |
| METAB89B2F80_95130 | −0.18 | −0.29 | 7.73E−01 | 1.00E+00 | GT5 |
| META16EBD6B2_171600 | 0.19 | 0.28 | 7.80E−01 | 1.00E+00 | GH77 |
| METAB478E8DD_17545 | 0.16 | 0.28 | 7.81E−01 | 1.00E+00 | GH23 |
| META36701F4D_185475 | −0.17 | −0.27 | 7.86E−01 | 1.00E+00 | GH77 |
| METAFEAC785B_209315 | −0.16 | −0.26 | 7.96E−01 | 1.00E+00 | GT28 |
| META81F3513C_36950 | −0.15 | −0.25 | 8.06E−01 | 1.00E+00 | CBM48-GH13_11 |
| METAB74DA83E_229395 | −0.14 | −0.22 | 8.22E−01 | 1.00E+00 | GH24 |
| META820EF1DE_193280 | 0.12 | 0.21 | 8.35E−01 | 1.00E+00 | GH127 |
| META8CCC7E52_21525 | −0.14 | −0.21 | 8.36E−01 | 1.00E+00 | GT28 |
| META9F4434E4_94285 | −0.11 | −0.20 | 8.41E−01 | 1.00E+00 | GH13_30 |
| METAC65E18AB_193890 | −0.11 | −0.20 | 8.43E−01 | 1.00E+00 | GT51 |
| META5FC9D67E_238500 | 0.11 | 0.20 | 8.43E−01 | 1.00E+00 | GH25 |
| META35DD442B_57545 | −0.10 | −0.16 | 8.73E−01 | 1.00E+00 | GH102 |
| METAAB43407A_171080 | 0.10 | 0.15 | 8.77E−01 | 1.00E+00 | GT26 |
| METABB96A33D_57825 | −0.09 | −0.15 | 8.80E−01 | 1.00E+00 | GH13_19 |
| META0A2F4A81_223835 | −0.08 | −0.12 | 9.03E−01 | 1.00E+00 | GT51 |
| METAF92B9794_104465 | −0.07 | −0.12 | 9.06E−01 | 1.00E+00 | GH103 |
| META3BB06B68_241650 | −0.06 | −0.10 | 9.20E−01 | 1.00E+00 | GT2 |
| META07F61BCF_115620 | −0.06 | −0.09 | 9.25E−01 | 1.00E+00 | GT2 |
| META3CE39FA7_198510 | −0.05 | −0.09 | 9.26E−01 | 1.00E+00 | GH5_18 |
| METAEB859A7C_322900 | −0.05 | −0.09 | 9.31E−01 | 1.00E+00 | GH37 |
| META21739DC6_33560 | 0.04 | 0.08 | 9.40E−01 | 1.00E+00 | CBM34-GH13_21 |
| META62D398DB_16880 | 0.05 | 0.07 | 9.45E−01 | 1.00E+00 | GH13_4 |
| METAFCC752B7_87710 | 0.04 | 0.07 | 9.47E−01 | 1.00E+00 | GH24 |
| METAC65E18AB_219620 | −0.04 | −0.06 | 9.50E−01 | 1.00E+00 | GT9 |
| META1BC448A1_176495 | −0.03 | −0.05 | 9.63E−01 | 1.00E+00 | GH23-CBM50-CBM50 |
| METADEAFF9B4_137430 | −0.02 | −0.04 | 9.70E−01 | 1.00E+00 | GT2 |
| META0C3933DC_88175 | 0.01 | 0.02 | 9.87E−01 | 1.00E+00 | CBM50 |

In certain embodiments, an edible composition comprising carbohydrates of the present disclosure is a composition comprising chickpea flour or a glycan equivalent thereof, peanut flour or a glycan equivalent thereof, soy flour or a glycan equivalent thereof, raw banana or a glycan equivalent thereof, and a micronutrient premix. The micronutrient premix provides at least 60% of the recommended daily allowance of vitamin A, vitamin C, vitamin D, vitamin E, vitamin B, calcium, copper, iron, magnesium, manganese, phosphorus, potassium, and zinc. Compositions of the present disclosure further comprise about 300 to about 560 kcal per 100 g of the composition, a protein energy ratio (PER) of about 8% to about 20%, and a fat energy ratio (FER) of about 30% to about 60%, and may further comprise about 20 g to about 36 g of fat per 100 g of the composition and about 11 g to about 16 g of protein per 100 g of the composition. Additional ingredients such as sweeteners, flavors and spices, flavor enhancers, fats, fat replacers, emulsifiers, and the like may be optionally included to create an organoleptically accepTable Eomposition. As used herein, an “organoleptically accepTable Eomposition” is a composition that is acceptable to a subject with respect to the senses such as small, appearance, taste and touch. These additional ingredients may affect the energy content, PER and FER of the composition; however compositions comprising one or more additional ingredient shall still have about 300to about 560 kcal per 100 g of the composition, a protein energy ratio (PER) of about 8% to about 20%, and a fat energy ratio (FER) of about 30% to about 60%.

Compositions of the present disclosure may be formulated into a beverage, a food or a supplement. Non-limiting examples include a bar, a paste, a gel, a cookie, a cracker, a powder, a pellet, a powdered drink to be reconstituted, a blended beverage, a carbonated beverage, and the like. When compositions of the present disclosure are intended to be administered and consumed by humans, the ingredients in the compositions are typically Food Chemicals Codex (FCC) purity or U.S. Pharmacopeia (USP)—National Formulary quality, as appropriate, and free from foreign materials. In some embodiments, a composition may be a therapeutic food. In some embodiments, a composition may be a ready-to-use food. The term “ready-to-use food” refers to a food that comes ready to use as provided. Specifically, a ready-to-use food doesn't require reconstitution or refrigeration, and stays fresh for at least 6 months, preferably one year, or more preferably two years. In some embodiments, a composition may be a ready-to-use therapeutic food, as defined in U.S. Department of Agriculture, “Commercial Item Description: Ready-to-Use Therapeutic Food (RUTF)” A-A-20363B (2012). In some embodiments, a composition may be animal food or animal feed. In some embodiments, a composition may be a supplement for animal food or animal feed.

### (a) Composition Comprising Chickpea Flour, Peanut Flour, Soy Flour, Raw Banana

In one aspect, a composition of the present disclosure comprises chickpea flour, peanut flour, soy flour, and raw banana, wherein the chickpea flour, the peanut flour, the soy flour, and the raw banana provide at least 8.5 g of protein per 100 g of the composition. In preferred embodiments, the composition contains no cow's milk or powdered cow's milk, or no milk or powdered milk of any kind, or no milk, powdered milk, or milk product of any kind. In still further embodiments, the composition also contains no seeds, nuts, nut butters, dried fruit, cocoa nibs, cocoa powder, chocolate, rice flour, lentil flour, or any combination thereof. For example, compositions of the present disclosure comprising chickpea flour, peanut flour, soy flour, and raw banana may contain no cow's milk or powdered cow's milk and (a) no seed, nuts, and nut butter, and/or (b) no cocoa nibs, cocoa powder or chocolate, and/or (c) no rice flour and lentil flour, and/or (d) no dried fruit. In another example, compositions of the present disclosure comprising chickpea flour, peanut flour, soy flour, and raw banana may contain no milk or powdered milk of any kind and (a) no seed, nuts, and nut butter, and/or (b) no cocoa nibs, cocoa powder or chocolate, and/or (c) no rice flour and lentil flour, and/or (d) no dried fruit.

In some embodiments, the chickpea flour, the peanut flour, the soy flour, and the raw banana, in total, provide 8.5 g to about 15 g of protein per 100 g of the composition. In some embodiments, the chickpea flour, the peanut flour, the soy flour, and the raw banana, in total, provide about 9 g to about 15 g of protein per 100 g of the composition. In some embodiments, the chickpea flour, the peanut flour, the soy flour, and the raw banana, in total, provide about 10 g to about 15 g of protein per 100 g of the composition. In some embodiments, the chickpea flour, the peanut flour, the soy flour, and the raw banana, in total, provide about 11 g to about 15 g of protein per 100 g of the composition. In some embodiments, the chickpea flour, the peanut flour, the soy flour, and the raw banana, in total, provide about 9 g to about 12 g of protein per 100 g of the composition. In some embodiments, the chickpea flour, the peanut flour, the soy flour, and the raw banana, in total, provide about 10 g to about 12 g of protein per 100 g of the composition. In some embodiments, the chickpea flour, the peanut flour, the soy flour, and the raw banana, in total, provide about 11 g to about 12 g of protein per 100 g of the composition. In some embodiments, the chickpea flour, the peanut flour, the soy flour, and the raw banana, in total, provide about 12 g to about 15 g of protein per 100 g of the composition. In some embodiments, the chickpea flour, the peanut flour, the soy flour, and the raw banana, in total, provide about 12 g to about 14 g of protein per 100 g of the composition. In some embodiments, the chickpea flour, the peanut flour, the soy flour, and the raw banana, in total, provide about 13 g to about 15 g of protein per 100 g of the composition. In other embodiments, the chickpea flour, the peanut flour, the soy flour, and the raw banana, in total, provide 8.5 g, about 9 g, about 9.5 g, about 10 g, about 10.5 g, about 11 g, about 11.5 g, about 12 g, about 12.5 g, about 13 g, about 13.5 g, about 14 g, about 14.5 g, or about 15 g of protein per 100 g of the composition.

In each of the above embodiments, the weight ratio of the chickpea flour to the peanut flour to the soy flour to the raw banana may vary. Typically, chickpea flour has about 20% protein by weight, peanut flour has about 50% protein by weight, soy flour has about 50% protein by weight, and raw banana has about 1% protein by weight. The weight percentages of protein in each ingredient may vary however, depending upon the varietal of plant and, in the case of the flours, the method used to manufacture the flour. In some embodiments, the weight ratio is about 1: about 1: about 0.8: about 1.9, respectively (chickpea flour: peanut flour: soy flour: raw banana), or a weight ratio adjusted as needed to reflect differences in the ingredients.

In an exemplary embodiment, a composition of the present disclosure comprises about 9-11 g of chickpea flour, about 9-11 g of peanut flour, about 7-9 g of soy flour, and about 17-21 g of raw banana. In preferred embodiments, the composition contains no cow's milk or powdered cow's milk, or no milk or powdered milk of any kind. In still further embodiments, the composition also contains no seeds, nuts, nut butters, dried fruit, cocoa nibs, cocoa powder, chocolate, rice flour, lentil flour, or any combination thereof. For example, compositions of the present disclosure comprising chickpea flour, peanut flour, soy flour, and raw banana may contain no cow's milk or powdered cow's milk and (a) no seed, nuts, and nut butter, and/or (b) no cocoa nibs, cocoa powder or chocolate, and/or (c) no rice flour and lentil flour, and/or (d) no dried fruit. In another example, compositions of the present disclosure comprising chickpea flour, peanut flour, soy flour, and raw banana may contain no milk or powdered milk of any kind and (a) no seed, nuts, and nut butter, and/or (b) no cocoa nibs, cocoa powder or chocolate, and/or (c) no rice flour and lentil flour, and/or (d) no dried fruit.

In another exemplary embodiment, a composition of the present disclosure comprises about 10 g of chickpea flour, about 10 g of peanut flour, about 8 g of soy flour, and about 19 g of raw banana. In preferred embodiments, the composition contains no cow's milk or powdered cow's milk, or no milk or powdered milk of any kind. In still further embodiments, the composition also contains no seeds, nuts, nut butters, dried fruit, cocoa nibs, cocoa powder, chocolate, rice flour, lentil flour, or any combination thereof. For example, compositions of the present disclosure comprising chickpea flour, peanut flour, soy flour, and raw banana may contain no cow's milk or powdered cow's milk and (a) no seed, nuts, and nut butter, and/or (b) no cocoa nibs, cocoa powder or chocolate, and/or (c) no rice flour and lentil flour, and/or (d) no dried fruit. In another example, compositions of the present disclosure comprising chickpea flour, peanut flour, soy flour, and raw banana may contain no milk or powdered milk of any kind and (a) no seed, nuts, and nut butter, and/or (b) no cocoa nibs, cocoa powder or chocolate, and/or (c) no rice flour and lentil flour, and/or (d) no dried fruit.

(b) composition comprising qlycan equivalents of chickpea flour, peanut flour, soy flour, raw banana

In another aspect, a composition of the present disclosure is a composition of Section 1(a), wherein some or all the chickpea flour, the peanut flour, the soy flour, and/or the raw banana is replaced with a glycan equivalent thereof. As used herein, a “glycan equivalent” refers to a composition with a similar glycan content. The term “similar” generally refers to a range of numerical values, for instance,±0.5-1%,±1-5% or±5-10% of the recited value, that one would consider equivalent to the recited value, for example, having the same function or result. Because a glycan equivalent has a similar glycan content to the ingredient it is replacing, it may be substituted about 1:1. For instance, if 3 g of chickpea flour is to be replaced with a glycan equivalent thereof, one of skill in the art would use about 3 g of the chickpea glycan equivalent. A glycan equivalent may be defined in terms of its monosaccharide content and optionally by an analysis of the glycosidic linkages. Methods for measuring monosaccharide content and analyzing glycosidic linkages are known in the art.

In some embodiments, some or all the chickpea flour is replaced with a glycan equivalent of chickpea flour. For instance, a composition of Section I(a) may comprise a glycan equivalent of about 0.5 g or more of chickpea flour. In another example, a composition of Section I(a) may comprise a glycan equivalent of about 1 g, about 2 g, about 3 g, about 4 g, about 5 g, about 6 g, about 7 g, about 8 g, about 9 g, or about 10 g of chickpea flour. In another example, a composition of Section I(a) may comprise a glycan equivalent of about 0.1 g to about 10 g of chickpea flour, or about 0.5 to about 5 g of chickpea flour. In another example, a composition of Section I(a) may comprise a glycan equivalent of about 1 g to about 10 g of chickpea flour, or about 1 g to about 5 g of chickpea flour, or about 2.5 g to about 7.5 g of chickpea flour, to about 5 g to about 10 g of chickpea flour. In further embodiments, some or all the peanut flour is also replaced with a glycan equivalent of peanut flour, some or all the soy flour is also replaced with a glycan equivalent of soy flour, and/or some or all the raw banana is also replaced with a glycan equivalent of raw banana.

In some embodiments, some or all the peanut flour is replaced with a glycan equivalent of peanut flour. For instance, a composition of Section I(a) may comprise a glycan equivalent of about 0.5 g or more of peanut flour. In another example, a composition of Section I(a) may comprise a glycan equivalent of about 1 g, about 2 g, about 3 g, about 4 g, about 5 g, about 6 g, about 7 g, about 8 g, about 9 g, or about 10 g of peanut flour. In another example, a composition of Section I(a) may comprise a glycan equivalent of about 0.1 g to about 10 g of peanut flour, or about 0.5 to about 5 g of peanut flour. In another example, a composition of Section I(a) may comprise a glycan equivalent of about 1 g to about 10 g of peanut flour, or about 1 g to about 5 g of peanut flour, or about 2.5 g to about 7.5 g of peanut flour, to about 5 g to about 10 g of peanut flour. In further embodiments, some or all the chickpea flour is also replaced with a glycan equivalent of chickpea flour, some or all the soy flour is also replaced with a glycan equivalent of soy flour, and/or some or all the raw banana is also replaced with a glycan equivalent of raw banana.

In some embodiments, some or all the soy flour is replaced with a glycan equivalent of soy flour. For instance, a composition of Section I(a) may comprise a glycan equivalent of about 0.5 g or more of soy flour. In another example, a composition of Section I(a) may comprise a glycan equivalent of about 1 g, about 2 g, about 3 g, about 4 g, about 5 g, about 6 g, about 7 g, or about 8 g of soy flour. In another example, a composition of Section I(a) may comprise a glycan equivalent of about 0.1 g to about 8 g of soy flour, or about 0.5 to about 5 g of soy flour. In another example, a composition of Section I(a) may comprise a glycan equivalent of about 1 g to about 8 g of soy flour, or about 1 g to about 4 g of soy flour, or about 2 g to about 6 g of soy flour, to about 4 g to about 8 g of soy flour. In further embodiments, some or all the chickpea flour is also replaced with a glycan equivalent of chickpea flour, some or all the peanut flour is also replaced with a glycan equivalent of peanut flour, and/or some or all the raw banana is also replaced with a glycan equivalent of raw banana.

In some embodiments, some or all the raw banana is replaced with a glycan equivalent of raw banana. For instance, a composition of Section I(a) may comprise a glycan equivalent of about 0.5 g or more of raw banana. In another example, a composition of Section I(a) may comprise a glycan equivalent of about 1 g, about 2 g, about 3 g, about 4 g, about 5 g, about 6 g, about 7 g, about 8 g of raw banana, about 9 g of raw banana, about 10 g of raw banana, about 11 g of raw banana, about 12 g of raw banana, about 13 g of raw banana, about 14 g of raw banana, about 15 g of raw banana, about 16 g of raw banana, about 17 g of raw banana, about 18 g of raw banana, or about 19 g of raw banana. In another example, a composition of Section I(a) may comprise a glycan equivalent of about 0.1 g to about 8 g of raw banana, or about 0.5 to about 5 g of raw banana. In another example, a composition of Section I(a) may comprise a glycan equivalent of about 1 g to about 8 g of raw banana, or about 1 g to about 4 g of raw banana, or about 2 g to about 6 g of raw banana, to about 4 g to about 8 g of raw banana. In further embodiments, some or all the chickpea flour is also replaced with a glycan equivalent of chickpea flour, some or all the peanut flour is also replaced with a glycan equivalent of peanut flour, and/or some or all the soy flour is also replaced with a glycan equivalent of soy flour.

### (c) Micronutrient Premix

A micronutrient premix in a composition of the present disclosure is present in an amount that provides at least 60% of the recommended daily allowance (RDA), for a given age group, of minimally vitamin A, vitamin C, vitamin D, vitamin E, vitamin B, calcium, copper, iron, magnesium, manganese, phosphorus, potassium, and zinc. The RDA of vitamin A, vitamin C, vitamin D, vitamin E, vitamin B, calcium, copper, iron, magnesium, manganese, phosphorus, potassium, and zinc, for various age groups, is known in the art. Given that different age groups may have different RDA's, it will be appreciated by a person of skill in the art that certain compositions may not be suiTable Hor subjects of all ages. For example, a composition with 60% of the Vitamin C RDA for a subject 7-12 months in age (e.g., 40 mg) will not contain at least 60% of the Vitamic C RDA for a subject 21 years of age (e.g., 75-90 mg). The term “vitamin” “B,” as used herein, is inclusive of all B vitamins, unless otherwise specified. Although compositions of the present disclosure are described as comprising a micronutrient premix, the addition of each vitamin and mineral separately, or the use of multiple premixes, is also contemplated and encompassed by the embodiments described herein. Similarly, in alternative embodiments, the micronutrient premix can be formulated separately and administered as a distinct composition in conjunction with a composition comprising chickpea flour or a glycan equivalent thereof, peanut flour or a glycan equivalent thereof, soy flour or a glycan equivalent thereof, raw banana or a glycan equivalent thereof.

In various embodiments, a micronutrient premix provides at least 60%, at least 61° A, at least 62%, at least 63%, at least 64%, at least 65%, at least 66%, at least 67%, at least 68%, at least 69%, at least 70%, at least 71° A, at least 72%, at least 73%, at least 74%, at least 75%, at least 76%, at least 77%, at least 77%, at least 78%, at least 79%, at least 80%, at least 81° A, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% of the recommended daily allowance (RDA), for a given age group,of minimally vitamin A, vitamin B, vitamin C, vitamin D, vitamin E, calcium, copper, iron, magnesium, manganese, phosphorous, potassium and zinc. In certain embodiments, a micronutrient premix provides more than 100% of the RDA, for a given age group,of minimally vitamin A, vitamin B, vitamin C, vitamin D, vitamin E, calcium, copper, iron, magnesium, manganese, phosphorous, potassium and zinc. In a specific embodiment, the micronutrient premix provides at least 75% of the recommended daily allowance (RDA), for a given age group,of minimally vitamins A, C, D and E, all B vitamins, calcium, copper, iron, magnesium, manganese, phosphorous, potassium and zinc. The RDA of vitamins and minerals for different age groups is well known in the art.

In a specific embodiment, a micronutrient premix provides at least 60%, at least 61%, at least 62%, at least 63%, at least 64%, at least 65%, at least 66%, at least 67%, at least 68%, at least 69%, at least 70%, at least 71° A, at least 72%, at least 73%, at least 74%, at least 75%, at least 76%, at least 77%, at least 77%, at least 78%, at least 79%, or at least 80% of the recommended daily allowance (RDA) for children aged 12-18 months of vitamin A, vitamin B, vitamin C, vitamin D, vitamin E, calcium, copper, iron, magnesium, manganese, phosphorous, potassium and zinc.

In another specific embodiment, the micronutrient premix provides at least 70% of the recommended daily allowance (RDA) for children aged 12-18 months of minimally vitamin A, vitamin B, vitamin C, vitamin D, vitamin E, calcium, copper, iron, magnesium, manganese, phosphorous, potassium and zinc.

In another specific embodiment, the micronutrient premix provides at least 75% of the recommended daily allowance (RDA) for children aged 12-18 months of minimally vitamin A, vitamin B, vitamin C, vitamin D, vitamin E, calcium, copper, iron, magnesium, manganese, phosphorous, potassium and zinc.

A micronutrient premix may further comprise vitamins and minerals in addition to the vitamin A, vitamin B, vitamin C, vitamin D, vitamin E, calcium, copper, iron, magnesium, manganese, phosphorous, potassium and zinc .

In an exemplary embodiment, a composition of the present disclosure contains vitamin A, vitamin C, vitamin D, vitamin E, vitamin B, calcium, copper, iron, magnesium, phosphorus, potassium, and zinc in the amounts listed in Table C and Table D. In a preferred embodiment, a composition of the present disclosure contains the nutrients of Table C in the amounts listed in Table C. In another preferred embodiment, a composition of the present disclosure contains the nutrients of Table D in the amounts listed in Table D. In yet another preferred embodiment, a composition of the present disclosure contains the nutrients of both Table C and Table D, in the amounts listed in Table C and Table D respectively.

TABLE C
Vitamin Premix

| | | | Units of |
|---|---|---|---|
| | | | Measurement |
| | Minimum | Maximum | per gram of the |
| Nutrients | Amount | Amount | Vitamin Premix |
| Vitamin A | 12655.013 | 16170.294 | IU |
| Thiamine Mononitrate | 6.765 | 8.644 | mg |
| Vitamin B12 | 11.700 | 17.550 | mcg |
| Vitamin B2 - Riboflavin | 5.485 | 7.008 | mg |
| Pyridoxine Hydrochloride | 6.153 | 7.863 | mg |
| Vitamin C | 236.250 | 301.875 | mg |
| Sodium | 29.213 | 37.327 | mg |
| Calcium D-Pantothenate | 20.798 | 26.574 | mg |
| Vitamin D3 | 7593.960 | 9703.599 | IU |
| Vitamin E (as E Acetate) | 120.690 | 154.215 | IU |
| Folic acid | 2531.007 | 3234.065 | mcg |
| Vitamin K1 | 405.009 | 584.991 | mcg |
| Niacinamide | 60.750 | 77.625 | mg |

For a 100 g composition, 160 mg of the Vitamin Premix is used. Accordingly, to calculate the amount of a given mineral in a 100 g composition, the amounts listed above are multiplied by 0.160.

In an exemplary embodiment, a composition of the present disclosure contains the micronutrients in Table D, in the amounts in Table D.

TABLE D
Mineral Premix

| | | | Units of |
|---|---|---|---|
| | | | Measurement |
| | Minimum | Maximum | per gram of the |
| Nutrients | Amount | Amount | mineral premix |
| Calcium | 170.000 | 216.000 | mg |
| Phosphorus | 93.000 | 118.000 | mg |
| Calcium | 0.000 | 0.000 | Q.S. |
| Copper | 0.181 | 0.231 | mg |
| Iodine | 52.945 | 67.652 | mcg |
| Iron | 3.169 | 4.049 | mg |
| Magnesium | 27.163 | 34.708 | mg |
| Manganese | 0.543 | 0.694 | mg |
| Potassium (K) | 89.342 | 114.159 | mg |
| Selenium | 11.770 | 15.040 | mcg |
| Zinc | 2.415 | 3.085 | mg |

For a 100 g composition, 2.982 g of the Mineral Premix is used. Accordingly, to calculate the amount of a given mineral in a 100 g composition, the amounts listed above are multiplied by 2.982.

### (d) Macronutrient Content

In each of the aforementioned embodiments, a composition may comprise about 300 kcal to about 560 kcal per 100 g of the composition, a protein energy ratio (PER) of about 8% to about 20%, and a fat energy ratio (FER) of about 30% to about 60%. In some embodiments, a composition may comprise about 350 kcal to about 560 kcal per 100 g of the composition, a protein energy ratio (PER) of about 8% to about 20%, and a fat energy ratio (FER) of about 30% to about 60%. In other embodiments, a composition may comprise about 400 kcal to about 560 kcal per 100 g of the composition, a protein energy ratio (PER) of about 8% to about 12%, and a fat energy ratio (FER) of about 45% to about 60%. In an exemplary embodiment, a composition may comprise about 400 to about 560 kcal per 100 g of the composition, about 20 g to about 36 g of fat per 100 g of the composition, about 11 g to about 16 g of protein per 100 g of the composition, a protein energy ratio (PER) of about 8% to about 12%, and a fat energy ratio (FER) of about 45% to about 60%. Carbohydrates and sugars may provide the remainder of the energy content. For instance, if a composition has a PER of 10% and a FER of 50%, then the carbohydrate+sugar-to-energy ratio may be 40%.

In one embodiment, a composition of the disclosure provides about 300 kcal, about 310 kcal, about 320 kcal, about 330 kcal, about 340 kcal, or about 350 kcal per 100 g of the composition. In another embodiment, a composition of the disclosure provides about 350 kcal, about 360 kcal, about 370 kcal, about 380 kcal, about 390 kcal, or about 400 kcal per 100 g of the composition. In another embodiment, a composition of the disclosure provides about 400 kcal, about 410 kcal, about 420 kcal, about 430 kcal, about 440 kcal, or about 450 kcal per 100 g of the composition. In another embodiment, a composition of the disclosure provides about 460 kcal, about 470 kcal, about 480 kcal, about 490 kcal, or about 500 kcal per 100 g of the composition. In another embodiment, a composition of the disclosure provides about 500 kcal, about 510 kcal, about 520 kcal, about 530 kcal, about 540 kcal, about 550 kcal, or about 560 kcal per 100 g of the composition. In another embodiment, a composition of the disclosure provides about 400 kcal to about 560 kcal, about 420 kcal to about 560 kcal, about 440 kcal to about 560 kcal, about 460 kcal to about 560 kcal, about 480 kcal to about 560 kcal or about 500 kcal to about 560 kcal per 100 g of the composition. In another embodiment, a composition of the disclosure provides about 300 kcal to about 450 kcal per 100 g of the composition. In another embodiment, a composition of the disclosure provides about 300 kcal to about 425 kcal per 100 g of the composition. In another embodiment, a composition of the disclosure provides about 300 kcal to about 400 kcal per 100 g of the composition. In another embodiment, a composition of the disclosure provides about 300 kcal to about 350 kcal per 100 g of the composition. In another embodiment, a composition of the disclosure provides about 350 kcal to about 450 kcal per 100 g of the composition. In another embodiment, a composition of the disclosure provides about 350 kcal to about 400 kcal per 100 g of the composition. In another embodiment, a composition of the disclosure provides about 325 kcal to about 425 kcal per 100 g of the composition. In another embodiment, a composition of the disclosure provides about 400 kcal to about 500 kcal per 100 g of the composition, about 420 kcal to about 500 kcal per 100 g of the composition, about 440 kcal to about 500 kcal per 100 g of the composition, about 460 kcal to about 500 kcal per 100 g of the composition, or about 480 kcal to about 500 kcal per serving 100 g of the composition. In still another embodiment, a composition of the disclosure provides about 400 kcal to about 480 kcal per 100 g of the composition, about 400 kcal to about 460 kcal per 100 g of the composition, or about 400 kcal to about 440 kcal per 100 g of the composition. In another embodiment, a composition of the present disclosure provides about 400 kcal to about 420 kcal, about 400 kcal to about 410 kcal, about 405 kcal to about 415 kcal, or about 410 kcal to about 420 kcal per 100 g of the composition. In another embodiment, a composition of the present disclosure provides about 400 kcal to about 415 kcal, about 400 kcal to about 410 kcal, or about 405 kcal to about 415 kcal per 100 g of the composition.

In each of the above embodiments, a composition may comprise about 11 g, about 12 g, about 13 g, about 14 g, about 15 g, or about 16 g of protein per 100 g of the composition. For instance, a composition may comprise about 11.1 g, about 11.2 g, about 11.3 g, about 11.4 g, about 11.5 g, about 11.6 g, about 11.7 g, about 11.8 g, about 11.9 g of protein per 100 g of the composition. In another example, a composition may comprise about 12 g, about 12.1 g, about 12.2 g, about 12.3 g, about 12.4 g, about 12.5 g, about 12.6 g, about 12.7 g, about 12.8 g, about 12.9 g, or about 13 g of protein per 100 g of the composition. In another example, a composition may comprise about 11 g to about 13 g, about 11 g to about 12.5 g, about 11 g to about 12 g, about 11.5 g to about 13 g, about 11.5 g to about 12.5 g, or about 11.5 g to about 12 g protein per 100 g of the composition.

In each of the above embodiments, a composition may comprise about 20, about 21, about 22, about 23, about 24 or about 25 g of fat per 100 g of the composition. In another example, a composition may comprise about 26 g, about 27 g, about 28 g, about 29 g, or about 30 g of fat per 100 g of the composition. In another example, a composition may comprise about 20 g, about 20.1 g, about 20.2 g, about 20.3 g, about 20.4 g, about 20.5 g, about 20.6 g, about 20.7 g, about 20.8 g, about 20.9 g of fat per 100 g of the composition. In another example, a composition may comprise about 21 g, about 21.1 g, about 21.2 g, about 21.3 g, about 21.4 g, about 21.5 g, about 21.6 g, about 21.7 g, about 21.8 g, about 21.9 g, or about 22 g fat per 100 g of the composition. In another example, a composition may comprise about 20 g to about 22 g, about 20 g to about 21.5 g, about 20 g to about 21 g, about 20.5 g to about 22 g, about 20.5 g to about 21.5 g, or about 20.5 g to about 21 g fat per 100 g of the composition.

As used herein, the term “protein energy ratio” is an expression of the protein content of a composition, expressed as the proportion of the total energy provided by the protein content. In each of the above embodiments, a composition of the disclosure may have a PER of about 8%, about 8.5%, about 9%, about 9.5%, about 10%, about 10.5%, about 11° A, about 11.5%, or about 12%. In another example, a composition may have a PER of about 11.1%, about 11.2%, about 11.3%, about 11.4%, about 11.5%, about 11.6%, about 11.7%, about 11.8%, or about 11.9%. In another example, a composition of the disclosure may have a PER of about 8.5% to about 12%, about 9% to about 12%, about 9.5% to about 12%, about 10% to about 12%, or about 10.5% to about 12%. In another example, a composition may have a PER of about 11° A to about 12%, about 11.1% to about 12%, about 11.2% to about 12%, about 11.3% to about 12%, about 11.4% to about 12%, about 11.5% to about 12%, about 11.6% to about 12%. In another example, a composition may have a PER of about 11% to about 11.6%, about 11.1% to about 11.6%, about 11.2% to about 11.6%, about 11.3% to about 11.6%, or about 11.4% to about 11.6%. In another example, a composition may have a PER of about 11% to about 11.8%, about 11.1% to about 11.8%, about 11.2% to about 11.8%, about 11.3% to about 11.8%, or about 11.4% to about 11.8%. In another example, a composition may have a PER of about 12%, about 12.5%, about 13%, about 13.5%, about 14%, about 14.5% or about 15%. In another example, a composition may have a PER of about 15%, about 15.5%, about 16%, about 16.5%, about 17%, about 17.5%, about 18%, about 18.5%, about 19%, about 19.5%, or about 20%. In another example, a composition may have a PER of about 8% to about 20%, about 8% to about 15%, or about 8% to about 12%. In another example, a composition may have a PER of about 10% to about 20%, about 10% to about 15%, or about 10% to about 12%. In another example, a composition may have a PER of about 12% to about 20%, or about 12% to about 15%

As used herein, the term “fat energy ratio” is an expression of the fat content of a composition, expressed as the proportion of the total energy provided by the fat content. In each of the above embodiments, a composition may have a FER of about 30%, about 31%, about 32%, about 33%, about 34%, or about 35%. In each of the above embodiments, a composition may have a FER of about 35%, about 36%, about 37%, about 38%, about 39%, or about 40%. In another example, a composition may have a FER of about 40%, about 41%, about 42%, about 43%, about 44%, or about 45%. In another example, a composition may have a FER of about 45%, about 46%, about 47%, about 48%, about 49%, or about 50%. In another example, a composition may have a FER of about 51%, about 52%, about 53%, about 54%, or about 55%. In another example, a composition may have a FER of about 56%, about 57%, about 58%, about 59%, or about 60%. In another example, a composition may have a FER of about 45.5%, about 45.6%, about 45.7%, about 45.8%, about 45.9%, or about 46%. In another example, a composition may have a FER of about 46.1%, about 46.2%, about 46.3%, about 46.4%, about 46.5% about 46.6%, about 46.7%, about 46.8%, about 46.9%. In another example, a composition may have a FER of about 47%, about 47.1%, about 47.2% about 47.3%, about 47.4%, about 47.5%, about 47.6%, about 47.7%, about 47.8%, about 47.9%, or about 48%. In another example, a composition of the disclosure may have a FER of about 30% to about 50% or about 30% to about 45%.

In another example, a composition of the disclosure may have a FER of about 30% to about 40% or about 30% to about 35%. In another example, a composition of the disclosure may have a FER of about 35% to about 50% or about 35% to about 45%. In another example, a composition of the disclosure may have a FER of about 45% to about 55% or about 45% to about 50%. In another example, a composition may have a FER of about 46% to about 55% or about 46% to about 50%. In another example, a composition may have a FER of about 46% to about 48%, or about 46% to about 47%. In another example, a composition of the disclosure may have a FER of about 45.5% to about 48%, about 45.5% to about 47.5%, or about 45.5% to about 47%. In another example, a composition of the disclosure may have a FER of about 46% to about 47.5%, or about 46% to about 46.5%.

In each of the above embodiments, a composition may comprise a varying amount of carbohydrate. In one example, a composition may comprise about 15 g, about 15.1 g, about 15.2 g, about 15.3 g, about 15.4 g, or about 15.5 g of carbohydrate per 100 g of the composition, excluding added sugar. In another example, a composition may comprise about 15.6 g, about 15.7 g, about 15.8 g, about 15.9 g, or about 16 g of carbohydrate per 100 g of the composition, excluding added sugar. In one example, a composition may comprise about 16 g, about 16.1 g, about 16.2 g, about 16.3 g, about 16.4 g, about 16.5 g, or about 16.6 g of carbohydrate per 100 g of the composition, excluding added sugar. In one example, a composition may comprise about 16.5 g, about 16.6 g, about 16.7 g, about 16.8 g, about 16.9 g, or about 17 g of carbohydrate per 100 g of the composition, excluding added sugar. In one example, a composition may comprise about 17.1 g, about 17.2 g, about 17.3 g, about 17.4 g, about 17.5 g, about 17.6 g, about 17.7 g, about 17.8 g, about 17.9 g, about 18 g of carbohydrate per 100 g of the composition, excluding added sugar. In one example, a composition may comprise about 15 g to about 18 g, about 15 g to about 17.5 g, about 15 g to about 17 g, or about 15 g to about 16.5 g of carbohydrate per 100 g of the composition, excluding added sugar. In one example, a composition may comprise about 15.5 g to about 18 g, about 15.5 g to about 17.5 g, about 15.5 g to about 17 g, about 15.5 g to about 16.5 g of carbohydrate per 100 g of the composition, excluding added sugar. In one example, a composition may comprise about 16 g to about 18 g, about 16 g to about 17.5 g, about 16 g to about 17 g carbohydrate, excluding added sugar. When added sugar is included in the amount of carbohydrate, the value increases by about 27-28 grams. So, for instance, a composition with about 15 g to about 18 g carbohydrate, excluding added sugar, will have about 42 g to about 46 g of carbohydrate per 100 g of the composition when sugar is included. The term “total carbohydrate” is used herein to refer to a carbohydrate amount that includes added sugar.

In each of the above embodiments, a composition may comprise a varying amount of fiber. In one example, a composition may comprise about 3.5 g, about 3.6 g, about 3.7 g, about 3.8 g, about 3.9 g, or about 4 g of fiber per 100 g of composition. In another example, a composition may comprise about 4.1 g, about 4.2 g, about 4.3 g, about 4.4 g, about 4.5 g, about 4.6 g, about 4.7 g, about 4.8 g, or about 4.9 g of fiber per 100 g of composition. In another example, a composition may comprise about 5 g, about 5.1 g, about 5.2 g, about 5.3 g, about 5.4 g, or about 5.5 g of fiber per 100 g of composition. In another example, a composition may comprise about 3.5 g to about 5.5 g, about 3.5 g to about 5 g, about 3.5 g to about 4.5 g of fiber per 100 g of composition. In another example, a composition may comprise about 4 g to about 5.5 g, about 4 g to about 5 g, about 4 g to about 4.5 g, about 4.5 g to about 5.5 g, or about 4.5 g to about 5 g of fiber per 100 g of composition.

### (e) Additional Ingredients

Compositions of the present disclosure may further comprise one or more additional ingredient listed in Table E.

TABLE E

| Ingredients | What They Do | Names Found on Product Labels |
|---|---|---|
| Preservatives | Prevent food spoilage from | Ascorbic acid, citric acid, sodium benzoate, |
| | bacteria, molds, fungi, or yeast | calcium propionate, sodium erythorbate, sodium |
| | (antimicrobials); slow or prevent | nitrite, calcium sorbate, potassium sorbate, BHA, |
| | changes in color, flavor, or texture | BHT, EDTA, tocopherols (Vitamin E) |
| | and delay rancidity (antioxidants); | |
| | maintain freshness | |
| Sweeteners | Add sweetness with or without the | Sucrose (sugar), glucose, fructose, sorbitol, |
| | extra calories | mannitol, corn syrup, high fructose corn syrup, |
| | | saccharin, aspartame, sucralose, acesulfame |
| | | potassium (acesulfame-K), neotame |
| Color Additives | Offset color loss due to exposure | FD&C Blue Nos. 1 and 2, FD&C Green No. 3, |
| | to light, air, temperature extremes, | FD&C Red Nos. 3 and 40, FD&C Yellow Nos. 5 |
| | moisture and storage conditions; | and 6, Orange B, Citrus Red No. 2, annatto |
| | correct natural variations in color; | extract, beta-carotene, grape skin extract, |
| | enhance colors that occur | cochineal extract or carmine, paprika oleoresin, |
| | naturally; provide color to | caramel color, fruit and vegetable juices, saffron |
| | colorless and “fun” foods | (Note: Exempt color additives are not required to |
| | | be declared by name on labels but may be |
| | | declared simply as colorings or color added) |
| Flavors and | Add specific flavors (natural and | Natural flavoring, artificial flavor, and spices |
| Spices | synthetic) | |
| Flavor Enhancers | Enhance flavors already present | Monosodium glutamate (MSG), hydrolyzed soy |
| | in foods (without providing their | protein, autolyzed yeast extract, disodium |
| | own separate flavor) | guanylate or inosinate |
| Fat Replacers (and | Provide expected texture and a | Olestra, cellulose gel, carrageenan, |
| components of | creamy “mouth-feel” in reduced-fat | polydextrose, modified food starch, |
| formulations used | foods | microparticulated egg white protein, guar gum, |
| to replace fats) | | xanthan gum, whey protein concentrate |
| Nutrients | Replace vitamins and minerals | Thiamine hydrochloride, riboflavin (Vitamin B₂), |
| | lost in processing (enrichment), | niacin, niacinamide, folate or folic acid, beta |
| | add nutrients that may be lacking | carotene, potassium iodide, iron or ferrous |
| | in the diet (fortification) | sulfate, alpha tocopherols, ascorbic acid, Vitamin |
| | | D, amino acids (L-tryptophan, L-lysine, L-leucine, |
| | | L-methionine) |
| Emulsifiers | Allow smooth mixing of | Soy lecithin, mono- and diglycerides, egg yolks, |
| | ingredients, prevent separation | polysorbates, sorbitan monostearate |
| | Keep emulsified products stable, | |
| | reduce stickiness, control | |
| | crystallization, keep ingredients | |
| | dispersed, and to help products | |
| | dissolve more easily | |
| Stabilizers and | Produce uniform texture, improve | Gelatin, pectin, guar gum, carrageenan, xanthan |
| Thickeners, | “mouth-feel” | gum, whey |
| Binders, | | |
| Texturizers | | |
| pH Control Agents | Control acidity and alkalinity, | Lactic acid, citric acid, ammonium hydroxide, |
| and acidulants | prevent spoilage | sodium carbonate |
| Leavening Agents | Promote rising of baked goods | Baking soda, monocalcium phosphate, calcium |
| | | carbonate |
| Anti-caking agents | Keep powdered foods free- | Calcium silicate, iron ammonium citrate, silicon |
| | flowing, prevent moisture | dioxide |
| | absorption | |
| Humectants | Retain moisture | Glycerin, sorbitol |
| Firming Agents | Maintain crispness and firmness | Calcium chloride, calcium lactate |
| Enzyme | Modify proteins, polysaccharides | Enzymes, lactase, papain, rennet, chymosin |
| Preparations | and fats | |
| Gases | Serve as propellant, aerate, or | Carbon dioxide, nitrous oxide |
| | create carbonation | |

In some embodiments, a composition further comprises at least one sweetener. In one embodiment, a composition further comprises sugar (i.e. sucrose), and optionally one or more additional sweetener. The amount of sugar may vary. In one example, a composition comprises up to about 30 g of sugar per 100 g of the composition. In another example, a composition comprises about 0.1 g to about 30 g of sugar, or about 1 g to about 30 g of sugar, per 100 g of the composition. In another example, a composition comprises about 10 g to about 30 g of sugar per 100 g of the composition. In another example, a composition comprises about 20 g to about 30 g of sugar per 100 g of the composition. In another example, a composition comprises about 25 g to about 30 g of sugar per 100 g of the composition. In another example, a composition comprises about 27 g to about 30 g of sugar, or about 28 g to about 30 g of sugar, per 100 g of the composition. In another example, a composition comprises about 27 g, 27.1 g, 27.2 g, 27.3 g, 27.4 g, 27.5 g, 27.6 g, 27.7 g, 27.8 g, 27.9 g or 28 g of sugar per 100 g of the composition. In another example, a composition of the disclosure comprises about 28 g, 28.1 g, 28.2 g, 28.3 g, 28.4 g, 28.5 g, 28.6 g, 28.7 g, 28.8 g, 28.9 g or 29 g of sugar per 100 g of the composition. In another example, a composition of the disclosure comprises about 29 g, 29.1 g, 29.2 g, 29.3 g, 29.4 g, 29.5 g, 29.6 g, 29.7 g, 29.8 g, 29.9 g or 30 g of sugar per 100 g of the composition.

In some embodiments, a composition further comprises at least one fat. A fat may be an animal fat, or more preferably a vegetable oil. In some embodiments, a fat is chosen from avocado oil, canola oil, coconut oil, corn oil, cottonseed oil, flaxseed oil, grape seed oil, hemp seed oil, olive oil, palm oil, peanut oil, rice bran oil, safflower oil, soybean oil, or sunflower oil. In further embodiments, one fat provides at least 50% by weight (wt %) of the total fat in the composition. For instance, one fat may provide about 50%, about 55%, about 60%, about 65%, about 70%, about 75%, about 80%, about 85%, about 90%, or about 95% by weight of the total fat in the composition. In one example the fat is soybean oil. In one example the fat is canola oil. In still further embodiments, two or more fats provide at least 50% by weight of the fat in the composition. For instance, two or more fats may provide about 50%, about 55%, about 60%, about 65%, about 70%, about 75%, about 80%, about 85%, about 90%, or about 95% by weight of the total fat in the composition. In one example, at least one fat is soybean oil or canola oil. In one example, the fat is soybean oil and canola oil.

In other embodiments, a composition further comprises soybean oil, and the soybean oil provides at least 50% by weight of the total fat in the composition. In further embodiments, the soybean oil provides at least 75% by weight of the total fat in the composition. In still further embodiments, the soybean oil provides at least 90% by weight of the total weight of fat in the composition. In still further embodiments, the soybean oil provides at least 95% by weight of the total fat in the composition. In each of the above embodiments, the composition may further comprise a fat chosen from animal fat or vegetable oil.

In still other embodiments, a composition further comprises about 20 g of soybean oil. In one embodiment, a composition comprises about 15 g, about 16 g, about 17 g, about 18 g, about 19 g, about 20 g, or about 21 g of soybean oil per 100 g of the composition. In another embodiment, a composition further comprises about 15 g to about 21 g, about 16 g to about 21 g, about 17 g to about 21 g, about 18 g to about 21 g, about 19 g to about 21 g, about 20 g to about 21 g, about 15 g to about 20 g, about 16 g to about 20 g, about 17 g to about 20 g, about 18 g to about 20 g, or about 19 g to about 20 g of soybean oil per 100 g of the composition. In still another embodiment, a composition of the disclosure comprises about 17 g, 17.1 g, 17.2 g, 17.3 g, 17.4 g, 17.5 g, 17.6 g, 17.7 g, 17.8 g, 17.9 g or 18 g of soybean oil per 100 g of the composition. In still yet another embodiment, a composition of the disclosure comprises about 18 g, 18.1 g, 18.2 g, 18.3 g, 18.4 g, 18.5 g, 18.6 g, 18.7 g, 18.8 g, 18.9 g or 19 g of soybean oil per 100 g of the composition. In still yet another different embodiment, a composition further comprises about 19 g, 19.1 g, 19.2 g, 19.3 g, 19.4 g, 19.5 g, 19.6 g, 19.7 g, 19.8 g, 19.9 g or 20 g of soybean oil. In a different embodiment, a composition of the disclosure comprises about 20 g, 20.1 g, 20.2 g, 20.3 g, 20.4 g, 20.5 g, 20.6, 20.7 g, 20.8 g, 20.9 g or 21 g of soybean oil per 100 g of the composition.

### (f) Exemplary Compositions

In one embodiment, a composition of the present disclosure may contain (per 100 g) about 10 g chickpea flour or a glycan equivalent thereof, about 10 g peanut flour or a glycan equivalent thereof, about 8 g soy flour or a glycan equivalent thereof, about 19 g raw banana or a glycan equivalent thereof, about 29.9 g sugar, about 20 g soybean oil, and about 3.1 g micronutrient premix. In another embodiment, a composition of the present disclosure may contain (per 100 g) about 10 g chickpea flour, about 10 g peanut flour, about 8 g soy flour, about 19 g raw banana, about 29.9 g sugar, about 20 g soybean oil, and about 3.1 g micronutrient premix. In preferred embodiments, the micronutrient premix referenced in this paragraph contains the nutrients listed in Table C and Table D in the amount specified in Table C and Table D, respectively.

In some embodiments, a composition of the present disclosure as described in this section (Section V)), has total protein of about 11.6 g, total fat of about 20.8 g, total carbohydrate of about 46.2 g, and total fiber of about 4.5 g. For example, a composition of the present disclosure may contain (per 100 g) about 10 g chickpea flour or a glycan equivalent thereof, about 10 g peanut flour or a glycan equivalent thereof, about 8 g soy flour or a glycan equivalent thereof, about 19 g raw banana or a glycan equivalent thereof, about 29.9 g sugar, about 20 g soybean oil, and about 3.1 g micronutrient premix, and have total protein of about 11.6 g, total fat of about 20.8 g, total carbohydrate of about 46.2 g, and total fiber of about 4.5 g. In another example, a composition of the present disclosure may contain (per 100 g) about 10 g chickpea flour, about 10 g peanut flour, about 8 g soy flour, about 19 g raw banana, about 29.9 g sugar, about 20 g soybean oil, and about 3.1 g micronutrient premix, and have total protein of about 11.6 g, total fat of about 20.8 g, total carbohydrate of about 46.2 g, and total fiber of about 4.5 g. In preferred embodiments, the micronutrient premix referenced in this paragraph contains the nutrients listed in Table C and Table D in the amount specified in Table C and Table D, respectively.

In exemplary embodiments, a composition of the present disclosure as described in this section (Section V), has a protein energy ratio (PER) of about 11.4, a fat energy ratio (FER) of about 46.0, and total calories of about 400 to about 560 kcal per 100 g of the composition. For example, a composition of the present disclosure may contain (per 100 g) about 10 g chickpea flour or a glycan equivalent thereof, about 10 g peanut flour or a glycan equivalent thereof, about 8 g soy flour or a glycan equivalent thereof, about 19 g raw banana or a glycan equivalent thereof, about 29.9 g sugar, about 20 g soybean oil, and about 3.1 g micronutrient premix, wherein the composition has a protein energy ratio (PER) of about 11.4, a fat energy ratio (FER) of about 46.0, and total calories of about 400 to about 560 kcal per 100 g of the composition. In another example, a composition of the present disclosure may contain (per 100 g) about 10 g chickpea flour, about 10 g peanut flour, about 8 g soy flour, about 19 g raw banana, about 29.9 g sugar, about 20 g soybean oil, and about 3.1 g micronutrient premix, wherein the composition has a protein energy ratio (PER) of about 11.4, a fat energy ratio (FER) of about 46.0, and total calories of about 400 to about 560 kcal per 100 g of the composition. In yet another example, a composition of the present disclosure may contain (per 100 g) about 10 g chickpea flour or a glycan equivalent thereof, about 10 g peanut flour or a glycan equivalent thereof, about 8 g soy flour or a glycan equivalent thereof, about 19 g raw banana or a glycan equivalent thereof, about 29.9 g sugar, about 20 g soybean oil, and about 3.1 g micronutrient premix, and have total protein of about 11.6 g, total fat of about 20.8 g, total carbohydrate of about 46.2 g, and total fiber of about 4.5 g, wherein the composition has a protein energy ratio (PER) of about 11.4, a fat energy ratio (FER) of about 46.0, and total calories of about 400 to about 560 kcal per 100 g of the composition. In still another example, a composition of the present disclosure may contain (per 100 g) about 10 g chickpea flour, about 10 g peanut flour, about 8 g soy flour, about 19 g raw banana, about 29.9 g sugar, about 20 g soybean oil, and about 3.1 g micronutrient premix, and have total protein of about 11.6 g, total fat of about 20.8 g, total carbohydrate of about 46.2 g, and total fiber of about 4.5 g, wherein the composition has a protein energy ratio (PER) of about 11.4, a fat energy ratio (FER) of about 46.0, and total calories of about 400 to about 560 kcal per 100 g of the composition. In preferred embodiments, the micronutrient premix referenced in this paragraph contains the nutrients listed in Table C and Table D in the amount specified in Table C and Table D, respectively.

In exemplary embodiments, an edible composition comprising carbohydrates of the present disclosure increases abundance of nucleic acids encoding proteins in about 95%, 96%, 97%, 98%, 99%, or 100% of the CAZyme families indicated in Table A and decreases abundance of nucleic acids encoding proteins in about 95%, 96%, 97%, 98%, 99%, or 100% of the CAZyme families indicated in Table B in the gut microbiome of a subject, has a protein energy ratio (PER) of about 11.4, a fat energy ratio (FER) of about 46.0, and total calories of about 400 to about 560 kcal per 100 g of the composition. In additional exemplary embodiments, an edible composition comprising carbohydrates of the present disclosure increases abundance of nucleic acids encoding proteins in about 95%, 96%, 97%, 98%, 99%, or 100% of the CAZyme families indicated in Table A and decreases abundance of nucleic acids encoding proteins in about 95%, 96%, 97%, 98%, 99%, or 100% of the CAZyme families indicated in Table B in the gut microbiome of the subject, has a protein energy ratio (PER) of about 11.4, a fat energy ratio (FER) of about 46.0, and total calories of about 400 to about 560 kcal per 100 g of the composition, while having total protein of about 11.6 g, total fat of about 20.8 g, total carbohydrate of about 46.2 g, and total fiber of about 4.5 g. The edible compositions referenced in this paragraph may optionally include a micronutrient premix. In preferred embodiments, the micronutrient premix provides at least 60% of the recommended daily allowance for the age of the subject.

In exemplary embodiments, an edible composition comprising carbohydrates of the present disclosure modulates the relative abundances of at least 11, 12, 13, 14, 15, 16, 17, 18, 19, 20, 21, 22, or 23 WLZ-associated taxa of FIG. 18C in a statistically significant manner towards chronologically age-matched healthy subjects, has a protein energy ratio (PER) of about 11.4, a fat energy ratio (FER) of about 46.0, and total calories of about 400 to about 560 kcal per 100 g of the composition. In additional exemplary embodiments, an edible composition comprising carbohydrates of the present disclosure modulates the relative abundances of at least 11, 12, 13, 14, 15, 16, 17, 18, 19, 20, 21, 22, or 23 WLZ-associated taxa of FIG. 18C in a statistically significant manner towards chronologically age-matched healthy subjects, has a protein energy ratio (PER) of about 11.4, a fat energy ratio (FER) of about 46.0, and total calories of about 400 to about 560 kcal per 100 g of the composition, while having total protein of about 11.6 g, total fat of about 20.8 g, total carbohydrate of about 46.2 g, and total fiber of about 4.5 g. The edible compositions referenced in this paragraph may optionally include a micronutrient premix. In preferred embodiments, the micronutrient premix contains at least 60% of the recommended daily allowance for the age of the subject.

### (q) Repair of a Subject's Gut Microbiota

In certain embodiments, compositions of the present invention may repair the gut microbiota of a subject in need thereof and/or improve the subject's health.

The “health” of a subject's gut microbiota may be defined by relative abundances of microbial community members, expression of microbial genes, and/or biomarkers/mediators of gut barrier function. To “repair the gut microbiota of a subject,” which is synonymous with “improve gut microbiota health,” means to change the microbiota of a subject, in particular the relative abundances of age- and health-discriminatory taxa, in a statistically significant manner towards chronologically-age matched reference healthy subjects. The term encompasses complete repair (i.e., the measure of gut microbiota health does not deviate by 1.5 standard deviation or more) and levels of repair that are less than complete. The term also encompasses preventing or lessening a change in the relative abundances of age-and health-discriminatory taxa, wherein the change would have been significantly greater absent intervention. A subject with a gut microbiota in need of repair (e.g., a microbiota in “disrepair”, an “immature” gut microbiota, etc.) has a measure of gut microbiota health that deviates by 1.5 standard deviation or more (e.g., 2 std. deviation, 2.5 std. deviation, 3 std. deviation, etc.) from that of chronologically-age matched subjects, wherein the term “chronological age” means the amount of time a subject has lived from birth. Subjects five years or younger are grouped (or binned) by month. Subjects older than 5 years may be grouped by longer intervals of time (e.g., months or years). In some embodiments, a subject with a gut microbiota in need of repair is a subject with malnutrition, a subject at risk of malnutrition, a subject with a diarrheal disease, a subject recently treated for diarrheal disease (e.g., within 1 week, 2 weeks, 3 weeks, 4 weeks, 5 weeks, 6 weeks, 7 weeks, or 8 weeks), a subject recently treated with antibiotics (e.g., within 1 week, 2 weeks, 3 weeks, 4 weeks, 5 weeks, 6 weeks, 7 weeks, or 8 weeks), a subject undergoing treatment with an antibiotic, a subject who will be undergoing treatment with an antibiotic with about 1-4 weeks or about 1-2 weeks.

To “improve a subject's health” means to change one or more aspects of a subject's health in a statistically significant manner towards chronologically-age matched reference healthy subjects, as well as to prevent or lessen a change in one or more aspects of the subject's health wherein the change would have been significantly greater absent intervention. The improved aspect of the subject's health may be growth or rate of growth, for example as measured by a score on an anthropometric index; signs or symptoms of disease; relative abundances of health discriminatory plasma proteins, including but not limited to biomarkers/mediators of gut barrier function, bone growth, neurodevelopment, acute and inflammation, and the like. Those in need of treatment to improve their health include those already with a disease, condition, or disorder as well as those prone to have the disease, condition or disorder or those in which the disease, condition or disorder is to be prevented.

Further details may be found in Section III, which is incorporated by reference herein.

In a specific embodiment, 50 g of a composition per day, when administered for 1, 2 3, 4 weeks or more to a child that is 6 months of age or older with malnutrition, repairs the gut microbiota of the malnourished child. In some examples, a subject may be six months to five years of age, six months to 2 years of age, or six months to 18 months of age.

In a specific embodiment, 50 g of a composition per day, when administered for 1, 2 3, 4 weeks or more to a child that is 6 months of age or older with moderate malnutrition, repairs the gut microbiota of the malnourished child. In some examples, a subject may be six months to five years of age, six months to 2 years of age, or six months to 18 months of age.

In another specific embodiment, 100 g of a composition, when fed twice daily for at least 4 weeks to a child that is 6 months of age or older with moderate acute malnutrition and an immature gut microbiota, repairs the gut microbiota of the malnourished child.

In another specific embodiment, 100 g of a composition, when fed twice daily for at least 4 weeks to a child that is 6 months of age or older with moderate acute malnutrition and an immature gut microbiota, repairs the gut microbiota of the malnourished child as defined by microbiota-for-age Z score. In a further embodiment, the microbiota-for-age Z score is calculated from an RF-derived model comprising the abundances of F. prausnitzii (OTU 514940), Clostridiales sp. (OTU 1078587), B. longum (OTU 559527), S. aureus (OTU 1084865), D. longicatena (OTU 1111191), D. formicigenerans (OTU 1076587), Blautia sp. (OTU 370183), E. desmolans (OTU 551902), L. ruminis (OTU 1107027), Pasteurellaceae sp. (OTU 865469), Bifidobacterium sp. (OTU 997439), C. mitsuokai (OTU 330294), P. copri (OTU 840914), R. torques (OTU 369429), Clostridiales sp. (OTU 555945), Bifidobacterium sp. (OTU 484304), Actinomyces sp. (OTU 1108638), F. prausnitzii (OTU 514523), B. bifidum (OTU 365385), Ruminococcaceae sp. (OTU 367213), R. obeum (OTU 523934), S. thermophilus (OTU 579608), F. prausntizii (OTU 370287), Dialister sp. (OTU 583746), Streptococcus sp. (OTU 1083194), P. copri (OTU 588929), Bifidobacterium sp. (OTU 3528448), E. faecalis (OTU 1111582), Streptococcus sp. (OTU 349024), R. gnavus (summing relative abundance for all OTUs assigned to this species), and C. symbiosum (OTU 535601).

In another specific embodiment, 100 g of a composition, when fed twice daily for at least 4 weeks to a child that is 6 months of age or older with moderate acute malnutrition and an immature gut microbiota, repairs the gut microbiota of the malnourished child as defined by the co-variance of bacterial taxa in an ecogroup. In a further embodiment, the ecogroup comprises B. longum (OTU 559527), S. gallolyticus (OTU 349024), L. ruminis (OTU 1107027), Bifidobacterium (OTU 484304), F. prausnitzii (OTU 514940), E. coli (OTU 1111294), F. prausnitzii (OTU 851865), P. copri (OTU 588929), E. rectale (OTU 708680), Clostridiales (OTU 1078587), P. copri (OTU 840914), S. thermophilus (OTU 579608), Prevotella (OTU 591785), E. faecalis (OTU 1111582), and Dialister (OTU 583746).

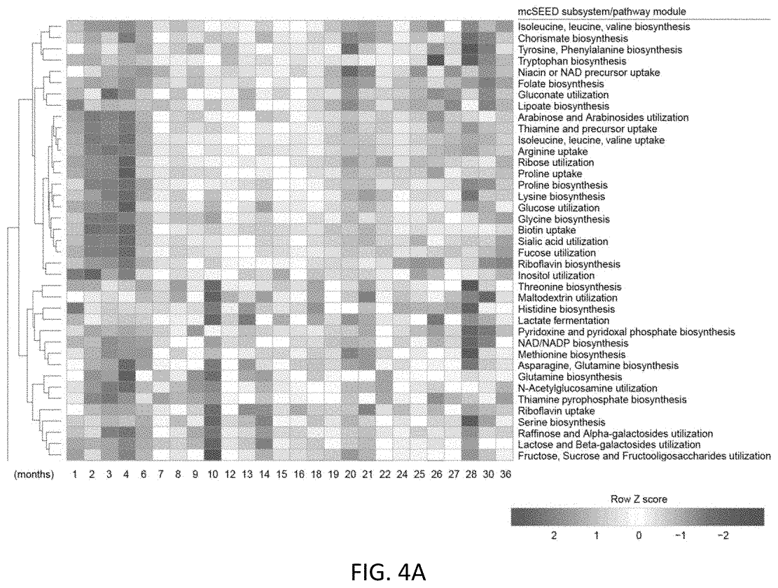

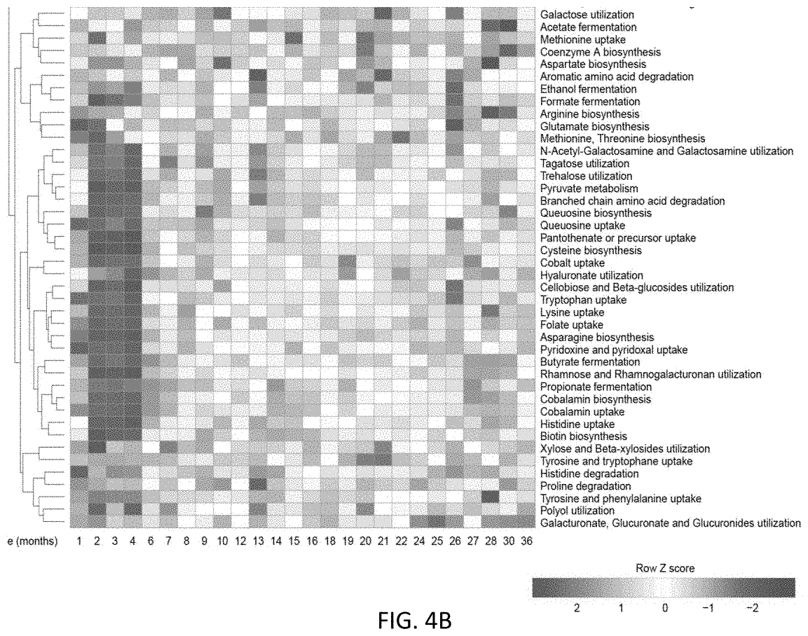

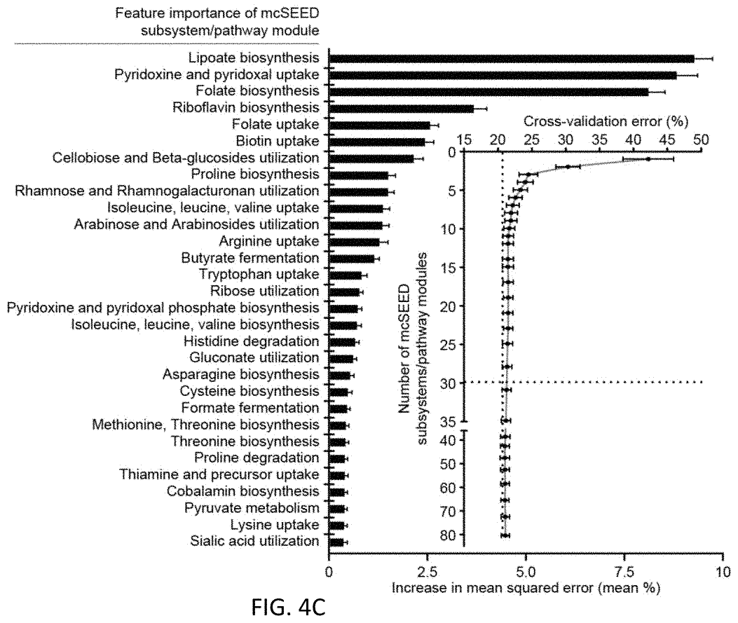

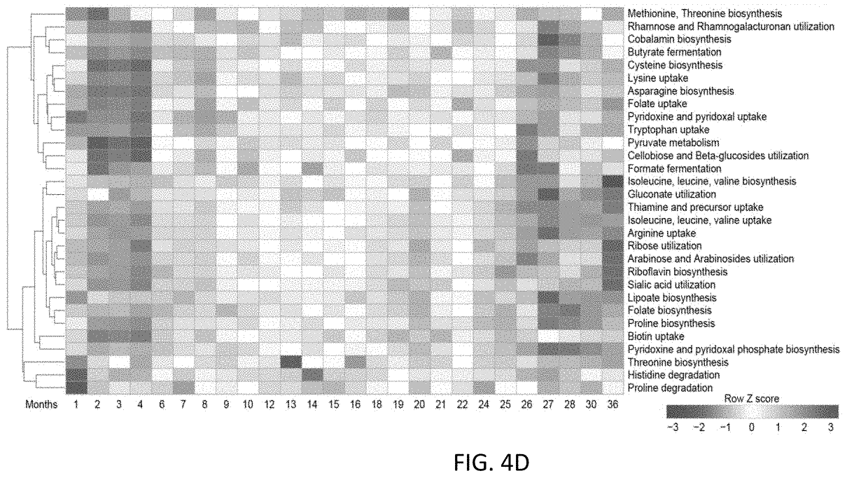

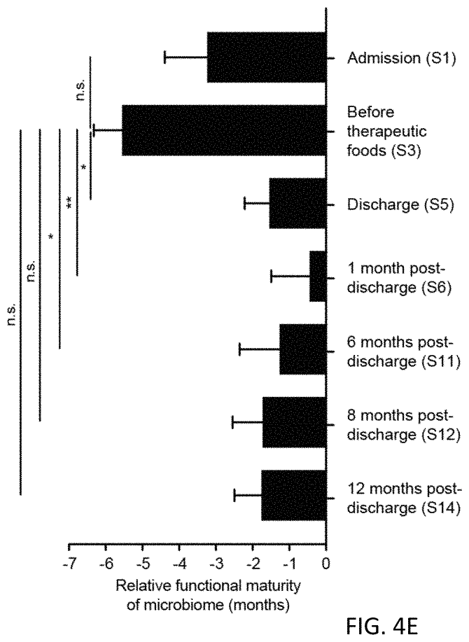

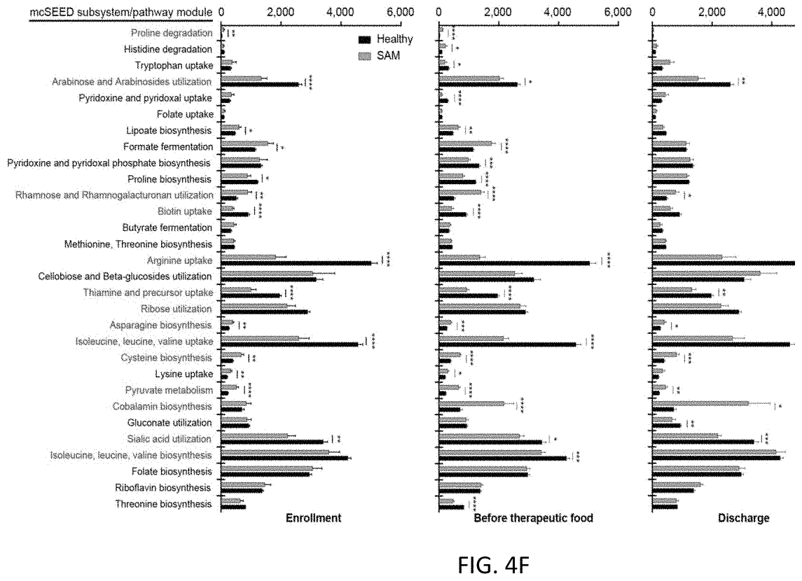

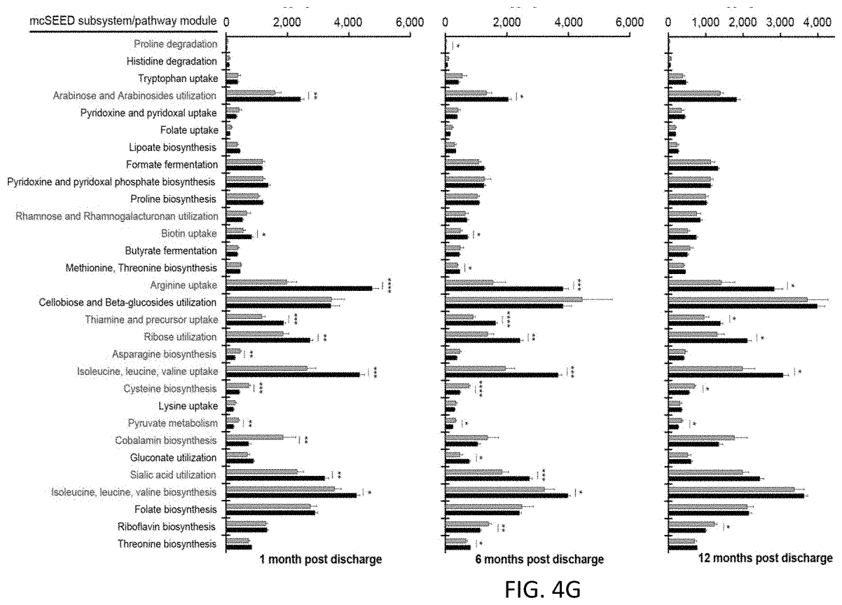

In another specific embodiment, 100 g of a composition, when fed twice daily for at least 4 weeks to a child that is 6 months of age or older with moderate acute malnutrition and an immature gut microbiota, repairs the gut microbiota of the malnourished child as defined by a statistically significant change, in a manner towards chronologically-age matched reference healthy children, in the relative abundance of one or more protein that map to pathways in the microbial communities SEED (mcSEED) database that are listed in FIG. 4A.

In another specific embodiment, 50 g of a composition per day, when administered for 1, 2 3, 4 weeks or more to a child that is 6 months of age or older with malnutrition, improves the growth of the malnourished child as defined by a statistically significant change in one or more anthropometric measurement in a manner towards chronologically-age matched reference healthy subjects. In a further embodiment, an anthropometric measurement is chosen from LAZ, WLZ, WAZ, or MUAC. In still a further embodiment, an anthropometric measurement is chosen from WLZ, WAZ, or MUAC. In still yet another embodiment, improvement in the child's growth is defined by a statistically significant change, in a manner towards healthy children of a similar chronological age, in (a) WLZ, WAZ, and MUAC; (b) WLZ and WAZ; (c) WAZ and MUAC; or (d) WLZ and MUAC. In each of the above embodiments, a subject may be six months to five years of age, six months to 2 years of age, or six months to 18 months of age.

In another specific embodiment, 100 g of a composition, when fed twice daily for at least 4 weeks to a child that is 6 months of age or older with moderate acute malnutrition and an immature gut microbiota, improves the growth of the malnourished child as defined by a statistically significant change in one or more anthropometric measurement in a manner towards chronologically-age matched reference healthy subjects. In a further embodiment, an anthropometric measurement is chosen from HAZ, WHZ, WAZ, or MUAC. In still a further embodiment, an anthropometric measurement is chosen from WHZ, WAZ, or MUAC. In still yet another embodiment, improvement in the child's growth is defined by a statistically significant change, in a manner towards healthy children of a similar chronological age, in (a) WHZ, WAZ, and MUAC; (b) WHZ and WAZ; (c) WAZ and MUAC; or (d) WHZ and MUAC.

In another specific embodiment, 50 g of a composition per day, when administered for 1, 2 3, 4 weeks or more to a child that is 6 months of age or older with malnutrition, improves the health of the malnourished child as defined by a statistically significant change in the relative abundance of one or more protein in Table 18, in a manner towards chronologically-age matched reference healthy children. In each of the above embodiments, a subject may be six months to five years of age, six months to 2 years of age, or six months to 18 months of age.

In another specific embodiment, 100 g of a composition, when fed twice daily for at least 4 weeks to a child that is 6 months of age or older with moderate acute malnutrition and an immature gut microbiota, improves the health of the malnourished child as defined by a statistically significant change, in a manner towards chronologically-age matched reference healthy children, in the relative abundance of one or more protein in Table F, one or more protein in Table G, or one or more protein in Table H.

Further details may be found in Section IV, which is incorporated by reference herein.

II. Methods for Treating and/or Preventing Malnutrition

In another aspect, the present disclosure provides methods for treating malnutrition in a subject in need thereof, the method comprising administering to the subject an effective amount of a composition of Section I. In a preferred embodiment, the composition is a composition of Section 1(f). In an exemplary embodiment, the composition is MDCF-2. Treating malnutrition refers to both therapeutic treatment, and prophylactic or preventative measures wherein the object is to slow down (lessen) or prevent an undesired physiological change. Methods for treating malnutrition disclosed herein provide measurable and beneficial effects for the subject as compared to lack of treatment and also to current standard of care (e.g., RUTF).

The aforementioned methods are not limited to subjects of a particular age, although suitable subjects are preferably able to eat some form of a solid food (e.g., a puree, a gel, a bar, etc.) in order to consume a composition of the disclosure. In one example, a subject may be at least six months of age. In another example, a subject may be eighteen years or younger. In still other examples, a subject may be ≤15 years, ≤14 years, ≤13 years, ≤12 years, ≤11 years, ≤10 years, ≤9 years, ≤8 years, ≤7 years, ≤6 years, ≤5 years, ≤4 years, ≤3 years, ≤2 years. In still other examples, a subject may be six months to five years of age, six months to 2 years of age, or six months to 18 months of age.

A subject in need of treatment for malnutrition may have a LAZ≤1, a MUAC a WAZ≤1, a WLZ≤1, deficiencies in vitamins and minerals, or any combination thereof. In some embodiments, a subject in need of treatment for malnutrition has a LAZ≤1, ≤2, or ≤3. In some embodiments, a subject in need of treatment for malnutrition has a MUAC≤1, ≤2, or ≤3. In some embodiments, a subject in need of treatment for malnutrition has a WAZ≤1, ≤2, or ≤3. In some embodiments, a subject in need of treatment for malnutrition has a WLZ≤1, ≤2, or ≤3. In some embodiments, a subject in need of treatment for malnutrition has a LAZ≤2, a MUAC≤2, a WAZ≤2, a WLZ≤2, or any combination thereof. In some embodiments, a subject in need of treatment for malnutrition has a WAZ≤1.5 and a WLZ≤1.5. In some embodiments, a subject in need of treatment for malnutrition has a WAZ≤2 and a WLZ≤2. In some embodiments, the subject has moderate acute malnutrition. In some embodiments, the subject has severe acute malnutrition.

In some embodiments, treating malnutrition comprises changing relative abundances of a plurality (e.g., 50% or more) of health discriminatory gut taxa in a statistically significant manner towards chronologically age-matched healthy subjects. “Health discriminatory gut taxa” are gut microbial strains significantly associated with a measurable indicator of health (e.g., weight, height, ponderal growth rate, biomarkers, etc.). As a non-limiting example, health discriminatory taxa may be gut microbial strains significantly associated with WLZ (“WLZ-associated taxa”). Methods for identifying WLZ-associated taxa are described in detail in the examples, and WLZ-associated taxa for subjects 6 months to 18 months are identified in FIG. 18C. The same approach, or a substantially similar approach, may be used to identify WLZ-associated taxa for other age groups and to identify other health discriminatory taxa including but not limited to gut microbial strains significantly associated with WAZ (“WAZ-associated taxa”), LAZ (“LAZ-associated taxa”), MUAC (“MUAC-associated taxa”), or any combination thereof.

In some embodiments, treating malnutrition may comprise changing relative abundances of at least 11 WLZ-associated taxa of FIG. 18C in a statistically significant manner towards chronologically age-matched healthy subjects. In further embodiments, treating malnutrition may comprise changing relative abundances of at least 11 WLZ-associated taxa of FIG. 18C in a statistically significant manner towards chronologically age-matched healthy subjects, wherein at least six of the taxa are ASV_9, ASV_13, ASV_15, ASV_14, ASV_1, and ASV_3. In still further embodiments, treating malnutrition may comprise changing relative abundances of at least 11 WLZ-associated taxa of FIG. 18C in a statistically significant manner towards chronologically age-matched healthy subjects, wherein at least seven of the taxa are ASV_41, ASV_236, ASV_22, ASV_31, ASV_13, ASV_37, and ASV_1. In still further embodiments, treating malnutrition may comprise changing relative abundances of at least 11 WLZ-associated taxa of FIG. 18C in a statistically significant manner towards chronologically age-matched healthy subjects, wherein at least five of the taxa are ASV_15, ASV_13, ASV_14, ASV_21, and ASV_377. In the above embodiments, treating may comprise changing relative abundances of 11, 12, 13, 14, 15, 16, or 17 WLZ-associated taxa in a statistically significant manner. Alternatively, treating may comprise changing relative abundances of 18, 19, 20, 21, 22, or 23 WLZ-associated taxa in a statistically significant manner. In exemplary embodiments, a subject may be six months to five years of age, six months to 2 years of age, or six months to 18 months of age.

In some embodiments, treating malnutrition may comprise changing relative abundances of health-discriminatory plasma proteins in a statistically significant manner towards chronologically age-matched healthy subjects. “Health-discriminatory plasma proteins” are proteins measurable in a plasma sample obtained from a subject that are significantly associated with a measurable indicator of health (e.g., weight, height, ponderal growth rate, etc.). As a non-limiting example, health-discriminatory plasma proteins may be plasma proteins significantly correlated (positively or negatively) with β-WLZ. Methods for identifying these proteins are described in detail in Example 7, and plasma proteins significantly correlated (positively or negatively) with β-WLZ following supplementation with MDCF-2 in subjects 6 months to 18 months with MAM are identified in Table 18. The same approach, or a substantially similar approach, may be used to identify plasma proteins significantly correlated with β-WLZ for other age groups and to identify other health-discriminatory plasma proteins including but not limited to plasma proteins positively or negatively correlated with β-WAZ, β-LAZ, β-MUAC, or any combination thereof.

In some embodiments, treating malnutrition may comprise changing relative abundances of a plurality of plasma proteins listed in Table 18 in a statistically significant manner towards chronologically age-matched healthy subjects. For a positively correlated plasma protein, treatment comprises increasing the protein's relative abundance. For a negatively correlated plasma protein, treatment comprises decreasing the protein's relative abundance. The plurality of plasma proteins changed may belong to same, or similar, “GO term”. “GO terms” are known in the art and further described in Example 7. For instance, treatment may result in increasing relative abundance of a plurality of plasma protein listed in Table 18 that are mediators of bone growth and ossification (e.g., COMP, SFRP4, LEP, IGF1, IGF acid-labile subunit, etc.) and/or CNS development (e.g., SLIT, SLITRK5, NTRK3, ROBO2, etc.). Alternatively or in addition, treatment may result in decreasing relative abundance of a plurality of plasma protein listed in Table 18 that are mediators of acute phase reactants and actuators of immune activation (e.g., HAMP, RANKL, GNLY, IFIT3, IGHA1, etc.). In exemplary embodiments, a subject may be six months to five years of age, six months to 2 years of age, or six months to 18 months of age.

In some embodiments, treating malnutrition may comprise a statistically significant increase (change towards zero) in LAZ, WAZ, WLZ, MUAC, or any combination thereof, as compared to untreated subjects or subjects treated with a current standard of care (e.g., RUTF). In further embodiments, treating malnutrition may comprise a statistically significant increase in WAZ and WLZ. In further embodiments, treating malnutrition may comprise a statistically significant increase in WAZ, WLZ, and MUAC. In exemplary embodiments, a subject may be six months to five years of age, six months to 2 years of age, or six months to 18 months of age.

In some embodiments, treating malnutrition may comprise a statistically significant increase in β-LAZ, β-WAZ, β-WLZ, β-MUAC, or any combination thereof, as compared to untreated subjects or subjects treated with a current standard of care (e.g., RUTF). In further embodiments, treating malnutrition may comprise a statistically significant increase in β-WAZ and β-WLZ. In further embodiments, treating malnutrition may comprise a statistically significant increase in β-WAZ, β-WLZ, and β-MUAC. In exemplary embodiments, a subject may be six months to five years of age, six months to 2 years of age, or six months to 18 months of age.

In some embodiments, treating malnutrition may comprise improving a symptom associated with malnutrition. Non-limiting examples of symptoms associated with malnutrition include fever, cough, rhinorrhea, diarrhea, tiredness, irritability, inability to concentrate, etc. In some embodiments, treating malnutrition may comprise improving a symptom associated with malnutrition selected from fever, cough, rhinorrhea, and diarrhea. In some embodiments, treating malnutrition may comprise improving a symptom associated with malnutrition selected from fever, cough, and rhinorrhea. In some embodiments, treating malnutrition may comprise improving a symptom associated with malnutrition selected from cough, and rhinorrhea. In exemplary embodiments, a subject may be six months to five years of age, six months to 2 years of age, or six months to 18 months of age.

A subject in need of malnutrition prevention may have a LAZ>1, a MUAC>1, a WAZ>1, a WLZ>1 or any combination thereof. In some embodiments, a subject in need of malnutrition prevention may have a LAZ less than zero but greater than one, a MUAC less than zero but greater than one, a WAZ less than zero but greater than one, a WLZ less than zero but greater than one, or any combination thereof. In further embodiments, a subject in need of malnutrition prevention may also have cultural, socionomic and/or economic risk factors that put the subject at risk for malnutrition, a family history of malnutrition, a genetic predisposition to malnutrition, or the like.

In some embodiments, preventing malnutrition comprises preventing or lessening a change in relative abundances of a plurality (e.g., 50% or more) of health discriminatory gut taxa, wherein the amount of change would have been significantly greater absent intervention. “Health discriminatory gut taxa” are described above.

In some embodiments, preventing malnutrition may comprise preventing or lessening a change in relative abundances of at least 11 WLZ-associated taxa of FIG. 18C, wherein the amount of change would have been significantly greater absent intervention. In further embodiments, preventing malnutrition may comprise preventing or lessening a change in abundances of at least 11 WLZ-associated taxa of FIG. 18C, wherein at least six of the taxa are ASV_9, ASV_13, ASV_15, ASV_14, ASV_1, and ASV_3, and wherein the amount of change would have been significantly greater absent intervention. In still further embodiments, preventing malnutrition may comprise preventing or lessening a change in relative abundances of at least 11 WLZ-associated taxa of FIG. 18C, wherein at least seven of the taxa are ASV_41, ASV_236, ASV_22, ASV_31, ASV_13, ASV_37, and ASV_1, and wherein the amount of change would have been significantly greater absent intervention. In still further embodiments, preventing malnutrition may comprise preventing or lessening a change in relative abundances of at least 11 WLZ-associated taxa of FIG. 18C, wherein at least five of the taxa are ASV_15, ASV_13, ASV_14, ASV_21, and ASV_377, and wherein the amount of change would have been significantly greater absent intervention. In the above embodiments, preventing may comprise preventing or lessening a change in relative abundances of 11, 12, 13, 14, 15, 16, or 17 WLZ-associated taxa. Alternatively, preventing may comprise preventing or lessening a change in relative abundances of 18, 19, 20, 21, 22, or 23 WLZ-associated taxa. In exemplary embodiments, a subject may be six months to five years of age, six months to 2 years of age, or six months to 18 months of age.

In some embodiments, preventing malnutrition may comprise preventing or lessening a change in relative abundances of health-discriminatory plasma proteins, wherein the amount of change would have been significantly greater absent intervention. “Health-discriminatory plasma proteins” are described above.

In some embodiments, preventing malnutrition may comprise preventing or lessening a change in relative abundances of a plurality of plasma proteins listed in Table 18, wherein the amount of change would have been significantly greater absent intervention. For a positively correlated plasma protein, preventing malnutrition may comprise preventing or lessening a decrease in the protein's relative abundance. For a negatively correlated plasma protein, preventing malnutrition may comprise preventing or lessening a change an increase in the protein's relative abundance. The plurality of plasma proteins changed may belong to same, or similar, “GO term”, as described above. In exemplary embodiments, a subject may be six months to five years of age, six months to 2 years of age, or six months to 18 months of age.

In some embodiments, preventing malnutrition may comprise preventing or lessening a decrease in LAZ, WAZ, WLZ, MUAC, or any combination thereof, wherein the amount of change would have been significantly greater absent intervention. In further embodiments, preventing malnutrition may comprise preventing or lessening a decrease in WAZ and WLZ, wherein the amount of change would have been significantly greater absent intervention. In further embodiments, preventing malnutrition may comprise preventing or lessening a decrease WAZ, WLZ, and MUAC, wherein the amount of change would have been significantly greater absent intervention. In exemplary embodiments, a subject may be six months to five years of age, six months to 2 years of age, or six months to 18 months of age.

In some embodiments, preventing malnutrition may comprise preventing or lessening a decrease in β-LAZ, β-WAZ, β-WLZ, β-MUAC, or any combination thereof, wherein the amount of change would have been significantly greater absent intervention. In further embodiments, preventing malnutrition may comprise preventing or lessening a decrease in β-WAZ and β-WLZ, wherein the amount of change would have been significantly greater absent intervention. In further embodiments, preventing malnutrition may comprise preventing or lessening a decrease in β-WAZ, β-WLZ, and β-MUAC, wherein the amount of change would have been significantly greater absent intervention. In exemplary embodiments, a subject may be six months to five years of age, six months to 2 years of age, or six months to 18 months of age.

In some embodiments, preventing malnutrition may comprise preventing the development or worsening of a symptom associated with malnutrition. Non-limiting examples of symptoms associated with malnutrition include fever, cough, rhinorrhea, diarrhea, tiredness, irritability, inability to concentrate, etc. In some embodiments, preventing malnutrition may comprise preventing the development or worsening of a symptom associated with malnutrition selected from fever, cough, rhinorrhea, and diarrhea. In some embodiments, preventing malnutrition may comprise preventing the development or worsening of a symptom associated with malnutrition selected from fever, cough, and rhinorrhea. In some embodiments, preventing malnutrition may comprise preventing the development or worsening of a symptom associated with malnutrition selected from cough, and rhinorrhea. In exemplary embodiments, a subject may be six months to five years of age, six months to 2 years of age, or six months to 18 months of age.

Typically, compositions of the present disclosure are administered orally. The amount of the composition administered can vary. For example, larger amounts may be administered for treatment of malnutrition as compared to preventing malnutrition. Amounts may also vary by age of the subject. For example, the energy needs from complementary foods (such as a composition of the present disclosure) for infants with “average” breast milk intake in developing countries (WHO/UNICEF, 1998) are approximately 200 kcal per day at 6-8 months of age, 300 kcal per day at 9-11 months of age, and 550 kcal per day at 12-23 months of age. In industrialized countries these estimates differ somewhat (130, 310 and 580 kcal/d at 6-8, 9-11 and 12-23 months respectively) because of differences in average breast milk intake. In various embodiments, compositions of the present disclosure may be administered per day in amounts ranging from about 10 g to about 1000 g (inclusive). In some embodiments, the amount administered per day may be about 10 g to about 1000 g, about 10 g to about 750 g, or about 10 g to about 500 g. In some embodiments, the amount administered per day may be about 10 g to about 500 g, about 10 g to about 300 g, or about 10 g to about 200 g. In some embodiments, the amount administered per day may be about 10 g to about 200 g, about 10 g to about 150 g, or about 10 g to about 100 g. In some embodiments, the amount administered per day may be about 30 g to about 200 g, about 30 g to about 150 g, or about 30 g to about 100 g. The daily amount of the composition may be administered as a single serving or may be divided into multiple servings and administered throughout the day.

The duration of treatment (i.e., administration of a composition of Section I) may vary depending upon a variety of factors, including the severity of malnutrition and the rate of improvement. Typically, a composition may be administered once or multiple times daily for at least one week, at least two weeks, at least three weeks, or at least four weeks. In some examples, a composition may be administered once or multiple times daily for about 1 month, about 2 months, about 3 months, about 4 months or more. In some examples, a composition may be administered once or multiple times daily for about 6 months, about 12 months, or more. In some examples, a composition may be administered once or multiple times daily for about 1 month to about 6 months. In some examples, a composition may be administered once or multiple times daily for about 6 months to about 12 months.

III. Methods for Repairing a Subject's Gut Microbiota and/or Improving a Subject's Health

In another aspect, the present disclosure provides methods for repairing a subject's gut microbiota and/or improving a subject's health, the method comprising administering to the subject an effective amount of a composition of Section I. In a preferred embodiment, the composition is a composition of Section 1(f). In an exemplary embodiment, the composition is MDCF-2. Compositions of the present disclosure can also be used prophylactically or preventatively to slow down (lessen) or prevent an undesired physiological change. Accordingly, in another aspect, the present disclosure provides methods to lessen or prevent disrepair of a subject's gut microbiota and/or to lessen or prevent a decline in a subject's health, the method comprising administering to the subject an effective amount of a composition of Section I. In preferred embodiments, the composition is a composition of Section 1(e). In exemplary embodiments, the composition is MDCF-2.

The aforementioned methods are not limited to subjects of a particular age, although suitable subjects are preferably able to eat some form of a solid food (e.g., a puree, a gel, a bar, etc.) in order to consume a composition of the disclosure. In one example, a subject may be at least six months of age. In another example, a subject may be eighteen years or younger. In still other examples, a subject may be 15 years, 14 years, 13 years, 12 years, 11 years, 10 years, 9 years, 8 years, 7 years, 6 years, 5 years, 4 years, 3 years, 2 years. In still other examples, a subject may be six months to five years of age, six months to 2 years of age, or six months to 18 months of age.

To “repair the gut microbiota of a subject” or to “improve gut microbiota health” means to change the microbiota of a subject, in particular the relative abundances of age- and health- discriminatory taxa, in a statistically significant manner towards chronologically-age matched reference healthy subjects, as well as to prevent or lessen a change in the relative abundances of age-and health-discriminatory taxa wherein the change would have been significantly greater absent intervention. In preferred embodiments, the microbiota of a subject is changed with regards to relative abundances of microbial community members and/or expression of microbial genes (e.g., microbial genes in mcSEED metabolic pathways, or microbial genes encoding CAZYMES). A subject with a gut microbiota in need of repair (e.g. a microbiota in “disrepair”, an “immature” gut microbiota, etc.) has a measure of gut microbiota health that deviates by 1.5 standard deviation or more (e.g. 2 std. deviation, 2.5 std. deviation, 3 std. deviation, etc.) from that of chronologically-age matched subjects, wherein the term “chronological age” means the amount of time a subject has lived from birth. Subjects five years or younger are grouped (or binned) by month. Subjects older than 5 years may be grouped by longer intervals of time. In some embodiments, a subject with a gut microbiota in need of repair is a subject with malnutrition, a subject at risk of malnutrition, a subject with a diarrheal disease, a subject recently treated for diarrheal disease (e.g., within 1 week, 2 weeks, 3 weeks, 4 weeks, 5 weeks, 6 weeks, 7 weeks, or 8 weeks), a subject recently treated with antibiotics (e.g., within 1 week, 2 weeks, 3 weeks, 4 weeks, 5 weeks, 6 weeks, 7 weeks, or 8 weeks), a subject undergoing treatment with an antibiotic, a subject who will be undergoing treatment with an antibiotic with about 1-4 weeks or about 1-2 weeks.

To “improve a subject's health” means to change one or more aspects of a subject's health in a statistically significant manner towards chronologically-age matched reference healthy subjects, as well as to prevent or lessen a change in one or more aspects of the subject's health wherein the change would have been significantly greater absent intervention. The improved aspect of the subject's health may be growth or rate of growth, for example as measured by a score on an anthropometric index; signs or symptoms of disease; relative abundances of health discriminatory plasma proteins, including but not limited to biomarkers/mediators of gut barrier function, bone growth, neurodevelopment, acute and inflammation, and the like. Those in need of treatment to improve their health include those already with a disease, condition, or disorder as well as those prone to have the disease, condition or disorder or those in which the disease, condition or disorder is to be prevented.

Typically, compositions of the present disclosure are administered orally. The amount of the composition administered can vary. For example, larger amounts may be administered for treatment of malnutrition as compared to preventing malnutrition. Amounts may also vary by age of the subject. For example, the energy needs from complementary foods (such as a composition of the present disclosure) for infants with “average” breast milk intake in developing countries (WHO/UNICEF, 1998) are approximately 200 kcal per day at 6-8 months of age, 300 kcal per day at 9-11 months of age, and 550 kcal per day at 12-23 months of age. In industrialized countries these estimates differ somewhat (130, 310 and 580 kcal/d at 6-8, 9-11 and 12-23 months respectively) because of differences in average breast milk intake. In various embodiments, compositions of the present disclosure may be administered per day in amounts ranging from about 10 g to about 1000 g (inclusive). In some embodiments, the amount administered per day may be about 10 g to about 1000 g, about 10 g to about 750 g, or about 10 g to about 500 g. In some embodiments, the amount administered per day may be about 10 g to about 500 g, about 10 g to about 300 g, or about 10 g to about 200 g. In some embodiments, the amount administered per day may be about 10 g to about 200 g, about 10 g to about 150 g, or about 10 g to about 100 g. In some embodiments, the amount administered per day may be about 30 g to about 200 g, about 30 g to about 150 g, or about 30 g to about 100 g. The daily amount of the composition may be administered as a single serving or may be divided into multiple servings and administered throughout the day.

The duration of treatment (i.e., administration of a composition of Section I) may vary depending upon a variety of factors, including the severity of disrepair and/or the health of the subject. For instance, as described in Example 7, the rate of response may differ among subjects. Accordingly, the duration of intervention may be adjusted (e.g. lengthened for poor responders) as needed. Typically, a composition may be administered once or multiple times daily for at least one week, at least two weeks, at least three weeks, or at least four weeks. In some examples, a composition may be administered once or multiple times daily for about 1 month, about 2 months, about 3 months, about 4 months or more. In some examples, a composition may be administered once or multiple times daily for about 6 months, about 12 months, or more. In some examples, a composition may be administered once or multiple times daily for about 1 month to about 6 months. In some examples, a composition may be administered once or multiple times daily for about 6 months to about 12 months.

In a specific embodiment, a method of the present disclosure comprises administering a composition of Section Ito a subject that is malnourished in an amount that provides a caloric density appropriate for the subject's age. In certain embodiments, the subject has moderate acute malnutrition (MAM). In certain embodiments, the subject has severe acute malnutrition (SAM). In one example, the malnourished subject may be eighteen years or younger. In another example, the malnourished subject may be fifteen years or younger. In another example, the malnourished subject may be ten years or younger. In another example, the malnourished subject may be nine years or younger. In another example, the malnourished subject may be eight years or younger. In another example, the malnourished subject may be seven years or younger. In another example, the malnourished subject may be six years or younger. In another example, the malnourished subject may be five years or younger. In another example, the malnourished subject may be six months to five years of age. The composition is administered at least once daily (e.g., once daily, twice daily, or more) for about 2 weeks, about 3 weeks, about 4 weeks, about 5 weeks, about 6 weeks, about 7 weeks, or about 8 weeks or more prior to measuring a statistically significant change in the subject's gut microbiota and/or health. In some examples, the composition is administered about 1 month, about 2 months, about 3 months, about 4 months, about 5 months, about 6 months, about 7 months, about 8 months, about 9 months, about 10 months, about 11 months or about 12 months prior to measuring a statistically significant change in the subject's gut microbiota and/or health. In a specific embodiment, the composition is administered at least 4 weeks. In another specific embodiment, the composition is administered at least 8 weeks. In another specific embodiment, the composition is administered at least 3 months. In another specific embodiment, the composition is administered at least 6 months. Treatment may or may not continue after a statistically significant change in the subject's health or gut microbiota occurs. In certain embodiments, a further change may not occur even if treatment is continued.

### (a) Repairing a Subject's Gut Microbiota

In some embodiments, repairing a subject's gut microbiota comprises changing relative abundances of a plurality (e.g., 50% or more) of health discriminatory gut taxa in a statistically significant manner towards chronologically age-matched healthy subjects. “Health discriminatory gut taxa” are gut microbial strains significantly associated with a measurable indicator of health (e.g., weight, height, ponderal growth rate, biomarkers, etc.). As a non-limiting example, health discriminatory taxa may be gut microbial strains significantly associated with WLZ (“WLZ-associated taxa”). Methods for identifying WLZ-associated taxa are described in detail in the examples, and WLZ-associated taxa for subjects 6 months to 18 months are identified in FIG. 18C. The same approach, or a substantially similar approach, may be used to identify WLZ-associated taxa for other age groups and to identify other health discriminatory taxa including but not limited to gut microbial strains significantly associated with WAZ (“WAZ-associated taxa”), LAZ (“LAZ-associated taxa”), MUAC (“MUAC-associated taxa”), or any combination thereof.

In some embodiments, repairing a subject's gut microbiota comprises changing relative abundances of at least 11 WLZ-associated taxa of FIG. 18C in a statistically significant manner towards chronologically age-matched healthy subjects. In further embodiments, repairing a subject's gut microbiota comprises changing may comprise changing relative abundances of at least 11 WLZ-associated taxa of FIG. 18C in a statistically significant manner towards chronologically age-matched healthy subjects, wherein at least six of the taxa are ASV_9, ASV_13, ASV_15, ASV_14, ASV_1, and ASV_3. In still further embodiments, repairing a subject's gut microbiota comprises changing may comprise changing relative abundances of at least 11 WLZ-associated taxa of FIG. 18C in a statistically significant manner towards chronologically age-matched healthy subjects, wherein at least seven of the taxa are ASV_41, ASV_236, ASV_22, ASV_31, ASV_13, ASV_37, and ASV_1. In the above embodiments, repairing a subject's gut microbiota may comprise changing relative abundances of 11, 12, 13, 14, 15, 16, or 17 WLZ-associated taxa in a statistically significant manner. Alternatively, repairing a subject's gut microbiota may comprise changing relative abundances of 18, 19, 20, 21, 22, or 23 WLZ-associated taxa in a statistically significant manner. In exemplary embodiments, a subject may be six months to five years of age, six months to 2 years of age, or six months to 18 months of age, and/or may be malnourished, may be at risk of malnutrition, have a diarrheal disease, have recently been treated for diarrheal disease (e.g., within 2 weeks, or within 1 week), have recently been treated with antibiotics (e.g., within 2 weeks, or within 1 week), or be or will be undergoing treatment with an antibiotic.

In some embodiments, repairing a subject's gut microbiota comprises preventing or lessening a change in relative abundances of a plurality (e.g., 50% or more) of health discriminatory gut taxa, wherein the amount of change would have been significantly greater absent intervention. “Health discriminatory gut taxa” are described above.

In some embodiments, repairing a subject's gut microbiota may comprise preventing or lessening a change in relative abundances of at least 11 WLZ-associated taxa of FIG. 18C, wherein the amount of change would have been significantly greater absent intervention. In further embodiments, repairing a subject's gut microbiota may comprise preventing or lessening a change in abundances of at least 11 WLZ-associated taxa of FIG. 18C, wherein at least six of the taxa are ASV_9, ASV_13, ASV_15, ASV_14, ASV_1, and ASV_3, and wherein the amount of change would have been significantly greater absent intervention. In still further embodiments, repairing a subject's gut microbiota may comprise preventing or lessening a change in relative abundances of at least 11 WLZ-associated taxa of FIG. 18C, wherein at least seven of the taxa are ASV_41, ASV_236, ASV_22, ASV_31, ASV_13, ASV_37, and ASV_1, and wherein the amount of change would have been significantly greater absent intervention. In still further embodiments, repairing a subject's gut microbiota may comprise preventing or lessening a change in relative abundances of at least 11 WLZ-associated taxa of FIG. 18C, wherein at least five of the taxa are ASV_15, ASV_13, ASV_14, ASV_21, and ASV_377, and wherein the amount of change would have been significantly greater absent intervention. In the above embodiments, preventing may comprise preventing or lessening a change in relative abundances of 11, 12, 13, 14, 15, 16, or 17 WLZ-associated taxa. Alternatively, preventing may comprise preventing or lessening a change in relative abundances of 18, 19, 20, 21, 22, or 23 WLZ-associated taxa. In exemplary embodiments, a subject may be six months to five years of age, six months to 2 years of age, or six months to 18 months of age.

In some embodiments, repairing a subject's gut microbiota comprises improving gut microbiota health as defined by relative abundances of microbial community members, in particular age-discriminatory taxa. For example, a measure of gut microbiota health may be a microbiota-for-age Z score (“MAZ-score”). A MAZ-score measures the deviation in development of a child's microbiota from that of chronologically-age matched reference healthy children based on the representation of the ensemble of age-discriminatory strains contained in a Random Forest (RF)-derived model. SuiTable Cge-discriminatory strains and their use to determine a MAZ-score are described in the Examples; in S. Subramanian, et al., “Persistent gut microbiota immaturity in malnourished Bangladeshi children,” Nature 510, 417-421 (2014); and in PCT Publication No. WO2015066625A1, the disclosures of which are incorporated by reference in their entirety. In one embodiment, the RF-derived model is as described in the Examples (e.g. Table 3). In another specific embodiment, a subject has malnutrition and the RF-derived model comprises F. prausnitzii (OTU 514940), Clostridiales sp. (OTU 1078587), B. longum (OTU 559527), S. aureus (OTU 1084865), D. longicatena (OTU 1111191), D. formicigenerans (OTU 1076587), Blautia sp. (OTU 370183), E. desmolans (OTU 551902), L. ruminis (OTU 1107027), Pasteurellaceae sp. (OTU 865469), Bifidobacterium sp. (OTU 997439), C. mitsuokai (OTU 330294), P. copri (OTU 840914), R. torques (OTU 369429), Clostridiales sp. (OTU 555945), Bifidobacterium sp. (OTU 484304), Actinomyces sp. (OTU 1108638), F. prausnitzii (OTU 514523), B. bifidum (OTU 365385), Ruminococcaceae sp. (OTU 367213), R. obeum (OTU 523934), S. thermophilus (OTU 579608), F. prausnitzii (OTU 370287), Dialister sp. (OTU 583746), Streptococcus sp. (OTU 1083194), P. copri (OTU 588929), Bifidobacterium sp. (OTU 3528448), E. faecalis (OTU 1111582), Streptococcus sp. (OTU 349024), R. gnavus (OTU summing relative abundance for all OTUs assigned to this species), and C. symbiosum (OTU 535601).

In another embodiment, repairing a subject's gut microbiota comprises improving a measure of gut microbiota health as defined by co-variance of microbial community members, in particular health-discriminatory taxa. As used herein, an “ecogroup” is a group of significantly co-varying bacterial taxa depending on the health status of a subject. In one example, a subject has malnutrition and the group of significantly co-varying bacterial taxa comprises at least 1, at least 2, at least 3, at least 4, at least 5, at least 6, at least 7, at least 8, at least 9, at least 10, at least 11, at least 12, at least 13, at least 14, or at least 15 bacterial taxa selected from the group consisting of B. longum, S. gallolyticus, L. ruminis, Bifidobacterium, F. prausnitzii, E. coli, P. copri, E. rectale, Clostridiales, S. thermophilus, Prevotella, E. faecalis, and Dialister, wherein a listed taxa may comprise more than one OTU. As used herein, an “OTU” or “operational taxonomic unit” is a group of organisms with 97% similarity by bacterial V4-16S rDNA. In another example, a subject has malnutrition and the group of significantly co-varying bacterial taxa comprises B. longum, S. gallolyticus, L. ruminis, Bifidobacterium, F. prausnitzii, E. coli, P. copri, E. rectale, Clostridiales, S. thermophilus, Prevotella, E. faecalis, and Dialister, wherein the listed taxa may comprise more than one OTU. In still another example, a subject has malnutrition and the group of significantly co-varying bacterial taxa comprises B. longum, S. gallolyticus, L. ruminis, Bifidobacterium, F. prausnitzii, E. coli, P. copri, E. rectale, Clostridiales, S. thermophilus, Prevotella, E. faecalis, and Dialister, wherein F. prausnitzii and P. copri comprise more than one OTU. In a specific embodiment, a subject has malnutrition and the group of significantly co-varying bacterial taxa comprises B. longum (OTU 559527), S. gallolyticus (OTU 349024), L. ruminis (OTU 1107027), Bifidobacterium (OTU 484304), F. prausnitzii (OTU 514940), E. coli (OTU 1111294), F. prausnitzii (OTU 851865), P. copri (OTU 588929), E. rectale (OTU 708680), Clostridiales (OTU 1078587), P. copri (OTU 840914), S. thermophilus (OTU 579608), Prevotella (OTU 591785), E. faecalis (OTU 1111582), and Dialister (OTU 583746). In an exemplary embodiment, the group of significantly co-varying bacterial taxa consists of B. longum (OTU 559527), S. gallolyticus (OTU 349024), L. ruminis (OTU 1107027), Bifidobacterium (OTU 484304), F. prausnitzii (OTU 514940), E. coli (OTU 1111294), F. prausnitzii (OTU 851865), P. copri (OTU 588929), E. rectale (OTU 708680), Clostridiales (OTU 1078587), P. copri (OTU 840914), S. thermophilus (OTU 579608), Prevotella (OTU 591785), E. faecalis (OTU 1111582), and Dialister (OTU 583746).

In some embodiments, repairing a subject's gut microbiota comprises improving gut microbiota health as defined by a measure a gut microbiota's functional maturity. For example, a measure of a gut microbiota's functional maturity may be based on the abundances of microbial genes that map to pathways in the microbial communities SEED (mcSEED) database that are listed in FIG. 4A, as detailed in the Examples. Information regarding mcSEED database can be found in R. Overbeek, R. Olson, G. D. Pusch, G. J. Olsen, J. J. Davis, T. Disz et al. The SEED and the Rapid Annotation of microbial genomes using Subsystems Technology (RAST). Nucleic Acids Res. 42, D206—D214 (2014).

### (b) Improving a Subject's Health

In some embodiments, improving a subject's health may comprise changing relative abundances of health-discriminatory plasma proteins. “Health-discriminatory plasma proteins” are proteins measurable in a plasma sample obtained from a subject that are significantly associated with a measurable indicator of health (e.g., weight, height, ponderal growth rate, etc.). As a non-limiting example, health-discriminatory plasma proteins may be plasma proteins significantly correlated (positively or negatively) with β-WLZ. Methods for identifying these proteins are described in detail in Example 7, and plasma proteins significantly correlated (positively or negatively) with β-WLZ following supplementation with MDCF-2 in subjects 6 months to 18 months with MAM are identified in Table 18. The same approach, or a substantially similar approach, may be used to identify plasma proteins significantly correlated with β-WLZ for other age groups and to identify other health-discriminatory plasma proteins including but not limited to plasma proteins positively or negatively correlated with β-WAZ, β-LAZ, β-MUAC, or any combination thereof.

In some embodiments, improving a subject's health may comprise a statistically significant change in relative abundances of a plurality of plasma proteins listed in Table 18. For a positively correlated plasma protein, treatment comprises increasing the protein's relative abundance. For a negatively correlated plasma protein, treatment comprises decreasing the protein's relative abundance. The plurality of plasma proteins changed may belong to same, or similar, “GO term”. “GO terms” are known in the art and further described in Example 7. For instance, treatment may result in increasing relative abundance of a plurality of plasma protein listed in Table 18 that are mediators of bone growth and ossification (e.g., COMP, SFRP4, LEP, IGF1, IGF acid-labile subunit, etc.) and/or CNS development (e.g., SLIT, SLITRK5, NTRK3, ROBO2, etc.). Alternatively or in addition, treatment may result in decreasing relative abundance of a plurality of plasma protein listed in Table 18 that are mediators of acute phase reactants and actuators of immune activation (e.g., HAMP, RANKL, GNLY, IFIT3, IGHA1, etc.). In exemplary embodiments, a subject may be six months to five years of age, six months to 2 years of age, or six months to 18 months of age.

In some embodiments, improving a subject's health may comprise preventing or lessening a change in relative abundances of health-discriminatory plasma proteins, wherein the amount of change would have been significantly greater absent intervention. “Health-discriminatory plasma proteins” are described above.

In some embodiments, improving a subject's health may comprise preventing or lessening a change in relative abundances of a plurality of plasma proteins listed in Table 18, wherein the amount of change would have been significantly greater absent intervention. For a positively correlated plasma protein, improving a subject's health may comprise preventing or lessening a decrease in the protein's relative abundance. For a negatively correlated plasma protein, improving a subject's health may comprise preventing or lessening a change an increase in the protein's relative abundance. The plurality of plasma proteins changed may belong to same, or similar, “GO term”, as described above. In exemplary embodiments, a subject may be six months to five years of age, six months to 2 years of age, or six months to 18 months of age.

In some embodiments, a subject's health is improved, as defined by a statistically significant change in the relative abundances of health discriminatory plasma proteins, and/or biomarkers/mediators of gut barrier function, in a manner towards chronologically-age matched reference healthy subjects.

In one example, a subject is malnourished and the subject's health is improved, as defined by a statistically significant change in the relative abundance of one or more protein in Table F, in a manner towards chronologically-age matched reference healthy subjects. In some embodiments, a statistically significant change occurs in the relative abundance of about 10%, about 20%, about 25%, about 30%, about 40%, or about 50% of the protein in Table F. In other embodiments, a statistically significant change occurs in the relative abundance of about 60%, about 70%, about 75%, about 80%, about 90%, or about 1000% of the protein in Table F. In other embodiments, a statistically significant change occurs in the relative abundance of about 50% to about 100% of the proteins in Table F. In a specific embodiment, the subject has MAM or SAM. In further embodiments, the subjects is a child 6 months in age or older.

TABLE F
Plasma proteins with significant fold-changes in abundance following
administration of a composition of the disclosure for about 4 weeks.

| UniProt | Entrez Gene ID | Entrez Gene Symbol | Name |
|---|---|---|---|
| P02776 | 5196 | PF4 | Platelet factor 4 |
| P21741 | 4192 | MDK | Midkine |
| Q8N474 | 6422 | SFRP1 | Secreted frizzled-related protein 1 |
| P0C0S5 | 3015 | H2AFZ | Histone H2A.z |
| P02649 | 348 | APOE | Apolipoprotein E |
| P23280 | 765 | CA6 | Carbonic anhydrase 6 |
| P45452 | 4322 | MMP13 | Collagenase 3 |
| O60259 | 11202 | KLK8 | Kallikrein-8 |
| P02649 | 348 | APOE | Apolipoprotein E (isoform E3) |
| P26842 | 939 | CD27 | CD27 antigen |
| P28325 | 1473 | CST5 | Cystatin-D |
| O95825 | 9946 | CRYZL1 | Quinone oxidoreductase-like protein 1 |
| P41159 | 3952 | LEP | Leptin |
| P22004 | 654 | BMP6 | Bone morphogenetic protein 6 |
| Q9NPY3 | 22918 | CD93 | Complement component C1q receptor |
| P02649 | 348 | APOE | Apolipoprotein E (isoform E4) |
| P07949 | 5979 | RET | Proto-oncogene tyrosine-protein kinase |
| | | | receptor Ret |
| Q9UHX3 | 30817 | ADGRE2 | Adhesion G protein-coupled receptor E2 |
| O43155 | 23768 | FLRT2 | Leucine-rich repeat transmembrane |
| | | | protein FLRT2 |
| Q9UHF5 | 27190 | IL17B | Interleukin-17B |
| Q4KMG0 | 50937 | CDON | Cell adhesion molecule-related/down- |
| | | | regulated by oncogenes |
| Q15582 | 7045 | TGFBI | Transforming growth factor-beta-induced |
| | | | protein ig-h3 |
| P00749 | 5328 | PLAU | Urokinase-type plasminogen activator |
| Q96KN2 | 84735 | CNDP1 | Beta-Ala-His dipeptidase |
| P24593 | 3488 | IGFBP5 | Insulin-like growth factor-binding protein 5 |
| Q9UBG0 | 9902 | MRC2 | C-type mannose receptor 2 |
| Q9BU40 | 91851 | CHRDL1 | Chordin-like protein 1 |
| Q15648 | 5469 | MED1 | Mediator of RNA polymerase II |
| | | | transcription subunit 1 |
| P02649 | 348 | APOE | Apolipoprotein E (isoform E2) |
| P33151 | 1003 | CDH5 | Cadherin-5 |
| O14625 | 6373 | CXCL11 | C-X-C motif chemokine 11 |
| P62826 | 5901 | RAN | GTP-binding nuclear protein Ran |
| Q08752 | 5481 | PPID | Peptidyl-prolyl cis-trans isomerase D |
| P19784 | 1459 | CSNK2A2 | Casein kinase ∥ 2-alpha′:2-beta |
| P67870 | 1460 | CSNK2B | heterotetramer |
| Q9Y259 | 1120 | CHKB | Choline/ethanolamine kinase |
| Q9NYA1 | 8877 | SPHK1 | Sphingosine kinase 1 |
| P29965 | 959 | CD40LG | CD40 ligand |
| P68402 | 5049 | PAFAH1B2 | Platelet-activating factor acetylhydrolase |
| | | | IB subunit beta |
| P01011 | 0 | SERPINA3 | Alpha-1-antichymotrypsin complex |
| P07384 | 823 | CAPN1 | Calpain I |
| P04632 | 826 | CAPNS1 | |
| P43630 | 3812 | KIR3DL2 | Killer cell immunoglobulin-like receptor 3DL2 |
| O95727 | 56253 | CRTAM | Cytotoxic and regulatory T-cell molecule |
| O75594 | 8993 | PGLYRP1 | Peptidoglycan recognition protein 1 |
| Q05397 | 5747 | PTK2 | Focal adhesion kinase 1 |
| Q16719 | 8942 | KYNU | Kynureninase |
| Q99988 | 9518 | GDF15 | Growth/differentiation factor 15 |
| P49767 | 7424 | VEGFC | Vascular endothelial growth factor C |
| P29279 | 1490 | CTGF | Connective tissue growth factor |
| P55957 | 637 | BID | BH3-interacting domain death agonist |
| P18093 | 1724716 | Human-virus | Protein Rev_HV2BE |

In another example, a subject is malnourished and the subject's health is improved, as defined by a statistically significant increase in the relative abundance of one or more protein in Table G, in a manner towards chronologically-age matched reference healthy subjects. In some embodiments, a statistically significant increase occurs in the relative abundance of about 10%, about 20%, about 25%, about 30%, about 40%, or about 50% of the protein in Table G. In other embodiments, a statistically significant increase occurs in the relative abundance of about 60%, about 70%, about 75%, about 80%, about 90%, or about 1000% of the protein in Table G. In other embodiments, a statistically significant increase occurs in the relative abundance of about 50% to about 100% of the proteins in Table G. In a specific embodiment, the subject has MAM or SAM. In further embodiments, the subjects is a child 6 months in age or older.

In another example, a subject is malnourished and the subject's health is improved, as defined by a statistically significant decrease in the relative abundance of one or more protein in Table H, in a manner towards chronologically-age matched reference healthy subjects. In some embodiments, a statistically significant decrease occurs in the relative abundance of about 10%, about 20%, about 25%, about 30%, about 40%, or about 50% of the protein in Table H. In other embodiments, a statistically significant decrease occurs in the relative abundance of about 60%, about 70%, about 75%, about 80%, about 90%, or about 1000% of the protein in Table H. In other embodiments, a statistically significant decrease occurs in the relative abundance of about 50% to about 100% of the proteins in Table H. In a specific embodiment, the subject has MAM or SAM. In further embodiments, the subjects is a child 6 months in age or older.

TABLE G
Plasma proteins that are significantly higher in their abundances
in healthy children compared to those with SAM.

| | Entrez | Entrez Gene | |
|---|---|---|---|
| UniProt | Gene ID | Symbol | Name |
| Q9HBG7 | 4063 | LY9 | T-lymphocyte surface antigen Ly-9 |
| P10451 | 6696 | SPP1 | Osteopontin |
| Q9H773 | 79077 | DCTPP1 | dCTP pyrophosphatase 1 |
| P01374, | 4049 | LTA | Lymphotoxin alpha1:beta2 |
| Q06643 | 4050 | LTB | |
| P21709 | 2041 | EPHA1 | Ephrin type-A receptor 1 |
| Q969J5 | 116379 | IL22RA2 | Interleukin-22 receptor subunit |
| | | | alpha-2 |
| O94991 | 26050 | SLITRK5 | SLIT and NTRK-like protein 5 |
| P14778 | 3554 | IL1R1 | Interleukin-1 receptor type 1 |
| P08476 | 3624 | INHBA | Inhibin beta A chain |
| P52799 | 1948 | EFNB2 | Ephrin-B2 |
| Q9H2E6 | 57556 | SEMA6A | Semaphorin-6A |
| P21810 | 633 | BGN | Biglycan |
| P17813 | 2022 | ENG | Endoglin |
| P16112 | 176 | ACAN | Aggrecan core protein |
| P27918 | 5199 | CFP | Properdin |
| P17181 | 3454 | IFNAR1 | Interferon alpha/beta receptor 1 |
| P04083 | 301 | ANXA1 | Annexin A1 |
| Q9BWV1 | 91653 | BOC | Brother of CDO |
| P01215 | 1081 | CGA | Thyroid Stimulating Hormone |
| P01222 | 12372 | TSHB | |
| O60259 | 11202 | KLK8 | Kallikrein-8 |
| P23280 | 765 | CA6 | Carbonic anhydrase 6 |
| Q6YHK3 | 135228 | CD109 | CD109 antigen |
| P09619 | 5159 | PDGFRB | Platelet-derived growth factor |
| | | | receptor beta |
| P13671 | 729 | C6 | Complement component C6 |
| P45985 | 6416 | MAP2K4 | Dual specificity mitogen-activated |
| | | | protein kinase kinase 4 |
| Q15465 | 6469 | SHH | Sonic hedgehog protein |
| O15444 | 6370 | CCL25 | C-C motif chemokine 25 |
| P22079 | 4025 | LPO | Lactoperoxidase |
| P07355 | 302 | ANXA2 | Annexin A2 |
| Q02083 | 27163 | NAAA | N-acylethanolamine-hydrolyzing |
| | | | acid amidase |
| Q15181 | 5464 | PPA1 | Inorganic pyrophosphatase |
| P09104 | 2026 | ENO2 | Gamma-enolase |
| P17612 | 5566 | PRKACA | cAMP-dependent protein kinase |
| | | | catalytic subunit alpha |
| Q13219 | 5069 | PAPPA | Pappalysin-1 |
| P28325 | 1473 | CST5 | Cystatin-D |
| Q14974 | 3837 | KPNB1 | Importin subunit beta-1 |
| P22304 | 3423 | IDS | Iduronate 2-sulfatase |
| Q16288 | 4916 | NTRK3 | NT-3 growth factor receptor |
| P15586 | 2799 | GNS | N-acetylglucosamine-6-sulfatase |
| P22748 | 762 | CA4 | Carbonic anhydrase 4 |
| Q9H3T3 | 10501 | SEMA6B | Semaphorin-6B |
| Q9BY67 | 23705 | CADM1 | Cell adhesion molecule 1 |
| P29460, | 3593 | IL12B | Interleukin-23 |
| Q9NPF7 | 51561 | IL23A | |
| O43278 | 6692 | SPINT1 | Kunitz-type protease inhibitor 1 |
| Q9NZU0 | 23767 | FLRT3 | Leucine-rich repeat transmembrane |
| | | | protein FLRT3 |
| P09238 | 4319 | MMP10 | Stromelysin-2 |
| P05090 | 347 | APOD | Apolipoprotein D |
| P10768 | 2098 | ESD | S-formylglutathione hydrolase |
| Q8NHW4 | 388372 | CCL4L1 | C-C motif chemokine 4-like |
| P41182 | 604 | BCL6 | B-cell lymphoma 6 protein |

TABLE H
Plasma proteins that are significantly higher in their abundances
in children with SAM compared to healthy children.

| | Entrez | Entrez Gene | |
|---|---|---|---|
| UniProt | Gene ID | Symbol | Name |
| P18669 | 5223 | PGAM1 | Phosphoglycerate mutase 1 |
| P68400 | 1457 | CSNK2A1 | Casein kinase II 2-alpha:2-beta |
| P67870 | 1460 | CSNK2B | heterotetramer |
| P02776 | 5196 | PF4 | Platelet factor 4 |
| P67936 | 7171 | TPM4 | Tropomyosin alpha-4 chain |
| Q9NQU5 | 56924 | PAK6 | Serine/threonine-protein kinase PAK |
| | | | 6 |
| P29350 | 5777 | PTPN6 | Tyrosine-protein phosphatase non- |
| | | | receptor type 6 |
| Q01638 | 9173 | IL1RL1 | Interleukin-1 receptor-like 1 |
| Q16778 | 8349 | HIST2H2BE | Histone H2B type 2-E |
| P18065 | 3485 | IGFBP2 | Insulin-like growth factor-binding |
| | | | protein 2 |
| P21741 | 4192 | MDK | Midkine |
| Q9UK53 | 3621 | ING1 | Inhibitor of growth protein 1 |
| P04792 | 3315 | HSPB1 | Heat shock protein beta-1 |
| P42224 | 6772 | STAT1 | Signal transducer and activator of |
| | | | transcription 1-alpha/beta |
| P05451 | 5967 | REG1A | Lithostathine-1-alpha |
| P48047 | 539 | ATP5O | ATP synthase subunit O, |
| | | | mitochondrial |
| P31946, | 7529, | YWHAB, | 14-3-3 protein family |
| P62258, | 7531, | YWHAE, | |
| P61981, | 7532, | YWHAG, | |
| Q04917, | 7533, | YWHAH, | |
| P27348, | 10971, | YWHAQ, | |
| P63104, | 7534, | YWHAZ, | |
| P31947 | 2810 | SFN | |
| Q99988 | 9518 | GDF15 | Growth/differentiation factor 15 |
| O95219 | 8723 | SNX4 | Sorting nexin-4 |
| P56211 | 10776 | ARPP19 | cAMP-regulated phosphoprotein 19 |
| P13693 | 7178 | TPT1 | Translationally-controlled tumor |
| | | | protein |
| P61088 | 7334 | UBE2N | Ubiquitin-conjugating enzyme E2 N |
| P05771 | 5579 | PRKCB | Protein kinase C beta type (splice |
| | | | variant beta-II) |
| O75144 | 23308 | ICOSLG | ICOS ligand |
| P08133 | 309 | ANXA6 | Annexin A6 |
| Q9NP79 | 51534 | VTA1 | Vacuolar protein sorting-associated |
| | | | protein VTA1 homolog |
| P63104 | 7534 | YWHAZ | 14-3-3 protein zeta/delta |
| P31946 | 7529 | YWHAB | 14-3-3 protein beta/alpha |
| Q08ET2 | 100049587 | SIGLEC14 | Sialic acid-binding Ig-like lectin 14 |
| P60174 | 7167 | TPI1 | Triosephosphate isomerase |
| P30041 | 9588 | PRDX6 | Peroxiredoxin-6 |
| Q7Z4V5 | 84717 | HDGFRP2 | Hepatoma-derived growth factor- |
| | | | related protein 2 |
| P63241 | 1984 | EIF5A | Eukaryotic translation initiation |
| | | | factor 5A-1 |
| P37802 | 8407 | TAGLN2 | Transgelin-2 |
| Q9UQ80 | 5036 | PA2G4 | Proliferation-associated protein 2G4 |
| P07478 | 5645 | PRSS2 | Trypsin-2 |
| Q9HCN6 | 51206 | GP6 | Platelet glycoprotein VI |
| P0C0L4 | 720 | C4A | Complement C4b |
| P0C0L5 | 721 | C4B | |
| Q7L7L0 | 0 | HIST3H2A | Histone H2A type 3 |
| P11171 | 2035 | EPB41 | Protein 4.1 |
| P54920 | 8775 | NAPA | Alpha-soluble NSF attachment |
| | | | protein |
| O00299 | 1192 | CLIC1 | Chloride intracellular channel |
| | | | protein 1 |
| P55854 | 6613 | SUMO3 | Small ubiquitin-related modifier 3 |
| O43488 | 8574 | AKR7A2 | Aflatoxin B1 aldehyde reductase |
| | | | member 2 |
| P29401 | 7086 | TKT | Transketolase |
| P01009 | 5265 | SERPINA1 | Alpha-1-antitrypsin |
| Q9Y3A5 | 51119 | SBDS | Ribosome maturation protein SBDS |
| P30086 | 5037 | PEBP1 | Phosphatidylethanolamine-binding |
| | | | protein 1 |
| O43320 | 8823 | FGF16 | Fibroblast growth factor 16 |
| P62979 | 6233 | RPS27A | Ubiquitin + 1, truncated mutation for |
| | | | UbB |
| P15514 | 374 | AREG | Amphiregulin |

In some embodiments, improving a subject's health may comprise a statistically significant increase (changing towards zero) in LAZ, WAZ, WLZ, MUAC, or any combination thereof. In further embodiments, improving a subject's health may comprise increasing WAZ and WLZ. In further embodiments, improving a subject's health may comprise increasing WAZ, WLZ, and MUAC. In exemplary embodiments, a subject may be six months to five years of age, six months to 2 years of age, or six months to 18 months of age.

In some embodiments, improving a subject's health may comprise a statistically significant increase (changing towards zero) in β-LAZ, β-WAZ, β-WLZ, β-MUAC, or any combination thereof. In further embodiments, treating malnutrition may comprise increasing β-WAZ and β-WLZ. In further embodiments, treating malnutrition may comprise increasing β-WAZ, β-WLZ, and β-MUAC. In exemplary embodiments, a subject may be six months to five years of age, six months to 2 years of age, or six months to 18 months of age.

In some embodiments, improving a subject's health may comprise improving a symptom associated with malnutrition. Non-limiting examples of symptoms associated with malnutrition include fever, cough, rhinorrhea, diarrhea, tiredness, irritability, inability to concentrate, etc. In some embodiments, treating malnutrition may comprise improving a symptom associated with malnutrition selected from fever, cough, rhinorrhea, and diarrhea. In some embodiments, treating malnutrition may comprise improving a symptom associated with malnutrition selected from fever, cough, and rhinorrhea. In some embodiments, treating malnutrition may comprise improving a symptom associated with malnutrition selected from cough, and rhinorrhea. In exemplary embodiments, a subject may be six months to five years of age, six months to 2 years of age, or six months to 18 months of age.

In some embodiments, improving a subject's health may comprise preventing or lessening a decrease in LAZ, WAZ, WLZ, MUAC, or any combination thereof, wherein the amount of change would have been significantly greater absent intervention. In further embodiments, improving a subject's health may comprise preventing or lessening a decrease in WAZ and WLZ, wherein the amount of change would have been significantly greater absent intervention. In further embodiments, improving a subject's health may comprise preventing or lessening a decrease WAZ, WLZ, and MUAC, wherein the amount of change would have been significantly greater absent intervention. In exemplary embodiments, a subject may be six months to five years of age, six months to 2 years of age, or six months to 18 months of age.

In some embodiments, improving a subject's health may comprise preventing or lessening a decrease in β-LAZ, β-WAZ, β-WLZ, β-MUAC, or any combination thereof, wherein the amount of change would have been significantly greater absent intervention. In further embodiments, improving a subject's health may comprise preventing or lessening a decrease in β-WAZ and β-WLZ, wherein the amount of change would have been significantly greater absent intervention. In further embodiments, improving a subject's health may comprise preventing or lessening a decrease in β-WAZ, β-WLZ, and β-MUAC, wherein the amount of change would have been significantly greater absent intervention. In exemplary embodiments, a subject may be six months to five years of age, six months to 2 years of age, or six months to 18 months of age.

In some embodiments, improving a subject's health may comprise preventing the development or worsening of a symptom associated with malnutrition. Non-limiting examples of symptoms include fever, cough, rhinorrhea, diarrhea, tiredness, irritability, inability to concentrate, etc. In some embodiments, improving a subject's health may comprise preventing the development or worsening of a symptom selected from fever, cough, rhinorrhea, and diarrhea. In some embodiments, improving a subject's health may comprise preventing the development or worsening of a symptom selected from fever, cough, and rhinorrhea. In some embodiments, improving a subject's health may comprise preventing the development or worsening of a symptom selected from cough, and rhinorrhea. In exemplary embodiments, a subject may be six months to five years of age, six months to 2 years of age, or six months to 18 months of age.

In some embodiments, an improvement in a subject's health is improved growth, as defined by a statistically significant improvement in one or more anthropometric measurement including but not limited to height-for-age z-score (HAZ), weight-for-height z-score (WHZ), weight-for-age Z-score (WAZ), and mid upper arm circumference (MUAC). Alternatively, or in addition, an improvement in a subject's growth may be defined by a statistically significant change in the relative abundances of health discriminatory plasma proteins, and/or biomarkers/mediators of gut barrier function, in a manner towards chronologically-age matched reference healthy subjects. In certain embodiments, the subject is malnourished. In a specific embodiment, the subject has MAM or SAM.

In one example, improvement in the subject's growth may be measured by HAZ, wherein the change in HAZ is statistically significant. In further embodiments, the abundance of one or more protein positively correlated with HAZ may be increased and/or the abundance of one or more protein negatively correlated with HAZ may be decreased, wherein the abundance of a protein is measured in a biological sample obtained from the subject (e.g., blood, plasma, urine, etc.). Plasma proteins positively and negatively correlated with HAZ are described in the examples. In a specific embodiment, a protein positively correlated with HAZ is an IGF-1 binding protein (e.g., IGFBP-3), growth hormone receptor (GHR), or leptin (LEP). In a specific embodiment, a protein negatively correlated with HAZ is PYY or GDF15.

In another example, improvement in the subject's growth may be measured by WHZ, wherein the change in WHZ is statistically significant. In further embodiments, the abundance of one or more protein positively correlated with WHZ may be increased and/or the abundance of one or more protein negatively correlated with WHZ may be decreased, wherein the abundance of a protein is measured in a biological sample obtained from the subject (e.g., blood, plasma, urine, etc.). Plasma proteins positively and negatively correlated with WHZ are described in the examples.

In another example, improvement in the subject's growth may be measured by WAZ, wherein the change in WAZ is statistically significant. In further embodiments, the abundance of one or more protein positively correlated with WAZ may be increased and/or the abundance of one or more protein negatively correlated with WAZ may be decreased, wherein the abundance of a protein is measured in a biological sample obtained from the subject (e.g., blood, plasma, urine, etc.). Plasma proteins positively and negatively correlated with WAZ are described in the examples.

In another example, improvement in the subjects' growth may be measured by MUAC, wherein the change in MUAC is statistically significant. In further embodiments, the abundance of one or more protein positively correlated with MUAC may be increased and/or the abundance of one or more protein negatively correlated with MUAC may be decreased, wherein the abundance of a protein is measured in a biological sample obtained from the subject (e.g., blood, plasma, urine, etc.). Plasma proteins positively and negatively correlated with MUAC are described in the examples.

In certain embodiments, the present disclosure encompasses a method of improving the WAZ score of a malnourished subject, the method comprising administering an edible composition comprising carbohydrates that increases expression of nucleic acids encoding proteins in about 75%, 80%, 85%, 90%, 95%, 96%, 97%, 98%, 99%, or 100% of the CAZyme families indicated in Table A. The present disclosure also encompasses a method of improving the WAZ score of a malnourished subject, the method comprising administering an edible composition comprising carbohydrates that decreases expression of nucleic acids encoding proteins in about 75%, 80%, 85%, 90%, 95%, 96%, 97%, 98%, 99%, or 100% of the CAZyme families indicated in Table B. In preferred embodiments, the present disclosure encompasses a method of improving the WAZ score of a malnourished subject, the method comprising administering an edible composition comprising carbohydrates that increases expression of nucleic acids encoding proteins in about 75%, 80%, 85%, 90%, 95%, 96%, 97%, 98%, 99%, or 100% of the CAZyme families indicated in Table A and decreases expression of nucleic acids encoding proteins in about 75%, 80%, 85%, 90%, 95%, 96%, 97%, 98%, 99%, or 100% of the CAZyme families indicated in Table B. In a particular preferred embodiment, the present disclosure encompasses a method of improving the WAZ score of a malnourished subject, the method comprising administering an edible composition comprising carbohydrates that increases expression of nucleic acids encoding proteins in about 95%, 96%, 97%, 98%, 99%, or 100% of the CAZyme families indicated in Table A and decreases expression of nucleic acids encoding proteins in about 95%, 96%, 97%, 98%, 99%, or 100% of the CAZyme families indicated in Table B. In still another preferred embodiment, the present disclosure encompasses a method of improving the WAZ score of a malnourished subject, the method comprising administering an edible composition comprising carbohydrates that increases expression of nucleic acids encoding proteins in each of the CAZyme families indicated in Table A and decreases expression of nucleic acids encoding proteins in each of the CAZyme families indicated in Table B. In each of the above embodiments “increases expression” or “decreases expression” refers to a change in expression compared to the same subject before ingestion of the edible composition. Administration of the edible composition, as well as suitable subjects, are described herein in Section II. In certain exemplary embodiments, the edible composition referenced in this paragraph is a composition described in Section I herein.

### IV. Methods for Analyzing the Efficacy of a Therapeutic Intervention

In another aspect, the present disclosure provides methods for analyzing the efficacy of a therapeutic intervention on the nutritional status of a subject. In a specific embodiment, the subject is malnourished. In further embodiments, the subject has MAM or SAM. In still further embodiments, the subjects is a child 6 months in age or older. The method comprises (a) determining the concentration of a plurality of healthy-discriminatory proteins in a biological sample obtained from the subject, (b) administering the therapeutic intervention, (c) determining the post-therapeutic intervention concentration of each healthy-discriminatory protein from step (a), (d) determining if the concentration of each healthy-discriminatory protein was modified by the therapeutic intervention, and (e) categorizing the therapeutic intervention as efficacious in improving the nutritional status of the subject when the concentrations of more than 50% of the healthy-discriminatory proteins statistically change in a manner towards those encountered in healthy individuals after administration of the therapeutic intervention. The health-discriminatory proteins may be involved in aspects of the regulation of ponderal growth, linear growth, immune function, neurodevelopment and other determinants of physiologic status. The biological sample may be a blood sample, a urine same, a fecal sample, or a cecal sample. In one example, the biological sample is a blood sample and the concentration of one or more health-discriminatory proteins from Table 18 is measured. In one example, the biological sample is a blood sample and the concentration of one or more health-discriminatory proteins from Table F is measured. In one example, the biological sample is a blood sample and the concentration of one or more health-discriminatory proteins from Table G is measured. In one example, the biological sample is a blood sample and the concentration of one or more health-discriminatory proteins from Table H is measured.

In another aspect, the disclosure provides a method of analyzing the efficacy of a therapeutic intervention on the nutritional status of a subject. In a specific embodiment, the subject is malnourished. In further embodiments, the subject has MAM or SAM. In still further embodiments, the subjects is a child 6 months in age or older. The method comprises (a) determining the concentration of a plurality of SAM-discriminatory protein in a biological sample obtained from the subject, (b) administering the therapeutic intervention, (c) determining the post-therapeutic intervention concentration of each SAM-discriminatory protein measured in step (a), (d) determining if the concentration of each of the SAM-discriminatory proteins was modified by the therapeutic intervention, and (e) categorizing the therapeutic intervention as efficacious in improving the nutritional status of the subject when more than 50% of the SAM-discriminatory protein concentrations statistically change in a manner towards those encountered in healthy individuals. The SAM-discriminatory proteins may be involved in aspects of the regulation of ponderal growth, linear growth, immune function, neurodevelopment and other determinants of physiologic status. The biological sample may be a blood sample, a urine same, a fecal sample, or a cecal sample. In one example, the biological sample is a blood sample and the concentration of one or more health-discriminatory proteins from Table G and/or Table H is measured. In a specific embodiment, the concentration of about 10%, about 20%, about 25%, about 30%, about 40%, or about 50% of the protein in Table G and/or Table H is measured. In another specific embodiment, the concentration of about 60%, about 70%, about 75%, about 80%, about 90%, or about 1000% of the protein in Table G and/or Table H is measured. In another specific embodiment, the concentration of about 50% to about 100% of the proteins in Table G and/or Table H is measured.

In another aspect, the disclosure provides a method of analyzing the efficacy of a therapeutic intervention on the physical characteristics of a subject. In a specific embodiment, the subject is malnourished. In further embodiments, the subject has MAM or SAM. In still further embodiments, the subjects is a child 6 months in age or older. The method comprises (a) determining the concentration of a plurality of HAZ or WHZ-discriminatory proteins in a biological sample from the subject, (b) administering the therapeutic intervention, (c) determining the post-therapeutic intervention concentration of each HAZ or WHZ-discriminatory protein measured in step (a), (d) determining if the concentration of each of the HAZ or WHZ-discriminatory proteins was modified by the therapeutic intervention, and (e) categorizing the therapeutic intervention as efficacious in improving the physical characteristics of the subject when more than 50% of the positively correlated HAZ or WHZ-discriminatory protein concentrations rose after administration of the therapeutic intervention, or when more than 50% of the negatively correlated HAZ-discriminatory protein concentrations fell after administration of the therapeutic intervention. The biological sample may be a blood sample, a urine same, a fecal sample, or a cecal sample. In one example, the biological sample is a blood sample.

In another aspect, the disclosure provides a method of analyzing the efficacy of a therapeutic intervention on the maturity of a subject's gut microbiota. In a specific embodiment, the subject is malnourished. In further embodiments, the subject has MAM or SAM. In still further embodiments, the subjects is a child 6 months in age or older. The method comprises (a) measuring the subject's gut microbiota health by a method described in Section III(a); (b) administering the therapeutic intervention; (c) re-measuring the subject's gut microbiota health by the method used in step (a); and (d) categorizing the therapeutic intervention as efficacious the subject's gut microbiota health improved, as defined in Section III.

### (a) Therapeutic Intervention

A wide variety of therapeutic interventions are contemplated. In some embodiments, the therapeutic intervention is a drug. Drugs may be administered by orally, rectally, parenterally, or by inhalation. In other embodiments, the therapeutic intervention is a food, a prebiotic, a probiotic, or a nutritional supplement. A food, a prebiotic, a probiotic, or a nutritional supplement may be administered orally, parenterally, or rectally. In a specific embodiment, the therapeutic intervention is a therapeutic food.

The timing of administration of the therapeutic intervention and duration of treatment will be determined by the circumstances surrounding the case.

### (b) Protein Concentration

All suitable methods for measuring protein concentration in a biological sample known to one of skill in the art are contemplated within the scope of the invention. Non-limiting examples of suitable methods to assess protein concentration may include epitope binding agent-based methods and mass spectrometry based methods.

In some embodiments, the method to assess protein concentration is mass spectrometry. By exploiting the intrinsic properties of mass and charge, mass spectrometry (MS) can resolve and confidently identify a wide variety of complex compounds, including proteins. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000). In accordance with the present invention, one can use mass spectrometry to look for the protein concentration of each healthy-discriminatory protein or each SAM-discriminatory protein or each HAZ or WHZ-discriminatory protein.

In some embodiments, the method to assess protein concentration is an epitope binding agent-based method. As used herein, the term “epitope binding agent” refers to an antibody, an aptamer, a nucleic acid, an oligonucleic acid, an amino acid, a peptide, a polypeptide, a protein, a lipid, a metabolite, a small molecule, or a fragment thereof that recognizes and is capable of binding to a target gene protein. Nucleic acids may include RNA, DNA, and naturally occurring or synthetically created derivative.

As used herein, the term “antibody” generally means a polypeptide or protein that recognizes and can bind to an epitope of an antigen. An antibody, as used herein, may be a complete antibody as understood in the art, i.e., consisting of two heavy chains and two light chains, or may be any antibody-like molecule that has an antigen binding region, and includes, but is not limited to, antibody fragments such as Fab′, Fab, F(ab′)₂, single domain antibodies, Fv, and single chain Fv. The term antibody also refers to a polyclonal antibody, a monoclonal antibody, a chimeric antibody and a humanized antibody. The techniques for preparing and using various antibody-based constructs and fragments are well known in the art. Means for preparing and characterizing antibodies are also well known in the art (See, e.g. Antibodies: A Laboratory Manual, Cold Spring Harbor Laboratory, 1988; herein incorporated by reference in its entirety).

As used herein, the term “aptamer” refers to a polynucleotide, generally a RNA or DNA that has a useful biological activity in terms of biochemical activity, molecular recognition or binding attributes. Usually, an aptamer has a molecular activity such as binging to a target molecule at a specific epitope (region). It is generally accepted that an aptamer, which is specific in it binding to a polypeptide, may be synthesized and/or identified by in vitro evolution methods. Means for preparing and characterizing aptamers, including by in vitro evolution methods, are well known in the art (See, e.g. U.S. Pat. No. 7,939,313; herein incorporated by reference in its entirety).

In general, an epitope binding agent-based method of assessing protein concentrations comprises contacting a sample comprising a polypeptide with an epitope binding agent specific for the polypeptide under conditions effective to allow for formation of a complex between the epitope binding agent and the polypeptide. Epitope binding agent-based methods may occur in solution, or the epitope binding agent or sample may be immobilized on a solid surface. Non-limiting examples of suitable surfaces include microtitre plates, test tubes, beads, resins, and other polymers.

An epitope binding agent may be attached to the substrate in a wide variety of ways, as will be appreciated by those in the art. The epitope binding agent may either be synthesized first, with subsequent attachment to the substrate, or may be directly synthesized on the substrate. The substrate and the epitope binding agent may be derivatized with chemical functional groups for subsequent attachment of the two. For example, the substrate may be derivatized with a chemical functional group including, but not limited to, amino groups, carboxyl groups, oxo groups or thiol groups. Using these functional groups, the epitope binding agent may be attached directly using the functional groups or indirectly using linkers.

The epitope binding agent may also be attached to the substrate non-covalently. For example, a biotinylated epitope binding agent may be prepared, which may bind to surfaces covalently coated with streptavidin, resulting in attachment. Alternatively, an epitope binding agent may be synthesized on the surface using techniques such as photopolymerization and photolithography. Additional methods of attaching epitope binding agents to solid surfaces and methods of synthesizing biomolecules on substrates are well known in the art, i.e. VLSIPS technology from Affymetrix (e.g., see U.S. Pat. No. 6,566,495, and Rockett and Dix, Xenobiotica 30(2):155-177, both of which are hereby incorporated by reference in their entirety).

Contacting the sample with an epitope binding agent under effective conditions for a period of time sufficient to allow formation of a complex generally involves adding the epitope binding agent composition to the sample and incubating the mixture for a period of time long enough for the epitope binding agent to bind to any antigen present. After this time, the complex will be washed and the complex may be detected by any method well known in the art. Methods of detecting the epitope binding agent-polypeptide complex are generally based on the detection of a label or marker. The term “label”, as used herein, refers to any substance attached to an epitope binding agent, or other substrate material, in which the substance is detectable by a detection method. Non-limiting examples of suitable labels include luminescent molecules, chemiluminescent molecules, fluorochromes, fluorescent quenching agents, colored molecules, radioisotopes, scintillants, biotin, avidin, stretpavidin, protein A, protein G, antibodies or fragments thereof, polyhistidine, Ni2+, Flag tags, myc tags, heavy metals, and enzymes (including alkaline phosphatase, peroxidase, and luciferase). Methods of detecting an epitope binding agent-polypeptide complex based on the detection of a label or marker are well known in the art.

In some embodiments, an epitope binding agent-based method is an immunoassay. Immunoassays can be run in a number of different formats. Generally speaking, immunoassays can be divided into two categories: competitive immmunoassays and non-competitive immunoassays. In a competitive immunoassay, an unlabeled analyte in a sample competes with labeled analyte to bind an antibody. Unbound analyte is washed away and the bound analyte is measured. In a non-competitive immunoassay, the antibody is labeled, not the analyte. Non-competitive immunoassays may use one antibody (e.g. the capture antibody is labeled) or more than one antibody (e.g. at least one capture antibody which is unlabeled and at least one “capping” or detection antibody which is labeled.) Suitable labels are described above.

In an embodiment, the epitope binding agent method is an immunoassay. In another embodiment, the epitope binding agent method is selected from the group consisting of an enzyme linked immunoassay (ELISA), a fluorescence based assay, a dissociation enhanced lanthanide fluoroimmunoassay (DELFIA), a radiometric assay, a multiplex immunoassay, and a cytometric bead assay (CBA). In some embodiments, the epitope binding agent-based method is an enzyme linked immunoassay (ELISA). In other embodiments, the epitope binding agent-based method is a radioimmunoassay. In still other embodiments, the epitope binding agent-based method is an immunoblot or Western blot. In alternative embodiments, the epitope binding agent-based method is an array. In another embodiment, the epitope binding agent-based method is flow cytometry.

### (c) Modification of a Protein Concentration by a Therapeutic Intervention

The post-therapeutic intervention concentration of a protein may be compared to the pre-therapeutic intervention concentration of the protein. Generally speaking, expression of a protein is modified by a therapeutic intervention when there is a statistically significant increase or decrease in the concentration of the post-therapeutic intervention protein concentration compared to the pre-therapeutic intervention concentration of the respective protein.

### V. Ecogroup

In another aspect, the disclosure provides a method of categorizing a subject according to the maturity of their gut microbiota. The method comprises (a) measuring the representation (abundances) of 15 significantly co-varying bacterial taxa, termed an ecogroup, whose network development normally occurs in a programmatic fashion during the first 2 years of postnatal life in healthy infants/children, with young and mature ecogroup configurations showing sparse and more complex organization, respectively, and (b) a comparison of abundances of these taxa in a subject's fecal microbiota relative to their representation in the microbiota of members of the reference healthy control population.

In another aspect, the disclosure provides a method of visualizing the impact of perturbations on a gut microbiota ecogroup. The method comprises creation of a space by computing information based on ecogroup member profiles using principal components analysis where distance between any two points in the space represents the extent of similarity or dissimilarity between the ecogroup profiles of bacterial communities present in two respective fecal samples.

In another aspect, the disclosure provides a method of selecting a gut microbiota ecogroup. The method comprises the application of statistical methods of co-variance and principal components analysis to bacterial DNA sequence data obtained from fecal samples collected in a longitudinal birth cohort study of between 2 and 5 years duration, the result of which yields 15 reproducibly co-varying bacterial taxa.

Embodiments of the disclosure related to generating an ecogroup and analyses performed therewith may be described in the context of computer-executable instructions, such as program modules, executed by one or more computers or other devices, as described in U.S. Provisional Application Ser. No. 62/859,455, filed Jul. 10, 2019, for which at least one inventor, Dr. Jeffery Gordon, is a co-inventor; the disclosures of which are hereby incorporated by reference in their entirety.

### (a) Creating and Comparing an Ecogroup

According to the disclosure, an initial ecogroup analysis of a subject's gut microbiome is created. Additionally, according to the disclosure a post-therapeutic intervention ecogroup analysis of a subject's gut microbiome is created. Methods of conducting an initial and post-therapeutic intervention ecogroup analysis are described in the Examples. Specifically, fecal samples are collected prior to initiation of a therapeutic intervention and fecal samples are collected post-therapeutic intervention. In the instance of fecal samples collected post-therapeutic intervention, the fecal samples may be collected during and/or after completion of administration of the therapeutic intervention. In an embodiment, fecal samples may be collected about 1 week, about 2 weeks, about 3 weeks, about 4 weeks, about 5 weeks, about 6 weeks, about 7 weeks, and/or about 8 weeks after initiation of the therapeutic intervention. In another embodiment, the fecal samples may be collected about 2 months, about 3 months, about 4 months, about 5 months, about 6 months, about 7 months, about 8 months, about 9 months, about 10 months, about 11 months, or about 12 months after initiation of the therapeutic intervention. In still another embodiment, the fecal samples may be collected about 1 year, about 2 years, about 3 years, about 4 years, or about 5 years after initiation of the therapeutic intervention.

Once the fecal samples have been collected, amplicons may be generated from bacterial 16S rRNA genes present in the fecal sample and sequenced. More specifically, amplicons may be generated from variable region 4 (V4) of bacterial 16S rRNA genes present in the fecal sample and sequenced. The resulting reads may then be assigned to operational taxonomic units (OTUs) with greater than or equal to 97% nucleotide sequence identity. In an embodiment, amplicons may be generated from ecogroup-specific bacterial 16S rRNA genes present in the fecal sample. In an embodiment, the ecogroup-specific bacterial strains comprise B. longum (OTU 559527), S. gallolyticus (OTU 349024), L. ruminis (OTU 1107027), Bifidobacterium (OTU 484304), F. prausnitzii (OTU 514940), E. coli (OTU 1111294), F. prausnitzii (OTU 851865), P. copri (OTU 588929), E. rectale (OTU 708680), Clostridiales (OTU 1078587), P. copri (OTU 840914), S. thermophilus (OTU 579608), Prevotella (OTU 591785), E. faecalis (OTU 1111582), and Dialister (OTU 583746). In an exemplary embodiment, the ecogroup-specific bacterial strains consist of B. longum (OTU 559527), S. gallolyticus (OTU 349024), L. ruminis (OTU 1107027), Bifidobacterium (OTU 484304), F. prausnitzii (OTU 514940), E. coli (OTU 1111294), F. prausnitzii (OTU 851865), P. copri (OTU 588929), E. rectale (OTU 708680), Clostridiales (OTU 1078587), P. copri (OTU 840914), S. thermophilus (OTU 579608), Prevotella (OTU 591785), E. faecalis (OTU 1111582), and Dialister (OTU 583746).

The abundance of bacterial strains within the ecogroup may be calculated using the formulas described in U.S. Provisional Application Ser. No. 62/859,455.

A method of the disclosure comprises, in part, analyzing whether the post-therapeutic intervention ecogroup analysis of the subject's gut microbiome is statistically more similar to an age-matched healthy subject's gut microbiome ecogroup than the initial gut microbiota ecogroup analysis of the subject, wherein if the post-therapeutic intervention ecogroup analysis is more similar to a healthy ecogroup than the initial ecogroup analysis, the therapeutic intervention is efficacious. In a specific embodiment, the therapeutic intervention is a composition of the disclosure as described in Section I. If the post-therapeutic intervention ecogroup analysis of the subject's gut microbiome is statistically more similar to an age-matched healthy subject's gut microbiome ecogroup than the initial gut microbiota ecogroup analysis of the subject, then the difference between the post-therapeutic intervention ecogroup analysis and the age-matched healthy subject's gut microbiome ecogroup has a p-value of greater than 0.001, greater than 0.01, or greater than 0.05 and/or the difference between the post-therapeutic intervention ecogroup analysis and the initial ecogroup analysis is has a p-value of less than 0.05, or less than 0.01, or less than 0.001, or less than 0.0001.

### (b) Method of Categorizing a Subject According to the Maturity of Their Gut Microbiota

A method of categorizing a subject according to the maturity of their gut microbiota comprises, in part, an analysis of the representation (abundances) in a subject's fecal microbiota of 15 significantly co-varying bacterial taxa, termed an ecogroup, whose network development normally occurs in a programmatic fashion during the first 2 years of postnatal life in healthy infants/children, with young and mature ecogroup configurations showing sparse and more complex organization, respectively. In an embodiment, the 15 significantly co-varying bacterial taxa comprises B. longum, S. gallolyticus, L. ruminis, Bifidobacterium, F. prausnitzii, E. coli, P. copri, E. rectale, Clostridiales, S. thermophilus, Prevotella, E. faecalis, and Dialister, wherein a listed taxa may comprise more than one OTU. In another embodiment, the 15 significantly co-varying bacterial taxa comprises B. longum, S. gallolyticus, L. ruminis, Bifidobacterium, F. prausnitzii, E. coli, P. copri, E. rectale, Clostridiales, S. thermophilus, Prevotella, E. faecalis, and Dialister, wherein F. prausnitzii and P. copri comprise more than one OTU. In a specific embodiment, the 15 significantly co-varying bacterial taxa comprises B. longum (OTU 559527), S. gallolyticus (OTU 349024), L. ruminis (OTU 1107027), Bifidobacterium (OTU 484304), F. prausnitzii (OTU 514940), E. coli (OTU 1111294), F. prausnitzii (OTU 851865), P. copri (OTU 588929), E. rectale (OTU 708680), Clostridiales (OTU 1078587), P. copri (OTU 840914), S. thermophilus (OTU 579608), Prevotella (OTU 591785), E. faecalis (OTU 1111582), and Dialister (OTU 583746). In an exemplary embodiment, the 15 significantly co-varying bacterial taxa consists of B. longum (OTU 559527), S. gallolyticus (OTU 349024), L. ruminis (OTU 1107027), Bifidobacterium (OTU 484304), F. prausnitzii (OTU 514940), E. coli (OTU 1111294), F. prausnitzii (OTU 851865), P. copri (OTU 588929), E. rectale (OTU 708680), Clostridiales (OTU 1078587), P. copri (OTU 840914), S. thermophilus (OTU 579608), Prevotella (OTU 591785), E. faecalis (OTU 1111582), and Dialister (OTU 583746).

Once the fecal samples have been collected, amplicons may be generated from bacterial 16S rRNA genes present in the fecal sample and sequenced. More specifically, amplicons may be generated from variable region 4 (V4) of bacterial 16S rRNA genes present in the fecal sample and sequenced. The resulting reads may then be assigned to operational taxonomic units (OTUs) with greater than or equal to 97% nucleotide sequence identity. In an embodiment, amplicons may be generated from ecogroup-specific bacterial 16S rRNA genes present in the fecal sample. The abundance of bacterial taxa within the ecogroup may be calculated using the formulas described in the Raman et al. example.

A method of categorizing a subject according to the maturity of their gut microbiota also comprises, in part, a comparison of abundances of 15 significantly co-varying bacterial taxa in a subject's fecal microbiota relative to their representation in the microbiota of members of the reference healthy control population. Based on the abundances of the 15 significantly co-varying bacterial taxa, the maturity of the subject's gut microbiota may be identified. Accordingly, the subject may be categorized as having an immature gut microbiota if the abundances of the subject's 15 significantly co-varying bacterial taxa are more similar to a chronologically younger healthy control population.

### (c) Method of Visualizing the Impact of Perturbations on a Gut Microbiota Ecogroup

A method of visualizing the impact of perturbations on a gut microbiota ecogroup comprises creation of a space by computing information based on ecogroup member profiles using principal components analysis where distance between any two points in the space represents the extent of similarity or dissimilarity between the ecogroup profiles of bacterial communities present in two respective fecal samples. In an embodiment, the smaller the space between the points, the more similar the ecogroups and the larger the space between the points, the more dissimilar the ecogroups. In an exemplary embodiment, visualizing the impact of perturbations on a gut microbiota ecogroup may result in an output similar to FIG. 55.

### (d) Method of Selecting a Gut Microbiota Ecogroup

A method of selecting a gut microbiota ecogroup comprises the application of statistical methods of co-variance and principal components analysis to bacterial DNA sequence data obtained from fecal samples collected in a longitudinal birth cohort study of between 2 and 5 years duration, the result of which yields 15 reproducibly co-varying bacterial taxa. In an embodiment, the duration of a longitudinal birth cohort study may be between 1 and 6 years, 1 and 5 years, 1 and 4 years, 1 and 3 years, 2 and 6 years, 2 and 4 years, 3 and 6 years, or 3 and 5 years. In an embodiment, the 15 reproducibly co-varying bacterial taxa comprises B. longum, S. gallolyticus, L. ruminis, Bifidobacterium, F. prausnitzii, E. coli, P. copri, E. rectale, Clostridiales, S. thermophilus, Prevotella, E. faecalis, and Dialister, wherein a listed taxa may comprise more than one OTU. In another embodiment, the 15 reproducibly co-varying bacterial taxa comprises B. longum, S. gallolyticus, L. ruminis, Bifidobacterium, F. prausnitzii, E. coli, P. copri, E. rectale, Clostridiales, S. thermophilus, Prevotella, E. faecalis, and Dialister, wherein F. prausnitzii and P. copri comprise more than one OTU. In a specific embodiment, the 15 reproducibly co-varying bacterial taxa comprises B. longum (OTU 559527), S. gallolyticus (OTU 349024), L. ruminis (OTU 1107027), Bifidobacterium (OTU 484304), F. prausnitzii (OTU 514940), E. coli (OTU 1111294), F. prausnitzii (OTU 851865), P. copri (OTU 588929), E. rectale (OTU 708680), Clostridiales (OTU 1078587), P. copri (OTU 840914), S. thermophilus (OTU 579608), Prevotella (OTU 591785), E. faecalis (OTU 1111582), and Dialister (OTU 583746). In an exemplary embodiment, the 15 reproducibly co-varying bacterial taxa consists of B. longum (OTU 559527), S. gallolyticus (OTU 349024), L. ruminis (OTU 1107027), Bifidobacterium (OTU 484304), F. prausnitzii (OTU 514940), E. coli (OTU 1111294), F. prausnitzii (OTU 851865), P. copri (OTU 588929), E. rectale (OTU 708680), Clostridiales (OTU 1078587), P. copri (OTU 840914), S. thermophilus (OTU 579608), Prevotella (OTU 591785), E. faecalis (OTU 1111582), and Dialister (OTU 583746).

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Introduction to Examples 1-6

Examples 1-6 describe and execute an approach for integrating preclinical gnotobiotic animal models with human studies to understand the contributions of impaired gut microbial community development to childhood undernutrition. Combining metabolomic and proteomic analyses of serially collected plasma samples with metagenomic analyses of fecal samples, the biological state of Bangladeshi children with severe acute malnutrition (SAM) was characterized as they transitioned, following standard treatment, to moderate acute malnutrition (MAM) with persistent microbiota immaturity. Gnotobiotic mice were subsequently colonized with a defined consortium of bacterial strains representing different stages of microbiota development in healthy children. Administering different combinations of Bangladeshi complementary food ingredients to colonized and germ-free mice revealed diet-dependent changes in the relative abundance and metabolism of weaning-phase bacterial taxa underrepresented in SAM and MAM microbiota, plus diet- and colonization-dependent effects on host metabolism and growth-associated signaling pathways. Host and microbial effects of microbiota-directed complementary food (MDCF) prototypes were subsequently examined in gnotobiotic mice colonized with post-SAM MAM microbiota and in gnotobiotic piglets colonized with a defined consortium of targeted age- and growth-discriminatory bacteria. Finally, a randomized, double-blind study identified a lead MDCF that changes the abundances of targeted bacterial taxa and increases plasma levels of biomarkers and mediators of growth, bone formation, neurodevelopment, and immune function in children with MAM.

### Example 1

### Effects of Current Therapeutic Foods on the Biological State of Children with SAM

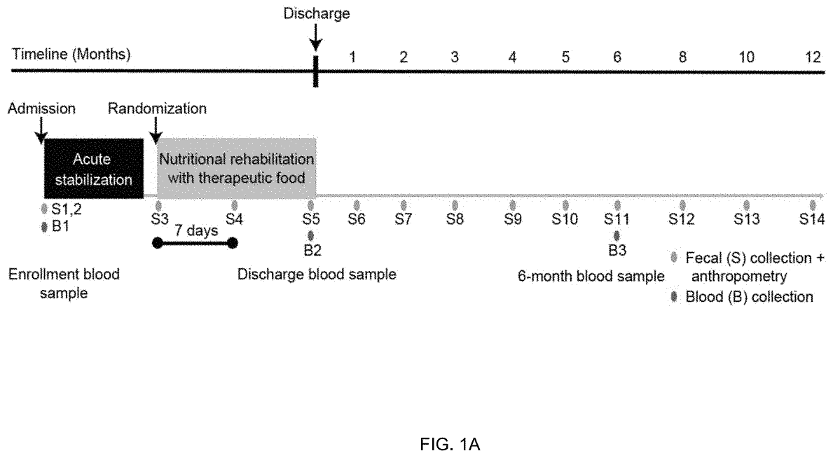

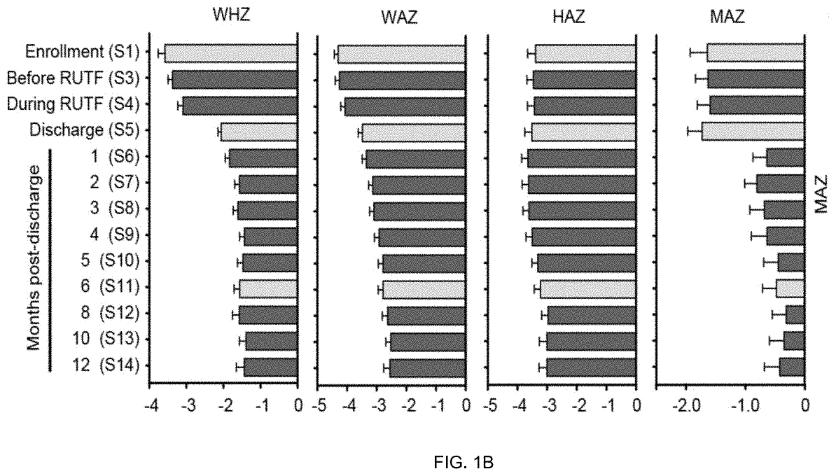

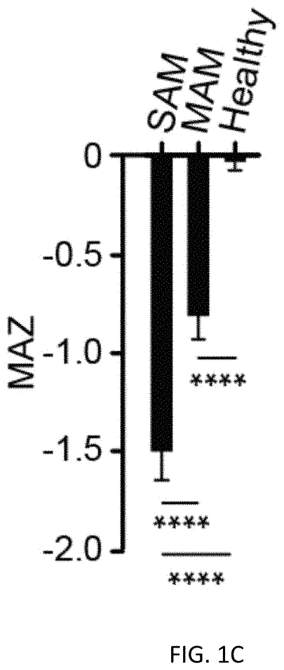

A total of 343 children aged 6-36 months with SAM were enrolled in a multi-center, randomized, double-blind ‘non-inferiority’ study designed to compare two locally produced therapeutic foods (see Methods) with a commercially available, ready-to-use therapeutic food (RUTF) (7) used throughout the world (see Table 1 for the compositions of these therapeutic foods and FIG. 1A for study design). Children received standard management for SAM during the acute stabilization phase of in-hospital treatment that included a short course of antibiotics (see Methods). Eligible children were then randomized to one of the three therapeutic food arms (˜200 kcal/kg/day, mean duration 16.1±10.3 days). Children were discharged after meeting criteria described in Methods. In a subset of 54 children, fecal samples were collected at enrollment [age 15.2±5.1 months (mean±SD)], prior to randomization, twice during treatment with a therapeutic food, and at regular intervals up to 12 months following discharge (FIG. 1A). Blood samples were also obtained at enrollment, discharge, and 6 months post-discharge for targeted mass spectrometry-based metabolic profiling; a sufficient quantity of blood was obtained from eight children at all three time points for DNA aptamer-based proteomics analysis (8-10). Forty-four percent of these children had MAM [weight-for-height Z-score (WHZ)≤−2.0] at 12 months of follow-up. None of the therapeutic foods produced a significant effect on their severe stunting (height-for-age Z-score (HAZ, FIG. 1B).

TABLE 1

| Therapeutic food | Ingredients | Energy (kcal) |
|---|---|---|
| Rice lentil | milk powder | 500 kcal/92g |
| | sugar | |
| | soybean oil | |
| | rice powder | |
| | red lentils | |
| | micronutrient mix | |
| Chickpea | milk powder | 500 kcal/92g |
| | sugar | |
| | soybean oil | |
| | chickpea | |
| | micronutrient mix | |
| Plumpy’Nut | milk powder | 500 kcal/92g |
| | sugar | |
| | vegetable oil | |
| | peanut paste | |
| | micronutrient mix | |

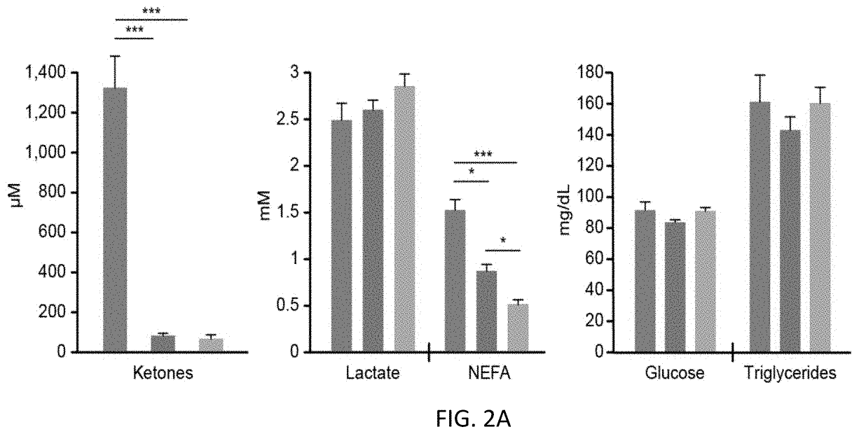

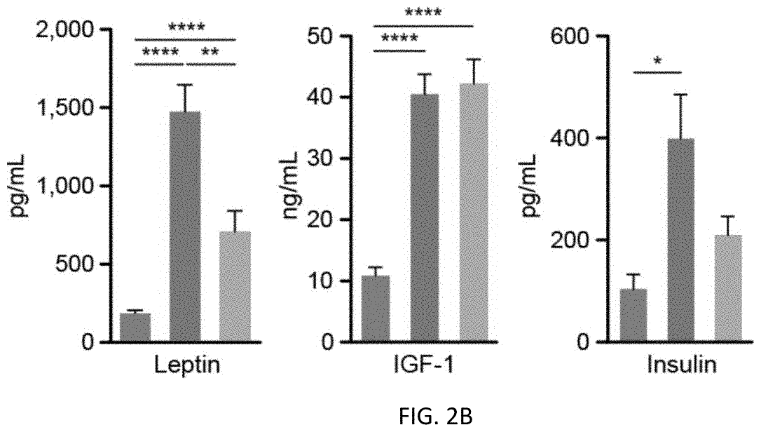

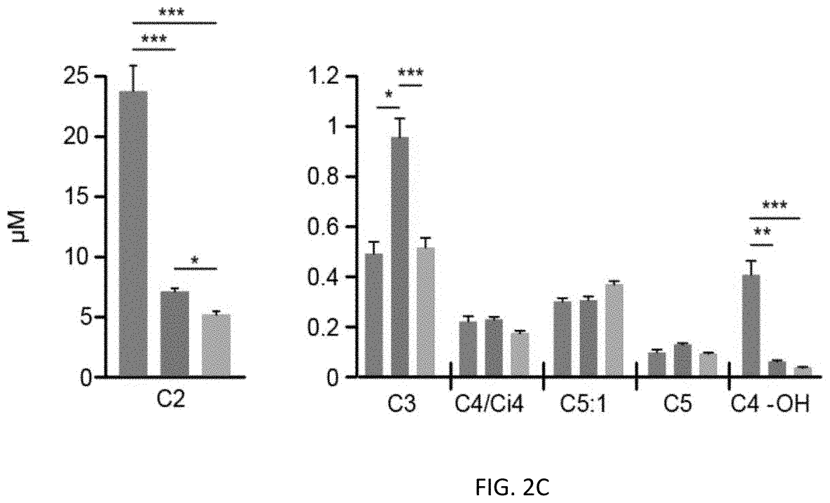

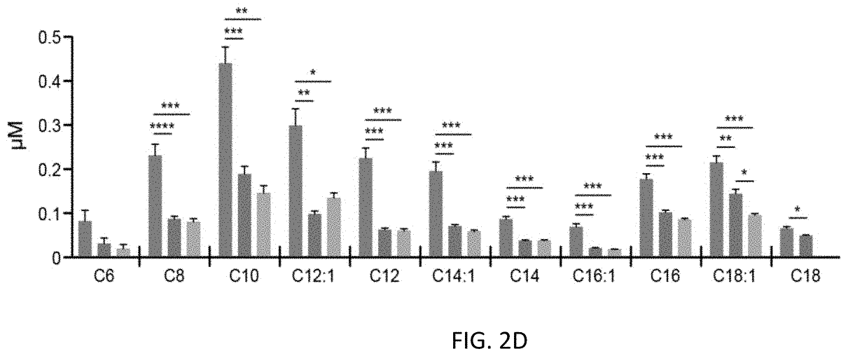

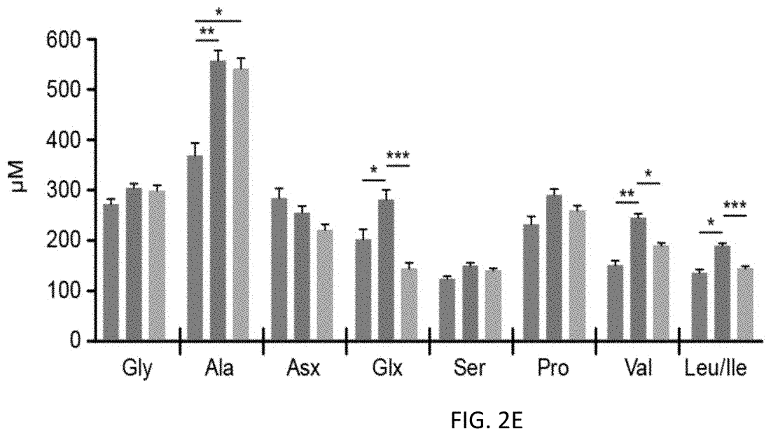

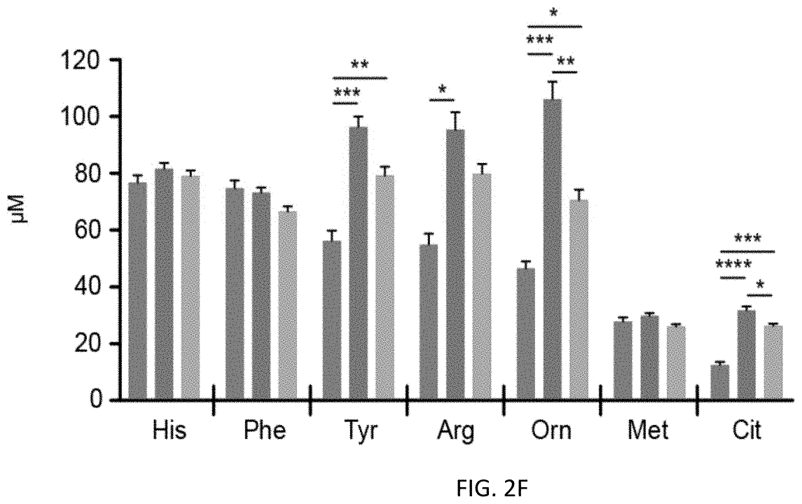

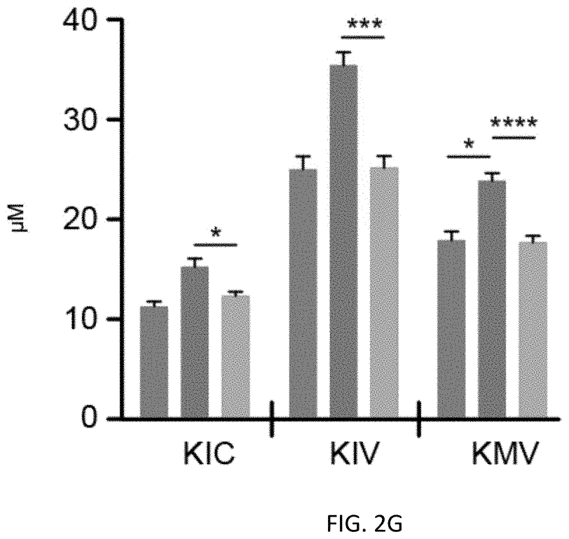

Metabolic phenotypes—Targeted mass spectrometry of plasma samples obtained at enrollment revealed high levels of ketones, non-esterified fatty acids (NEFA) and mid- to long-even-chain acylcarnitines (FIG. 2), consistent with the known acute malnutrition-induced lipolytic response that raises circulating fatty acids and activates fatty acid oxidation (11). By discharge this metabolic feature had normalized, while levels of a number of amino acids had increased significantly, including the gluconeogenic amino acid alanine, the branched-chain amino acids leucine, isoleucine and valine, plus products of branched-chain amino acid metabolism [C3 (propionyl)-carnitine and their ketoacids] (FIG. 2). These findings suggest that the increased protein provided by the therapeutic foods prompted a switch from fatty acid to amino acid oxidation, leading to repletion of fat depots, increases in plasma leptin (FIG. 2) and weight gain. However, 6 months after treatment, multiple plasma amino acids and their metabolites had declined to levels comparable to those at admission, while fatty acids and fatty acid-derived metabolites remained at similar concentrations to those observed at discharge (FIG. 2). IGF-1 and insulin levels did not change significantly during this period (FIG. 2), potentially explaining the absence of a signature of pronounced lipolysis that had been observed at enrollment. Although the suppression of lipolysis at six months post-discharge suggests a sustained effect of nutritional resuscitation, the fall in essential amino acids and the lower level of IGF-1 compared to that found in similarly-aged healthy children from the same community, may contribute to the observed failure to achieve catch-up growth.

The plasma proteome—Significant correlations were identified between levels of plasma proteins, anthropometric indices, plasma metabolites, and host signaling pathways regulating key facets of growth (Table 2; see for example, components of the GH-IGF axis, including soluble growth hormone receptor [also known as growth hormone binding protein (GHBP)], multiple IGF binding proteins (IGFBPs), and regulators of IGFBP turnover (the metalloprotease pappalysin-1 and its inhibitor stanniocalcin-1).

TABLE 2A
Spearman Correlations between plasma proteins and WHZ

| | Entrez | Entrez Gene | | Spearman | p-value |
|---|---|---|---|---|---|
| UniProt | Gene ID | Symbol | Target Name | r | (correlation) |
| P51884 | 4060 | LUM | Lumican | 0.692 | 1.82E−04 |
| P02751 | 2335 | FN1 | Fibronectin | 0.616 | 0.001 |
| P10912 | 2690 | GHR | Growth hormone receptor | 0.605 | 0.002 |
| Q76M96 | 151887 | CCDC80 | Coiled-coil domain-containing | 0.581 | 0.003 |
| | | | protein 80 (URB) | | |
| P02751 | 2335 | FN1 | Fibronectin Fragment 3 | 0.568 | 0.004 |
| | | | (FN1.3) | | |
| Q96NZ8 | 117166 | WFIKKN1 | WAP, kazal, immunoglobulin, | 0.564 | 0.004 |
| | | | kunitz and NTR domain-containing | | |
| | | | protein 1 (WFKN1) | | |
| P49862 | 5650 | KLK7 | Kallikrein-7 (Kallikrein 7) | 0.557 | 0.005 |
| Q14956 | 10457 | GPNMB | Transmembrane glycoprotein | 0.547 | 0.006 |
| | | | NMB (GPNMB) | | |
| Q16610 | 1893 | ECM1 | Extracellular matrix protein 1 | 0.540 | 0.006 |
| | | | (ECM1) | | |
| Q9UGM5 | 26998 | FETUB | Fetuin-B (FETUB) | 0.535 | 0.007 |
| Q9NYY1 | 50604 | IL20 | Interleukin-20 (IL-20) | 0.505 | 0.012 |
| O14793 | 2660 | MSTN | Growth/differentiation factor 8 | 0.504 | 0.012 |
| | | | (Myostatin) | | |
| P10915 | 1404 | HAPLN1 | Hyaluronan and proteoglycan | 0.499 | 0.013 |
| | | | link protein 1 (HPLN1) | | |
| P23280 | 765 | CA6 | Carbonic anhydrase 6 | 0.498 | 0.013 |
| | | | (Carbonic anhydrase 6) | | |
| Q9BU40 | 91851 | CHRDL1 | Chordin-like protein 1 | 0.496 | 0.014 |
| | | | (CRDL1) | | |
| Q07011 | 3604 | TNFRSF9 | Tumor necrosis factor receptor | 0.492 | 0.015 |
| | | | superfamily member 9 (4-1BB) | | |
| Q4KMG0 | 50937 | CDON | Cell adhesion molecule- | 0.488 | 0.016 |
| | | | related/down-regulated by | | |
| | | | oncogenes (CDON) | | |
| P09871 | 716 | C1S | Complement C1s | 0.487 | 0.016 |
| | | | subcomponent (C1s) | | |
| P02751 | 2335 | FN1 | Fibronectin Fragment 4 | 0.482 | 0.017 |
| | | | (FN1.4) | | |
| P08603 | 3075 | CFH | Complement factor H (Factor | 0.482 | 0.017 |
| | | | H) | | |
| P60604 | 7327 | UBE2G2 | Ubiquitin-conjugating enzyme | 0.472 | 0.020 |
| | | | E2 G2 (UB2G2) | | |
| P07949 | 5979 | RET | Proto-oncogene tyrosine- | 0.466 | 0.022 |
| | | | protein kinase receptor Ret | | |
| | | | (RET) | | |
| P01298 | 5539 | PPY | Pancreatic hormone (PH) | 0.464 | 0.023 |
| Q9NR71 | 56624 | ASAH2 | Neutral ceramidase (ASAH2) | 0.462 | 0.023 |
| Q9UBG0 | 9902 | MRC2 | C-type mannose receptor 2 | 0.453 | 0.026 |
| | | | (MRC2) | | |
| P35442 | 7058 | THBS2 | Thrombospondin-2 (TSP2) | 0.453 | 0.026 |
| P41159 | 3952 | LEP | Leptin | 0.450 | 0.027 |
| P25445 | 355 | FAS | Tumor necrosis factor receptor | 0.443 | 0.030 |
| | | | superfamily member 6 (Fas, | | |
| | | | soluble) | | |
| P13497 | 649 | BMP1 | Bone morphogenetic protein 1 | 0.440 | 0.031 |
| | | | (BMP-1) | | |
| Q8NBP7 | 255738 | PCSK9 | Proprotein convertase | 0.439 | 0.032 |
| | | | subtilisin/kexin type 9 | | |
| | | | (PCSK9) | | |
| P08253 | 4313 | MMP2 | 72 kDa type IV collagenase | 0.430 | 0.036 |
| | | | (MMP-2) | | |
| P22692 | 3487 | IGFBP4 | Insulin-like growth factor-binding | 0.429 | 0.037 |
| | | | protein 4 (IGFBP-4) | | |
| Q16674 | 8190 | MIA | Melanoma-derived growth | 0.428 | 0.037 |
| | | | regulatory protein (MIA) | | |
| P45985 | 6416 | MAP2K4 | Dual specificity mitogen- | 0.421 | 0.040 |
| | | | activated protein kinase | | |
| | | | kinase 4 (MP2K4) | | |
| Q9Y240 | 6320 | CLEC11A | Stem cell growth factor-alpha | 0.421 | 0.040 |
| | | | (SCGF-alpha) | | |
| O15444 | 6370 | CCL25 | C-C motif chemokine 25 | 0.420 | 0.041 |
| | | | (TECK) | | |
| P43652 | 173 | AFM | Afamin | 0.414 | 0.044 |
| Q12841 | 11167 | FSTL1 | Follistatin-related protein 1 | 0.411 | 0.046 |
| | | | (FSTL1) | | |
| P01854 | 3497, | IGHE, | Immunoglobulin E (IgE) | 0.405 | 0.050 |
| | 50802, | IGK@, | | | |
| | 3535 | IGL@ | | | |
| P01019 | 183 | AGT | Angiotensinogen | −0.702 | 1.30E−04 |
| P01567 | 3444 | IFNA7 | Interferon alpha-7 (IFNA7) | −0.591 | 0.002 |
| P02741 | 1401 | CRP | C-reactive protein (CRP) | −0.563 | 0.004 |
| P07359 | 2811 | GP1BA | Platelet glycoprotein Ib alpha | −0.539 | 0.007 |
| | | | chain (GP1BA) | | |
| P04818 | 7298 | TYMS | Thymidylate synthase (TS) | −0.531 | 0.008 |
| P07478 | 5645 | PRSS2 | Trypsin-2 | −0.524 | 0.009 |
| P18065 | 3485 | IGFBP2 | Insulin-like growth factor- | −0.518 | 0.010 |
| | | | binding protein 2 (IGFBP-2) | | |
| P09683 | 6343 | SCT | Secretin | −0.502 | 0.012 |
| Q04609 | 2346 | FOLH1 | Glutamate carboxypeptidase 2 | −0.499 | 0.013 |
| | | | (PSMA) | | |
| P26038 | 4478 | MSN | Moesin | −0.489 | 0.015 |
| Q8TEU8 | 124857 | WFIKKN2 | WAP, Kazal, immunoglobulin, | −0.480 | 0.018 |
| | | | Kunitz and NTR domain- | | |
| | | | containing protein 2 (WFKN2) | | |
| P56199, | 3672, | ITGA1, | Integrin alpha-I:beta-1 | −0.466 | 0.022 |
| P05556 | 3688 | ITGB1 | complex (Integrin a1b1) | | |
| P04054 | 5319 | PLA2G1B | Phospholipase A2 (GIB) | −0.438 | 0.032 |
| P06681 | 717 | C2 | Complement C2 (C2) | −0.437 | 0.033 |
| P07858 | 1508 | CTSB | Cathepsin B | −0.434 | 0.034 |
| Q9HD89 | 56729 | RETN | Resistin | −0.421 | 0.040 |
| P24158 | 5657 | PRTN3 | Myeloblastin (Proteinase-3) | −0.416 | 0.043 |

TABLE 2B
Spearman Correlations between plasma proteins and WAZ

| | Entrez | Entrez Gene | | Spearman | p-value |
|---|---|---|---|---|---|
| UniProt | Gene ID | Symbol | Target Name | r | (correlation) |
| Q12884 | 2191 | FAP | Prolyl endopeptidase FAP | 0.693 | 1.75E−04 |
| | | | (SEPR) | | |
| P0CG48 | 7316 | UBC | PolyUbiquitin K63-linked | 0.691 | 1.87E−04 |
| | | | (PolyUbiquitin K63) | | |
| O00626 | 6367 | CCL22 | C-C motif chemokine 22 | 0.666 | 3.87E−04 |
| | | | (MDC) | | |
| O15467 | 6360 | CCL16 | C-C motif chemokine 16 | 0.650 | 5.84E−04 |
| | | | (HCC-4) | | |
| O14793 | 2660 | MSTN | Growth/differentiation factor 8 | 0.643 | 7.06E−04 |
| | | | (Myostatin) | | |
| Q9UBG0 | 9902 | MRC2 | C-type mannose receptor 2 | 0.640 | 7.52E−04 |
| | | | (MRC2) | | |
| Q969J5 | 116379 | IL22RA2 | Interleukin-22 receptor | 0.637 | 8.16E−04 |
| | | | subunit alpha-2 (IL-22BP) | | |
| P05546 | 3053 | SERPIND1 | Heparin cofactor 2 | 0.612 | 1.48E−03 |
| P16109 | 6403 | SELP | P-selectin | 0.606 | 0.002 |
| P07949 | 5979 | RET | Proto-oncogene tyrosine- | 0.604 | 0.002 |
| | | | protein kinase receptor Ret | | |
| | | | (RET) | | |
| P01374, | 4049, | LTA, | Lymphotoxin alpha2:beta1 | 0.598 | 0.002 |
| Q06643 | 4050 | LTB | (Lymphotoxin a2/b1) | | |
| Q4KMG0 | 50937 | CDON | Cell adhesion molecule- | 0.597 | 0.002 |
| | | | related/down-regulated by | | |
| | | | oncogenes (CDON) | | |
| Q07507 | 1805 | DPT | Dermatopontin (DERM) | 0.595 | 0.002 |
| P17936 | 3486 | IGFBP3 | Insulin-like growth factor- | 0.593 | 0.002 |
| | | | binding protein 3 (IGFBP-3) | | |
| P10915 | 1404 | HAPLN1 | Hyaluronan and proteoglycan | 0.588 | 0.003 |
| | | | link protein 1 (HPLN1) | | |
| Q9UGM5 | 26998 | FETUB | Fetuin-B (FETUB) | 0.586 | 0.003 |
| P35030 | 5646 | PRSS3 | Trypsin-3 (TRY3) | 0.562 | 0.004 |
| P29622 | 5267 | SERPINA4 | Kallistatin | 0.557 | 0.005 |
| P39060 | 80781 | COL18A1 | Endostatin | 0.554 | 0.005 |
| O95750 | 9965 | FGF19 | Fibroblast growth factor 19 | 0.545 | 0.006 |
| | | | (FGF-19) | | |
| P02647 | 335 | APOA1 | Apolipoprotein A-I (Apo A-I) | 0.543 | 0.006 |
| P42892 | 1889 | ECE1 | Endothelin-converting | 0.541 | 0.006 |
| | | | enzyme 1 | | |
| Q15648 | 5469 | MED1 | Mediator of RNA polymerase | 0.540 | 0.006 |
| | | | II transcription subunit 1 | | |
| | | | (MED-1) | | |
| P08253 | 4313 | MMP2 | 72 kDa type IV collagenase | 0.537 | 0.007 |
| | | | (MMP-2) | | |
| Q96BQ1 | 131177 | FAM3D | Protein FAM3D (FAM3D) | 0.529 | 0.008 |
| O43866 | 922 | CD5L | CD5 antigen-like (CD5L) | 0.523 | 0.009 |
| Q8IWV2 | 152330 | CNTN4 | Contactin-4 (Contactin-4) | 0.520 | 0.009 |
| P48061 | 6387 | CXCL12 | Stromal cell-derived factor 1 | 0.517 | 0.010 |
| | | | (SDF-1) | | |
| P09486 | 6678 | SPARC | SPARC (ON) | 0.517 | 0.010 |
| Q9Y240 | 6320 | CLEC11A | Stem Cell Growth Factor-beta | 0.515 | 0.010 |
| | | | (SCGF-beta) | | |
| P45985 | 6416 | MAP2K4 | Dual specificity mitogen- | 0.514 | 0.010 |
| | | | activated protein kinase | | |
| | | | kinase 4 (MP2K4) | | |
| P22223 | 1001 | CDH3 | Cadherin-3 (P-Cadherin) | 0.502 | 0.012 |
| Q76M96 | 151887 | CCDC80 | Coiled-coil domain-containing | 0.493 | 0.014 |
| | | | protein 80 (URB) | | |
| Q16674 | 8190 | MIA | Melanoma-derived growth | 0.491 | 0.015 |
| | | | regulatory protein (MIA) | | |
| P10912 | 2690 | GHR | Growth hormone receptor | 0.489 | 0.015 |
| P58417 | 30010 | NXPH1 | Neurexophilin-1 (NXPH1) | 0.486 | 0.016 |
| P20231 | 64499 | TPSB2 | Tryptase beta-2 (TPSB2) | 0.484 | 0.017 |
| P01374, | 4049, | LTA, | Lymphotoxin alpha1:beta2 | 0.483 | 0.017 |
| Q06643 | 4050 | LTB | (Lymphotoxin a1/b2) | | |
| Q6ZVN8 | 148738 | HFE2 | Hemojuvelin (RGM-C) | 0.471 | 0.020 |
| Q9Y240 | 6320 | CLEC11A | Stem cell growth factor-alpha | 0.470 | 0.021 |
| | | | (SCGF-alpha) | | |
| P13501 | 6352 | CCL5 | C-C motif chemokine 5 | 0.464 | 0.022 |
| | | | (RANTES) | | |
| P43652 | 173 | AFM | Afamin | 0.461 | 0.023 |
| P39900 | 4321 | MMP12 | Macrophage metalloelastase | 0.459 | 0.024 |
| | | | (MMP-12) | | |
| Q16620 | 4915 | NTRK2 | BDNF/NT-3 growth factors | 0.459 | 0.024 |
| | | | receptor (TrkB) | | |
| Q14956 | 10457 | GPNMB | Transmembrane glycoprotein | 0.459 | 0.024 |
| | | | NMB (GPNMB) | | |
| P05067 | 351 | APP | Amyloid beta A4 protein | 0.454 | 0.026 |
| | | | (amyloid precursor protein) | | |
| P23280 | 765 | CA6 | Carbonic anhydrase 6 | 0.450 | 0.027 |
| | | | (Carbonic anhydrase 6) | | |
| Q96NZ8 | 117166 | WFIKKN1 | WAP, kazal, immunoglobulin, | 0.444 | 0.030 |
| | | | kunitz and NTR domain- | | |
| | | | containing protein 1 (WFKN1) | | |
| P01127 | 5155 | PDGFB | Platelet-derived growth factor | 0.441 | 0.031 |
| | | | subunit B (PDGF-BB) | | |
| P05156 | 3426 | CFI | Complement factor I (Factor | 0.440 | 0.031 |
| | | | I) | | |
| P24394 | 3566 | IL4R | Interleukin-4 receptor subunit | 0.433 | 0.035 |
| | | | alpha (IL-4 sR) | | |
| Q9NRJ3 | 56477 | CCL28 | C-C motif chemokine 28 | 0.430 | 0.036 |
| | | | (CCL28) | | |
| P01215, | 1081, | CGA, | Thyroid Stimulating Hormone | 0.430 | 0.036 |
| P01222 | 12372 | TSHB | (TSH) | | |
| Q9NR71 | 56624 | ASAH2 | Neutral ceramidase (ASAH2) | 0.429 | 0.036 |
| O94779 | 53942 | CNTN5 | Contactin-5 (Contactin-5) | 0.429 | 0.037 |
| Q07021 | 708 | C1QBP | Complement component 1 Q | 0.422 | 0.040 |
| | | | subcomponent-binding | | |
| | | | protein, mitochondrial | | |
| | | | (C1QBP) | | |
| P02751 | 2335 | FN1 | Fibronectin Fragment 4 | 0.416 | 0.043 |
| | | | (FN1.4) | | |
| P14778 | 3554 | IL1R1 | Interleukin-1 receptor type 1 | 0.414 | 0.045 |
| | | | (IL-1 sRI) | | |
| P00742 | 2159 | F10 | Coagulation Factor X | 0.410 | 0.047 |
| | | | (Coagulation Factor X) | | |
| P12956 | 2547 | XRCC6 | X-ray repair cross- | −0.741 | 3.45E−05 |
| | | | complementing protein 6 | | |
| | | | (Ku70) | | |
| O95631 | 9423 | NTN1 | Netrin-1 (NET1) | −0.718 | 7.88E−05 |
| P18065 | 3485 | IGFBP2 | Insulin-like growth factor- | −0.700 | 1.41E−04 |
| | | | binding protein 2 (IGFBP-2) | | |
| Q99988 | 9518 | GDF15 | Growth/differentiation factor | −0.691 | 1.84E−04 |
| | | | 15 (GDF15) | | |
| P05413 | 2170 | FABP3 | Fatty acid-binding protein, | −0.631 | 9.38E−04 |
| | | | heart (FABP) | | |
| P07478 | 5645 | PRSS2 | Trypsin-2 | −0.593 | 0.002 |
| Q9UJ70 | 55577 | NAGK | N-acetyl-D-glucosamine | −0.587 | 0.003 |
| | | | kinase (NAGK) | | |
| P10643 | 730 | C7 | Complement component C7 | −0.582 | 0.003 |
| | | | (C7) | | |
| P61328 | 2257 | FGF12 | Fibroblast growth factor 12 | −0.553 | 0.005 |
| | | | (FGF-12) | | |
| P56199, | 3672, | ITGA1, | Integrin alpha-I:beta-1 | −0.548 | 0.006 |
| P05556 | 3688 | ITGB1 | complex (Integrin a1b1) | | |
| P14210 | 3082 | HGF | Hepatocyte growth factor | −0.545 | 0.006 |
| | | | (HGF) | | |
| Q02750 | 5604 | MAP2K1 | Dual specificity mitogen- | −0.532 | 0.007 |
| | | | activated protein kinase | | |
| | | | kinase 1 (MEK1) | | |
| P68402 | 5049 | PAFAH1B2 | Platelet-activating factor | −0.527 | 0.008 |
| | | | acetylhydrolase IB subunit | | |
| | | | beta (PAFAH beta subunit) | | |
| P51512 | 4325 | MMP16 | Matrix metalloproteinase-16 | −0.521 | 0.009 |
| | | | (MMP-16) | | |
| Q99729 | 3182 | HNRNPAB | Heterogeneous nuclear | −0.500 | 0.013 |
| | | | ribonucleoprotein A/B | | |
| | | | (hnRNP A/B) | | |
| Q01638 | 9173 | IL1RL1 | Interleukin-1 receptor-like 1 | −0.491 | 0.015 |
| | | | (IL-1 R4) | | |
| Q99616 | 6357 | CCL13 | C-C motif chemokine 13 | −0.482 | 0.017 |
| | | | (MCP-4) | | |
| P52823 | 6781 | STC1 | Stanniocalcin-1 | −0.477 | 0.019 |
| P02794, | 2495, | FTH1, | Ferritin | −0.468 | 0.021 |
| P02792 | 2512 | FTL | | | |
| Q16719 | 8942 | KYNU | Kynureninase (KYNU) | −0.468 | 0.021 |
| P10144 | 3002 | GZMB | Granzyme B | −0.464 | 0.023 |
| O75144 | 23308 | ICOSLG | ICOS ligand (B7-H2) | −0.462 | 0.023 |
| P35247 | 6441 | SFTPD | Pulmonary surfactant- | −0.461 | 0.024 |
| | | | associated protein D (SP-D) | | |
| P01033 | 7076 | TIMP1 | Metalloproteinase inhibitor 1 | −0.460 | 0.024 |
| | | | (TIMP-1) | | |
| P11142 | 3312 | HSPA8 | Heat shock cognate 71 kDa | −0.458 | 0.024 |
| | | | protein (HSP70 protein 8) | | |
| P07900, | 3320, | HSP90AA1, | Heat shock protein HSP 90- | −0.455 | 0.025 |
| P08238 | 3326 | HSP90AB1 | alpha/beta (HSP 90a/b) | | |
| P04278 | 6462 | SHBG | Sex hormone-binding globulin | −0.451 | 0.027 |
| | | | (SHBG) | | |
| P68431 | 8350 | HIST1H3A | Histone H3.1 (H31) | −0.449 | 0.028 |
| P05114 | 3150 | HMGN1 | Non-histone chromosomal | −0.448 | 0.028 |
| | | | protein HMG-14 (HMGN1) | | |
| P40925 | 4190 | MDH1 | Malate dehydrogenase, | −0.437 | 0.033 |
| | | | cytoplasmic (MDHC) | | |
| P00750 | 5327 | PLAT | Tissue-type plasminogen | −0.432 | 0.035 |
| | | | activator (tPA) | | |
| P18510 | 3557 | IL1RN | Interleukin-1 receptor | −0.431 | 0.035 |
| | | | antagonist protein (IL-1Ra) | | |
| P50461 | 8048 | CSRP3 | Cysteine and glycine-rich | −0.429 | 0.037 |
| | | | protein 3 (CSRP3) | | |
| P17213 | 671 | BPI | Bactericidal permeability- | −0.427 | 0.037 |
| | | | increasing protein (BPI) | | |
| P55773 | 6368 | CCL23 | Ck-beta-8-1 (Ck-b-8-1) | −0.417 | 0.042 |
| P07451 | 761 | CA3 | Carbonic anhydrase 3 | −0.417 | 0.043 |
| P08238 | 3326 | HSP90AB1 | Heat shock protein HSP 90- | −0.416 | 0.043 |
| | | | beta (HSP 90b) | | |
| P05451 | 5967 | REG1A | Lithostathine-1-alpha (PSP) | −0.411 | 0.046 |
| O60506 | 10492 | SYNCRIP | Heterogeneous nuclear | −0.407 | 0.049 |
| | | | ribonucleoprotein Q (HNRPQ) | | |
| P06702 | 6280 | S100A9 | Protein S100-A9 (calgranulin | −0.406 | 0.049 |
| | | | B) | | |

TABLE 2C
Spearman Correlations between plasma proteins and HAZ

| | Entrez | Entrez Gene | | Spearman | p-value |
|---|---|---|---|---|---|
| UniProt | Gene ID | Symbol | Target Name | r | (correlation) |
| P07359 | 2811 | GP1BA | Platelet glycoprotein Ib alpha | 0.665 | 3.90E−04 |
| | | | chain (GP1BA) | | |
| P09683 | 6343 | SCT | Secretin | 0.525 | 0.008 |
| P42575 | 835 | CASP2 | Caspase-2 | 0.521 | 0.009 |
| P01019 | 183 | AGT | Angiotensinogen | 0.451 | 0.027 |
| P20810 | 831 | CAST | Calpastatin (Calpastatin) | 0.448 | 0.028 |
| O75556 | 4246 | SCGB2A1 | Mammaglobin-B | 0.443 | 0.030 |
| | | | (Mammaglobin 2) | | |
| P01567 | 3444 | IFNA7 | Interferon alpha-7 (IFNA7) | 0.440 | 0.031 |
| P24394 | 3566 | IL4R | Interleukin-4 receptor subunit | 0.418 | 0.042 |
| | | | alpha (IL-4 sR) | | |
| Q04609 | 2346 | FOLH1 | Glutamate carboxypeptidase | 0.417 | 0.043 |
| | | | 2 (PSMA) | | |
| P14151 | 6402 | SELL | L-Selectin (sL-Selectin) | 0.405 | 0.050 |
| P25445 | 355 | FAS | Tumor necrosis factor | −0.549 | 0.005 |
| | | | receptor superfamily member | | |
| | | | 6 (Fas, soluble) | | |
| P35442 | 7058 | THBS2 | Thrombospondin-2 (TSP2) | −0.515 | 0.010 |
| P00750 | 5327 | PLAT | Tissue-type plasminogen | −0.477 | 0.018 |
| | | | activator (tPA) | | |
| P11142 | 3312 | HSPA8 | Heat shock cognate 71 kDa | −0.426 | 0.038 |
| | | | protein (HSP70 protein 8) | | |
| Q9Y6Q6 | 8792 | TNFRSF11A | Tumor necrosis factor | −0.425 | 0.038 |
| | | | receptor superfamily member | | |
| | | | 11A (RANK) | | |
| Q8NBP7 | 255738 | PCSK9 | Proprotein convertase | −0.423 | 0.039 |
| | | | subtilisin/kexin type 9 | | |
| | | | (PCSK9) | | |
| Q92956 | 8764 | TNFRSF14 | Tumor necrosis factor | −0.408 | 0.048 |
| | | | receptor superfamily member | | |
| | | | 14 (HVEM) | | |

Analysis of the proteomic dataset revealed significant correlations between plasma proteins and anthropometric indices (see Table 2 for p-values). WHZ scores were positively correlated with circulating levels of the soluble proteolytic cleavage product of the membrane-bound growth hormone receptor [GHR, also known as growth hormone binding protein (GHBP), r=0.6]. Approximately 50% of GH is bound to GHBP, which serves to prolong its half-life and modulate its biological activity (83). GHBP is increased in obese adults and reduced after weight loss (84). In the children treated for SAM, plasma GHBP was also positively correlated with the adipokine leptin (Spearman r=0.6), consistent with the notion that increased fat mass, driven by nutritional recovery, leads to changes in leptin and GH signaling. WHZ scores were also positively correlated with downstream GH-responsive biomarkers, including lumican, extracellular matrix protein 1 (ECM1) and fibronectin (85).

A number of plasma proteins exhibited strong negative correlations with WHZ scores, including angiotensinogen (AGT; Spearman r=−0.70), a key component of the renin-angiotensin system (RAS) that regulates blood pressure and other aspects of cardio-metabolic function. Malnutrition has been reported to induce a pro-inflammatory state with increased expression of RAS components, analogous to responses observed in mouse models of diet-induced obesity (86). There was also an inverse correlation between plasma levels of C-reactive protein (CRP), an acute phase reactant and biomarker of systemic inflammation, and WHZ scores (Spearman r=−0.56).

Plasma proteins with significant correlations with plasma NEFA, ketones, lactate, glucose, triglycerides, branched-chain amino acids and C3 acylcarnitine were identified. They include growth differentiation factor 15/macrophage inhibitory cytokine −1 (GDF15/MIC-1), which was significantly correlated with NEFA (r=0.78) and ketones (r=0.58), and negatively correlated with WAZ (r=−0.69; Table 2). This TGF-b superfamily member is implicated in anorexia and muscle wasting associated with cancer (87), and with chronic heart failure in children (88).

Circulating IGFs (IGF-1 and IGF-2) are complexed with binding proteins (IGFBPs), primarily IGFBP-3. Binding to IGFBPs affects the half-life of IGFs and their interactions with extracellular matrix components and cell surface receptors (89). The IGFBPs have unique functions and are regulated in distinct ways. Unlike IGFBP-3, IGFBP-1 and IGFBP-2 are suppressed by GH and are implicated in adaptive changes in glucose and lipid metabolism (90). Pappalysin-1 (pregnancy-associated plasma protein-A, PAPP-A) is a metalloprotease that selectively cleaves IGFBP-2, -4, and -5, resulting in release of sequestered IGF, thereby promoting its ability to bind to its receptor (91). In mice, overexpression of PAPP-A in osteoblasts results in a marked increase in the rate of bone formation (92), while overexpression in muscle increases skeletal muscle weight and fiber area (93). Stanniocalcin-1 (STC1) is a potent physiological inhibitor of IGFBP proteolysis by PAPP-A (94). Transgenic mice engineered to overexpress STC1 exhibit severely reduced growth (95).

In children treated for SAM, levels of IGFBP-1 and IGFBP-2 were positively correlated with NEFA (r=0.74 and 0.68, respectively) and ketones (r=0.49 and 0.60) while IGFBP-3 exhibited an inverse relationship with both analytes (r=-0.62 and -0.52), and IGFBP-4 with ketones (r=-0.56). IGFBP-4, another component of the GH-IGF axis positively correlated with WHZ, is highly expressed in adipocytes and is a proposed regulator of adipose tissue development and maintenance (96). PAPP-A was positively correlated with levels of branched-chain amino acids (valine r=0.59, leucine/isoleucine r=0.50). STC1 was strongly positively correlated with NEFA (r=0.70) and negatively correlated with ponderal growth (WAZ; r=−0.48). In summary, these results reveal that elevated plasma levels of IGFBP-1 and IGFBP-2 are associated with the acutely malnourished state, whereas IGFBP-3 and IGFBP-4 are associated with the metabolic normalization and ponderal growth that characterize the recovery phase of treatment. The observed changes in PAPP-A, together with reciprocal changes in its physiological inhibitor, STC1, may serve to regulate IGF-1 bioavailability, thereby affecting a range of anabolic processes (97).

Correlations between abundances of age-discriminatory taxa and plasma proteins—We performed Spearman's rank correlations between (i) the abundance of OTUs identified at enrollment (time point S1 in FIG. 1A), at discharge (S5) and 6 months following discharge (S11), and (ii) levels of plasma proteins at the corresponding time points. Integrating the plasma proteomics dataset with changes in the relative abundances of OTUs allowed us to identify statistically significant correlations between age- and growth-discriminatory bacterial taxa and mediators of host biological state/functions. For example, Faecalibacterium prausnitzii (OTU 514940), the taxon with the highest feature importance score in the sparse RF-derived model of microbiota maturation in healthy members of the Mirpur birth cohort, exhibited strong positive correlations with a number of proteins involved in or regulated by GH signaling, including GHR, lumican, fibronectin, and ECM1. Multiple age-discriminatory OTUs had significant negative correlations with GDF15, including F. prausnitzii, Clostridiales sp., Dorea longicatena, Dorea formicigenerans, Blautia sp., Eubacterium desmolans, and two members of Ruminococcaceae (Ruminococcaceae sp. and R. torques). F. prausnitzii and a number of other age-discriminatory strains were also significantly negatively correlated with plasma CRP levels.

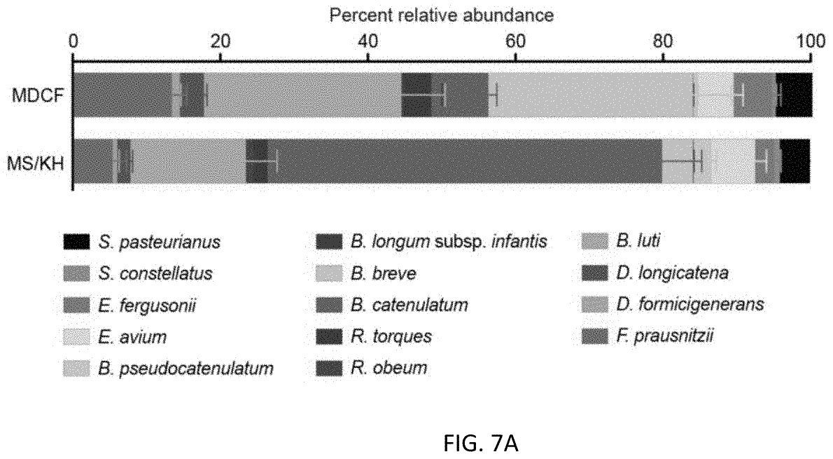

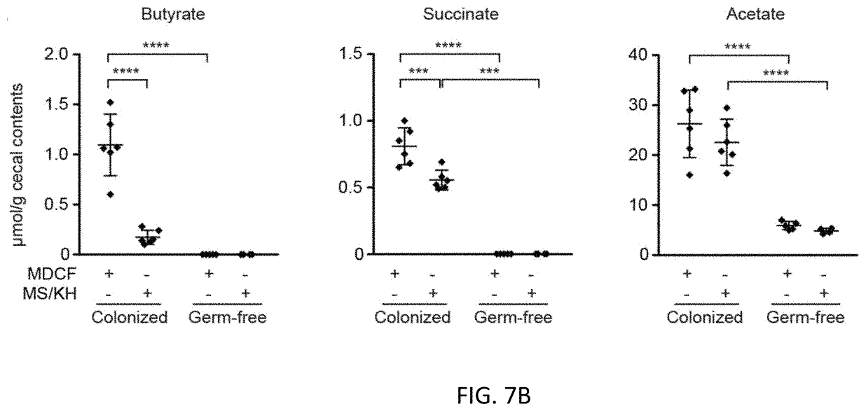

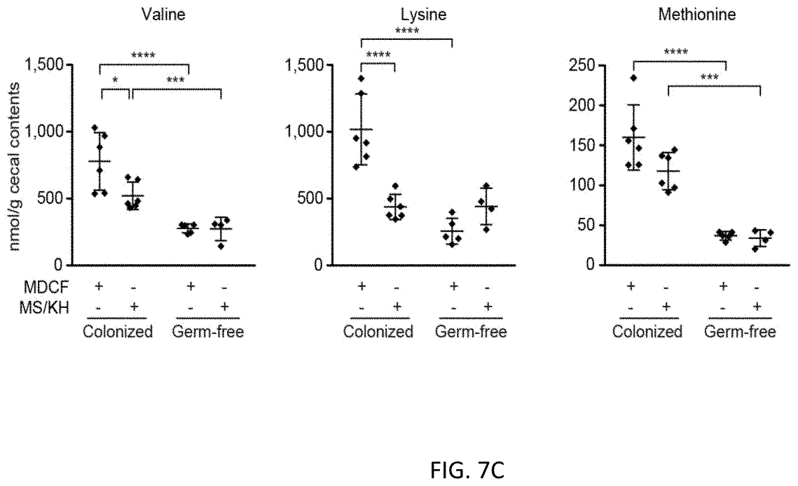

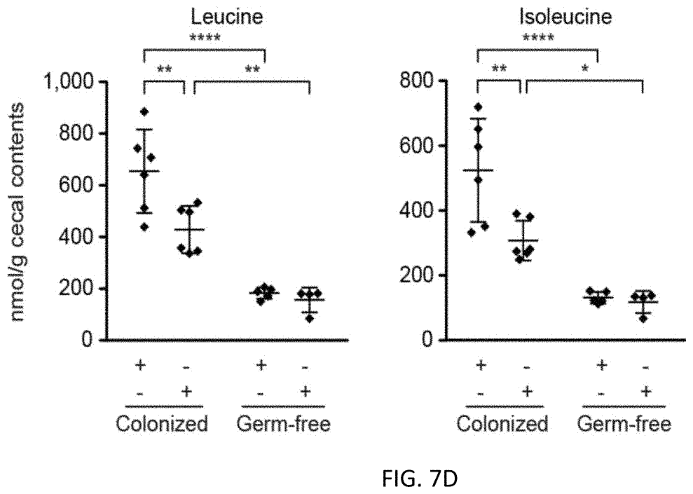

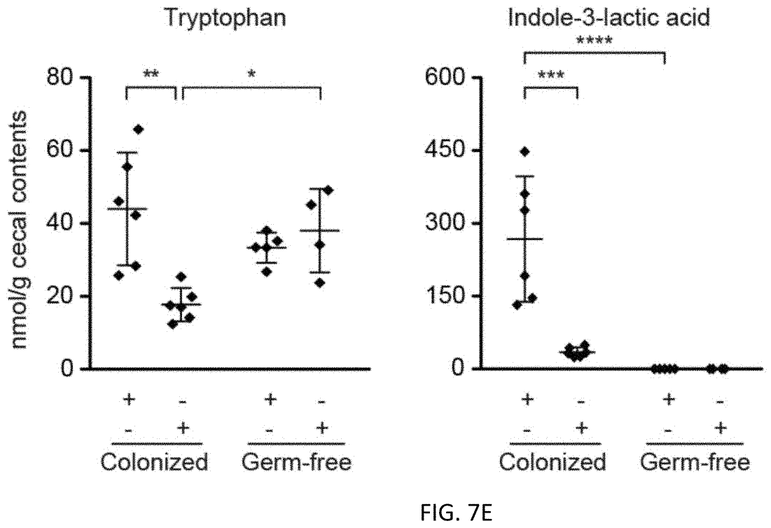

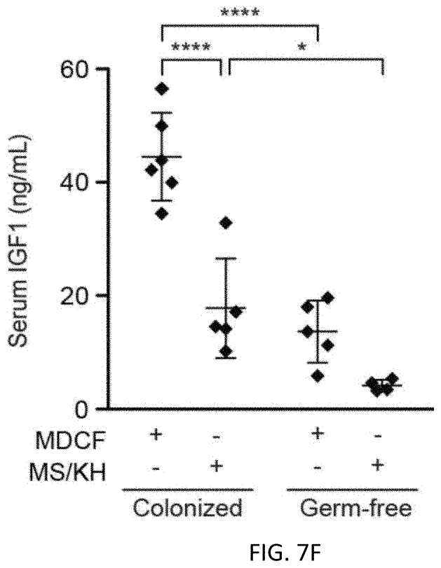

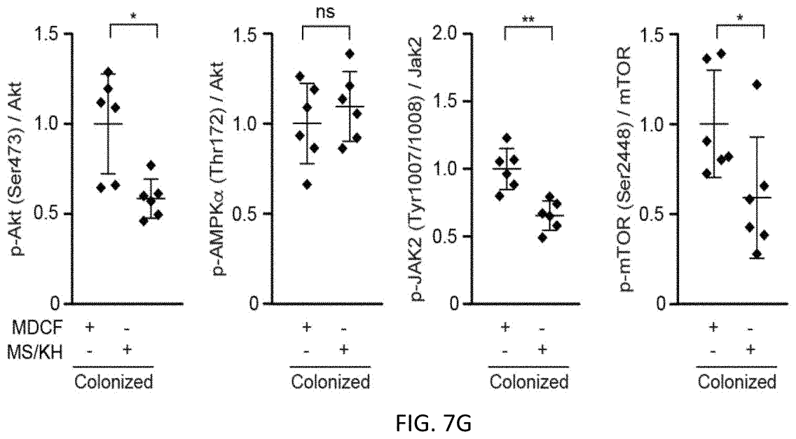

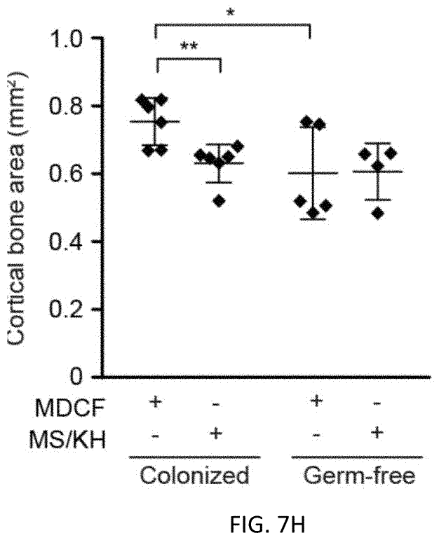

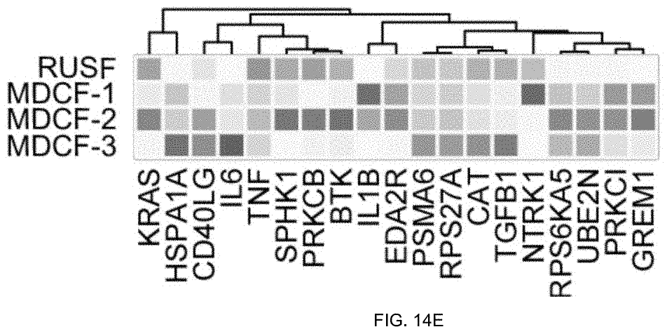

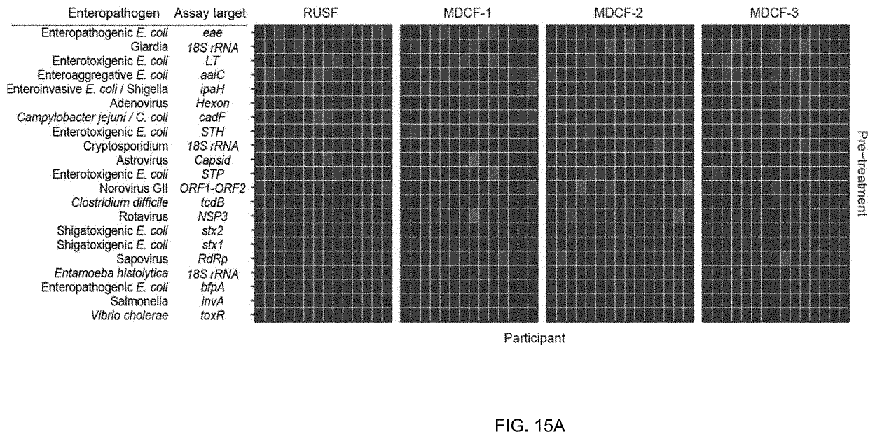

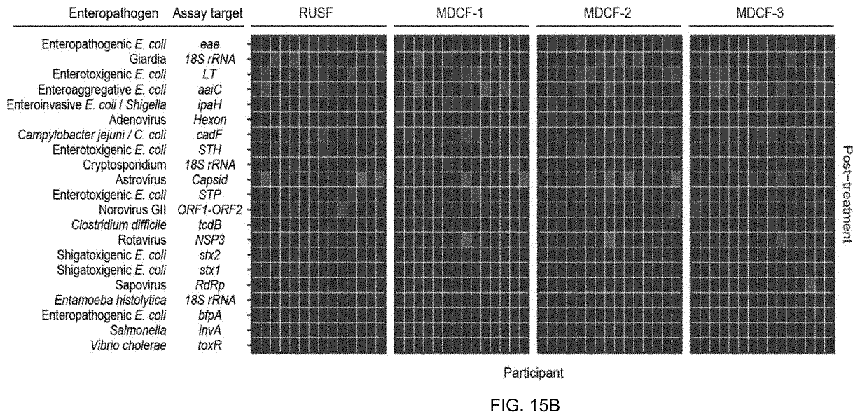

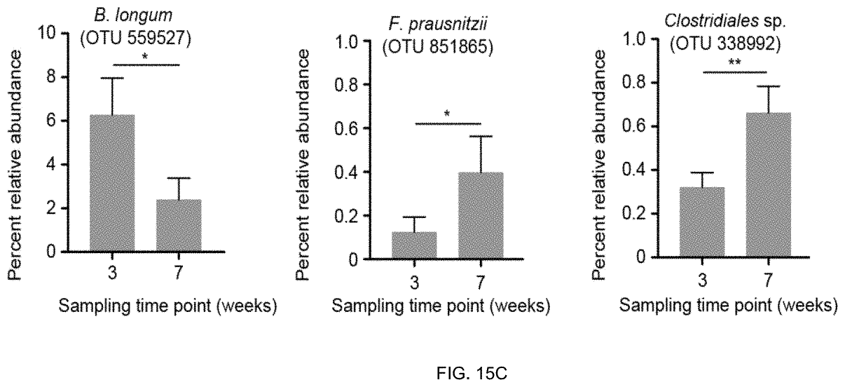

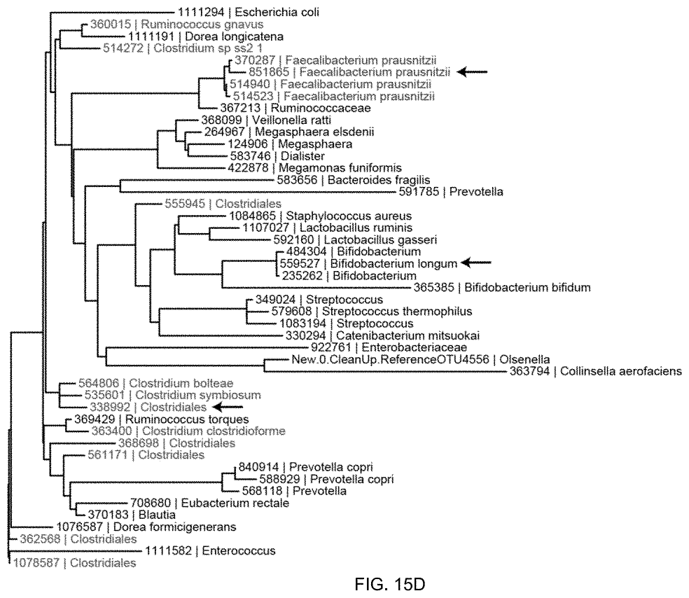

Bifidobacterium longum is a dominant member of the microbiota of breastfed infants; its presence is associated with numerous beneficial effects on the gut barrier and immune function (98). B. longum (OTU 559527) has the third highest feature importance in the sparse Bangladeshi RF-derived model and is a key component of the 15-member network of co-varying bacterial taxa ('ecogroup') described in (14). It is also responsive to MDCF formulations containing the four lead complementary food ingredients tested in gnotobiotic mice and piglets as well as in children with MAM (FIG. 15B in this report and FIG. 7 in (14)).

B. longum OTU 559527 was significantly correlated with 114 plasma proteins—the greatest number of significant correlations among the age-discriminatory OTUs. These proteins are involved in a wide range of biological processes. The two most strongly correlated proteins, legumain (an asparaginyl endopeptidase) and matrix metalloproteinase-2 (MMP-2/ gelatinase A) which is proteolytically cleaved and activated by legumain, are involved in remodeling the extracellular matrix. Among its other functions, MMP2 has been shown to cleave the chemokine CCL7 (MCP-3), converting it from a leukocyte chemoattractant to an antagonist, reducing cell infiltration, and dampening inflammation (99). Three cadherins (2, 3 and 6) that function as calcium-dependent cell adhesion molecules were positively correlated with B. longum abundance.

B. longum was also correlated with plasma levels of WNT1-inducible-signaling pathway protein 3 (WISP-3), a member of the CCN family of secreted proteins that regulate cell proliferation/survival, migration and adhesion, and differentiation in connective tissues. WISP-3 is secreted by chondrocytes where it can act in an autocrine fashion to induce collagen and aggrecan production and promote expression of superoxide dismutase (100). WISP-3 contains an IGFBP-like motif and has been demonstrated to modulate IGF-1 signaling in breast cancer (101).

B. longum was positively correlated with plasma levels of CDON (cell adhesion molecule-related/down-regulated by oncogenes). CDON and BOC (Brother of CDON) promote Hedgehog signaling through calcium-dependent interactions with Hedgehog ligands as co-receptors on the surface of target cells (102). There is considerable cross-talk between the Hedgehog pathway and Notch, WNT, EGF, FGF, TGF-beta and BMP signaling cascades. A number of these pathways are prominently represented by proteins that show significant correlations with the abundance of B. longum , including positive correlations with jagged-2 (JAG2), a Notch ligand involved in hematopoiesis, and BMP6, which is involved in growth of bone and cartilage (103). Another Notch ligand, delta-like protein 4 (DLL4), exhibits a strong negative correlation with B. longum. Inflammation has been reported to upregulate DLL4 in endothelial cells. In conjunction with IL-6 (which is also negatively correlated with B. longum), DLL4 promotes differentiation of blood monocytes into proinflammatory M1 macrophages (104). Blockade of DLL4 produces a marked reduction in inflammatory T cell responses and associated tissue damage (105).

In the SAM study, plasma levels of TNFSF15/TL1A (tumor necrosis factor ligand superfamily member 15) were inversely correlated with B. longum abundance. TNFSF15/TL1A is a member of the TNF superfamily that binds to death domain receptor 3 (DR3, TNFRSF25), activates NF-KB, and co-stimulates IFN-γ production in T cells (106). TNFSF15/TL1A and DR3 expression are increased in T cells and macrophages in the gut mucosa of patients with inflammatory bowel disease (107).

Biomarkers of systemic inflammation are a hallmark of children with undernutrition and growth faltering (108). F. prausnitzii (OTU 514940, 514523, 370287), D. formicigenerans (1076587), a weaning-phase Bifidobacterium sp. (484304), and Ruminococcus gnavus (360015) were all negatively correlated with C-reactive protein (CRP), an acute phase protein which is secreted by the liver during infection and systemic inflammation. Other acute phase proteins were also negatively correlated with the abundance of F. prausnitzii OTUs, including serum amyloid A-1 protein (SAA1) and complement C2. These opsonins target microbes for clearance and aid in the recruitment of immune cells to sites of infection. The negative correlation between these proteins and F. prausnitzii, D. formicigenerans, R. gnavus and the OTU ranked second in feature importance (1078587; Clostridiales sp.) in the sparse RF-derived model of microbiota maturation suggests that (i) a deficiency of these weaning-phase taxa may be conducive to developing or sustaining a state of local and systemic inflammation in children with SAM, and/or (ii) such a state reduces their fitness. A causal role for F. prausnitzii in suppressing gut inflammation is supported by the finding that it produces anti-inflammatory compounds that have protective effects in mouse models of DNBS- and DSS-induced colitis through inhibition of the NF-KB pathway (109, 110).

MMP12 is a macrophage-specific metalloelastase whose expression was strongly correlated with the abundance of F. prausnitzii and several other age-discriminatory taxa (including Clostridiales sp., D. formicigenerans, Blautia sp. and R. torques). MMP12 binding to the IKBα promoter is essential for transcriptional up-regulation of IKBα, which is required for IFNα secretion by leukocytes and antiviral immunity. Outside the cell, MMP12 cleavage also forms a feedback loop to down-regulate IFNα by degrading it, thereby limiting systemic effects of prolonged IFNα elevation (111). A similar negative feedback role has been described for macrophage MMP12 in the proteolysis and inactivation of pro-inflammatory CXC and CC cytokines released by LPS stimulation of polymorphonuclear leukocytes (112).

Plasma levels of heat shock proteins Hsp90aa1 and Hsp90ab1 were strongly negatively correlated with F. prausnitzii levels in the gut microbiota. The observed relationship between F. prausnitzii and Hsp90 in plasma suggest that there is an extracellular or secreted form of Hsp90. There is a growing appreciation of the role of extracellular heat shock proteins as ‘danger’ signals that stimulate innate and adaptive immune responses (113-115).

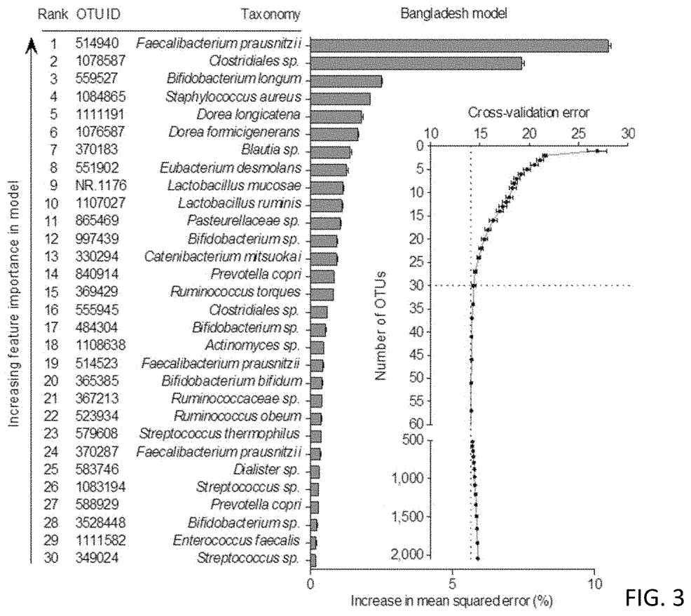

The gut microbiota/microbiome—A sparse 30 OTU RF-derived model of normal gut microbiota development, obtained from 25 healthy-growing members of a birth cohort living in Mirpur, an urban slum in Dhaka Bangladesh (1,2; Table 3 and FIG. 3), was applied to bacterial V4-16S rDNA datasets generated from fecal samples serially collected from the children in the SAM study (n=501).

TABLE 3

| Feature | | |
|---|---|---|
| importance | | |
| rank | OTU | Taxonomy |
| 1 | 514940 | Faecalibacterium prausnitzii |
| 2 | 1078587 | Clostridiales sp. |
| 3 | 559527 | Bifidobacterium longum |
| 4 | 1084865 | Staphylococcus aureus |
| 5 | 1111191 | Dorea longicatena |
| 6 | 1076587 | Dorea formicigenerans |
| 7 | 370183 | Blautia sp. |
| 8 | 551902 | Eubacterium desmolans |
| 9 | New.0.ReferenceOTU1176 | Lactobacillus mucosae |
| 10 | 1107027 | Lactobacillus ruminis |
| 11 | 865469 | Pasteurellaceae sp. |
| 12 | 997439 | Bifidobacterium sp. |
| 13 | 330294 | Catenibacterium mitsuokai |
| 14 | 840914 | Prevotella copri |
| 15 | 369429 | Ruminococcus torques |
| 16 | 555945 | Clostridiales sp. |
| 17 | 484304 | Bifidobacterium sp. |
| 18 | 1108638 | Actinomyces sp. |
| 19 | 514523 | Faecalibacterium prausnitzii |
| 20 | 365385 | Bifidobacterium bifidum |
| 21 | 367213 | Ruminococcaceae sp. |
| 22 | 523934 | Ruminococcus obeum |
| 23 | 579608 | Streptococcus thermophilus |
| 24 | 370287 | Faecalibacterium prausnitzii |
| 25 | 583746 | Dialister sp. |
| 26 | 1083194 | Streptococcus sp. |
| 27 | 588929 | Prevotella copri |
| 28 | 3528448 | Bifidobacterium sp. |
| 29 | 1111582 | Enterococcus faecalis |
| 30 | 349024 | Streptococcus sp. |
| 31 | 564704 | Weissella cibaria |
| 32 | 368698 | Clostridiales |
| 33 | 334459 | Escherichia sp. |
| 34 | 583656 | Bacteroides fragilis |
| 35 | 851865 | Faecalibacterium prausnitzii |
| 36 | 922761 | Enterobacteriaceae sp. |
| 37 | 235262 | Bifidobacterium sp. |
| 38 | 422878 | Megamonas funiformis |
| 39 | 1111294 | Escherichia coli |
| 40 | New.0.ReferenceOTU1101 | Coriobacteriaceae sp. |
| 41 | 368099 | Veillonella ratti |
| 42 | 470382 | Eubacterium hallii |
| 43 | 628226 | Intestinibacter bartlettii |
| 44 | 568118 | Prevotella sp. |
| 45 | 370225 | Bifidobacterium sp. |
| 46 | 692154 | Lactobacillus reuteri |
| 47 | 298050 | Veillonellaceae sp. |
| 48 | 813479 | Bifidobacterium sp. |
| 49 | 403068 | Staphylococcus sp. |
| 50 | 1059729 | Granulicatella adiacens |
| 51 | 262095 | Erysipelatoclostridium ramosum |
| 52 | 553611 | Bifidobacterium sp. |
| 53 | New.0.ReferenceOTU1133 | Lactobacillus fermentum |
| 54 | 1142029 | Bifidobacterium sp. |
| 55 | 646800 | Olsenella sp. |
| 56 | 524884 | Eubacterium biforme |
| 57 | 708680 | Eubacterium rectale |
| 58 | 72820 | Bifidobacterium sp. |
| 59 | 289709 | Escherichia sp. |
| 60 | 685156 | Lactobacillus mucosae |

This model allowed us to define microbiota-for-age Z (MAZ)-scores as a function of treatment arm and time [9.3±3.7 samples/child (mean±SD)]. The MAZ-score measures the deviation in development of a child's microbiota from that of chronologically-age matched reference healthy children based on the representation of the ensemble of age-discriminatory strains contained in the RF-derived model (2). Significant microbiota immaturity was apparent in the SAM and post-SAM MAM groups (FIG. 1C). Moreover, MAZ-scores in this SAM cohort were significantly correlated with WHZ, HAZ, and WAZ (Pearson r=0.16, p=0.0004; r=0.13, p=0.003; r=0.10, p=0.02; respectively). The MAZ did not change significantly at discharge but improved significantly by 1-month post-discharge (p=0.0051 versus admission, Mann-Whitney test); this improvement could reflect several changes a child is exposed to when returning to their home environments including, for example, increased dietary diversity and reduced antibiotic usage. MAZ did not change significantly thereafter (FIG. 1 B).

A number of the age-discriminatory strains were significantly correlated with anthropometric indices as well as with plasma proteins/biological processes that mediate growth. We also identified significant negative correlations between these taxa and mediators of systemic inflammation and anorexia/cachexia [note that B. longum (OTU 559527) had the greatest number of significant correlations; n=114].

The effects of the therapeutic food interventions on the representation of metabolic pathways in the gut microbiome were defined by shotgun sequencing of 331 fecal DNA samples obtained from 30 members of the Mirpur birth cohort with consistently healthy anthropometry and 15 of the 54 children enrolled in the SAM study; these latter children were selected based on their age (12-18 months) and the fact that we had corresponding plasma metabolomic and proteomic datasets for at least two of the three time points sampled. The abundances of microbial genes that mapped to pathways in the microbial communities SEED (mcSEED) database (12) related to metabolism of amino acids, carbohydrates, fermentation products and B vitamins/related cofactors were first defined in healthy children sampled monthly from birth to two years of age. A set of age-discriminatory metabolic pathways (mcSEED ‘subsystems’/pathway modules) was identified using RF. The resulting sparse RF-derived model (Methods, FIG. 4) allowed us to assign a state of development (functional age or ‘maturity’) to the fecal microbiomes of the 15 children treated for SAM. Relative functional maturity was significantly correlated with MAZ, WHZ, and WAZ scores during the course of the trial (Pearson r and p-values: MAZ, r=0.55, p<0.0001; WHZ, r=0.30, p=0.0011; WAZ, r=0.23, p=0.013). At enrollment, and just prior to administration of therapeutic foods, children with SAM had more immature microbiomes (one-way ANOVA p=0.0002; Dunnett's multiple comparisons test for healthy vs. SAM adjusted p-values at the two time points, 0.027 and 0.0001, respectively). There was a statistically significant improvement in functional maturity from initiation of therapeutic food treatment to discharge, and at one and six months post-discharge (Tukey's multiple comparisons test; adjusted p-values =0.039, 0.0028 and 0.025, respectively). However, this improvement was not sustained at later time points (FIG. 4). Comparing the relative abundances of the 30 most age-discriminatory pathways at six time points revealed that the SAM microbiome had significantly reduced representation of (i) amino acid metabolic pathways, including those involved in isoleucine, leucine, valine biosynthesis and uptake, (ii) several carbohydrate utilization pathways (arabinose and arabinosides, rhamnose and rhamnogalacturonan, and sialic acid) and (iii) multiple pathways involved in B-vitamin metabolism, including cniacin/NADP biosynthesis' (FIG. 4). The observed underrepresentation of age-discriminatory OTUs and metabolic pathways in the gut communities of children with post-SAM MAM provided the rationale for developing a pipeline to test complementary food ingredients for their ability to repair this immaturity.

### Example 2

### Screening Complementary Food Ingredients

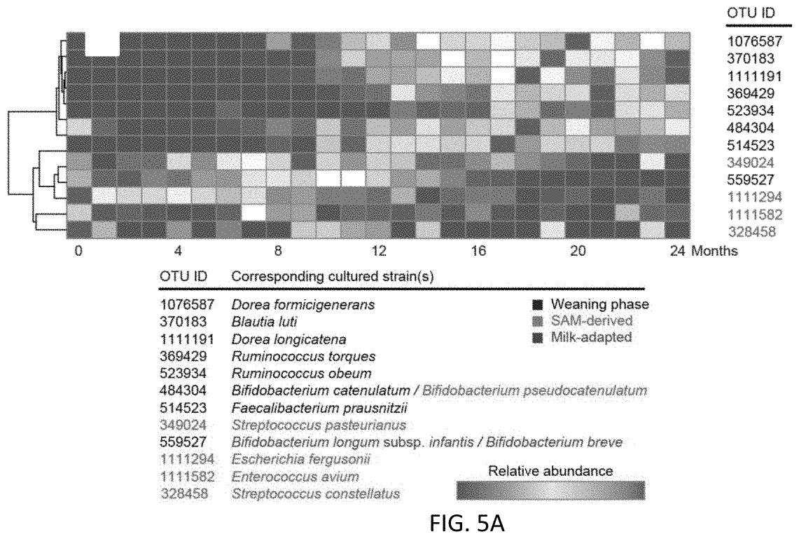

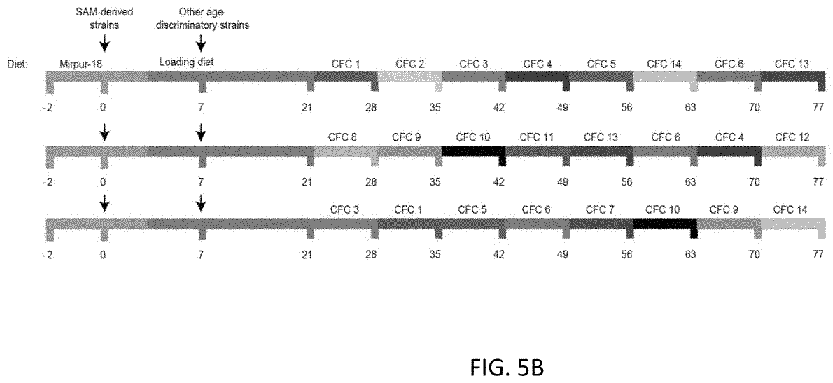

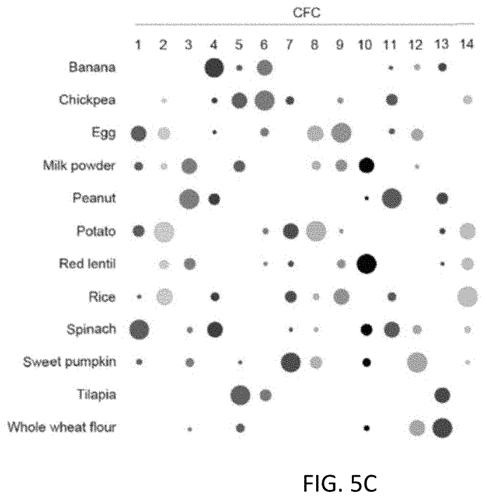

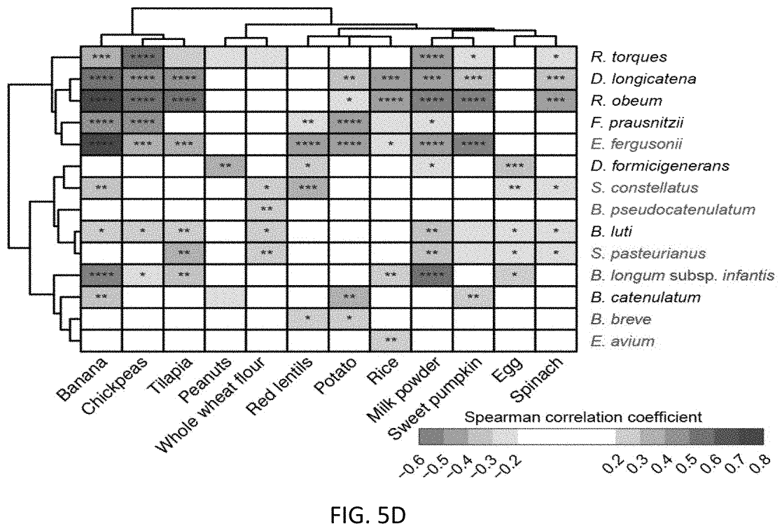

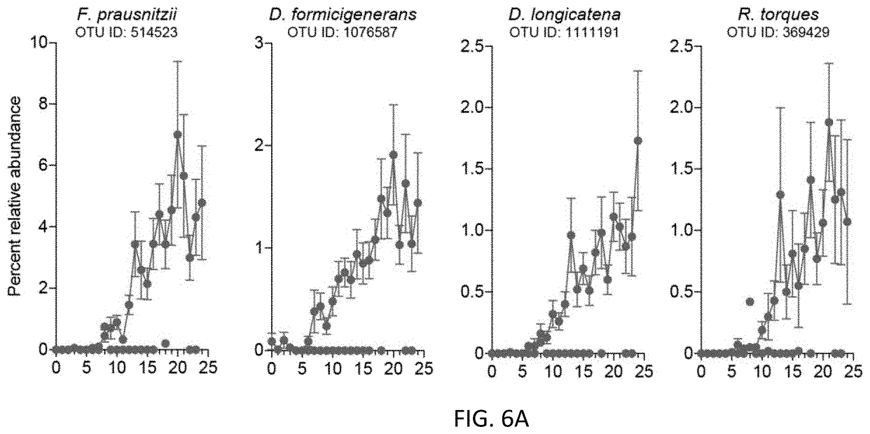

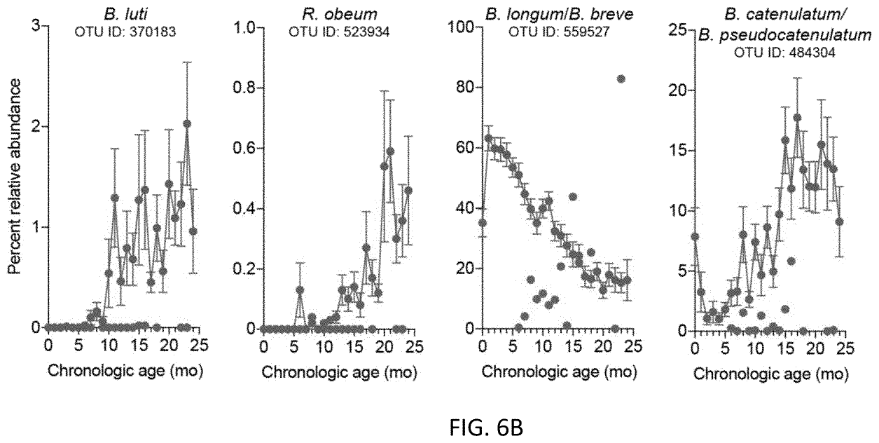

Nine age-discriminatory bacterial strains were cultured from the fecal microbiota of three healthy children, aged 6-23 months, who lived in Mirpur, and genomes of these isolates were sequenced (Table 4). Seven of these nine isolates had V4-16S rDNA sequences that corresponded to age-discriminatory OTUs whose representation is associated with the period of complementary food consumption (‘weaning-phase’ OTUs) (FIG. 5A) while two, Bifidobacterium longum subsp. infantis and Bifidobacterium breve, are most prominent during the period of exclusive/predominant milk feeding (FIG. 5A; (13)). OTUs representing seven of the nine cultured strains were significantly depleted in the fecal microbiota of Bangladeshi children with SAM prior to treatment (FIG. 6). Seven additional age-discriminatory strains were cultured from the immature fecal microbiota of a 24-month-old child with SAM enrolled in the same study as the subcohort shown in FIG. 1 (Table 4). Together, the consortium of 16 strains represented OTUs that directly matched 65.6±22.8% (mean±SD) of V4-16S rDNA sequences identified in 1039 fecal samples collected from 53 healthy members of the Mirpur birth cohort during their first 2 postnatal years, and 74.2±25.2% of the sequences in fecal samples collected from 38 children with SAM. Importantly, the weaning-phase OTUs are not unique to the Bangladeshi population (see (14)).

TABLE 4

| | Bacterial strain |
|---|---|
| Bangladeshi | Bifidobacterium catenulatum JG_Bg468 |
| weaning | Blautia luti SSTS_Bg7063 |
| phase age- | Dorea formicigenerans SSTS_Bg7063 |
| discriminatory | Dorea longicatena SSTS_Bg7063 |
| strains (from | Faecalibacterium prausnitzii SSTS_Bg7063 |
| healthy donors) | Ruminococcus obeum SSTS_Bg7063 |
| | Ruminococcus torques SSTS_Bg7063 |
| Strains from a | Bifidobacterium pseudocatenulatum SS_Bg39 |
| 24 month old | Enterococcus avium SS_Bg39 |
| SAM donor | Escherichia fergusonii SS_Bg39 |
| (normally | *Streptococcus constellatus SS_Bg39 |
| prominent in | Streptococcus pasteurianus SS_Bg39 |
| first 8-11 | |
| postnatal | |
| months) | |
| Milk-adapted | Bifidobacterium longum subsp. infantis JG_Bg463 |
| strains | Bifidobacterium breve JG_Bg463 |
| (from 6- | |
| month-old | |
| healthy donor) | |
| Malawian | Clostridium symbiosum TS_8243C |
| weaning | Clostridium nexile TS_8243C |
| phase age- | Ruminococcus gnavus TS_8243C |
| discriminatory | |
| strains (from | |
| healthy donors) | |
| Bangladeshi | Clostridium amygdalinum SV_Bg7063 |
| children | Eggerthella lenta SV_Bg7063 |
| with post-SAM | *Lactobacillus gasseri SV_Bg7063 |
| MAM | |

*These strains did not colonize recipient mice

To identify complementary foods that selectively increase the representation of weaning-phase age-discriminatory strains deficient in immature SAM-associated microbiota, we colonized 5-week-old, germ-free C57BI/6J mice with the consortium of cultured, sequenced bacterial strains. Following colonization, an 8-week period of diet ‘oscillations’ was initiated (FIG. 5B). We incorporated 12 complementary food ingredients commonly consumed in Mirpur (6) into 14 different diets using a random sampling strategy (see Methods and Tables s8A-E of Gehrig et al. Science, 2019, 365(6449):eaau4732, which is incorporated by reference in its entirety). The composition of these complementary food combinations (CFCs) and their order of administration to mice were based on considerations described in the legend to FIG. 5B, FIG. 5C.

Spearman's rank correlation coefficients were calculated between the relative abundances of the 14 bacterial strains that colonized mice and levels of complementary food ingredients in the 14 CFCs tested (FIG. 5D). Chickpea and banana had statistically significant strong positive correlations with the greatest number of strains representing weaning-phase age-discriminatory OTUs. Tilapia had a narrower range of significant positive effects (FIG. 5D). Chickpea, banana, and tilapia also had significant negative correlations with levels of the pre-weaning, milk-adapted B. longum subsp. infantis isolate. A sobering observation was that a number of complementary food ingredients typically represented in diets consumed by 18-month-old children living in Mirpur had significant negative correlations with six of the weaning-phase age-discriminatory strains, including rice, milk powder, potato, spinach and sweet pumpkin (FIG. 5D). Rice gruel with milk is the most common first complementary food given to Bangladeshi children (15). Moreover, egg, which is included in a number of regimens for nutritional rehabilitation of children with acute malnutrition (16), was negatively correlated with the abundance of two weaning-phase strains, D. formicigenerans and B. luti.

### Example 3

### Testing an Initial MDCF Prototype

Khichuri-Halwa (KH) is a therapeutic food commonly administered together with Milk-Suji (MS) to Mirpur children with SAM. A previous study documented the inability of this intervention to repair gut microbiota immaturity (2). We prepared a diet that mimicked MS/KH (see, Table s8D-E of Gehrig et al. Science, 2019, 365(6449):eaau4732, which is incorporated by reference in its entirety); 7 of its 16 ingredients are commonly consumed complementary foods that had little, if any, effect on the representation of weaning-phase age-discriminatory strains (i.e., rice, red lentils, potato, pumpkin, spinach, whole wheat flour and powdered milk; FIG. 5D). The effects of MS/KH on members of the 14-member consortium and the host were compard to those produced by an initial MDCF prototype containing chickpeas, banana, and tilapia (see, Table s9B of Gehrig et al. Science, 2019, 365(6449):eaau4732, which is incorporated by reference in its entirety). Five-week-old germ-free C57BI/6J mice colonized with the consortium were monotonously fed either of the two diets ad libitum for 25 days.

Microbial community responses—COPRO-Seq of cecal DNA revealed that compared to MS/KH, consumption of the MDCF prototype resulted in significantly higher relative abundances of a number of weaning-phase age-discriminatory taxa including F. prausnitzii, D. longicatena, and B. luti (p<0.01; Mann-Whitney test; FIG. 7). This prototype did not promote the fitness of the SAM donor-derived strains, with the exception of E. fergusonii.

We used targeted mass spectrometry to quantify cecal levels of carbohydrates, short-chain fatty acids, plus amino acids and their catabolites (Table 5A-D). Germ-free animals served as reference controls to define levels of cecal nutrients that, by inference, would be available for bacterial utilization in the different diet contexts. Noteworthy findings include: (i) levels of butyrate and succinate were significantly higher in colonized animals consuming MDCF compared to MS/KH (FIG. 7, Table 5B); (ii) there were no statistically significant diet-associated differences in levels of any of the amino acids measured in germ-free animals but when compared to their colonized MS/KH-fed counterparts, colonized MDCF-consuming animals had significantly elevated cecal levels of six amino acids classified as essential in humans (the three branched-chain amino acids plus phenylalanine and tryptophan; FIG. 7, Table 5C), and (iii) two tryptophan-derived microbial metabolites that play important roles in suppressing inflammation and are neuroprotective, 3-hydroxyanthranillic acid (3-HAA) and indole-3-lactic acid (17-22), were significantly elevated in colonized animals fed MDCF compared to their MS/KH-treated counterparts (Table 5D).

Table 5—Diet- and colonization-dependent effects on levels of cecal metabolites in mice colonized with the defined consortium of age-discriminatory strains and monotonously fed the initial MDCF prototype versus Milk Suji/Khichuri-Halwa (MS/KH) (see FIG. 3).

TABLE 5A
Diet- and microbiota-dependent differences: Carbohydrates

| Diet- and microbiota-dependent | FDR-corrected |
|---|---|
| differences | p-value |
| Sorbitol | 4.6E−24 |
| Mannitol | 7.3E−23 |
| Rhamnose | 8.7E−11 |
| Sucrose | 1.1E−10 |
| N-Acetyl-D-galactosamine | 2.1E−09 |
| Maltose | 3.3E−09 |
| Fructose | 5.7E−08 |
| Lactose | 6.0E−08 |
| Galactosamine hydrochloride | 6.8E−08 |
| Fucose | 5.7E−07 |
| Glucuronic acid | 2.5E−06 |
| Ribitol | 5.1E−06 |
| Galactitol | 8.5E−06 |
| Arabinose | 5.0E−05 |
| Glucose | 0.0002 |
| Glycerol | 0.01 |
| Galacturonic acid | 0.05 |
| Galactose | 0.05 |
| Mannose | 0.05 |
| Xylose | 0.06 |
| N-acetyl-D-mannosamine | 0.1 |
| Maltitol | 0.3 |
| N-Acetyl-D-glucosamine | 0.7 |
| Ribose | 0.8 |

TABLE 5B
Diet- and microbiota-dependent differences: Short-chain fatty acids

| Diet- and microbiota-dependent | FDR-corrected |
|---|---|
| differences | p-value |
| Butyrate | 5.0E−05 |
| Lactate | 0.0001 |
| Succinate | 0.005 |
| Acetate | 0.5 |

TABLE 5C
Diet- and microbiota-dependent differences: Amino acids

| Diet- and microbiota-dependent | FDR-corrected |
|---|---|
| differences | p-value |
| Lysine | 0.0015 |
| Proline | 0.03 |
| Phenylalanine | 0.05 |
| Asparagine | 0.06 |
| Isoleucine | 0.07 |
| Tyrosine | 0.08 |
| Valine | 0.09 |
| Leucine | 0.09 |
| Methionine | 0.2 |
| Serine | 0.4 |
| Threonine | 0.4 |
| Alanine | 0.4 |
| Glutamine | 0.4 |
| Glycine | 0.7 |
| Histidine | 0.7 |
| Glutamate | 0.9 |

TABLE 5D
Diet- and microbiota-dependent differences:
Aromatic amino acid metabolites

| Diet- and microbiota-dependent | FDR-corrected |
|---|---|
| differences | p-value |
| Shikimic acid | 3.8E−11 |
| 4-Hydroxybenzoic acid | 1.6E−05 |
| NMN | 3.0E−03 |
| 3-Hydroxyanthranilic acid | 4.2E−03 |
| Indole-3-lactic Acid | 4.2E−03 |
| 4-hydroxyphenylacetic acid | 5.6E−03 |
| L-Tryptophan | 5.9E−03 |
| Anthranilic acid | 1.8E−02 |

To further characterize the responses of the 14-member consortium of age-discriminatory bacterial strains to the initial MDCF prototype, 5-week-old germ-free C57BI/6J mice (n=6 animals/group; 3 cages of dually-housed mice/group) were placed on MDCF or MS/KH and three days later gavaged with the 14-member consortium. All mice were monotonously fed their designated diets for an additional 40 days. There were no significant differences in microbial community biomass [2.9±0.8 pg DNA/g cecal contents (MDCF) versus 2.7±0.3 pg/g (MS/KH); p=0.48, Mann-Whitney test]. COPRO-Seq analysis disclosed that as in the previous experiment shown in FIG. 7, the weaning-phase age-discriminatory strains R. torques, R. obeum, F. praunitzii and D. longicatena exhibited the largest and most statistically significant elevations in their relative abundances in the ceca of MDCF- compared to MS/KH-fed mice.

Microbial RNA-Seq datasets were generated from cecal contents and the results were interpreted based on KEGG and SEED-based annotations of the 40,735 predicted protein-coding genes present in consortium members, plus in silico predictions of the abilities of bacterial strains to produce, utilize and/or share nutrients. Community-level analysis revealed specific community members manifested MDCF-associated increases in expression of genes involved in (i) biosynthesis of the essential amino acids, including branched-chain amino acids (R. obeum, R. torques) and (ii) generation of aromatic amino acid metabolites (R. obeum, R. torques, F. prausnitzii).

Three weaning-phase age-discriminatory strains, F. prausnitzii, R. obeum, and R. torques, had the greatest number of genes with statistically significant differences in their expression between the two diets (320, 308 and 184, respectively). Given its high feature importance scores in the Bangladeshi and other RF-derived models of microbiota development (see Table 3, FIGS. 3 and (14)), its consistent increase in fitness with MDCF compared to MS/KH across experiments, its multiple predicted nutrient requirements, and the large number of genes that are differentially expressed between the two diets, F. praunitzii represents an attractive ‘model’ for investigating how the MDCF prototype affects its weaning-phase age-discriminatory microbial targets.

Among the F. prausnitzii genes with significantly higher levels of expression in the ceca of mice fed MDCF versus MS/KH were an alpha-glucosidase belonging to CAZyme glycoside hydrolase family (GH) 31 (EC:3.2.1.20; encoded by FPSSTS7063_00084), a GH 13 oligo-1,6-glucosidase (EC:3.2.1.10; FPSSTS7063_00083), a glycosyltransferase (GT) family 35 starch/glycogen phosphorylase (EC:2.4.1.1; FPSSTS7063_00079), and three linked genes in the maltose/maltodextrin transport system (FP SSTS7063_00085-87). Increased expression of F. prausnitzii genes encoding enzymes that hydrolyze 1,4- and 1,6-alpha-glucosidic linkages suggests that starch serves as a preferred substrate. In contrast, R. torques exhibits increased expression of the agaEFG-rafA genes involved in uptake and hydrolysis of alpha-galactosides such as raffinose (RTSSTS7063_01731-01735); this pathway is absent from F. prausnitzii. These differentially expressed genes might reflect adaptations to chickpea and banana, two of the three complementary food leads represented in the inital MCDFprototype; both complementary foods are rich in raffinose and stachyose while banana is also enriched in resistant starch (116, 117). In contrast, a set of 20 F. prausnitzii genes represented in several predicted operons involved in utilization of hexuronates (D-glucuronic and D-galacturonic acids) exhibit 2 to 23-fold lower levels of expression in mice fed the MDCF diet compared to MS/KH. These latter findings are consistent with observed differences in the availability of these nutrients in the cecum (e.g., glucuronic acid is present at lower levels in germ-free mice fed MDCF vs. MS/KH).

Host effects—Serum levels of IGF-1 were significantly higher in colonized mice consuming the initial MDCF prototype compared to those consuming MS/KH. This effect was diet- and colonization-dependent, with germ-free animals exhibiting significantly lower levels of IGF-1 in both diet contexts (FIG. 7). Serum insulin levels were also higher in colonized animals consuming MDCF compared to MS/KH [800.7±302.9 ng/mL (mean±SD) versus 518.7±135.1 ng/mL, respectively; p=0.06; unpaired t-test].

IGF-1 binding to its receptor tyrosine kinase, IGF-1R, affects a variety of signal transduction pathways, including one involving the serine/threonine kinase Akt/PKB, phosphatidylinositol-3 kinase (PI-3K) and the mammalian target of rapamycin (mTOR). Absorption of several amino acids from the gut, notably branched-chain amino acids and tryptophan, leads to activation of mTOR (23). Colonized animals fed MDCF had significantly higher levels of hepatic phosphoSer473-Akt, consistent with activation of Akt by IGF-1 signaling via the PI-3K pathway (FIG. 7; Mann-Whitney test). Levels of phospho-AMPK were not significantly affected by diet (FIG. 7), suggesting that Akt phosphorylation is not caused indirectly by altered hepatic energy status. Phosphorylation of hepatic Jak 2 (Tyr1007/1008) and mTOR (Ser2448), which are involved in IGF-1 production, was significantly increased in colonized mice consuming MDCF (FIG. 7), while phosphorylation of STATS, also implicated in IGF-1 production, was not significantly altered.

Previous studies of adult germ-free mice reported increases in serum IGF-1 after their colonization with gut microbiota from conventionally-raised mice; increased IGF-1 levels were also associated with increased bone formation (24, 25). Micro-computed tomography of mouse femurs revealed a significant increase in femoral cortical bone area in MDCF-fed animals; the effect was both diet- and microbiota-dependent (FIG. 7).

We used targeted mass spectrometry to quantify levels of amino acids, acylCoAs, acylcarnitines, and organic acids in serum, liver, and gastrocnemius muscle. Products of non-oxidative metabolism of glucose and pyruvate (lactate via glycolysis, and alanine via transamination of pyruvate, respectively) were significantly lower in mice fed MDCF compared to mice fed MS/KH; this was true for alanine in serum, skeletal muscle and liver and for lactate in liver. Oxidative metabolism of glucose is associated with nutritionally replete, anabolic conditions. These findings are consistent with the observed elevations of the anabolic hormone IGF-1 in MDCF-fed compared to MS/KH-fed mice. MDCF-fed mice had significantly higher circulating levels of valine and leucine/isoleucine than their MS/KH-fed counterparts (FIG. 7). Skeletal muscle C5 carnitine and the closely related metabolite C5-OH/C3 carnitine were significantly higher in animals consuming MDCF (FIG. 7). In liver, C3 and C5 acylcarnitines were significantly lower in MDCF-treated mice (FIG. 7), suggesting that the more nutritionally replete state associated with MDCF may act to limit branched-chain amino acid oxidation in this tissue.

### Example 4

### Testing Additional MDCF Prototypes in Gnotobiotic Mice

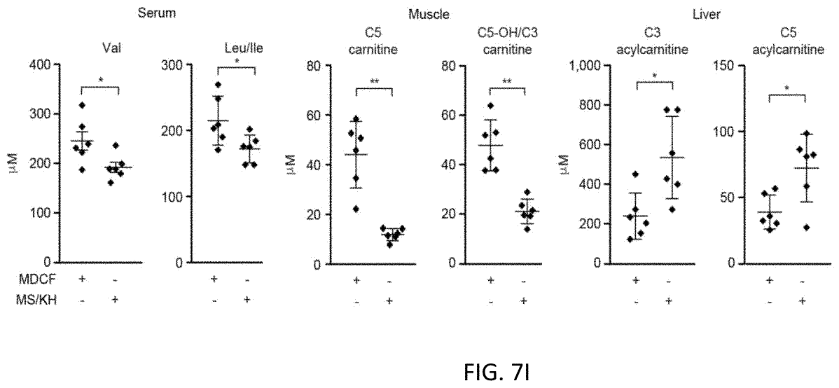

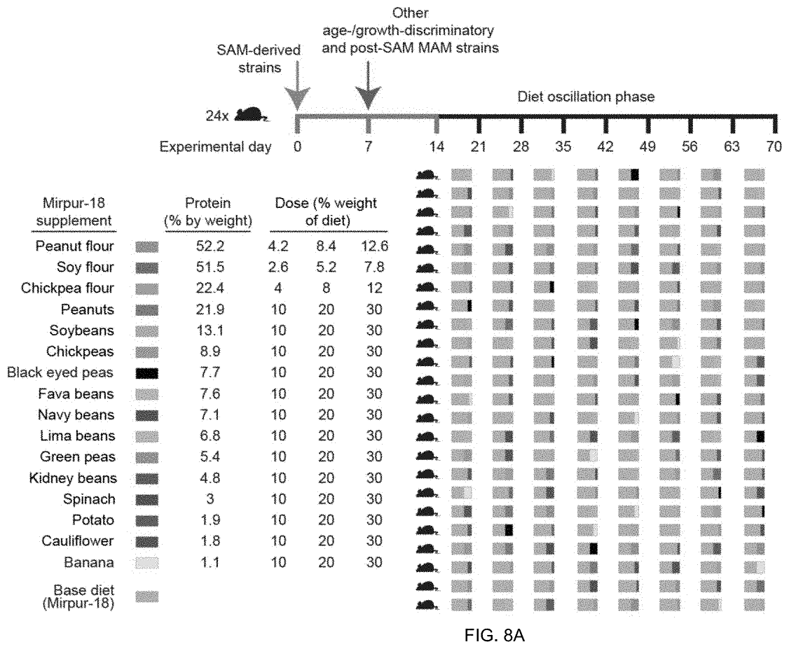

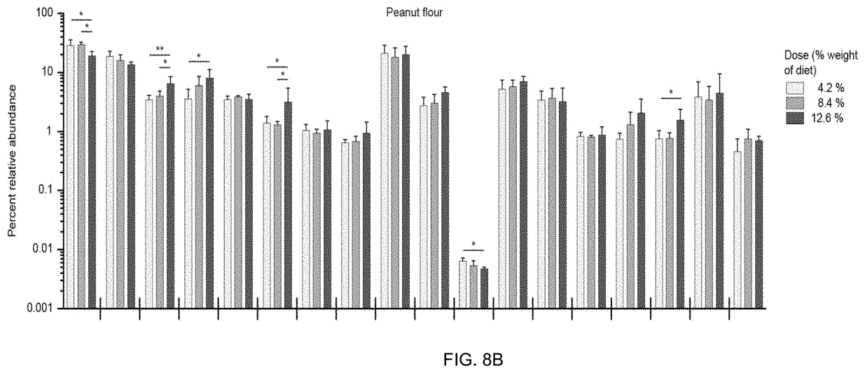

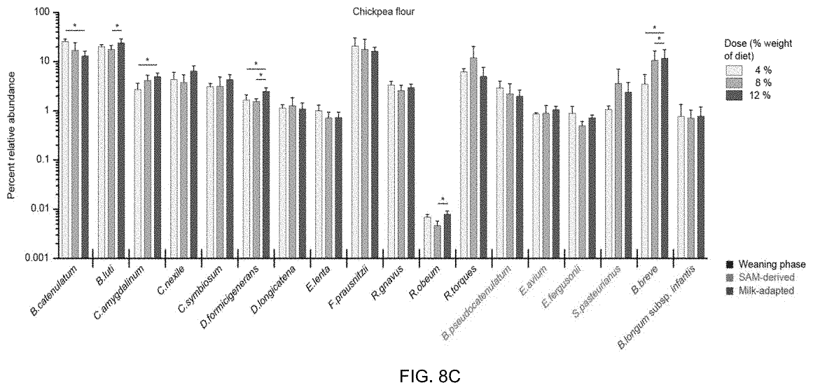

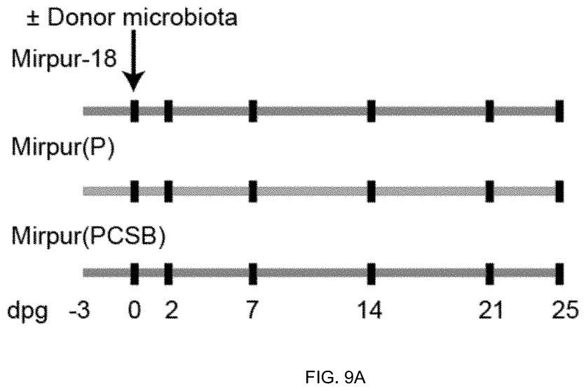

Incorporating tilapia into MDCF prototypes poses several problems: its organoleptic properties are not desirable, and its cost is greater than that of commonly consumed plant-based sources of protein. To identify alternatives to tilapia, we selected an additional 16 plant-derived complementary food ingredients with varied levels and quality of protein (26), that are culturally acceptable, affordable and readily available (FIG. 8A). Their effects were tested in gnotobiotic mice colonized with a defined, expanded consortium of 18 age- and growth-discriminatory bacterial strains (Table 4). We generated 48 mouse diets by supplementing a prototypic base diet representative of that consumed by 18-month-old children living in Mirpur ('Mirpur-18′), with each of the individual ingredients incorporated at three different concentrations (Table 6A; FIG. 8A; Methods). The results revealed that in this defined community context, peanut flour had the greatest effect on the largest number of targeted weaning-phase age-discriminatory taxa, followed by chickpea flour (FIG. 8B). Soy flour, which promoted the representation of two of these taxa, had the second-highest percentage protein after peanut flour (FIG. 8A) and its protein quality was among the highest of the ingredients tested. (Amino acid scores were calculated based on the recommendations of the 1985 FAO/WHO report for healthy growth of a preschool-age child [D. J. Millward, Br. J. Nutr. 108, (Suppl. 2), S31-43 (2012)].

$$Amino acid score = \frac{mg of essential amino acid per gram of test protein}{\begin{matrix}\begin{matrix}mg per gram of amino acid recommended \\ by the 1985 FAO / WHO report for healthy growth\end{matrix} \\ of a preschool - age child\end{matrix}} \times 100 )$$

Based on these observations, we chose soy and peanut flours as replacements for tilapia in subsequent MDCF formulations.

Table 6—Testing 16 plant-derived complementary food ingredients in gnotobiotic mice colonized with an 18-member consortium of age- and growth-discriminatory bacterial taxa.

TABLE 6A
composition and diets used in the screen. The plant-derived CF ingredients used to supplement
Mirpur-18 are peanut flour, soy flour, chickpea flour, soybeas, chickpeas, black-eyed peas,
fava beans, lima beans, green peas, kidney peas, spinach, potato, cauliflower, banana

| | | | Base Mirpur-18 | Base Mirpur-18 | Base Mirpur-18 |
|---|---|---|---|---|---|
| | | | ingredient | ingredient | ingredient |
| | | | levels with | levels with | levels with |
| | | | 10% plant- | 20% plant- | 30% plant- |
| | | | derived CF | derived CF | derived CF |
| | | Mirpur-18 | ingredient | ingredient | ingredient |
| | | unsup. | supplementation | supplementation | supplementation |
| Ingredient type | Component | g of ingredient/100 g diet | | | |
| Complementary | Red lentils | 16.2 | 14.6 | 13.0 | 11.4 |
| food (CF) | Milk powder | 13.9 | 12.5 | 11.1 | 9.7 |
| | Potato | 6.9 | 6.2 | 5.5 | 4.8 |
| | Pumpkin | 6.0 | 5.4 | 4.8 | 4.2 |
| | Rice | 41.8 | 37.6 | 33.4 | 29.2 |
| | Spinach | 6.3 | 5.7 | 5.1 | 4.4 |
| Other | Garlic | 0.5 | 0.4 | 0.4 | 0.3 |
| ingredients | Iodized salt | 0.5 | 0.4 | 0.4 | 0.3 |
| | Onion | 3.7 | 3.3 | 3.0 | 2.6 |
| | Soybean oil | 3.7 | 3.3 | 3.0 | 2.6 |
| | Turmeric | 0.5 | 0.4 | 0.4 | 0.3 |

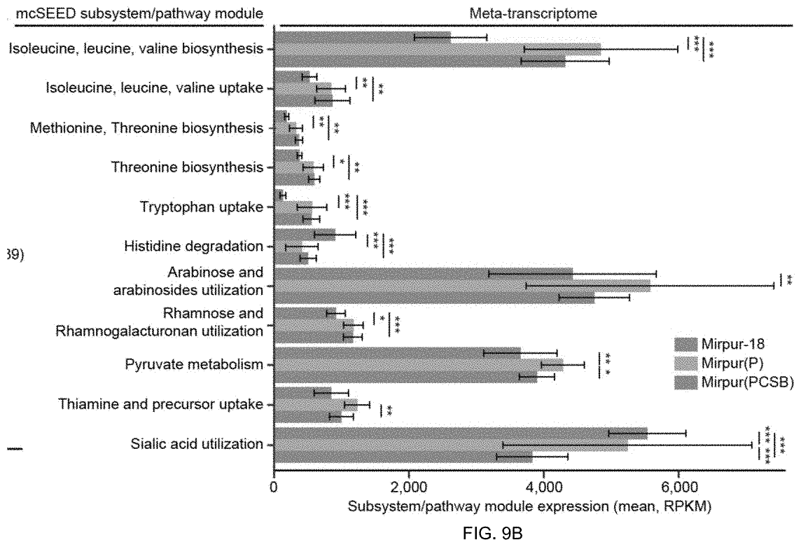

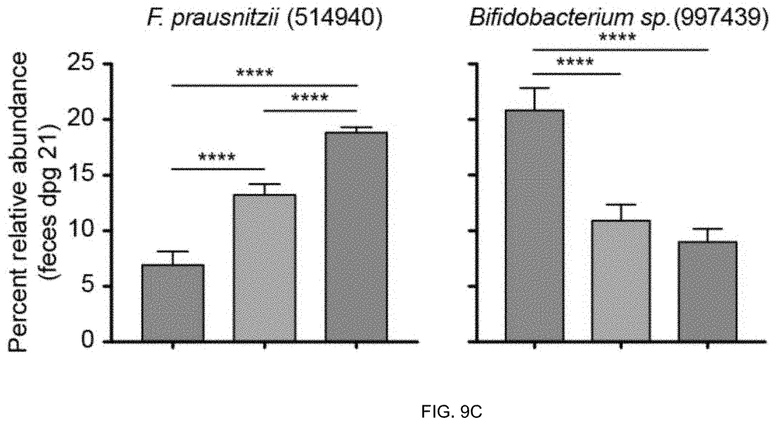

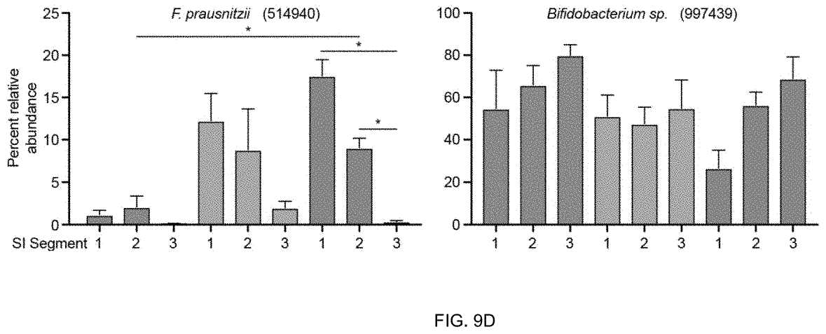

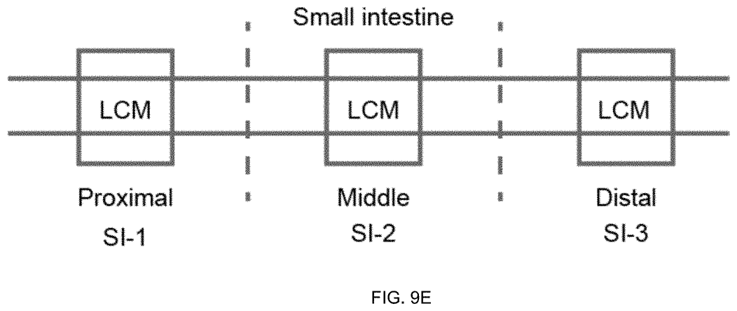

We reasoned that by transplanting a representative immature intact microbiota into young, germ-free mice, we could investigate whether gut ‘health’ (defined by relative abundances of community members, expression of microbial genes in mcSEED metabolic pathways, and biomarkers/mediators of gut barrier function), was improved by supplementing the Mirpur-18 diet with one or more complementary food ingredients that target weaning-phase age-discriminatory taxa. Fifteen fecal samples from 12 different children, obtained during or after treatment for SAM, were screened in gnotobiotic mice to identify communities containing the greatest number of transmissible weaning-phase age-discriminatory taxa and to assess their response to supplementation of Mirpur-18 (Table 7). We selected a sample obtained from a donor (PS.064) who had post-SAM MAM; in addition to the successful transmission of targeted taxa, 88.7±1.3% (mean±SD) of the recipient animals' gut communities consisted of OTUs that were detected at >0.1% relative abundance in the donor sample. Three groups of mice were colonized with this microbiota and monotonously fed one of three diets; unsupplemented Mirpur-18, Mirpur-18 supplemented with peanut flour [Mirpur(P)], or Mirpur-18 supplemented with four of the lead ingredients [Mirpur(PCSB), with peanut flour, chickpea flour, soy flour and banana] (FIG. 9A. Three control groups were maintained as germ-free; each group was fed one of the three diets. Diet-associated differences in levels of cecal branched-chain and other amino acids in colonized animals were not accompanied by significant differences in levels of the corresponding serum amino acids.

TABLE 7
A screen of the effects of complementary food ingredients on age-discriminatory
strains present in 15 intact uncultured microbiota samples from children
enrolled in the SAM study - Composition of diets used to screen the
microbiota after transplantation into gnotobiotic mice.

| | | Screening 15 SAM/post-SAM MAM microbiota in | | |
|---|---|---|---|---|
| | | gnotobiotic mice | | |
| | | | PCBT (Peanut, | |
| | Components | | chickpea, banana, | |
| Ingredient type | (g/100 g diet) | Mirpur-18 | tilapia) | Mirpur(PCBT) |
| Lead | Banana | — | 25.0 | 20.0 |
| complementary | Banana powder | — | — | — |
| food | Chickpea | — | 50.0 | 24.5 |
| | Chickpea flour | — | — | — |
| | Peanut | — | 12.5 | 5.0 |
| | Peanut flour | — | — | — |
| | Soy flour | — | — | — |
| Complementary | Egg | — | — | — |
| food | Red lentils | 16.2 | — | 6.5 |
| | Milk powder | 13.9 | 5.0 | 13.9 |
| | Potato | 6.9 | — | 2.7 |
| | Pumpkin | 6.0 | — | 2.4 |
| | Pumpkin powder | — | — | — |
| | Rice | 41.8 | — | 10.2 |
| | Spinach | 6.3 | — | 2.5 |
| | Tilapia | — | 5.5 | 9.3 |
| Other | Cardamom powder | — | — | — |
| ingredients | Garlic | 0.5 | — | 0.5 |
| | Garlic powder | — | — | — |
| | Iodized salt | 0.5 | — | 0.5 |
| | Lecithin | — | — | — |
| | Onion | 3.7 | — | 1.5 |
| | Onion powder | — | — | — |
| | Palm oil | — | — | — |
| | Powdered sugar | — | — | — |
| | Soybean oil | 3.7 | 2.0 | — |
| | Turmeric | 0.5 | — | 0.5 |
| | Vitamin Mineral | — | — | — |
| | Blend | | | |
| | kcal/100 g diet | 183.9 | 227.3 | 194.4 |
| | g protein/100 g diet | 6.9 | 10.2 | 10.7 |
| | g lipids/100 g diet | 7.7 | 11.1 | 7.2 |
| | g | 21.8 | 24.3 | 23.1 |
| | carbohydrates/100 g | | | |
| | diet | | | |
| | g fiber/100 g diet | 2.2 | 5.2 | 3.6 |

TABLE 8

| | | PS.064.S7-colonized gnotobiotic mice | | |
|---|---|---|---|---|
| | | | Mirpur-18 + | Mirpur-18 + |
| | | | Peanut | (Peanut/Chickpea/Soy |
| | Components | | flour | flours, Banana) |
| Ingredient type | (g/100 g diet) | Mirpur-18 | [Mirpur(P)] | [Mirpur(PCSB)] |
| Lead | Banana | — | — | 7.5 |
| complementary | Chickpea flour | — | — | 3.0 |
| food | Peanut flour | — | 5.1 | 3.2 |
| | Soybean flour | — | — | 2.0 |
| Complementary | Red lentils | 16.2 | 15.4 | 13.7 |
| food | Milk powder | 13.9 | 13.2 | 11.8 |
| | Potato | 6.9 | 6.5 | 5.8 |
| | Pumpkin | 6.0 | 5.7 | 5.1 |
| | Rice | 41.8 | 39.6 | 35.3 |
| | Spinach | 6.3 | 6.0 | 5.3 |
| Other | Garlic | 0.5 | 0.4 | 0.4 |
| ingredients | Iodized salt | 0.5 | 0.4 | 0.4 |
| | Onion | 3.7 | 3.5 | 3.1 |
| | Soybean oil | 3.7 | 3.5 | 3.1 |
| | Turmeric | 0.5 | 0.4 | 0.4 |
| | kcal/100 g diet | 183.9 | 191.3 | 190.2 |
| | g protein/100 g diet | 6.9 | 9.2 | 9.2 |
| | g lipids/100 g diet | 7.7 | 7.3 | 6.5 |
| | g | 21.8 | 21.1 | 19.1 |
| | carbohydrates/100 g | | | |
| | diet | | | |
| | g fiber/100 g diet | 2.2 | 2.9 | 3.2 |

The effects of diet supplementation on expression of genes in microbial metabolic pathways were defined by RNA-Seq of cecal contents harvested from mice after 25 days of consumption of the different diets (Table 9). The mcSEED categories ‘Amino Acid Metabolism’, ‘Vitamin and Cofactor Metabolism’, ‘Carbohydrate Utilization’, and ‘Fermentation Products’ and their subsystems/pathway modules were assigned ranks, calculated by dividing the total number of differentially expressed genes in a category/subsystem/pathway module by the total number of genes in that category/subsystem/pathway module. Higher rank corresponds to a greater proportion of differentially expressed genes in that category/subsystem/pathway module. The results (FIG. 9B) disclosed ‘Amino Acid Metabolism’ as the category with highest rank. Eighteen of the 20 subsystems/pathway modules belonging to this category, including all of the biosynthetic subsystems/pathway modules, were upregulated in mice consuming Mirpur(PCSB) or Mirpur(P) compared to Mirpur-18; the most upregulated subsystem/pathway module was ‘isoleucine, leucine, valine biosynthesis’ (60 genes significantly upregulated, and 4 significantly downregulated with Mirpur(PCSB) versus Mirpur-18; p-values based on gene set enrichment analysis). This subsystem/pathway module was also the most upregulated on Mirpur(P) compared to Mirpur-18 (27 genes compared to zero downregulated). See FIG. 9B and FIG. 4 for other age-discriminatory mcSEED pathway modules that showed significantly lower abundances in the microbiomes of Bangladeshi individuals with SAM and whose expression was increased by Mirpur(PCSB) or Mirpur(P). Serum levels of a product of branched-chain amino acid metabolism, C5:1-acylcarnitine, were significantly higher in mice consuming Mirpur(PCSB) compared to unsupplemented Mirpur-18 (0.148±0.015 vs. 0.086±0.0098 μM, respectively; p=0.014, unpaired t-test).

TABLE 9
Abundance (RPKM) of the 30 most age-discriminatory mcSEED subsystems/pathway
modules represented in the cecal microbiomes of mice subjected
to the three different diet treatments.
(A) Diet Group

| | Feature | | | |
|---|---|---|---|---|
| | importance | Diet Group | | |
| Subsystem | rank | Mirpur-18 | Mirpur(P) | Mirpur(PCSB) |
| Lipoate biosynthesis | 1 | 242 ± 12 | 253 ± 6 | 288 ± 16 |
| Pyridoxine and pyridoxal uptake | 2 | 184 ± 25 | 182 ± 20 | 185 ± 21 |
| Folate biosynthesis | 3 | 1766 ± 74 | 1842 ± 44 | 1971 ± 74 |
| Riboflavin biosynthesis | 4 | 916 ± 35 | 929 ± 59 | 1017 ± 38 |
| Folate uptake | 5 | 87 ± 11 | 93 ± 9 | 80 ± 12 |
| Biotin uptake | 6 | 312 ± 18 | 314 ± 27 | 358 ± 11 |
| Cellobiose and Beta-glucosides | 7 | 3653 ± 310 | 3287 ± 218 | 3925 ± 428 |
| utilization | | | | |
| Proline biosynthesis | 8 | 740 ± 31 | 759 ± 18 | 689 ± 27 |
| Rhamnose and Rhamnogalacturonan | 9 | 1963 ± 149 | 2206 ± 73 | 2474 ± 36 |
| utilization | | | | |
| Isoleucine, leucine, valine uptake | 10 | 779 ± 78 | 796 ± 63 | 677 ± 61 |
| Arabinose and Arabinosides utilization | 11 | 2257 ± 50 | 2247 ± 53 | 2055 ± 26 |
| Arginine uptake | 12 | 871 ± 163 | 749 ± 129 | 395 ± 44 |
| Butyrate fermentation | 13 | 1058 ± 81 | 1058 ± 90 | 965 ± 139 |
| Tryptophan uptake | 14 | 394 ± 23 | 389 ± 32 | 361 ± 38 |
| Ribose utilization | 15 | 1411 ± 39 | 1547 ± 33 | 1497 ± 44 |
| Pyridoxine and pyridoxal phosphate | 16 | 845 ± 58 | 828 ± 47 | 667 ± 9 |
| biosynthesis | | | | |
| Isoleucine, leucine, valine | 17 | 3083 ± 162 | 2978 ± 125 | 2550 ± 60 |
| biosynthesis | | | | |
| Histidine degradation | 18 | 361 ± 21 | 408 ± 12 | 453 ± 27 |
| Gluconate utilization | 19 | 226 ± 11 | 243 ± 12 | 234 ± 11 |
| Asparagine biosynthesis | 20 | 386 ± 11 | 415 ± 11 | 437 ± 19 |
| Cysteine biosynthesis | 21 | 511 ± 24 | 544 ± 24 | 557 ± 30 |
| Formate fermentation | 22 | 1170 ± 46 | 1127 ± 29 | 1102 ± 14 |
| Methionine, Threonine biosynthesis | 23 | 361 ± 22 | 350 ± 7 | 302 ± 18 |
| Threonine biosynthesis | 24 | 516 ± 36 | 515 ± 35 | 426 ± 24 |
| Proline degradation | 25 | 62 ± 4 | 62 ± 3 | 57 ± 3 |
| Thiamine and precursor uptake | 26 | 1152 ± 106 | 1046 ± 102 | 759 ± 14 |
| Cobalamin biosynthesis | 27 | 2034 ± 149 | 2134 ± 136 | 2016 ± 156 |
| Pyruvate metabolism | 28 | 250 ± 11 | 270 ± 10 | 283 ± 16 |
| Lysine uptake | 29 | 207 ± 21 | 232 ± 15 | 213 ± 22 |
| Sialic acid utilization | 30 | 2977 ± 94 | 3030 ± 63 | 3042 ± 24 |

(B) Dunn's Post-Hoc (values are adjusted p-valued (FDR)); Subsystem categories
from (A) are not copied over, but can be identified by feature importance rank).

| Feature | | | | |
|---|---|---|---|---|
| importance | Kruskal- | Mirpur(PCSB) vs | Mirpur(PCSB) vs | Mirpur(P) vs |
| rank | Wallis | Mirpur-18 | Mirpur(P) | Mirpur-18 |
| 1 | 0.03 | 0.0029 | 0.0375 | 0.1680 |
| 2 | 0.95 | 1.0000 | 0.5920 | 0.4929 |
| 3 | 0.03 | 0.0029 | 0.0654 | 0.1128 |
| 4 | 0.03 | 0.0061 | 0.0195 | 0.3152 |
| 5 | 0.34 | 0.2028 | 0.1518 | 0.2943 |
| 6 | 0.03 | 0.0122 | 0.0095 | 0.5000 |
| 7 | 0.12 | 0.1822 | 0.0323 | 0.1117 |
| 8 | 0.04 | 0.0309 | 0.0101 | 0.2165 |
| 9 | 0.03 | 0.0017 | 0.0561 | 0.0998 |
| 10 | 0.05 | 0.0143 | 0.0280 | 0.4433 |
| 11 | 0.03 | 0.0098 | 0.0121 | 0.4363 |
| 12 | 0.03 | 0.0037 | 0.0303 | 0.2113 |
| 13 | 0.64 | 0.3043 | 0.5595 | 0.4574 |
| 14 | 0.44 | 0.2981 | 0.2020 | 0.4574 |
| 15 | 0.03 | 0.0441 | 0.1346 | 0.0058 |
| 16 | 0.03 | 0.0061 | 0.0195 | 0.3152 |
| 17 | 0.03 | 0.0037 | 0.0303 | 0.2113 |
| 18 | 0.03 | 0.0013 | 0.0745 | 0.0679 |
| 19 | 0.22 | 0.1491 | 0.2606 | 0.0958 |
| 20 | 0.03 | 0.0022 | 0.0850 | 0.0787 |
| 21 | 0.15 | 0.0514 | 0.2664 | 0.1276 |
| 22 | 0.15 | 0.0425 | 0.1507 | 0.2261 |
| 23 | 0.03 | 0.0048 | 0.0442 | 0.1865 |
| 24 | 0.03 | 0.0098 | 0.0121 | 0.4363 |
| 25 | 0.17 | 0.0616 | 0.1037 | 0.4293 |
| 26 | 0.03 | 0.0029 | 0.0375 | 0.1680 |
| 27 | 0.49 | 0.5000 | 0.1962 | 0.3925 |
| 28 | 0.05 | 0.0098 | 0.1818 | 0.0731 |
| 29 | 0.34 | 0.3251 | 0.1851 | 0.1692 |
| 30 | 0.44 | 0.2604 | 0.3946 | 0.2324 |

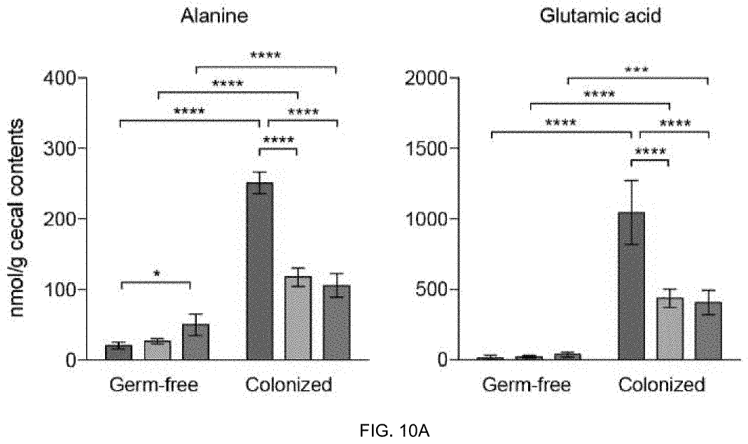

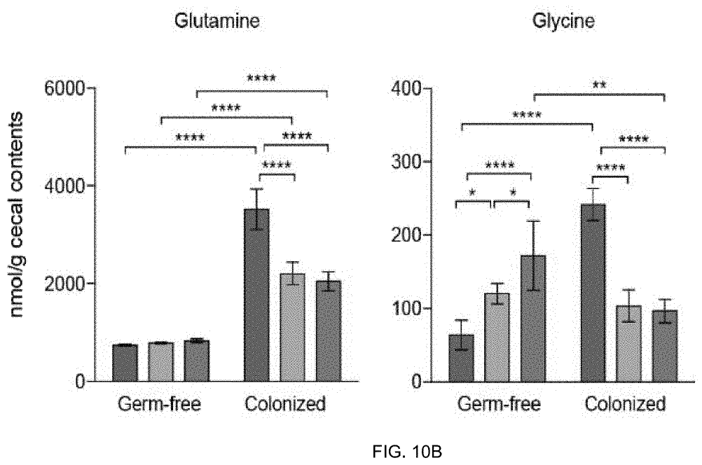

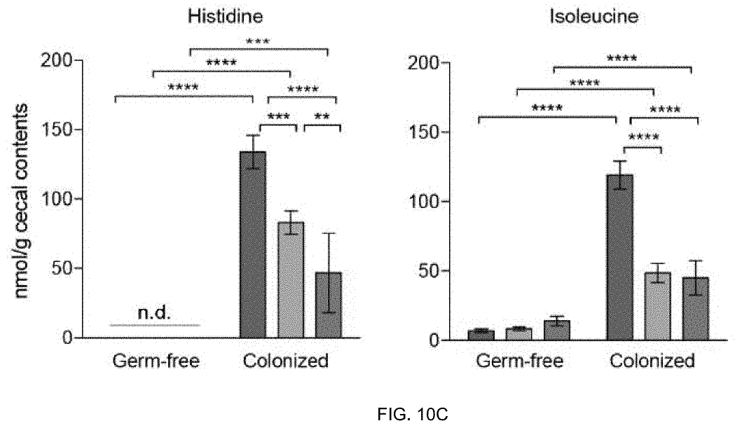

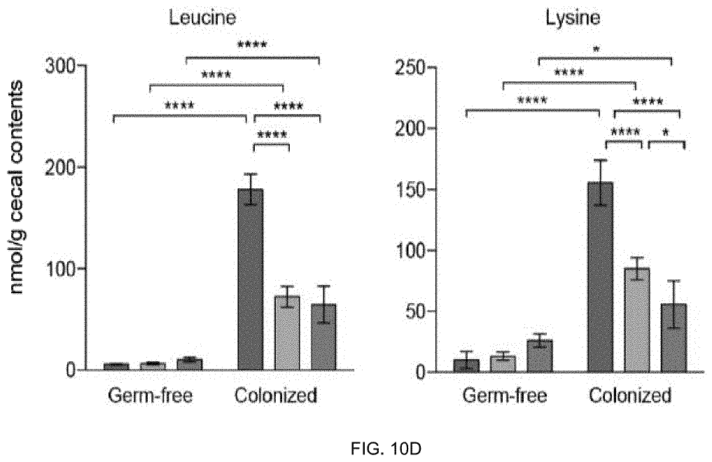

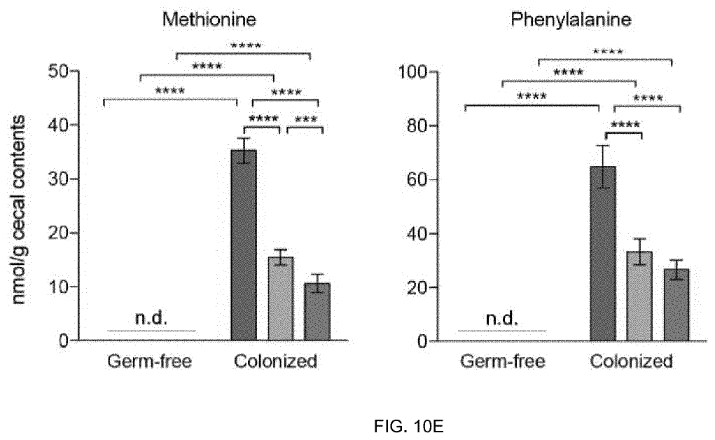

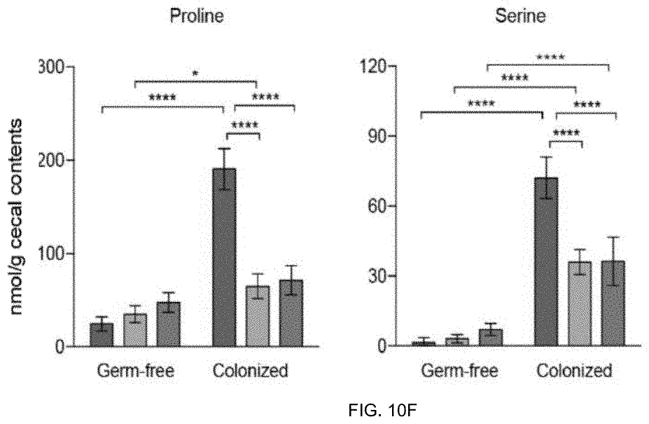

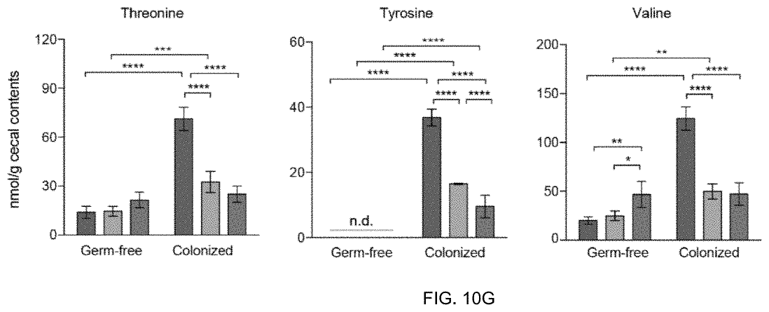

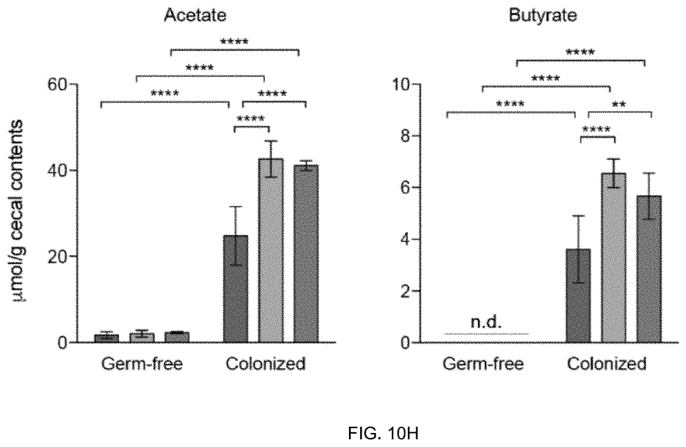

Targeted mass spectrometry of cecal contents disclosed that levels of all 15 free amino acids measured were significantly higher in colonized mice consuming the unsupplemented compared to supplemented Mirpur-18 diets. These differences were not observed in their germ-free counterparts (see FIG. 10). The origin of these colonization-dependent effects on cecal amino acid levels is unknown; for example, higher levels of free amino acids in the unsupplemented Mirpur-18 could reflect a more active proteolysis of dietary proteins or less active salvage/degradation of amino acids by the microbial community. However, the results are consistent with the observed differential expression patterns; i.e., accumulation of amino acids typically leads to repression of bacterial regulons controlling their biosynthesis. Diet-associated differences in levels of cecal branched-chain and other amino acids in colonized animals were not accompanied by significant differences in levels of the corresponding serum amino acids.

F. prausnitzii OTU 514940 was a prominent member of the cecal microbiota in these mice (15-17% mean relative abundance across the different diets; FIG. 9C and Table 10). We cultured an isolate representing this OTU from the transplanted post-SAM MAM donor community and mapped reads from the microbial RNA-Seq dataset onto its sequenced genome to identify the effects of diet on expression of genes belonging to various mcSEED categories and subsystems/pathway modules. Among the 271 differentially expressed genes, the largest number belong to the mcSEED category ‘Amino Acid Metabolism’ (35 genes), followed by ‘Carbohydrate Utilization’ (18 genes). All 35 differentially regulated genes involved in amino acid metabolism exhibited increased expression in the supplemented compared to unsupplemented Mirpur-18 diet treatment groups, with the most significantly upregulated subsystem/pathway module being ‘isoleucine, leucine, valine biosynthesis’ and ‘glutamate biosynthesis’. No significant differences in expression of these pathways were observed between the two supplemented diets.

Metabolic reconstructions of the F. prausnitzii isolate genome confirmed its capacity to produce butyrate. Mass spectrometry of cecal contents from germ-free and colonized mice revealed microbiota- and diet-dependent effects on butyrate and acetate levels (significantly greater in colonized mice consuming the supplemented diets; FIG. 10). Cecal butyrate was also positively correlated with the relative abundance of F. prausnitzii (Pearson r=0.68, p=0.0074). Acetate production has been linked to resistance to certain enteropathogens (118), while butyrate is an important contributor to gut mucosal barrier function (119) (See the maintext for a discussion of how RNA-Seq analysis of laser capture microdissected small intestinal (jejunal) mucosa provided evidence for microbiota- and diet-dependent increases expression of genes involved in barrier function).

Gut mucosal barrier function—Epithelium and overlying mucus from the proximal, middle, and distal thirds of the small intestine were recovered by laser capture microdissection (LCM; FIG. 9D). Table 10 lists the 30 most abundant OTUs identified by V4-16S rDNA analysis of LCM mucosal DNA obtained from the different small intestinal segments within a given diet group and between similarly positioned segments across the different diet treatments. For example, Mirpur(PCSB) produced a statistically significant increase in the relative abundance of F. prausnitzii in the proximal two-thirds of the small intestine, without significantly affecting the proportional representation of a milk-associated age-discriminatory Bifidobacteria OTU (FIG. 9C, FIG. 9D).

TABLE 10
The 30 most abundant OTUs in the fecal microbiota (collected at 21 days post-
gavage, dpg 21) and cecal microbiota (dpg 25) as a function of diet treatment.

| | Taxonomic | | | | | | |
|---|---|---|---|---|---|---|---|
| OUT | assignment | 1 | 2 | 3 | 4 | 5 | 6 |
| 589277 | B. vulgatus | 22.1 ± 1.2 | 18.8 ± 1.2 | 24.2 ± 2.4 | 21.4 ± 1.3 | 21.6 ± 1 | 19.9 ± 0.8 |
| 583656 | B. thetaiotaomicron | 17.7 ± 0.5 | 15.1 ± 2.8 | 18.9 ± 1.2 | 15.4 ± 1.7 | 20.4 ± 0.4 | 21.8 ± 1.3 |
| 514940 | [symbol] | 6.8 ± 1.4 | 15.5 ± 2 | 13.1 ± 1.5 | 17.5 ± 1.4 | 19.1 ± 0.8 | 17.5 ± 0.7 |
| 997439 | [symbol] | 21.1 ± 2 | 17.5 ± 2.4 | 11.1 ± 1.7 | 11.8 ± 3.8 | 9.1 ± 1.3 | 6.2 ± 4.5 |
| 585914 | P. distasonis | 4.3 ± 0.5 | 5.3 ± 0.5 | 6.6 ± 0.8 | 5.7 ± 0.7 | 6.3 ± 0.9 | 6.5 ± 0.9 |
| 259772 | A. caccae | 2.2 ± 0.7 | 5.9 ± 1 | 3 ± 0.7 | 5.7 ± 0.8 | 2.6 ± 0.8 | 5.5 ± 1.4 |
| 586290 | C. hathewayi | 3.1 ± 0.4 | 3.1 ± 0.3 | 3.1 ± 0.4 | 2.7 ± 0.2 | 2.7 ± 0.3 | 2.8 ± 0.4 |
| 559527 | [symbol] | 4.5 ± 0.5 | 2.5 ± 0.7 | 1.9 ± 0.3 | 2.1 ± 0.8 | 0.8 ± 0.3 | 1.4 ± 0.6 |
| 696563 | B. producta | 1.1 ± 0.2 | 1.5 ± 0.3 | 1.5 ± 0.3 | 2.3 ± 0.2 | 2.8 ± 0.7 | 3.1 ± 0.8 |
| 338992 | Clostridiales | 1.2 ± 0.1 | 1.7 ± 0.4 | 1.2 ± 0.1 | 1.4 ± 0.2 | 0.6 ± 0.1 | 0.9 ± 0.1 |
| 309720 | Ruminococcaceae | 1.9 ± 0.1 | 0.7 ± 0.1 | 1.6 ± 0.2 | 0.9 ± 0.1 | 0.9 ± 0.1 | 0.8 ± 0.1 |
| 659361 | [symbol] | 1.7 ± 0.3 | 0.7 ± 0.1 | 1.5 ± 0.2 | 1 ± 0.1 | 0.8 ± 0.2 | 0.5 ± 0.2 |
| 628226 | I. bartlettii | 1 ± 0.2 | 1.5 ± 0.3 | 0.8 ± 0.2 | 0.9 ± 0.2 | 0.6 ± 0.2 | 0.6 ± 0.2 |
| 564961 | Lachnoclostridium | 0.6 ± 0.2 | 0.8 ± 0.1 | 0.7 ± 0 | 0.9 ± 0.2 | 0.9 ± 0.1 | 1.1 ± 0.1 |
| 514523 | [symbol] | 0.3 ± 0.1 | 0.9 ± 0.1 | 0.6 ± 0.2 | 1 ± 0.1 | 0.6 ± 0.1 | 1.1 ± 0.1 |
| 542096 | Lachnoclostridium | 0.4 ± 0.1 | 0.7 ± 0.1 | 0.3 ± 0.1 | 0.5 ± 0.1 | 1 ± 0.2 | 1.1 ± 0.2 |
| 581079 | F. plautii | 0.6 ± 0.1 | 0.6 ± 0.1 | 0.6 ± 0 | 0.6 ± 0.1 | 0.6 ± 0.1 | 0.6 ± 0 |
| 579851 | C. innocuum | 0.6 ± 0.2 | 0.9 ± 0.2 | 0.5 ± 0.1 | 0.7 ± 0.1 | 0.4 ± 0.1 | 0.7 ± 0.1 |
| 564806 | C. bolteae | 0.9 ± 0.1 | 0.1 ± 0 | 0.6 ± 0.1 | 0.6 ± 0.2 | 0.3 ± 0.1 | 0.5 ± 0.1 |
| 558444 | Lachnospiraceae | 0.3 ± 0.1 | 0.5 ± 0.1 | 0.4 ± 0 | 0.5 ± 0.1 | 0.4 ± 0.1 | 0.6 ± 0.1 |
| New.0.CleanUp.- | Ruminococcaceae | 1.2 ± 0.2 | 0.4 ± 0.1 | 0.5 ± 0.2 | 0.2 ± 0.1 | 0.2 ± 0.1 | 0.2 ± 0.1 |
| ReferenceOTU1424 | | | | | | | |
| 360015 | [symbol] | 0.2 ± 0.1 | 0.3 ± 0.2 | 0.5 ± 0.3 | 0.8 ± 0.2 | 0.2 ± 0.1 | 0.3 ± 0.2 |
| 262095 | E. ramosum | 0.2 ± 0.1 | 0.3 ± 0.1 | 0.2 ± 0.2 | 0.2 ± 0.2 | 0.6 ± 0.4 | 0.6 ± 0.2 |
| 535601 | [symbol] | 0.3 ± 0 | 0.2 ± 0 | 0.3 ± 0 | 0.2 ± 0.1 | 0.4 ± 0.1 | 0.4 ± 0.1 |
| New.1.CleanUp.- | Bacteroides | 0.3 ± 0 | 0.2 ± 0 | 0.5 ± 0.1 | 0.2 ± 0.1 | 0.5 ± 0.1 | 0.2 ± 0.1 |
| ReferenceOTU14662 | | | | | | | |
| 3472078 | B. fragilis | 0.4 ± 0 | 0.2 ± 0.1 | 0.4 ± 0.1 | 0.2 ± 0 | 0.5 ± 0.1 | 0.2 ± 0.1 |
| 577170 | B. vulgatus | 0.4 ± 0.1 | 0.1 ± 0 | 0.4 ± 0 | 0.2 ± 0 | 0.5 ± 0.1 | 0.2 ± 0.1 |
| 175485 | Bacteroides | 0.3 ± 0.1 | 0.2 ± 0.1 | 0.4 ± 0.1 | 0.1 ± 0 | 0.5 ± 0.1 | 0.2 ± 0.1 |
| 364538 | D. mossii | 0.4 ± 0.2 | 0.1 ± 0.1 | 0.3 ± 0 | 0.2 ± 0 | 0.4 ± 0.1 | 0.3 ± 0.1 |
| 370287 | [symbol] | 0.1 ± 0 | 0.2 ± 0 | 0.1 ± 0 | 0.3 ± 0 | 0.2 ± 0 | 0.3 ± 0.1 |

Age-/growth-discriminatory OTUs are in boldface.
1 = Mirpur-18 dpg 21 fecal (n = 5); 2 = Mirpur-18 dpg 25 cecal (n = 5); 3 = Mirpur(P) dpg 21 fecal (n = 4), 4 = Mirpur(P) dpg 25 cecal (n = 4); 5 = Mirpur(PCSB) dpg 21 fecal (n = 5); 6 = Mirpur(PCSB) dpg 25 cecal (n = 5).
Values are % relative abundance (mean ± SD).

Gene expression was characterized in the jejunal mucosa (SI-2 segment in FIG. 9D) recovered by LCM from mice belonging to all six treatment groups. Significant differences in expression were categorized based on enriched Gene Ontology (GO) terms for ‘Molecular Function’. In colonized mice, Mirpur(P) and Mirpur(PCSB) significantly upregulated genes assigned to ‘adherin binding’ (GO:0045296) and ‘cell adhesion molecule binding’ (GO:0050839) compared to Mirpur-18. The diet effect was colonization-dependent; i.e., there were no significant differences in expression of these genes or these GO categories in germ-free mice consuming supplemented versus unsupplemented diets.

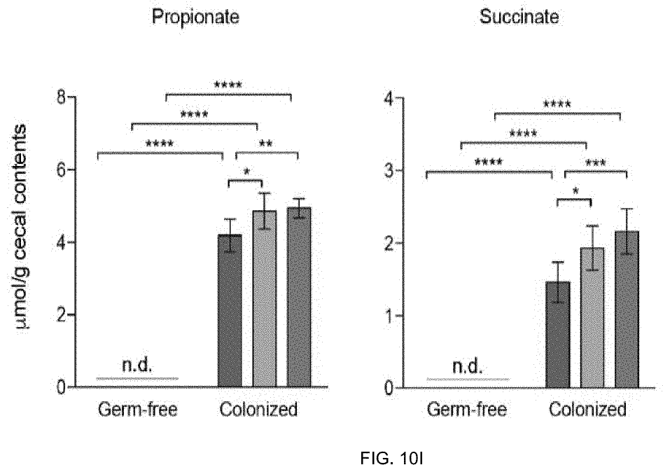

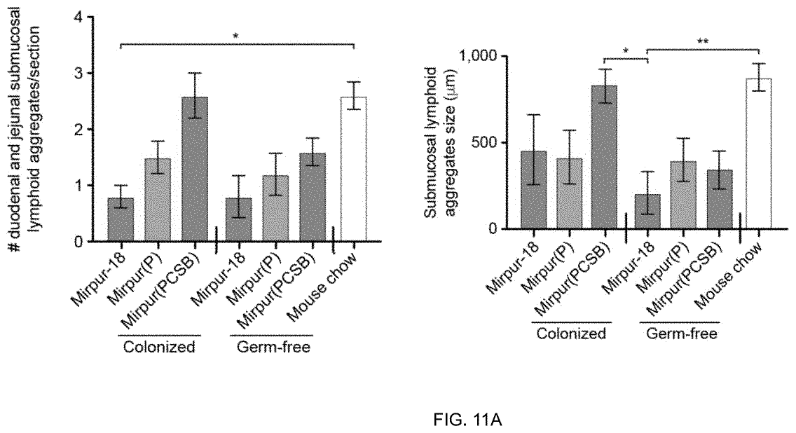

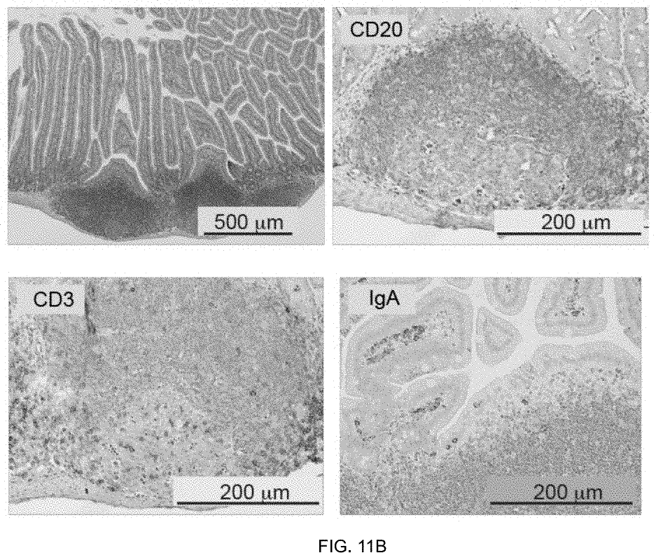

The different diets produced no statistically significant differences in the number of small intestinal goblet cells or Paneth cells, or crypt depth to villus height ratios, between mice colonized with the post-SAM MAM donor microbiota (Student's t-test). However, analysis of hematoxylin- and eosin-stained sections revealed a trend toward an increase in the number and size of submucosal lymphoid aggregates in the proximal and middle thirds of the small intestine in post-SAM MAM microbiota colonized animals consuming Mirpur(PCSB) compared to the other treatment groups (FIG. 11A). Immunostaining disclosed that these aggregates are B-cell dominant, with T-cell zones and associated rare IgA-positive plasma cells (FIG. 11B). The number and size of these submucosal lymphoid aggregates in Mirpur(PCSB) treated mice harboring the post-SAM MAM microbiota was not significantly different than the number and size of submucosal lymphoid aggregates in conventionally-raised mice harboring a native mouse microbiota fed a standard mouse chow (Kruskal-Wallis test with Dunn's correction for multiple comparisons; FIG. 11A).

Based on its effects on microbiota composition, microbiome gene expression and gut barrier function, we deemed Mipur-18 supplemented with the four lead complementary foods (Mirpur(PCSB)) superior to that supplemented with just peanut flour (Mirpur(P)).

### Example 5

### Characterizing MDCF Prototypes in Gnotobiotic Piglets

We examined the effects of MDCF prototypes in a second host species whose physiology and metabolism are more similar to that of humans. Gnotobiotic piglets provide an attractive model for these purposes; piglets manifest rapid growth rates in the weeks following birth (27) and methods for conducting experiments with gnotobiotic piglets have been described (28). Based on the results from the gnotobiotic mouse studies, we designed two MDCF prototypes. One prototype was formulated to be analogous to Mirpur-18 which contains milk powder; this prototype was supplemented with peanut flour, chickpea flour, soy flour and banana [MDCF(PCSB)]. The other diet lacked milk powder and was supplemented with just chickpea flour and soy flour [MDCF(CS)]. The two MDCFs were isocaloric, matched in lipid levels, total protein content (with equivalent representation of amino acids), and also met current ready-to-use therapeutic food guidelines for children with respect to macro- and micronutrient content (29) (Table 11).

TABLE 11

| | | 1 | | 2 | | | 3 | | |
|---|---|---|---|---|---|---|---|---|---|
| Ingredient | | MDCF | MDCF | Mirpur- | | Mirpur-18 + | Mirpur | Mirpur | 4 |
| type | Component | (PCSB) | (CS) | 18 | PCBT | PCBT | (P) | (PCSB) | MDCF |
| Lead | Banana | — | — | — | 25.0 | 20.0 | — | 7.5 | 19.6 |
| comple- | Banana | 23.9 | — | — | — | — | — | — | — |
| mentary | powder | | | | | | | | |
| food | Chickpea | — | — | — | 50.0 | 24.5 | — | — | 46.7 |
| | Chickpea | 10.6 | 22.3 | — | — | — | — | 3.0 | — |
| | flour | | | | | | | | |
| | Peanut | — | — | — | 12.5 | 5.0 | — | — | — |
| | Peanut | 2.7 | — | — | — | — | 5.1 | 3.2 | — |
| | flour | | | | | | | | |
| | Soy flour | 13.0 | 22.3 | — | — | — | — | 2.0 | — |
| Comple- | Egg | — | — | — | — | — | — | — | 8.4 |
| mentary | Red lentils | — | — | 16.2 | — | 6.5 | 15.4 | 13.7 | 0.9 |
| food | Milk | 9.7 | — | 13.9 | 5.0 | 13.9 | 13.2 | 11.8 | 4.7 |
| | powder | | | | | | | | |
| | Potato | — | — | 6.9 | — | 2.7 | 6.5 | 5.8 | 3.7 |
| | Pumpkin | — | — | 6.0 | — | 2.4 | 5.7 | 5.1 | — |
| | Pumpkin | 3.7 | — | — | — | — | — | — | — |
| | powder | | | | | | | | |
| | Rice | — | — | 41.8 | — | 10.2 | 39.6 | 35.3 | — |
| | Spinach | — | — | 6.3 | — | 2.5 | 6.0 | 5.3 | — |
| | Tilapia | — | — | — | 5.5 | 9.3 | — | — | 14.0 |
| Other | Cardamom | — | 0.1 | — | — | — | — | — | — |
| ingredient | powder | | | | | | | | |
| | Garlic | — | — | 0.5 | — | 0.5 | 0.4 | 0.4 | — |
| | Garlic | 0.2 | — | — | — | — | — | — | — |
| | powder | | | | | | | | |
| | Iodized | 0.4 | | 0.5 | — | 0.5 | 0.4 | 0.4 | — |
| | salt | | | | | | | | |
| | Lecithin | — | 0.3 | — | — | — | — | — | — |
| | Onion | — | — | 3.7 | — | 1.5 | 3.5 | 3.1 | — |
| | Onion | 0.9 | — | — | — | — | — | — | — |
| | powder | | | | | | | | |
| | Palm oil | 18.8 | 17.1 | — | — | — | — | — | — |
| | Powdered | — | 19.6 | — | — | — | — | — | — |
| | sugar | | | | | | | | |
| | Soybean | 12.8 | 14.7 | 3.7 | 2.0 | — | 3.5 | 3.1 | 1.9 |
| | oil | | | | | | | | |
| | Turmeric | 0.2 | — | 0.5 | — | 0.5 | 0.4 | 0.4 | — |
| | Vitamin | 3.1 | 3.6 | — | — | — | — | — | — |
| | Mineral | | | | | | | | |
| | Blend | | | | | | | | |
| | kcal/ | 568 | 545 | 183.9 | 227.3 | 194.4 | 191.3 | 190.2 | 168.6 |
| | 100 g diet | | | | | | | | |
| | g protein/ | 14.5 | 15 | 6.9 | 10.2 | 10.7 | 9.2 | 9.2 | 10.2 |
| | 100 g diet | | | | | | | | |
| | g lipids/ | 32.4 | 33.9 | 7.7 | 11.1 | 7.2 | 7.3 | 6.5 | 5.7 |
| | 100 g diet | | | | | | | | |
| | g carbohydrates/ | 40.77 | 40.92 | 21.8 | 24.3 | 23.1 | 21.1 | 19.1 | 20.2 |
| | 100 g diet | | | | | | | | |
| | g fiber/ | 6.7 | 7.9 | 2.2 | 5.2 | 3.6 | 2.9 | 3.2 | 4.2 |
| | 100 g diet | | | | | | | | |

1 = gnotobiotic piglets; 2 = Screening 15 SAM/post-SAM MAM microbiota in gnotobiotic mice; 3 = PS.064.S7-colonized gnotobiotic mice; 4 = MDCF vs. Milk Suji/Khichuri-Halwa (MS/KH) gnotobiotic mouse experiment (FIG. 7); PCBT = Peanut, chickpea, banana, tilapia; Mirpur(P) = Mirpur-18 + Peanut flour; Mirpur(PCSB) = Mirpur-18 + (Peanut/Chickpea/Soy flours, Banana)

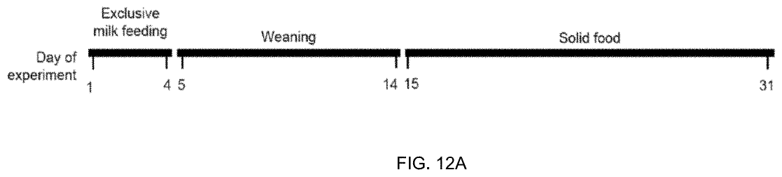

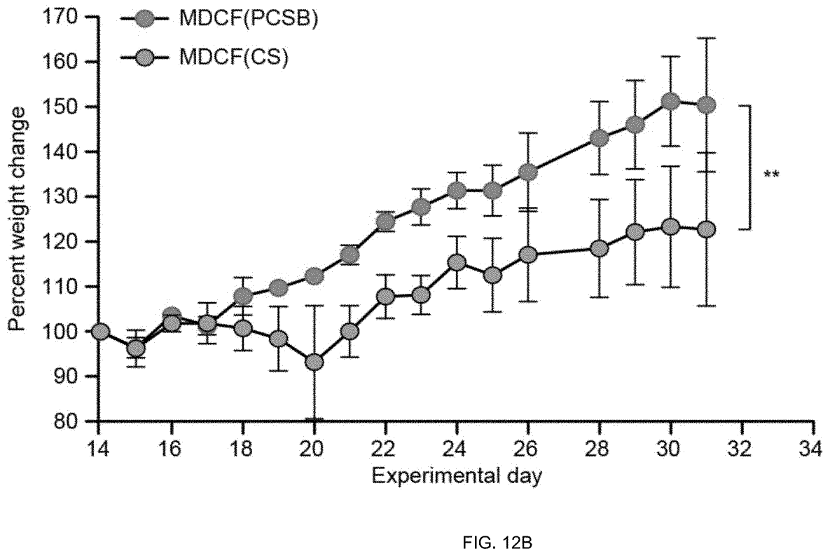

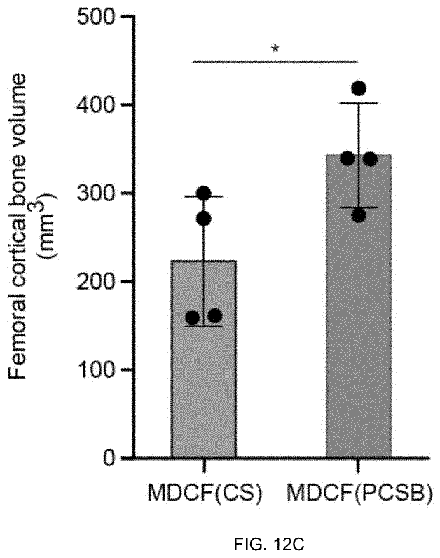

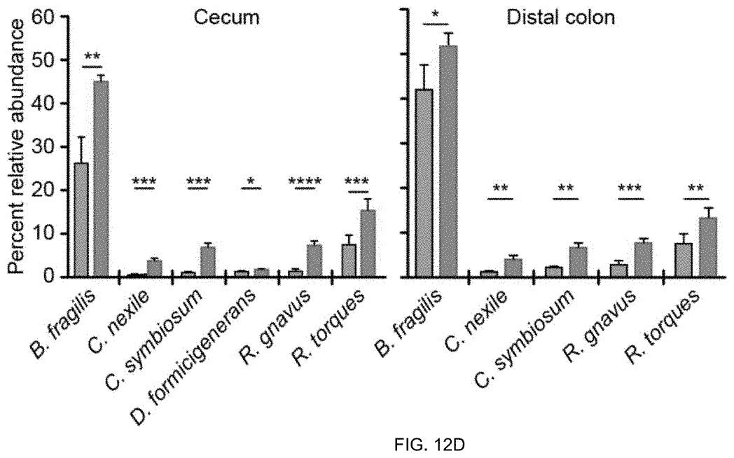

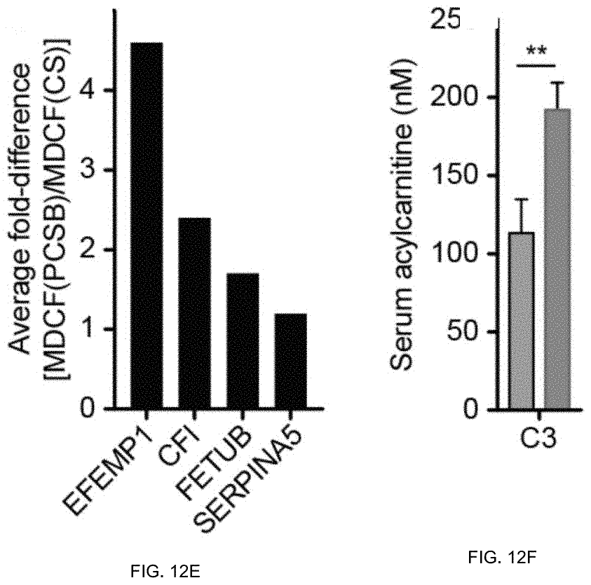

Four-day-old germ-free piglets fed a sow milk-based formula were colonized with a 14-member consortium of bacterial strains consisting of the same nine Bangladeshi age-discriminatory strains used for the diet oscillation experiments described in FIG. 5, plus five weaning-phase age-discriminatory strains cultured from Malawian children (Table 12). Several members of this consortium (B. longum, F. prausnitzii, C. symbiosum, R. gnavus, and D. formicigenerans) were classified as growth-discriminatory by a RF-based analysis of their representation in gnotobiotic mouse recipients of healthy and undernourished donor microbiota and the animals' weight/lean body mass gain phenotypes (3). After gavage, the two groups of piglets were weaned over the course of 10 days (Methods) onto one or the other irradiated MDCF prototypes, which they consumed ad libitum for the remainder of the experiment (n=4 piglets/treatment arm; FIG. 12A). Animals were euthanized on day 31 after a 6-hour fast, following AVMA guidelines.

TABLE 12
14-member consortium introduced into gnotobiotic piglets.

| | Bacterial strain |
|---|---|
| Bangladeshi weaning | Bifidobacterium catenulatum JG_Bg468 |
| phase age- | Blautia luti SSTS_Bg7063 |
| discriminatory | Dorea formicigenerans SSTS_Bg7063 |
| strains (from | Dorea longicatena SSTS_Bg7063 |
| healthy donors) | Faecalibacterium prausnitzii SSTS_Bg7063 |
| | Ruminococcus obeum SSTS_Bg7063 |
| | Ruminococcus torques SSTS_Bg7063 |
| Malawian weaning | Bacteroides fragilis MC_264A |
| phase age- | Clostridium symbiosum TS_8243C |
| discriminatory | Clostridium nexile TS_8243C |
| strains (from | Faecalibacterium prausnitzii TS3092C |
| healthy donors) | Ruminococcus gnavus TS_8243C |
| Milk-adapted | Bifidobacterium longum subsp. infantis |
| strains (from | JG_Bg463 |
| 6-month-old | Bifidobacterium breve JG_Bg463 |
| healthy donor) | |

Piglets fed MDCF(PCSB) exhibited significantly greater weight gain than those receiving MDCF(CS) (FIG. 12B). Microcomputed tomography of their femurs revealed that they also had significantly greater cortical bone volume (FIG. 12C). COPRO-Seq analysis disclosed that piglets treated with MDCF(PCSB) had significantly higher relative abundances of C. symbiosum, R. gnavus, D. formicigenerans, R. torques, and B. fragilis in their ceca and distal colon compared to piglets consuming MDCF(CS) (unpaired t-tests; FIG. 12D); all are weaning-phase age-discriminatory strains while the former three were, as noted above, also defined as growth-discriminatory. Conversely, the relative abundances of three members of Bifidobacteria (including two milk-associated age-discriminatory strains, B. breve and B. longum subsp. infantis) were significantly higher in the ceca and distal colons of piglets fed MDCF(CS). These findings led us to conclude that MDCF(PCSB) promoted a more weaning-phase-like (i.e., mature) community configuration than MDCF(CS).

Comparative microbial RNA-Seq of cecal contents harvested from piglets consuming MDCF(CS) or MDCF(PCSB) diets identified 2,021 differentially expressed genes with a complex distribution over 12 strains; 117 of these genes, from eight strains, mapped to mcSEED categories and associated subsystems/pathway modules.

Amino acid, mono- and disaccharide, organic acid, and short chain fatty acid levels in the ceca of piglets as a function of diet were compared. Of the 24 carbohydrates measured, only fructose exhibited a significant difference between the two groups [higher in MDCF(PCSB)-treated animals; p=0.02, unpaired t-test]. MDCF(PCSB) consumption was associated with lower cecal lactate and pyruvate levels (p=0.06 and 0.002, respectively), in concert with marked increases in the late TCA cycle intermediates malate and fumarate (p=0.0002 and 0.005, respectively), but not early intermediates (citrate, succinate, and a-ketoglutarate). These findings are consistent with a decrease in glycolytic metabolism of glucose and an increase in oxidative metabolism of glucose and other fuels.

The effects on host biology were also defined by mass spectrometry-based serum metabolomic and proteomic analyses. Because the aptamers used for quantitative proteomics analysis of human plasma samples do not have reported specificities for the corresponding porcine protein orthologs, we used mass spectrometry to compare the serum proteomes of piglets. Blood was obtained from animals after the 6 hour fast, just prior to euthanasia. We did not attempt to deplete serum samples of abundant proteins prior to two-dimensional liquid-chromatography MS/MS to avoid introducing biases in our analysis. Notable findings included significant increases in levels of tryptophan, methionine and C3-acylcarnitine with MDCF(PCSB), as well changes it produced in the serum proteome which are shared with children in the SAM trial.

Thirty-eight of the 398 detected proteins exhibited significant differences in their abundances between the two diet treatment groups. As in humans, the pig genome encodes seven IGFBP orthologs and an ALS (acid-labile subunit). ALS forms a ternary complex with IGF-1 and IGFBPs, prolonging the half-life of IGF-1, and in a rat model plays a role in growth promotion (120). IGF-1 was below the limits of detection in our LC-MS/MS analysis of non-depleted sera, and no significant differences were noted in the one IGFBP that was identified (IGFBP-2). However, levels of ALS in animals fed MDCF(PCSB) were 2.3-fold higher than in those consuming MDCF(CS).

Serum levels of EFEMP1 (fibulin-like extracellular matrix protein 1) in piglets consuming MDCF(PCSB) were 4.6-fold higher than in their MDCF(CS) fed counterparts (FIG. 12E). Genome-wide association studies have identified EFEMP1 as significantly associated with height in children (121). Mice with engineered deficiency of Efemp1 exhibit significant reductions in body mass and bone density (122).

Three other serum proteins that were significantly increased in MDCF(PCSB)-treated piglets are orthologs of human proteins significantly correlated with anthropometric and/or metabolic features in the SAM study: serpin family A member 5 (SERPINA5), complement factor I (CFI) and fetuin-B (FETUB) (FIG. 12E). In children recovering from SAM, levels of SERPINA5 were positively correlated with plasma C3-acylcarnitine, a marker of branched-chain amino acid oxidation (Spearman r=0.52).

While circulating serum levels of amino acids in piglets were comparable in the two treatment groups (with the exception of Trp and Met which were increased in the MDCF(PCSB) group), serum C3-acylcarnitine concentrations were significantly higher in the faster growing MDCF(PCSB)-treated animals than in those consuming MDCF(CS) (FIG. 12F). In the human study, plasma levels of fetuin-B, a member of the cystatin family of cysteine protease inhibitors produced by the liver, and CFI were positively correlated with ponderal growth [fetuin-B, r=0.59 (WAZ) and 0.54 (WHZ); CFI, r=0.44 (WAZ); Table 2]. Fetuin-B has been linked to glucose homeostasis (123) and fatty acid utilization (124). In addition to its correlation with anthropometric measures of growth, fetuin-B was also positively correlated with the relative abundance of R. gnavus (OTU 360015) and F. prausnitzii (OTU 514940) in fecal samples collected during the course of the SAM study [r=0.46 and 0.66, respectively; note that MDCF(PCSB) significantly augments the representation of R. gnavus, a growth-discriminatory bacterial species (FIG. 120)].

### Example 6

### Testing MDCFs in Bangledeshi Children with MAM

To assess the degree to which results obtained from the gnotobiotic mouse and piglet models translate to humans, we performed a pilot randomized, double-blind controlled feeding study of the effects of three MDCF formulations. The formulations (MDCF-1, -2 and -3) were designed to be matched in protein energy ratio and fat energy ratio and provide 250 kcal/day (divided over 2 servings). MDCF-2 contained all four lead ingredients (chickpea flour, soy flour, peanut flour and banana) at higher concentrations than in MDCF-1. MDCF-3 contained two lead ingredients (chickpea and soy flour). A rice- and lentil-based ready-to-use supplementary food (RUSF), included as a control arm, lacked all four ingredients but was otherwise similar in energy density, protein energy ratio, fat energy ratio and macro-/micronutrient content to the MDCFs (Table 13). Milk powder was included in MDCF-1 and RUSF. All formulations were supplemented with a micronutrient mixture designed to provide 70% of the recommended daily allowances for 12- to 18-month-old children. The formulations were produced locally and tested for organoleptic acceptability prior to initiating the trial.

TABLE 13

| Components (g/100 g) | RUSF | MDCF-1 | MDCF-2 | MDCF-3 |
|---|---|---|---|---|
| Chickpea flour | 0 | 8 | 10 | 30 |
| Peanut flour | 0 | 7 | 10 | 0 |
| Soy flour | 0 | 5 | 8 | 14 |
| Raw Banana | 0 | 19 | 19 | 0 |
| Rice | 18.9 | 0 | 0 | 0 |
| Lentil | 21.5 | 0 | 0 | 0 |
| Powdered Skimmed Milk | 10.5 | 11.5 | 0 | 0 |
| Sugar | 17 | 24.3 | 29.9 | 30.9 |
| Soybean oil | 29 | 22 | 20 | 22 |
| Micronutrient Premix | 3.14 | 3.14 | 3.1 | 3.1 |
| Protein | 10.2 | 12.4 | 11.6 | 13.9 |
| Fat | 29.5 | 22.8 | 20.8 | 24.1 |
| Total Carbohydrates | 48.8 | 42.9 | 46.2 | 52.9 |
| Fiber | 4.7 | 3.3 | 4.5 | 5.6 |
| Protein Energy Ratio (PER) | 8.2 | 11.8 | 11.4 | 11.7 |
| Fat Energy Ratio (FER) | 53.6 | 49.0 | 46.0 | 45.6 |
| Total Calories per 100 g | 494.6 | 418.1 | 406.8 | 475.8 |

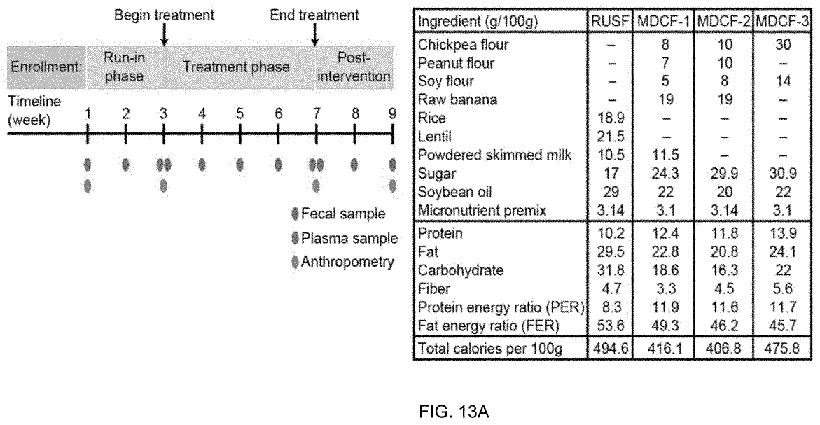

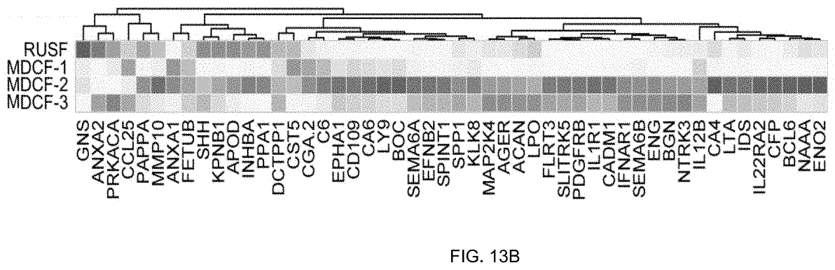

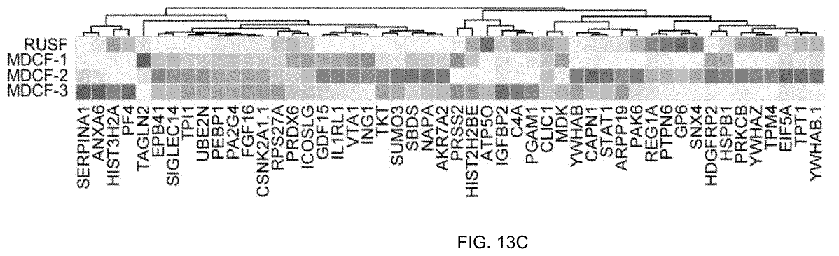

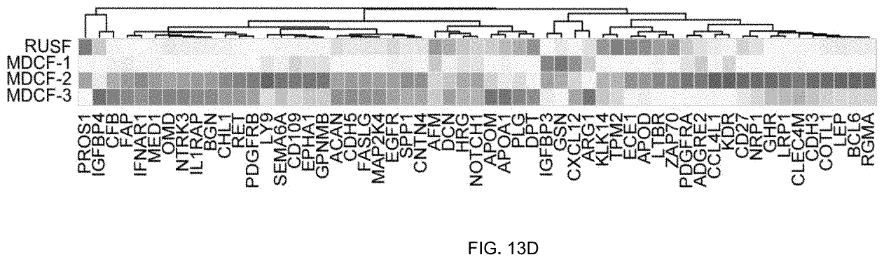

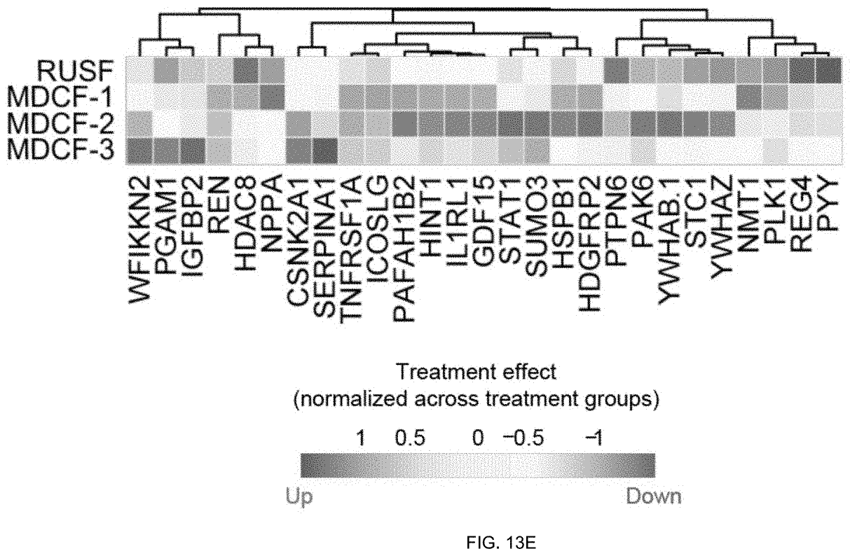

Children from Mirpur with MAM and no prior history of SAM were enrolled (mean age at enrollment, 15.2±2.1 months, mean WHZ −2.3±0.3). Participants were randomized into one of the four treatment arms (14-17 children per group) and received four weeks of twice daily feeding under supervision at the study center, preceded and followed by 2 weeks of observation and sample collection. Mothers were encouraged to continue their normal breastfeeding pactices throughout the study (FIG. 13, Methods). There were no significant differences in the mean daily amount of each MDCF or RUSF consumed per child, or in the mean incidence of morbidity across the four treatment groups. All three MDCFs and the RUSF control improved WHZ scores [−2.2±0.4 (mean ±SD) at the start of intervention compared to 1.9±0.5 at the completion of intervention, n=63 children, all groups combined; p=2.06×10⁻¹¹, paired t-test]. There were no statistically significant differences between the four interventions in the change in WHZ (one-way ANOVA p=0.31). Despite the small group size and the short length of the study, there were significant differences in treatment effects on another anthropometric indicator, with MDCF-2 producing a significantly greater increase in mid-upper arm circumference (MUAC) than MDCF-3 (one-way ANOVA p=0.022; with Tukey's multiple comparisons test; p=0.017).

Effects on biological state—To contextualize the biological effects of the dietary interventions, we first performed quantitative proteomics (using the same aptamer-based arrays described above) on plasma collected from twenty-one 12- to 24-month-old Mirpur children with healthy growth phenotypes (mean age 19.2±5.1 months; WHZ, 0.08±0.58; HAZ, −0.41±0.56, WAZ, −0.12±0.60) and 30 children with SAM prior to treatment ('B1′ sample in FIG. 1A; WHZ<−3; mean age 15.2±5.1 months). We rank-ordered all detected proteins based on fold-differences in their abundances in plasma collected from healthy children compared to children with untreated SAM. The top 50 differentially abundant proteins (p<10⁻⁷; R package “limma”) that were significantly higher in healthy children were designated ‘healthy growth-discriminatory’, while the top 50 differentially abundant proteins that were higher in children with SAM were designated ‘SAM-discriminatory’ (see, Table G and Table H, respectively, as well as Table s23A of Gehrig et al. Science, 2019, 365(6449):eaau4732, which is incorporated by reference in its entirety). We next compared the mean difference for each protein in the pre- versus post- intervention plasma samples for all children in each MDCF/RUSF treatment group. Proteins were then ranked based on the fold-differences of the pre- versus post-treatment levels in each of the 4 study arms (see, Table F (MDCF-2), as well as Table s23B of Gehrig et al. Science, 2019, 365(6449):eaau4732, which is incorporated by reference in its entirety) and these treatment effects were mapped onto the 50-most healthy growth-discriminatory and 50-most SAM-discriminatory proteins. Strikingly, MDCF-2 elicited a biological response characterized by a shift in the plasma proteome towards that of healthy reference children, and away from that of children with SAM; i.e., MDCF-2 increased the abundance of proteins that are higher in plasma from healthy children and reduced the levels of proteins elevated in SAM plasma samples (FIG. 13).

Aggregating proteomic datasets from the combined cohort of 113 children with SAM, MAM and healthy growth phenotypes for whom plasma samples were available, we identified a total of 27 plasma proteins that were significantly positively correlated with HAZ and 57 plasma proteins that were significantly negatively correlated with HAZ (absolute value of Pearson correlation >0.25, FDR-corrected p-value <0.05). Among the treatments, MDCF-2 was distinctive in its ability to increase the abundances of a broad range of proteins positively correlated with HAZ, including the major IGF-1 binding protein IGFBP-3, growth hormone receptor (GHR) and leptin (LEP) (FIG. 13). Growth differentiation factor 15 (GDF15) was reduced after four weeks of dietary supplementation with MDCF-2 (FIG. 13). This TGF-b superfamily member, which was negatively correlated with HAZ, is implicated in the anorexia and muscle wasting associated with cancer and with chronic heart failure in children; it was elevated in children with SAM, and positively correlated with their lipolytic biomarkers NEFA and ketones (see Supplementary Results). Peptide YY, an enteroendocrine cell product elevated in SAM plasma that reduces appetite and negatively correlated with HAZ, was also decreased by MDCF-2.

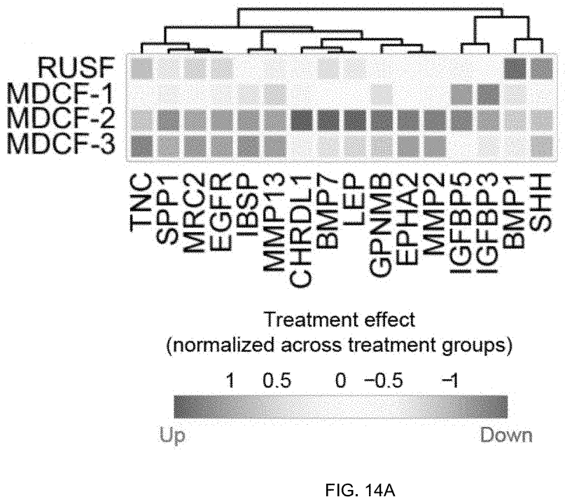

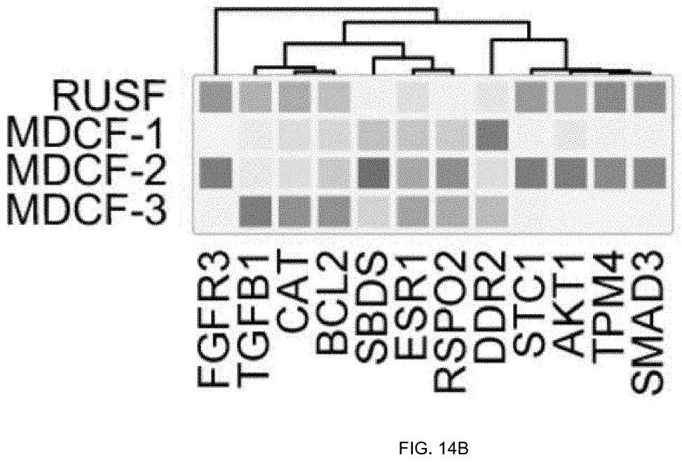

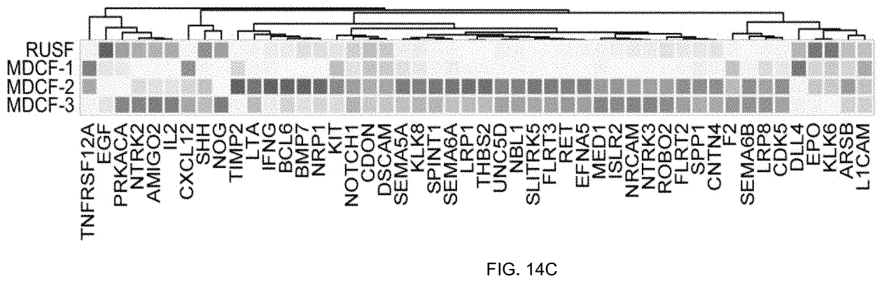

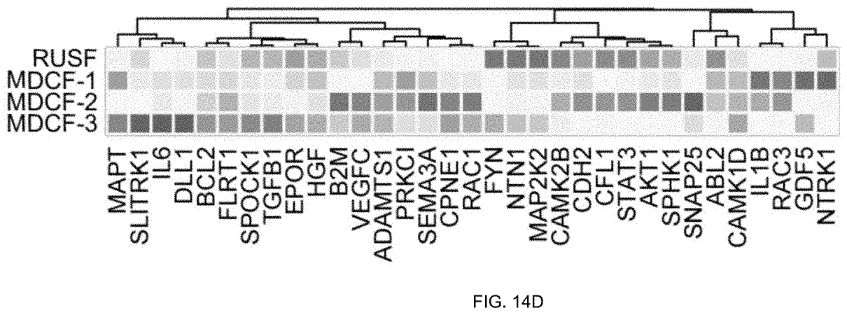

We identified Gene Ontology (GO) terms that were enriched among the group of treatment-responsive proteins and ranked them according to the p-value of their enrichment (see, Table s6F of Gehrig et al. Science, 2019, 365(6449):eaau4732, which is incorporated by reference in its entirety). Proteins belonging to GO terms significantly higher in healthy compared to SAM plasma samples were deemed ‘healthy growth-discriminatory’ while those that were significantly higher in SAM were deemed ‘SAM-discriminatory’ (threshold >30%; FDR adjusted p-value <0.05). This analysis revealed multiple healthy growth-discriminatory proteins associated with GO processes ‘osteoblast differentiation’ and ‘ossification’ that were increased by supplementation with MDCF-2 (FIG. 14, also see Table s23C of Gehrig et al. Science, 2019, 365(6449):eaau4732, which is incorporated by reference in its entirety). Examples include key markers/mediators of osteoblast differentiation [osteopontin (SPP1), bone sialoprotein 2 (IBSP), and bone morphogenetic protein 7 (BMP7)] as well as matrix metalloproteases (MMP-2 and MMP-13) involved in terminal differentiation of osteoblasts into osteocytes and bone mineralization.

A number of plasma proteins categorized under the GO process ‘CNS development’, including those involved in axon guidance and neuronal differentiation, were also affected by MDCF-2 supplementation. Levels of the SAM-discriminatory semaphorin SEMA3A, a potent inhibitor of axonal growth, decreased while healthy growth-discriminatory semaphorins (SEMASA, SEMA6A and SEMA6B) increased with this treatment (FIG. 14). Other healthy growth-discriminatory proteins whose abundances increased with MDCF-2 included receptors for neurotrophin (NTRK2 and NTRK3), the axonal guidance protein netrin (UNCSD), plus various ephrins (EFNAS) and ephrin receptors (EPHA1, EPHA2) (FIG. 14). Ephrin signaling is involved in axon guidance and synaptic development; ephrin expression is influenced by nutrient availability (30).

Compared to healthy children, the plasma proteome of children with SAM was characterized by elevated levels of acute phase proteins (e.g., CRP, IL-6) and inflammatory mediators, including several agonists and components of the NF-kB signaling pathway (FIG. 14). Pathway members include the pro-inflammatory cytokines IL-1β, TNF-α and CD40L, plus ubiquitin-conjugating enzyme E2 N (UBE2N) which is involved in induction of NF-kB- and MAPK-responsive inflammatory genes (31). MDCF-2 supplementation was associated with reductions in the levels of all of these SAM-associated proteins (FIG. 14).

Effects on the microbiota—Our analysis of fecal microbiota samples revealed no significant change in the representation of enteropathogens within and across the four treatment groups (FIG. 15). MDCF-2-induced changes in biological state were accompanied by increases in the relative abundances of several weaning-phase taxa, including OTUs assigned to F. prausnitzii (OTU 851865) and a Clostridiales sp. (OTU 338992), that are closely related to taxa ranked 1 and 2 in feature importance in the sparse Bangladeshi RF-derived model of gut microbiota maturation (FIG. 15). MDCF-2 supplementation was associated with a significant decrease in B. longum (OTU 559527; FIG. 15), ranked third in feature importance in the RF-derived model, and discriminatory for a young, milk-oriented microbiota. None of the other members of the 30 OTU model showed significant changes. By contrast, MDCF-1 did not produce significant increases in any of the taxa in the model. The other two formulations were each associated a significant change in one member [an increase in the relative abundance of an early age-discriminatory OTU (Streptococcus; ranked 30th) with MDCF-3 supplementation, and a decrease in another OTU (Enterococcus faecalis; ranked 29th) with RUSF supplementation; Table 3].

MAZ scores were not significantly different between groups at enrollment, nor were they significantly improved by any of the formulations. Interpretation of this finding was confounded by unexpectedly high baseline microbiota maturity scores in this group of children with MAM [MAZ, −0.01±1.12 (mean±SD)] compared to a small, previously characterized Mirpur cohort with untreated MAM and no prior history of SAM (2). Hence, we developed an additional measure of microbiota repair (see (14)). This involved a statistical analysis of covariance among bacterial taxa in the fecal microbiota of anthropometrically healthy members of a Mirpur birth cohort who had been sampled monthly over a 5-year period. Using approaches developed in the fields of econophysics and protein evolution to characterize the underlying organization of interacting systems with seemingly intractable complexity, such as financial markets, we found that the gut community in healthy children could be decomposed into a sparse unit of 15 co-varying bacterial taxa termed an ‘ecogroup’ (14). These ecogroup taxa include a number of age-discriminatory strains in the Bangladeshi RF-derived model (e.g., B. longum, F. prausnitzii and Prevotella copri). We used the ecogroup to show that in addition to its effects on host biological state, MDCF-2 was also the most effective in re-configuring the gut bacterial community to a mature state similar to that characteristic of healthy Bangladeshi children.

### References for Examples 1-6

1—T. Ahmed, et al., The MAL-ED cohort study in Mirpur, Bangladesh. Clin. Infect. Dis. 59, Suppl 4, S280-S286 (2014).

2—S. Subramanian, et al., Persistent gut microbiota immaturity in malnourished Bangladeshi children. Nature 510, 417-421 (2014).

3—L.V. Blanton, et al., Gut bacteria that prevent growth impairments transmitted by microbiota from malnourished children Science 351, doi: 10.1126/science.aad3311 2016).

4—World Health Organization Department of Nutrition for Health and Development. WHO child growth standards. Length/height-for-age, weight-for-age, weight-for-length, weight-for- height and body mass index-for-age: methods and development (2000). https://www.who.int/childgrowth/en/

5-World Health Organization, Infant and young child feeding; Fact sheet no. 342, 1-5 (2016).

6—L. Manikam, et al., A systematic review of complementary feeding practices in South Asian infants and young children: the Bangladesh perspective. BMC Nutr. 3, https://doi.org/10.1186/s40795-017-0176-9 (2017).

7—H. Sandige, M. J. Ndekha, P. Ashorn, M. J. Manary, Home-based treatment of malnourished Malawian children with locally produced or imported ready-to-use food. J. Pediatr. Gastroenterol. Nutr. 39, 141-146 (2004).

8—L. Gold, J. J. Walker, S. K. Wilcox, S. Williams, Advances in human proteomics at high scale with the SOMAscan proteomics platform. Nature Biotechnol. 29, 543-549 (2012).

9—B. Lollo, F. Steele, L. Gold, Beyond antibodies: New affinity reagents to unlock the proteome. Proteomics 14, 638-644 (2014).

10—J. Candia et al., Assessment of variability in the SOMAscan assay. Sci. Rep. 7, 1-13 (2017).

11—S. Bartz, et al. Severe acute malnutrition in childhood: hormonal and metabolic status at presentation, response to treatment, and predictors of mortality. J. Clin. Endocrinol. Metab. 99, 2128-2137 (2014).

12—R. Overbeek, R. Olson, G. D. Pusch, G. J. Olsen, J. J. Davis, T. Disz et al. The SEED and the Rapid Annotation of microbial genomes using Subsystems Technology (RAST). Nucleic Acids Res. 42, D206—D214 (2014).

13—D. A. Sela, et al., The genome sequence of Bifidobacterium longum subsp. infantis reveals adaptations for milk utilization within the infant microbiome, Proc. Natl. Acad. Sci. U.S.A. 105, 18964-18969 (2008).

14—A. S. Raman, et al., A sparse co-varying unit of the human gut microbiota that describes healthy and impaired community development. Science, doi (2018).

15—T. Ahmed, et al., Nutrition of children and women in Bangladesh: trends and directions for the future, J. Health Popul. Nutr. 30, 1-11 (2012).

16—L. L. lannotti, et al., Eggs in early complementary feeding and child growth: a randomized controlled trial. Pediatrics 140, e20163459 (2017).

17—W. R. Russell, et al., Major phenylpropanoid-derived metabolites in the human gut can arise from microbial fermentation of protein. Mol. Nutr. Food Res. 57, 523-535 (2013).

18—D. Krause, et al., The tryptophan metabolite 3-hydroxyanthranilic acid plays anti- inflammatory and neuroprotective roles during inflammation: role of hemeoxygenase-1. Am. J. Path. 179, 1360-1372 (2011).

19—L. Cervantes-Barragan, et al., Lactobacillus reuteri induces gut intraepithelial CD4+CD8aa+T cells. Science 357, 806-810 (2017).

20—G. Das, et al., An important regulatory role for CD4+CD8 alpha alpha T cells in the intestinal epithelial layer in the prevention of inflammatory bowel disease. Proc. Natl. Acad. Sci. U.S.A. 100, 5324-5329 (2003).

21—H. Cheroutre, et al., CD4 CTL: living up to the challenge. Semin. Immunol. 25, 273-281 (2013).

22—T. Sujino, et al., Tissue adaptation of regulatory and intraepithelial CD4+T cells controls gut inflammation. Science 352, 1581-1586 (2016).

23—R. A. Saxton and D. M. Sabatini, mTOR signaling in growth, metabolism, and disease. Cell. 169, 361-371 (2017).

24 —J. Yan, et al., Gut microbiota induce IGF-1 and promote bone formation and growth. Proc. Natl. Acad. Sci. U.S.A. 113, E7554—E7563 (2016).

25—M. Schwarzer, et al., Lactobacillus plantarum strain maintains growth of infant mice during chronic undernutrition. Science 351, 854-857 (2016).

26—D. J. Millward, Amino acid scoring patterns for protein quality assessment, Br. J. Nutr. 108, (Suppl. 2), S31-43 (2012).

27—P. L. Altman, D. S. Dittmer, Growth, including reproduction and morphological development. Federation of American Societies for Experimental Biology, Washington, D.C., (1962).

28—M. R. Charbonneau et al., Sialylated milk oligosaccharides promote microbiota-dependent growth in models of infant undernutrition. Cell. 164, 859-871 (2016).

29—U.S. Department of Agriculture, “Commercial Item Description: Ready-to-Use Therapeutic Food (RUTF)” A-A-20363B (2012).

30—I. Antonow-Schlorke, et al., Vulnerability of the fetal primate brain to moderate reduction in maternal global nutrient availability. Proc. Natl. Acad. Sci. U. S. A. 108, 3011-3016 (2011).

31—K. Taniguchi, M. Karin, NF-κB, inflammation, immunity and cancer: coming of age. Nat. Rev. Immunol. 18, 309-324 (2018).

32—T. Ahmed, et al., Mortality in severely malnourished children with diarrhoea and use of a standardised management protocol. Lancet 5, 353, 1919-1922 (1999).

33—N. Choudhury, T. Ahmed, M. I. Hossain, M. M. Islam, S. A. Sarker, M. Seilani, J. D. Clemens. Ready-to-Use Therapeutic food made from locally available food ingredients is well accepted by children having severe acute malnutrition in Bangladesh. Food and Nutrition Bulletin 39, 116-126 (2018).

34—C. B. Newgard, et al., A branched-chain amino acid-related metabolic signature that differentiates obese and lean humans and contributes to insulin resistance. Cell Metab. 9, 311-326 (2009).

35—C. T. Ferrara, et al., Genetic networks of liver metabolism revealed by integration of metabolic and transcriptional profiling. PLoS Genet. 4, e1000034 (2008).

36—M. V. Jensen, et al., Compensatory responses to pyruvate carboxylase suppression in islet beta-cells. Preservation of glucose-stimulated insulin secretion. J. Biol. Chem. 281, 22342-22351 (2006).

37—C. Magnes, et al., LC/MS/MS method for quantitative determination of long-chain fatty acyl-CoAs. Anal. Chem. 77, 2889-2894 (2005).

38—P. J. White et al., Branched-chain amino acid restriction in Zucker-fatty rats improves muscle insulin sensitivity by enhancing of efficiency of fatty acid oxidation and acyl-glycine export. Mol. Metab. 5, 538-551 (2016).

39—M. E. Ritchie et al., limma powers differential expression analyses for RNA-sequencing and microarray studies. Nucleic Acids Res. 43, e47 (2015)

40—C. Simillion, R. Liechti, H. E. L. Lischer, V. loannidis, R. Bruggmann, Avoiding the pitfalls of gene set enrichment analysis with SetRank. BMC Bioinformatics. 18, 1-14 (2017).

41—J.G. Caporaso, et al., QIIME allows analysis of high-throughput community sequencing data. Nat. Meth. 7, 335-336 (2010).

42—J. D. Planer, et al., Development of the gut microbiota and mucosal IgA responses in twins and gnotobiotic mice. Nature 534, 263-266 (2016).

43—M. Baym, et al. Inexpensive multiplexed library preparation for megabase-sized genomes. PLoS One 10, 1-15 (2015).

44—N. A Joshi, J. N. Fass, Sickle: A sliding-window, adaptive, quality-based trimming tool for FastQ files. (Version 1.33) [Software]. Available at https://github.com/najoshi/sickle.

45—M. Martin, Cutadapt removes adapter sequences from high-throughput sequencing reads. EMBnet.journal. 17, 10-12 (2011).

46—Y. Peng, H. C. M. Leung, S. M. Yiu, F. Y. L. Chin, IDBA-UD: a de novo assembler for single-cell and metagenomic sequencing data with highly uneven depth. Bioinformatics 28, 1420-1428 (2012).

47—T. Seemann, Prokka: rapid prokaryotic genome annotation. Bioinformatics 30, 2068— 2069 (2014).

48—Y. Liao, G. K. Smyth, W. Shi, featureCounts: an efficient general-purpose program for assigning sequence reads to genomic features. Bioinformatics 30, 923-930 (2014).

49—R Core Team, R: A language and environment for statistical computing. R. Foundation for Statistical Computing (2017) (available at https://www.R-project.org/).

50—M. Steinegger, J. Soding, MMseqs2 enables sensitive protein sequence searching for the analysis of massive data sets. Nat. Biotechnol. 35, 2-4 (2017).

51—B. Buchfink, C. Xie, D. H. Huson, Fast and Sensitive Protein Alignment using DIAMOND, Nature Methods 12, 59-60 (2015).

52—J. Liu et al., A laboratory-developed TaqMan array card for simultaneous detection of 19 enteropathogens. J. Clin. Microbiol. 51, 472-480 (2013).

53—MAL-ED Network Investigators. The Malnutrition and Enteric Disease Study (MAL-ED): Understanding the Consequences for Child Health and Development. Clin. Infect. Dis. 59, S193-206 (2014).

54—M. Kosek, et al., Assessment of environmental enteropathy in the MAL-ED cohort study: theoretical and analytic framework. Clin. Infect. Dis. 59, S239—S247 (2014).

55—A. L. Goodman, et al., Extensive personal human gut microbiota culture collections characterized and manipulated in gnotobiotic mice. Proc. Natl. Acad. Sci. U.S.A. 108, 6252— 6257 (2011).

56—N. Dey, et al., Regulators of gut motility revealed by a gnotobiotic model of diet-microbiome interactions related to travel. Cell 163, 95-107 (2015).

57—M. Wu, et al., Genetic determinants of in vivo fitness and diet responsiveness in multiple human gut Bacteroides. Science 350, aac5992 (2015).

58—S. Turner, et al., Investigating deep phylogenetic relationships among cyanobacteria and plastids by small subunit rRNA sequence analysis. J Eukaryot Microbiol. 46, 327-338 (1999).

59—S. Kurtz et al., Versatile and open software for comparing large genomes Genome Biol. 5, R12 (2004).

60—A. Bankevich, et al., SPAdes: a new genome assembly algorithm and its applications to single-cell sequencing. J. Comput. Biol. 19, 455-477 (2012).

61—M. Kanehisa and S. Goto, KEGG: kyoto encyclopedia of genes and genomes. Nucleic Acids Res. 28, 27-30 (2000).

62—R. Overbeek, et al., The subsystems approach to genome annotation and its use in the project to annotate 1000 genomes. Nucleic Acids Res. 33, 5691-5702 (2005).

63—R. K. Aziz, et al., The RAST Server: rapid annotations using subsystems technology. BMC Genomics doi: 10.1186/1471-2164-9-75 (2008).

64—M. Rajilić-Stojanović, W. M. de Vos, The first 1000 cultured species of the human gastrointestinal microbiota. FEMS Microbiol. Rev. 38, 996-1047. (2014).

65—P. S. Novichkov, RegPrecise 3.0—a resource for genome-scale exploration of transcriptional regulation in bacteria. BMC Genomics 14, 745 (2013).

66—C. Abreu-Goodger, E. Merino, RibEx: a web server for locating riboswitches and other conserved bacterial regulatory elements. Nucleic Acids Res. 33, W690-692 (2005).

67—N. P. McNulty et al., Effects of diet on resource utilization by a model human gut microbiota containing Bacteroides cellulosilyticus WH2, a symbiont with an extensive glycobiome. PLoS Biol. 11, e1001637 (2013).

68—M. C. Hibberd et al., The effects of micronutrient deficiencies on bacterial species from the human gut microbiota. Sci. Transl. Med. 9, eaa14069 (2017).

69—M. I. Love, W. Huber, S. Anders, Moderated estimation of fold change and dispersion for RNA-seq data with DESeq2. Genome Biol. 15, 550 (2014).

70—G. Yu, L.-G. Wang, Y. Han, Q.-Y. He, clusterProfiler: an R package for comparing biological themes among gene clusters. OMICS 16, 284-287 (2012).

71—W. Luo, M. S. Friedman, K. Shedden, K. D. Hankenson, P. J. Woolf, GAGE: generally applicable gene set enrichment for pathway analysis. BMC Bioinformatics 10, 161-17 (2009).

72—X. Wei, et al., Fatty acid synthesis configures the plasma membrane for inflammation in diabetes. Nature 539, 294-298 (2016).

73—T. Clavel, G. Henderson, W. Engst, J. Dore, M. Blaut, Phylogeny of human intestinal bacteria that activate the dietary lignan secoisolariciresinol diglucoside. FEMS Microbiol. Ecol. 55, 471-478 (2005).

74—R. Martin, S. Miguel, L. Benevides, C. Bridonneau, Functional characterization of novel Faecalibacterium prausnitzii strains isolated from healthy volunteers: A step forward in the use of F. prausnitzii as a next-generation probiotic isolation of novel extremely oxygen. Front. Microbiol. 8, 1-13 (2017).

75—H. Sokol, et al., Faecalibacterium prausnitzii is an anti-inflammatory commensal bacterium identified by gut microbiota analysis of Crohn disease patients. Proc. Natl. Acad. Sci. U.S.A. 105, 16731-16736 (2008).

76—P. Vassilyadi et al., Colitis, independent of macronutrient intake, compromises bone structure and strength in growing piglets. Pediatr. Res 80, 753-758 (2016).

77—T. Hildebrand and P. Rüegsegger, Quantification of bone microarchitecture with the structure model index. Comput. Methods Biomech. Biomed. Engin. 1, 15-23 (1997).

78—T. Masuda, M. Tomita, Y. Ishihama, Phase transfer surfactant-aided trypsin digestion for membrane proteome analysis. J. Proteome Res. 7, 731-740 (2008).

79—S. M. Clarkson et al., Construction and optimization of a heterologous pathway for protocatechuate catabolism in Escherichia coli enables bioconversion of model aromatic compounds. Appl. Environ. Microbiol. 83, e01313-17 (2017).

80—D. L. Tabb, C. G. Fernando, M. C. Chambers, MyriMatch: highly accurate tandem mass spectral peptide identification by multivariate hypergeometric analysis. J. Proteome Res. 6, 654-661 (2007).

81—Z. Q. Ma et al., IDPicker 2.0: Improved protein assembly with high discrimination peptide identification filtering. J. Proteome Res. 8, 3872-3881 (2009).

82—R. C. Edgar, Search and clustering orders of magnitude faster than BLAST, Bioinformatics 26, 2460-2461 (2010).

83—G. Baumann, Growth hormone binding protein. The soluble growth hormone receptor. Minerva Endocrinologica, 27, 265-276 (2002).

84—M. H. Rasmussen, K. K. Y. Ho, L. Kjems, J. Hilsted, Serum growth hormone-binding protein in obesity: effect of a short-term, very low calorie diet and diet-induced weight loss. J. Clin. Endocrinol. Metab. 81, 1519-1524 (1996).

85—S.-H. Tan, et al., Plasma biomarker proteins for detection of human growth hormone administration in athletes. Sci. Rep. 7, 10039 (2017).

86—T. A. Pinheiro, et al., Obesity and malnutrition similarly alter the renin-angiotensin system and inflammation in mice and human adipose. J. Nutr. Biochem. 48, 74-82 (2017).

87—L. Lerner et al., Plasma growth differentiation factor 15 is associated with weight loss and mortality in cancer patients. J. Cachexia. Sarcopenia Muscle 6, 317-324 (2015).

88—T. Wang et al., GDF 15 is a heart-derived hormone that regulates body growth. EMBO Mol. Med. 9, 1150-1164 (2017).

89—J. B. Allard and C. Duan, IGF-Binding Proteins: Why do they exist and why are there so many? Front. Endocrinol. 9,117 (2018)

90—A. Hoeflich, V. C. Russo, Physiology and pathophysiology of IGFBP-1 and IGFBP-2—consensus and dissent on metabolic control and malignant potential. Best Pract. Res. Clin. Endocrinol. Metab. 29, 685-700 (2015).

91—C. Gyrup, C. Oxvig, Quantitative analysis of insulin-like growth factor-modulated proteolysis of insulin-like growth factor binding protein-4 and -5 by pregnancy-associated plasma protein-A. Biochemistry 46, 1972-1980 (2007).

92—X. Qin, et al., Pregnancy-associated plasma protein-A increases osteoblast proliferation in vitro and bone formation in vivo. Endocrinology 147, 5653-5661 (2006).

93—M. Rehage, et al., Transgenic overexpression of pregnancy-associated plasma protein-A increases the somatic growth and skeletal muscle mass in mice. Endocrinology 148, 6176-6185 (2007).

94—S. Kløverpris et al., Stanniocalcin-1 potently inhibits the proteolytic activity of the metalloproteinase pregnancy-associated plasma protein-A. J. Biol. Chem. 290, 21915-21924 (2015).

95—R. Varghese, et al., Overexpression of human stanniocalcin affects growth and reproduction in transgenic mice. Endocrinology 143, 868-876 (2002).

96—M. F. Gude, et al., PAPP-A, IGFBP-4 and IGF-II are secreted by human adipose tissue cultures in a depot-specific manner. Eur. J. Endocrinol. 175, 509-519 (2016).

97—C. Oxvig, The role of PAPP-A in the IGF system: location, location, location. J. Cell Commun. Signal. 9, 177-187 (2015).

98—M. A. Underwood, J. B. German, C. B. Lebrilla, D. A. Mills, Bifidobacterium longum subspecies infantis: champion colonizer of the infant gut. Pediatr. Res. 77, 229-235 (2015).

99—G. A. McQuibban et al., Inflammation dampened by gelatinase A cleavage of monocyte chemoattractant protein-3. Science 289, 1202-1206 (2000).

100—L. Davis, Y. Chen, M. Sen, WISP-3 functions as a ligand and promotes superoxide dismutase activity. Biochem. Biophys. Res. Commun. 342, 259-265 (2006).

101—C. G. Kleer et al., WISP3 and RhoC guanosine triphosphatase cooperate in the development of inflammatory breast cancer. Breast Cancer Res. 6, R110-115 (2004).

102—J. Y. Song, A. M. Holtz, J. M. Pinskey, B. L. Allen, Distinct structural requirements for CDON and BOC in the promotion of Hedgehog signaling. Dev. Biol. 402, 239-252 (2015).

103—F. Kugimiya et al., Physiological role of bone morphogenetic proteins in osteogenesis. J Bone Mineral Metab. 24, 95-99 (2006).

104—A. Pabois et al., Notch signaling mediates crosstalk between endothelial cells and macrophages via D114 and IL6 in cardiac microvascular inflammation. Biochemical Pharmacology 104, 95-107 (2016).

105—L. Meng et al., The Notch ligand DLL4 defines a capability of human dendritic cells in regulating Th1 and Th17 differentiation. J. Immunol. 196, 1070-1080 (2016).

106—K. A. Papadakis et al., TL1A synergizes with IL-12 and IL-18 to enhance IFN-gamma production in human T cells and NK cells. J. Immunol. 172, 7002-7007 (2004).

107—J. L. Prehn et al., Potential role for TL1A, the new TNF-family member and potent costimulator of IFN-γ, in mucosal inflammation. Clinical Immunol. 112, 66-77 (2004).

108—Prendergast, et al., Stunting is characterized by chronic inflammation in Zimbabwean infants. PLoS One 9, e86928 (2014).

109—S. Miguel et al., Identification of metabolic signatures linked to anti-inflammatory effects of Faecalibacterium prausnitzii. mBio 6, e00300-15 (2015).

110 —N. M. Breyner et al., Microbial anti-inflammatory molecule (MAM) from Faecalibacterium prausnitzii shows a protective effect on DNBS and DSS-induced colitis model in mice through inhibition of NF-KB pathway. Front, MicrobioL 8, 114 (2017).

111—D. J. Marchant et al., A new transcriptional role for matrix metalloproteinase-12 in antiviral immunity. Nat. Med. 20, 493-502 (2014).

112—R. A. Dean et al., Macrophage-specific metalloelastase (MMP-12) truncates and inactivates ELR+CXC chemokines and generates CCL2, -7, -8, and -13 antagonists: potential role of the macrophage in terminating polymorphonuclear leukocyte influx. Blood 112, 3455-3464 (2008).

113—S. Basu, R. J. Binder, R. Suto, K. M. Anderson, P. K. Srivastava, Necrotic but not apoptotic cell death releases heat shock proteins, which deliver a partial maturation signal to dendritic cells and activate the NF-kappa B pathway. Int. Immunol. 12, 1539-46 (2000).

114—A. Murshid, J. Gong, S. K. Calderwood, Heat Shock Protein 90 Mediates Efficient Antigen Cross Presentation through the Scavenger Receptor Expressed by Endothelial Cells-I. J. Immunol. 185, 2903-2917 (2010).

115—Y. Tamura, A. Yoneda, N. Takei, K. Sawada. Spatiotemporal regulation of Hsp90—ligand complex leads to immune activation. Frontiers Immunol. 7, 201 (2016).

116—M. Yapo, Pineapple and banana pectins comprise fewer homogalacturonan building blocks with a smaller degree of polymerization as compared with yellow passion fruit and lemon pectins: implication for gelling properties. Biomacromolecules 10, 717-721 (2009).

117—W. M. U. Fernando, et al., Diets supplemented with chickpea or its main oligosaccharide component raffinose modify faecal microbial composition in healthy adults. Benef. Microbes 1, 197-207 (2010).

118—S. Fukuda et al., Bifidobacteria can protect from enteropathogenic infection through production of acetate. Nature 469, 543-547 (2011).

119—C. J. Kelly et al., Crosstalk between microbiota-derived short-chain fatty acids and intestinal epithelial HIF augments tissue barrier function. Cell Host Microbe 17, 662-671 (2016).

120—P. J. Fielder et al., Differential long-term effects of insulin-like growth factor-I, growth hormone (GH), and IGF-I plus GH on body growth and IGF binding proteins in hypophysectomized Rats. Endocrinology 137, 1913-1920 (1996).

121—J. Zhao et al., The role of height-associated loci identified in genome wide association studies in the determination of pediatric stature. BMC Med. Genet. 11, 96 (2010).

122—P. J. McLaughlin et al., Lack of fibulin-3 causes early aging and herniation, but not macular degeneration in mice. Human Mol. Gen.16, 3059-3070 (2007).

123—R. C. Meex, et al., Fetuin-B is a secreted hepatocyte factor linking steatosis to impaired glucose metabolism. Cell Metab. 22, 1078-1089 (2015).

124—J. W. Choi, H. Liu, R. Mukherjee, J. W. Yun, Downregulation of fetuin-B and zinc-a2-glycoprotein is linked to impaired fatty acid metabolism in liver cells. Cell Physiol. Biochem. 30, 295-306 (2012).

### Methods for Examples 1-6

### (a) SAM Trial: Study Population and Study Protocol

The human study entitled Development and field testing of ready-to-use therapeutic foods (RUTF) made of local ingredients in Bangladesh for the treatment of children with severe acute malnutrition' was approved by the Ethical Review Committee at the icddr,b and conducted between April 2013 and December 2015 (ClinicalTrials.gov identifier: NCT01889329). The goal was to determine whether therapeutic food prototypes developed by icddr,b and made from locally available food ingredients are non-inferior in efficacy compared to a standard, commercially available RUTF used for treating children with SAM (Plumpy'Nut; Nutriset). A total of 343 children, aged 6 to 59 months, were enrolled with SAM [defined by WHZ <−3 and/or having bipedal edema, and/or a mid-upper arm circumference (MUAC) <11.5 cm] and for whom written informed consent was obtained from their parent or guardian. Children from urban or peri-urban areas of Dhaka were recruited to the study from Dhaka Hospital of icddr,b, and from two clinics (TDH, Kurigram and RADDA MCH FP Center Mirpur, Dhaka).

After acute stabilization, entailing rehydration and a short course of antibiotics (32), children were transferred for follow-up treatment to the Nutrition Rehabilitation Unit (NRU) of Dhaka Hospital or to TDH and RADDA [For children without signs or symptoms of infection other than those of diarrhea, antibiotic therapy began with intramuscular or intravenous ampicillin 100 mg/kg daily with doses every 6 h, and gentamicin 5 mg/kg daily with doses every 12 h. If there was no evidence of septicemia after 48 h, ampicillin and gentamicin were discontinued and amoxicillin was administered orally (100 mg/kg) every 8 h for 3 more days. Children with pneumonia were treated with intravenous chloramphenicol (100 mg/kg) every 6 h for 24 h and then orally for a total of 7 days. If septicemia was suspected, ampicillin was given (200 mg/kg/day) and gentamicin was continued for 7-10 days].

An appetite test was performed prior to randomization to one of the three therapeutic food arms; a rice-lentil formulation, a chickpea-containing formulation (both locally produced) (33), or Plumpy'Nut. The therapeutic food was provided each morning at a dose of ˜200 kcal/kg/d. Children who were breast-fed continued breastfeeding. Subjects were discharged from the study upon fulfillment of the following graduation criteria: (i) an edema-free WHZ ≥−2 for those admitted with WHZ <−3 and/or edema, or (ii) MUAC ≥115 mm with edema-free weight gain of 15% for those admitted with a MUAC <115 mm. Before discharge, children were treated with anti-helm inthic medication as per national guidelines (200 mg albendazole for children aged 12-23 months; 400 mg for children aged >24 months), and their parents received nutritional counseling.

As described in the main text and FIG. 1A, a subset of 54 children at the Dhaka Hospital site were enrolled in a sub-study that included regular fecal sampling and three blood draws for up to 1 year after discharge from the Nutritional Research Unit (n=20, Plumpy'Nut arm; n=19, rice-lentil based therapeutic food arm; n=15, chickpea-containing therapeutic food arm). Fecal samples were collected and within 10 minutes transferred to liquid nitrogen pre-charged dry shippers (Taylor Wharton, CX-100) for transport back to the lab where they were stored at −80° C. EDTA plasma was prepared from blood using standard procedures and placed in a −80° C. freezer. Coded biospecimens were shipped to Washington University on dry ice where they were stored at −80° C., along with associated clinical metadata, in a dedicated biospecimen repository with approval from the Washington University Human Research Protection Office.

### (b) MDCF Trial: Study Population and Study Protocol

Sixty-three 12-18-month-old children diagnosed with MAM (WHZ <−2) who were no longer exclusively breastfed were enrolled in a double-blind, randomized, four group, parallel assignment interventional trial study (ClinicalTrials.gov identifier NCT03084731) conducted in Dhaka, Bangladesh and approved by the Ethical Review Committee at the icddr,b. The study was designed to test the effects of three locally produced MDCF prototypes described in FIG. 13 and a locally produced rice-lentil-based ready-to-use supplemental food (RUSF). Experiments for developing recipes and preparation of samples were performed at the icddr,b Food Processing Laboratory; a standardized production procedure was followed to control the quality of MDCFs from each production batch. Linear programming was used to design the MDCF prototypes, with a target energy density of 250 kcal/50 g, and a caloric distribution of 45-55 percent from fat and 8-12 percent from protein. All diets were supplemented with multiple micronutrient premix that provided 70% of the RDA, for children aged 12-18 months, of vitamins A, C, D and E, all B vitamins, calcium, copper, iron, magnesium, manganese, phosphorous, potassium and zinc.

After obtaining informed consent, socio-demographic data was collected for all participants, and enrolled children were randomized into one of four treatment groups (n=14-17 per group). After 2 weeks on their home diet, with weekly fecal sample collection and anthropometry, children and their mothers attended a local community health clinic every morning and afternoon where the child was provided 25 g of their assigned MDCF/RUSF for consumption per session (total daily energy intake from the MDCF/RUSF, ˜200-250 kcal). Mothers were asked not to give any food or breast milk during the 2 hours preceding the prescribed feeding session. Diets were freshly prepared each day at icddr,b, and the quantity of food consumed by each child was recorded at each visit. Mothers were instructed to continue normal breastfeeding and home-based complementary feeding practices outside of clinic visits. A blood sample was collected and EDTA-plasma was prepared from each child at the beginning of the intervention phase (week 3) and again at the end of the 4-week intervention (week 7) for targeted mass-spectrometry-based metabolomic and proteomic analyses. Fecal samples, together with anthropometric and morbidity data, were collected weekly from each child, including during a 2-week post-intervention period. A separate reference cohort of thirty 12- to 24-month-old healthy children (WHZ and HAZ scores >−1) were also consented to provide a single blood and fecal sample to compare with those collected from the children with MAM enrolled in the intervention study. All biospecimens were rapidly cryopreserved after collection (see above), coded and stored at −80° C. prior to transfer to Washington University, with approval from the Washington University Human Research Protection Office.

### (c) Targeted Mass Spectrometry-Based Metabolomics Analysis of Plasma Samples

Clinical chemistry analytes, including glucose, lactate, triglycerides, total ketones, and non-esterified fatty acids (NEFA), were measured using a UniCel DxC600 clinical analyzer (Beckman). Reagents for the first three analytes were provided by Beckman (Brea, Calif.) while those for ketones and NEFA were obtained from Wako (Mountain View, Calif.). Amino acids, acylcarnitines, organic acids, and acylCoAs were analyzed using stable isotope dilution techniques. Amino acids and acylcarnitines were measured by flow injection tandem mass spectrometry with specific internal standards (34, 35); data were acquired using a Waters AcquityTM UPLC system equipped with a triple quadrupole detector and a data system controlled by MassLynx 4.1 OS (Waters, Milford, Mass.). Organic acids were quantified using Trace Ultra GC coupled to ISQ MS operating under Xcalibur 2.2 (Thermo Fisher Scientific) (36). AcylCoAs were extracted, purified and measured by flow injection analysis using positive electrospray ionization on a Xevo TQ-S triple quadrupole MS (Waters) (37); heptadecanoyl CoA was employed as an internal standard (38).

### (d) Quantitative Proteomics

Plasma levels of leptin and insulin were quantified by using the MILLIPLEX MAP Human Bone Magnetic Bead Panel (MilliporeSigma). IGF-1 was measured using the Human IGF-1 Quantikine ELISA (R&D Systems).Plasma levels of leptin and insulin were quantified by using the MILLIPLEX MAP Human Bone Magnetic Bead Panel (MilliporeSigma). IGF-1 was measured using the Human IGF-1 Quantikine ELISA (R&D Systems).

The SOMAscan 1.3K Proteomic Assay plasma/serum kit (SomaLogic, Boulder, Colo., USA) was used to measure 1,305 proteins in plasma samples (50 μL aliquots). Following the manufacturer's protocol and utilizing SOMAmer reagents immobilized on streptavidin beads, proteins from plasma samples were tagged with NHS-biotin reagent, captured as a SOMAmer reagent/protein complex, cleaved, denatured, eluted and hybridized to a custom Agilent DNA microarray. Microarrays were scanned with an Agilent SureScan scanner at 5 tm resolution, and the Cy3 fluorescence readout was quantified. Raw signal values were processed using Somalogic's SOMAscan standardization procedures, including hybridization normalization, plate scaling, median scaling, and final somamer calibration, each of which generates a SOMAscan ‘.adat’ data file. The R package ‘limma’ (Bioconductor) was used to analyze differential protein abundances. In limma, signal data are subject to linear model fitting and empirical Bayesian statistics for group comparisons (39). Spearman correlation analyses were performed between measured SOMAscan analytes (proteins) and anthropometric scores, plasma metabolites, as well as the abundances of bacterial OTUs in fecal samples.

Proteins measured in the plasma of children with healthy growth phenotypes or with SAM (prior to treatment) were rank-ordered according to the fold-difference in their levels between these two groups. As noted in the main text, the top 50 most differentially abundant proteins in healthy compared to SAM were designated as healthy growth-discriminatory proteins, and the top 50 most differentially abundant in SAM compared to healthy were designated as SAM-discriminatory proteins. The average fold-change for these healthy growth- and SAM-discriminatory proteins was then calculated for each treatment arm in the MDCF trial (pre- versus post- MDCF/RUSF treatment) and normalized to the mean fold-change across all four arms (column normalization in FIG. 14). Limma was used to calculate statistical significance.

Proteins with an absolute Pearson's r>0.25 and FDR corrected p-value <0.05 for HAZ were identified. The average fold-change in abundance for these ‘HAZ-discriminatory proteins’ was calculated for each treatment arm in the MDCF trial (pre-versus post-treatment) and normalized to the mean fold-change across all four arms (column normalization in FIG. 14).

Proteins measured by the SOMAscan 1.3k Proteomic Assay platform were mapped to all Gene Ontology (GO) ‘Biological Processes’ in the GO database (www.geneontology.org). SetRank, a gene set enrichment analysis (GSEA) algorithm (40), was employed to identify GO ‘Biological Processes’ that were significantly enriched for proteins that exhibited changes in abundance from pre- to post-treatment with MDCF/RUSF. Enrichment was calculated using the setRankAnalysis function in the SetRank R library (parameters: use.ranks=TRUE; setPCutoff=0.01; and fdrCutoff=0.05). The average fold-change for each protein in the statistically significant Biological Process category was calculated for each treatment arm and normalized to the mean fold-change across all four arms (FIG. 14). We defined proteins within the GO Biological Process as ‘healthy growth-discriminatory’ if they were increased by at least 30% in healthy individuals compared to those with SAM, and ‘SAM-discriminatory’ if they were increased by at least 30% in children with SAM compared to those who classified as healthy.

(e) Characterizing Human Fecal Microbial Communities as a Function of Host Nutritional Status: V4-16S rRNA Gene Sequencing and Data Analysis

V4-16S rRNA gene sequencing and data analysis—Frozen fecal samples were pulverized in liquid nitrogen. DNA was extracted from an aliquot of the pulverized material (˜50 mg) by bead-beating with 500 tL of 0.1 mm diameter zirconia/silica beads in a solution consisting of 500 tL phenol:chloroform:isoamyl alcohol (25:24:1), 210 tL 20% SDS, and 500 tL buffer A (200 mM NaCl, 200 mM Trizma base, 20 mM EDTA). DNA was purified (Qiaquick columns, Qiagen), eluted in 70 tLTris-EDTA (TE) buffer, and quantified (Quant-iT dsDNA broad range kit; Invitrogen). Each DNA sample was adjusted to a concentration of 1 ng/tL and subjected to PCR using barcoded primers directed against variable region 4 of the bacterial 16S rRNA gene and the following cycling conditions: denaturation (94° C. for 2 minutes) followed by 26 cycles of 94° C. for 15 seconds, 50° C. for 30 seconds and 68° C. for 30 seconds, followed by incubation at 68° C. for 2 minutes (2). Amplicons were quantified, pooled and sequenced (Illumina MiSeq instrument, paired-end 250 nt reads). Paired-end reads (trimmed to 200 nt) were merged (FLASH, version 1.2.6), demultiplexed, clustered into 97% ID OTUs and aligned against the GreenGenes 2013 reference database using QIIME version 1.9.0 (41). Taxonomy was assigned to 97% ID OTUs with RDP 2.4, as described previously (42). The resulting OTU table was filtered to include only OTUs with ≥0.1% relative abundance in at least two samples.

As recent studies have produced newer methods for processing 16S rDNA data, a sensitivity analysis was performed comparing OTU assignments derived from QIIME with ASVs generated from DADA2. This analysis, described in (14), confirmed the concordance between the QIIME and DADA2 outputs.

### (f) Characterizing Human Fecal Microbial Communities as a Function of Host Nutritional Status: Microbiota-for-Age Z-Scores

MAZ scores (2) were calculated using the sparse RF-derived Bangladeshi model of normal gut microbiota development, and the median and standard deviation of the predicted microbiota ages of the reference cohort of chronologically age-matched healthy Mirpur infants/children (binned by month).

### (q) Characterizing Human Fecal Microbial Communities as a Function of Host Nutritional Status: Functional Microbiome Maturity

DNA was extracted from frozen fecal samples, quantified (Qubit), and each preparation was normalized to a concentration of 0.75 ng/μL. Libraries were generated from each DNA sample using the Nextera XT kit (Illumina) with the reaction volume scaled down 10-fold to 2.5 μL (43). Samples were pooled and sequenced (Illumina NextSeq instrument; paired-end 150 nt reads). A defined consortium of 16 human gut bacterial strains was included in each sequencing run as a reference control. Reads were quality filtered with Sickle (44) and Nextera adapter sequences were trimmed using cutadapt (45). Bowtie2 and the hG19 build of the H. sapiens genome were employed to identify and remove host sequences prior to further processing. Reads were subsequently assembled using IDBA-UD (46) and initially annotated with Prokka (47). Paired-end sequencing reads generated from each sample were mapped to contigs that had been assembled from that sample. Duplicate reads (optical- and PCR-generated) were identified and removed from mapped data using the Picard MarkDuplicates tool (v 2.9.3). Counts were aggregated for each gene (featureCounts; Subread v. 1.5.3 package) (48) and normalized (reads per kilobase per million, RPKM) in R (v. 3.4.1; (49)).

Functional profiles for each fecal microbiome sample were generated by assigning microbiome-encoded proteins to a collection of 58 mcSEED subsystems/pathway modules that capture core metabolism of 75 nutrients/metabolites in four major categories (19 amino acids, 10 vitamins, 40 sugars, and 6 fermentation products) projected over 2,313 annotated reference bacterial genomes. The meta-proteomes from all samples were clustered at 90% identity [MMSeqs2 (50); —min-seq-id 0.9]. One representative protein sequence was randomly selected from each cluster. Clustering and representative protein sequence selection was performed in the same manner for all proteins in the 58 mcSEED subsystems/pathway modules. Representative proteins from the fecal meta-proteomes were queried against representative proteins from these mcSEED subsystems/pathway modules using DIAMOND (51) with the threshold for best hits set to ≥80% identity. As a result of this mapping, all members of a given cluster of microbiome-encoded proteins were assigned the best-hit annotation of the representative mcSEED protein.

A sparse RF-derived model was built using the aggregated mcSEED subsystem/pathway module abundances for all fecal samples collected from 10 healthy Bangladeshi children who had been sampled monthly from birth to 2 years of age. Applying this model to a separate test set of 20 healthy children sampled at 6, 12, 18, and 24 months of age gave a prediction of functional microbiome age. A smoothing spline function was fit between the predicted functional microbiome age and chronologic age of each individual at the time of fecal sample collection for these 20 healthy children. Limiting the model to the 30 subsystems/pathway modules with the highest feature importance scores did not significantly impact its accuracy. The resulting sparse RF-derived model explained 69.1% of the variance associated with age. The model was applied to a separate test set of 20 healthy Bangladeshi individuals sampled at 6-, 12-, 18-, and 24-months-of-age. The correlation (Pearson's r) between chronological age and functional microbiome age in this test set was 0.66 (p=4.4×10⁻⁶), with a mean absolute error (MAE) of 3.9 months and root mean square error (RMSE) of 5.1 months (29.2% of the mean).

The sparse RF-derived model was then applied to the mcSEED subsystem/pathway module abundance profiles of fecal samples obtained from children with SAM prior to, during and after treatment. Relative functional maturity for each sample was calculated by subtracting the functional microbiome age of that sample from the spline fit functional microbiome age of samples obtained from healthy children of similar chronologic age.

(h) Characterizing Human Fecal Microbial Communities as a Function of Host Nutritional Status: Quantifying Enteropathogen Burden by Multiplex qPCR

Quantifying enteropathogen burden by multiplex qPCR—Nucleic acids were isolated from fecal samples and adjusted to 2ng/μL. Levels of 18 bacterial and viral pathogens and parasites were determined by using a microfluidic-based digital PCR system with 96.96 Dynamic Arrays (Fluidigm Corp. San Francisco, Calif.). TaqMan primers and probes (52) were used to construct the 24 different assays employed for this analysis. cDNAs were prepared from 50 ng of total RNA using Life Technologies High Capacity cDNA Reverse Transcription Kit (Applied Biosystems, Foster City, CA). The resulting products were subjected to Specific Target Amplification (STA) using TaqMan PreAmp Mastermix (Applied Biosystems), 50 nM of each primer, and the following cycling conditions; 10 minutes at 95° C. followed by 14 cycles of 95° C. for 15 seconds and then 60° C. for 1 minute. At the conclusion of this step, the reaction mixture was diluted 1:4 in low EDTA DNA suspension buffer (10 mM Tris, 0.1 mM EDTA, pH 8.0) combined with TaqMan Universal PCR Master Mix (Applied Biosystems) and 20X Gene Expression Sample Loading Reagent (Fluidigm Corp.). Assay mixtures containing 9 μM of each primer and 2 pM of the probe in Dynamic Array Assay Loading Reagent (Fluidigm Corp) (52) were loaded into appropriate inlets on the primed 96.96 Dynamic Array chip before it was placed on the NanoFlex-4 Integrated Fluidic Circuit Controller for distribution of the sample and assay mixture. The loaded Dynamic Array was then inserted into the BioMarkTM Reverse-Transcription-PCR System. The qPCR program consisted of the following steps: 50° C. for 2 minutes, 95° C. for 2 minutes, and 40 cycles of 95° C. for 15 seconds and 60° C. for 1 minute. Enteropathogen abundance was calculated by comparing cycle threshold to standards of known concentration, yielding absolute measurements of pg genomic DNA (bacterial enteropathogens and parasites), copy number (RNA viruses) and mass of viral DNA per lysate mass (Adenovirus).

### (i) General Mouse Studies

All mouse experiments were performed using protocols approved by Washington University Animal Studies Committee. Mice were housed in plastic flexible film gnotobiotic isolators (Class Biologically Clean Ltd., Madison, WI) at 23° C. under a strict 12-hour light cycle (lights on a 0600h). Male germ-free C57BL/6 mice were initially weaned onto an autoclaved, low-fat, high-plant polysaccharide chow that was administered ad libitum (B&K Universal, East Yorkshire, U.K; diet 7378000). Animals were maintained on this diet until 3 days prior to the beginning of experiments involving tests of the effects of complementary food ingredients. Using a disposable sterile gavage needle, defined consortia of sequenced age-discriminatory bacterial strains cultured from Bangladeshi children, or intact uncultured microbiota from donors with post-SAM MAM were introduced into recipient mice at 5 weeks of age. All animals were euthanized by cervical dislocation without prior fasting.

### (j) Screen of CFCs Described in FIG. 5 and the Monotonous Feeding Experiments Involving the Initial MDCF Prototype and MS/KF Described in FIG. 7.: Design and Preparation of Diets

Bangladeshi diets were constructed using extensive knowledge of Bangladeshi complementary feeding practices, including quantitative 24-hour dietary recall surveys conducted at the Mirpur site as part of the MAL-ED study [see (53) for a description of methods]. All diets were prepared by Dyets, Inc. (Bethlehem, Pa.). The compositions and quantities of each ingredient used to prepare each diet are provided in Table s8 of Gehrig et al. Science, 2019, 365(6449):eaau4732, which is incorporated by reference in its entirety.

To prepare the Mirpur-18 diet (Table 5D), rice (parboiled, long grain) and red lentils (masoor dal) were each cooked separately with an equal weight of water at 100° C. in a steam jacketed kettle until ‘par-cooked’ (grains cooked, but still firm) and then set aside. Fresh market white potatoes, spinach and yellow onions were washed, chopped in a vertical cutter mixer and cooked in the kettle without added water at 70° C. until soft. Sweet pumpkin (Calabaza variety) was cut and boiled in the steam jacketed kettle until soft, and then strained. At this point, all of the cooked ingredients were combined, whole bovine milk powder (Franklin Farms East, Bethlehem, Pa.), soybean oil, salt, turmeric and garlic were added and the resulting diet was mixed extensively.

To prepare the CFC diets, rice, red lentils, potato, spinach and sweet pumpkin were cooked as described above for Mirpur-18. Canned garbanzo chickpeas and ground peanuts were roasted separately in a small amount of water for 8-10 minutes and blended into a paste prior to use. Tilapia (frozen fillets) was placed in the kettle with a small amount of water to steam until cooked thoroughly (˜20 minutes). Eggs were scrambled. Banana, whole milk powder and whole-wheat flour (atta) were not cooked. Individual CFC diets were prepared by combining the various component ingredients in the quantities listed in Table 5B and mixed thoroughly using a planetary mixer prior to pelleting.

To generate Khichuri-Halwi, Khichuri (Table 5D) was prepared by first cooking rice and red lentils in a steam kettle (Groen) at 100° C. with an equal weight of water until the grains were cooked but still firm. White potato, spinach and yellow onions were washed and chopped in a vertical cutter mixer and cooked with the spices in the steam kettle without added water at 70° C. until soft. Sweet pumpkin was cut and boiled in the kettle until soft, and then strained. Cooked ingredients were then combined on a weight basis in the proportions shown in Table 5D. To prepare Halwa (Table 5D), jaggery was added to the steam kettle with water and heated (70° C.) until it was fully dissolved, after which time cooked lentils were added. This hot mix was added to the bowl of a planetary mixer in which atta flour (pre-roasted for 5 minutes with a small amount of water) and soybean oil had been pre-mixed. The Halwa was blended extensively into a uniform thick paste. Milk suji was prepared by combining whole bovine milk powder, rice powder, sugar and soya oil along with minerals in the amounts listed in Table 5D. The individual components (milk suji, Khichuri, and Halwa) were then combined at a ratio of 28:36:36 (to simulate the relative contributions of these components to dietary intake during the nutritional rehabilitation phase of treatment of children with SAM at Dhaka Hospital) and homogenized.

Once all diets had been prepared, they were spread on trays, dried overnight at 30° C., and pelleted by extrusion (1/2″ diameter; California Pellet Mill, CL5). Dried pellets were weighed into ˜250 g portions, placed in a paper bag with an inner wax lining, which in turn was placed in a plastic bag. The material was vacuumed sealed and sterilized by gamma irradiation (30-50 kGy; Sterigenics, Rockaway, NJ). Sterility was assessed by culturing irradiated pellets in Brain Heart Infusion (BHI) broth, Nutrient broth, and Sabouraud-dextran broth (all from Difco) for one week at 37° C. under aerobic conditions, and in Tryptic Soy broth (Difco) under anaerobic conditions (atmosphere of 75% N2, 20% CO2 and 5% H2). Additionally, cultures of all diets were plated on BHI agar supplemented with 10% horse blood (Difco). The irradiated diet pellets were subjected to nutritional analysis (Nestlé Purina Analytical Laboratories; St. Louis, Mo.) (see, Table s6F of Gehrig et al. Science, 2019, 365(6449):eaau4732, which is incorporated by reference in its entirety). All diets were stored at −20° C. prior to use.

### (k) Screen of CFCs Described in FIG. 5 and the Monotonous Feeding Experiments Involving the Initial MDCF Prototype and MS/KF Described in FIG. 7: Isolation and Culturing of Age-Discriminatory and SAM-Associated Bacterial Strains

Bacterial strains were cultured from fecal samples collected from a 24-month-old child with SAM enrolled in the SAM clinical study at icddr,b described above [Development and Field Testing of Ready-to-Use-Therapeutic Foods Made of Local Ingredients in Bangladesh for the Treatment of Children with SAM' (ClinicalTrials.gov Identifier, NCT01889329)] and from three donors aged 6-24 months that exhibited healthy growth as defined by serial anthropometry. These latter three children were members of two previously completed Bangladeshi birth cohort studies; (i) ‘Field Studies of Amebiasis in Bangladesh’ [(ClinicalTrials.gov identifier: NCT02734264) and (ii) ‘Interactions of Enteric Infections and Malnutrition and the Consequences for Child Health and Development’ (abbreviated ‘Malnutrition and Enteric Disease Study’ (MAL-ED) ClinicalTrials.gov identifier; NCT02441426)] (53, 54). The research protocols for these two studies was approved by the Ethical Review Committee at the icddr,b. Informed consent was obtained from the mother/guardian of each child. Collection and use of biospecimens from each of the human studies was approved by the Washington University Human Research Protection Office (HRPO).

Collections of cultured anaerobic bacterial strains were generated from frozen fecal samples according to previously published methods (55, 56). All procedures were performed under an atmosphere of 75% N2, 20% CO2, and 5% H2 in vinyl anaerobic chambers (Coy Laboratory Products, Grass Lake, Mich.). Fecal samples (˜0.1 g) were weighed, brought into the Coy chamber, diluted 1:10 (wt/vol) with reduced PBS (PBS/0.05% L-cysteine-HCI) in 50 mL conical plastic tubes containing 5 mL of 2 mm-diameter glass beads (VWR, catalogue number 26396-506). Tubes were gently vortexed and the resulting slurry was passed through a 100 pm-pore diameter nylon cell strainer (BD Falcon). The clarified stool sample was then combined with an equal volume of a solution of PBS/0.05% L-cysteine-HCl/30% glycerol and aliquoted into 1.8 mL glass vials (E-Z vials, Wheaton). Tubes were crimped with covers containing a PTFE/grey butyl liner (Wheaton), and stored at −80° C.

Frozen stocks were brought into the Coy chamber, thawed and serially diluted over a 1000-fold range with PBS/0.05% L-cysteine-HCI. 100 μL of each dilution were spread on agar plates containing MegaMedium and 0.05% L-cysteine-HCl (55, 57). Plates were incubated at 37° C. under anaerobic conditions for 48 h. Single colonies were handpicked into 96-deep-well plates (Thermo Fisher Scientific) containing 600 μL of MegaMedium broth. Deep-well plates were subsequently incubated at 37° C. under anaerobic conditions for 48 h, at which point a 50 μL aliquot from each deep well was robotically transferred into a well of a 96-well shallow plate containing an equal volume of PBS/0.05% L-cysteine-HCl/30% glycerol (n=2 replicate stock plates; stored at −80° C.). The deep well plate with the remaining 500 μL in each well was removed from the Coy chamber and subjected to centrifugation (3220×g for 20 min at 4° C.). Using a liquid handling robot, the resulting supernatant was removed and DNA was extracted from cell pellets with phenol:chloroform. V4-16S rDNA amplicons were generated by PCR and sequenced (IIlumina MiSeq; paired-end 250 nt reads).

Isolates whose V4-16S rDNA sequences shared 97% sequence identity with age-discriminatory 97%ID OTUs and/or were enriched in the microbiota of children with SAM were selected for an additional round of colony purification. Full-length 16S rDNA gene amplicons were generated from these isolates using primers 8F and 1391R (58). Isolates sharing 99% nucleotide sequence identity in their full length 16S rRNA genes, and 96% nucleotide sequence identity throughout their genomes [NUCmer; (59); (see next paragraph)], were defined as unique strains. Taxonomy was assigned based on the full-length 16S rDNA sequences (RDP version 2.4 classifier; Table 4). Purified, sequenced strains were each grown to mid-log phase in MegaMedium; stocks were then prepared (15% glycerol/MegaMedium) in crimped vials and stored at -80° C.

### (I) Screen of CFCs Described in FIG. 5 and the Monotonous Feeding Experiments Involving the Initial MDCF Prototype and MS/KF Described in FIG. 7: Bacterial Genome Sequencing, Assembly, Annotation, In Silico Reconstructions and Phenotype Predictions

Barcoded, paired-end genomic libraries were prepared for each bacterial isolate DNA sample, and the libraries were sequenced in multiplex (IIlumina MiSeq instrument; paired-end 150 nt or 250 nt reads). Reads were de-multiplexed and assembled using SPAdes (60). Contigs with greater than 10× coverage were initially annotated using Prokka (47). Genes in each genome were also annotated at various levels by mapping protein sequences to the Prokaryotic Peptide Sequence database of the Kyoto Encyclopedia of Genes and Genomes (KEGG, 4 March 2017 release; 1x10⁻⁵ E-value threshold for BLASTP searches; 61). Subsystems-based, context-driven functional assignments of genes, curation and metabolic reconstructions were performed in the web-based mcSEED (microbial communities SEED) environment, a private clone of the publicly available SEED platform (62, 63). The mcSEED platform currently includes (i) ˜6,000 bacterial genomes carefully selected for phylogenetic diversity, including a subset of 2,300 reference mammalian gut microbial genomes representing 690 species (64), and (ii) a collection of curated metabolic subsystems. These subsystems include a subset of 58 biosynthetic, salvage and utilization pathway modules for amino acids, B-vitamins and related cofactors, carbohydrates, central carbon metabolism and fermentation, projected over ˜200 genomes representing the cultured strains described in this report and their nearest phylogenetic neighbors.

In silico reconstructions of selected metabolic pathways (captured in respective subsystems) were based on functional gene annotation and prediction using homology-based methods supplemented by three genome context techniques: (i) clustering of functionally-related genes on the chromosome (operons) compared to closely-related annotated genomes, (ii) predicted co-regulation of genes by a common regulator (regulons), and (iii) co-occurrence of genes in a set of related genomes. Context-based techniques are particularly helpful in (i) disambiguating paralogs with related but distinct functions (characteristic for sugar utilization pathways, most notably transporters and transcriptional regulators), (ii) filling in gaps (“missing genes”) in known pathway variants, including functional assignments (predictions) of previously uncharacterized protein families (e.g., non-orthologous gene replacements), and (iii) inferring alternative biochemical routes. Initial training sets of transcription factor binding sites (TFBSs) and co-regulated genes were taken from the RegPrecise database of bacterial regulons ((65); http://regprecise.lbl.gov/). RNA regulatory elements (riboswitches) were determined using RibEx (66). Note that mcSEED pathways may be more granular than a subsystem, splitting it to certain aspects (e.g. uptake of a nutrient separately from its metabolism). mcSEED subsystems/pathway modules are presented as lists of assigned genes and their annotations.

Predicted phenotypes are generated from the collection of mcSEED subsystems/pathway modules represented in a microbial genome. Phenotypes correspond to a specific metabolite (or several related metabolites) that are either a starting point (as in sugar utilization pathways) or an endpoint (as in amino acid biogenesis pathways). Predictions were generated in the form of a Binary Phenotype Matrix, showing the supporting evidence (presence/absence of genes in a pathway). Information from the Carbohydrate Active Enzyme (CAZy) database (http://www.cazy.org) was integrated into the annotations to expand subsystem/pathway module coverage for utilization of complex carbohydrates.

### (m) Screen of CFCs Described in FIG. 5 and the Monotonous Feeding Experiments Involving the Initial MDCF Prototype and MS/KF Described in FIG. 7: Community Profiling by Sequencing (COPRO-Seq)

The effects of diet on the structure of the defined consortium of cultured strains was defined by COPRO-Seq (67). Briefly, DNA was isolated by subjecting fecal pellets or cecal contents, collected from gnotobiotic mice, to bead-beading for 3 minutes in a mixture containing 500 μL Buffer A (200 mM NaCl, 200 mM Tris, 20 mM EDTA), 210 μL 20% SDS, 500 μL phenol:chloroform:isoamyl alcohol (25:24:1, pH 7.9), and 250 μL of 0.1 mm diameter zirconium beads. Bead-beating was performed in 2 mL screw cap tubes (Axygen) using Mini-Beadbeater-8 (Biospec). The aqueous phase was collected after centrifugation at 4° C. for 5 min at 8,000×g. Nucleic acids were purified with QIAquick columns (Qiagen) and eluted with nuclease-free water (Ambion).

COPRO-Seq libraries were prepared by first sonicating 100 μL of a 5 ng/μL solution of DNA from each sample [Bioruptor Pico (Diagenode, New Jersey, USA); 10 cycles of 30 seconds on/30 seconds off at 4° C]. Fragmented DNA was concentrated in MinElute 96 UF PCR Purification plates (Qiagen). Fragments were blunted, an “A”-tail was added, and the reaction products were ligated to Illumina paired-end sequencing adapters containing sample-specific, 8 bp in-line barcodes. Size selection was performed (1% agarose gels), 250-350 bp fragments were excised from the gel, and the DNA was purified by MinElute Gel Extraction (Qiagen). Adapter-linked fragments were enriched by a 20-cycle PCR using Illumina PCR Primers PE 1.0 and 2.0 followed by MinElute PCR Purification. Barcoded libraries were quantified (Qubit dsDNA HS kit), pooled and subjected to multiplex sequencing [Illumina NextSeq instrument; unidirectional 75 nt reads; n=162 samples; 5.4×10⁶±4.7×10⁶ reads/sample (mean±SD)]. Data were demultiplexed and mapped to the reference genomes of community members, plus six “distractor” genomes (Lactobacillus ruminis ATCC 27782, Megasphaera elsdenii DSM 20460, Olsenella uli DSM 7084, Pasteurella multocida subsp. multocida str. 3480, Prevotella dentalis DSM 3688, and Staphylococcus saprophyticus subsp. saprophyticus ATCC 15305). The proportion of reads mapping to “distractor” genomes in each sample was used to set a conservative threshold cutoff (mean +2 SD), indicating the presence/absence of an organism in the community on a per-sample basis. Normalized counts for each bacterial strain in each sample were used to produce a relative abundance table.

### (n) Screen of CFCs Described in FIG. 5 and the Monotonous Feeding Experiments Involving the Initial MDCF Prototype and MS/KF Described in FIG. 7: Microbial RNA-Seq

The effects of KH/MS and the initial MDCF prototype containing three selected complementary foods (chickpea, banana and tilapia) were tested in monotonous feeding experiments involving mice that had been colonized at 5 weeks of age with the defined consortium of 14 bacteerial strains (n=3 cages of dually-housed animals/treatment group). Methods used for isolation of RNA from cecal contents, processing of transcripts for microbial RNA-Seq, sequencing [Illumina NextSeq instrument; unidirectional 75 nt reads; 1.2×10⁷±2.7×10⁶ reads/sample (mean±SD); 24 samples], and analysis of the resulting datasets are described in (68). Briefly, sequence data were mapped to the genomes of community members. Raw counts were subsetted, normalized and analyzed by two complementary strategies. To analyze data at the community level (‘top-down’ view of the meta-transcriptome), or to obtain a strain-level view of transcriptional responses (‘bottom-up’ analysis), raw count data for each comparison of samples were filtered at a low abundance threshold of three raw reads and for consistent representation in biological replicates (present in ≥66% of samples in a given treatment group, or present in all samples in one group and in none of the other). The resulting dataset was then imported into R and differential expression analysis was performed using DESeq2 (69).

KEGG-annotated gene lists for each organism (or the community in aggregate) were processed into gene sets in R (v3.4.1; (49)), and subsequently used for complementary pathway enrichment analyses with the R packages clusterProfiler [v3.4.4; (70)) and GAGE (v2.26.1; (71)]. For hypergeometric enrichment tests, lists of differentially expressed genes were supplied to the clusterProfiler ‘enricher’ function along with corresponding gene set information. DESeq2-normalized counts were supplied along with corresponding gene set information to GAGE, with settings to order genes by the non-parametric Wilcoxon Rank Sum statistic (“rank.test=T, saaTest=gs.tTest”) and to allow genes displaying both increased and decreased expression in each tested level of the KEGG hierarchy to be considered (“same.dir=F”). P-values were adjusted to control false discovery rate (Benjamini-Hochberg method).

### (o) Screen of CFCs Described in FIG. 5 and the Monotonous Feeding Experiments Involving the Initial MDCF Prototype and MS/KF Described in FIG. 7: Targeted Mass Spectrometry

Aliquots of cecal contents taken from the same animals used to compare microbial gene expression in mice monotonously fed KH/MS and the initial MDCF prototype, plus comparable fed germ-free controls were subjected to targeted mass spectrometry (n=4 treatment groups; 3 cages of dually-housed mice/group). Quantification of targeted metabolites was performed using the external standard method based on peak areas of analytes. For cecal amino acids, monosaccharides and disaccharides, flash frozen cecal contents were homogenized in 20 vol/wt of HPLC grade water. Homogenates were centrifuged (4,000×g for 10 minutes at 4° C.). A 200j.tL aliquot of each supernatant was combined with ice-cold methanol (400 j.tL). The mixture was vortexed, centrifuged (8,000×g at 4° C.), and a 500j.tL aliquot of the resulting supernatant was evaporated to dryness. Dried samples were derivatized by adding methoxylamine (80 j.tL of a 15 mg/mL stock solution prepared in pyridine) to methoximate reactive carbonyls (incubation for 16 h at 37° C.), followed by replacement of exchangeable protons with trim ethylsilyl groups using N-methyl-N-(trimethylsilyl) trifluoroacetamide (MSTFA) together with a 1% v/v catalytic admixture of trimethylchlorosilane (1 h incubation at 70° C.). Heptane (160 j.tL) was added and a 1-j.tL aliquot of each derivatized sample was injected into an Agilent 7890B/5977B GC/MS system.

Tryptophan and its metabolites were quantified using an ion pair-based reverse phase (IP-RP) chromatographic method. Chromatographic separation was achieved using an Agilent ZORBAX Extend C18 RRHD 2.1×150 mm, 1.8 j.tm column with the ion-pairing agent tributylamine added to the mobile phases. A Model 1290 Infinity II UHPLC Quaternary Pump was coupled to an Agilent 6470 Triple Quadrupole LC/MS system equipped with a Jet Stream electrospray ionization source. dMRM parameters including precursor, product ions and retention times were determined using chemical standards. MassHunter Optimizer Software was used to determine optimal collision energies and fragmentor voltages for each metabolite.

The protocol for GC-MS of short chain fatty acids is described in a previous publication (56).

To measure amino acids, acylcarnitines, organic acids and acylCoAs in liver, gastrocnemius muscle and serum, samples were weighed while frozen and homogenized in 50% acetonitrile containing 0.3% formic acid (50 mg wet weight tissue/mL solution) using a high-speed homogenizer (IKA #EW-04739-21) set at maximum speed for 30 seconds. Amino acid and acylcarnitine measurements were made by flow injection tandem mass spectrometry, and with specific internal standards (34, 35). Data were acquired using a Waters AcquityTM UPLC system equipped with a triple quadrupole detector and a data system controlled by MassLynx 4.1 OS (Waters, Milford, Mass.). Organic acids were quantified using Trace Ultra GC coupled to ISQ MS operating under Xcalibur 2.2 (Thermo Fisher Scientific, Austin, Tex.) (36). AcylCoAs were extracted, purified, and analyzed by flow injection using positive electrospray ionization on a Xevo TQ-S triple quadrupole mass spectrometer (Waters, Milford, Mass.) (37). Heptadecanoyl CoA was employed as an internal standard (38).

(p) Screen of CFCs Described in FIG. 5 and the Monotonous Feeding Experiments Involving the Initial MDCF Prototype and MS/KF Described in FIG. 7: Western Blot Analysis of IGF-1 Pathway Components in Liver

Liver proteins were isolated, quantified, separated by electrophoresis (4-20% gradient SDS-polyacrylamide gels) and subjected to Western blotting (72). The same amount of total protein was analyzed from each liver sample. The following primary antibodies, all generated in rabbits except for anti-Akt(pan), were purchased from Cell Signaling Technology; anti-phospho-AMPKa(Thr172) [catalog number 2531], anti-Akt(pan) [catalog number 2920], anti-phospho-Akt(Ser473) [catalog number 4060], anti-Jak2 [catalog number 3230], anti-phospho-Jak2(Tyr1007/1008) [catalog number 3776], anti-mTOR [catalog number 2983], anti-phospho-mTOR(Ser2448) [catalog number 5536], anti-Stat 5 [catalog number 9363], and anti-phospho-Stat 5(Tyr694) [catalog number 9351]. Primary antibodies were incubated with Western blots overnight at 4° C. in a solution of Tris-buffered saline containing 0.1% Tween-20 (TBST) plus 1% (vol/vol) non-fat milk, followed by addition of secondary antibodies against rabbit or mouse immunoglobulins and a 1 h incubation at room temperature in TBST/1% nonfat milk. Protein bands were detected by chemiluminescence (Western Lightning® Plus-ECL, PerkinElmer) using the LI COR Odyssey® FC imaging system, and quantified by densitometry. The amount of phosphorylated protein was normalized to the total amount of non-phosphorylated protein or to GAPDH.

(q) Screen of CFCs Described in FIG. 5 and the Monotonous Feeding Experiments Involving the Initial MDCF Prototype and MS/KF Described in FIG. 7: Micro-Computed Tomography (mCT) of Bone

Femurs were harvested from mice at the time of euthanasia and soft tissue was removed. Bones were fixed for 24 hours in 70% ethanol and stored at 4° C. prior to scanning. Micro-computed tomography was performed using a pCT 40 desktop cone-beam instrument (ScanCO Medical, BrUttisellen, Switzerland). For cortical bone analysis, 200-300 slices were taken for each sample in the transverse plane with a 6 μm voxel size (high resolution); slices began at the midpoint of the femur and extended toward the distal femur. For trabecular scans, slices were quantified from the proximal end of the growth plate towards the proximal femur until no further trabeculae were observed. Boundaries of, and thresholds for bone were drawn manually using pCT 40 software. Volumetric parameters (bone volume/tissue volume, bone mineral density and cortical thickness) were calculated using custom scriptsD.

### (r) Screen of CFCs Described in FIG. 5 and the Monotonous Feeding Experiments Involving the Initial MDCF Prototype and MS/KF Described in FIG. 7: IGF-1 ELISA

IGF-1 levels were measured in mouse serum samples using the R&D Systems DuoSet ELISA kit, according to the manufacturer's instructions. Samples were diluted 1:100 in Reagent Diluent and assayed in duplicate. Optical density was quantified on a BioTek Synergy 2 plate reader, and the resulting data were analyzed with GraphPad Prism software (version 7.00 for Mac).

### (s) Screening 16 Plant-Derived Complementary Food Ingredients in Gnotobiotic Mice

We generated 48 diets by supplementing the Mirpur-18 base diet with 16 different plant-based ingredients at three different concentrations (Table 6A). We also prepared diets in which three of the 16 complementary food ingredients (chickpea, peanut and soybean) were incorporated as ‘flours’ to compare the effects of raw versus processed forms (the levels of each of these flours were matched to the protein content of the corresponding unprocessed forms). The ingredients for each diet were cooked, homogenized, extruded as pellets, dried, sterilized, and sterility was assessed as described above.

Five-week-old germ-free male C57BL/6J mice were gavaged with a consortium of 20 cultured, sequenced bacterial strains consisting of (i) seven weaning-phase age-discriminatory strains from healthy Mirpur donors, (ii) five strains from a 24-month-old SAM donor, four of which are prominent in the first 8-11 months of postnatal life, (iii) two milk-adapted strains from a 6-month-old healthy Mirpur donor, (iv) three strains prevalent in the Bangladeshi children with post-SAM MAM (Clostridium amygdalinum, Eggerthella lenta, Lactobacillus gasseri), and (v) three weaning-phase ‘growth-discriminatory’ strains recovered from the fecal microbiota of Malawian children (Clostridium symbiosum, Ruminococcus gnavus, Clostridium nexile) (3). In silico metabolic reconstructions of the requirements of these cultured strains for amino acids and B-vitamins, plus their capacity to utilize mono- and disaccharides were generated.

Colonized mice (n=24; singly-housed in cages containing paper houses for environmental enrichment) were subjected to an 8-week diet oscillation. To minimize the effects of hysteresis, each mouse was fed a different diet every week, and no mouse was given the two forms of an ingredient (i.e., raw and flour forms), in consecutive weeks. Replication was achieved by presenting each diet four times to different mice.

The relative abundances of bacterial community members were determined by COPRO-Seq analysis of DNA isolated from fecal samples collected at the end of each week of the diet oscillation protocol [Illumina Nextera DNA Library Prep Kit; Illumina NextSeq instrument; 75 nt unidirectional reads; n=192 samples; 2.2×10⁶±3.3×10⁵ reads/sample (mean±SD)]. Eighteen strains were detected in all animals at all time points surveyed; one of the three post-SAM MAM strains and one of the SAM-derived strains failed to colonize recipient mice (L. gasseri and S. constellatus, respectively; abundance <0.001%).

### (t) Effects of Complementary Food Ingredients In Mice Harboring a Post-SAM MAM Microbiota

Fifteen different microbial communities from 12 different participants in the SAM trial, collected during and/or after treatment, were introduced into separate groups of 5-week-old germ-free mice (n=4/donor microbiota; dually-housed). Half of the animals in each recipient group were given Mirpur-18 without supplementation, while the other half were given a complementary food formulation composed of peanut, chickpea, banana, tilapia, and milk powder (PCBT diet). Ten days later all animals in the two groups were switched to Mirpur-18 supplemented with peanut, chickpea, banana, and tilapia [Mirpur(PCBT)] and maintained on that diet for 10 days (see Table 7 for the composition of these diets). The goal was to identify those fecal microbiota samples that contained the greatest number of transmissible weaning-phase age-discriminatory bacterial taxa and that when transplanted into mice exhibited increases in the relative abundances of these targeted organisms with supplementation of the Mirpur-18 diet. Based on the results from this screen, we selected a sample obtained from a donor (PS.064) at the S7 time point with post-SAM MAM for a follow-up gnotobiotic mouse study. A 350 mg aliquot of this frozen fecal sample was brought into an anaerobic Coy chamber, vortexed in PBS with glass beads, filtered, and the clarified sample was aliquoted into glass vials prior to storage at −80° C. as described above.

In the follow-on study, mice received an oral gavage of 100 pL sterile 1M sodium bicarbonate followed by 100 μL of the clarified human fecal sample. Animals were given unsupplemented Mirpur-18 diet, or Mirpur-18 supplemented with peanut flour [Mirpur(P)], or Mirpur-18 supplemented with peanut flour, chickpea flour, soy flour (substitute for tilapia) and banana [Mirpur(PCSB)] ad libitum. The supplemented diets were matched for total protein content (Table 8). Age- and sex-matched germ-free C57BL/6J mice fed the same diets served as controls (n=5).

(u) Effects of Complementary Food Ingredients in Mice Harboring a Post-SAM MAM Microbiota: Characterizing the Transplanted Fecal Microbiome from a Donor with Post-SAM MAM in Recipient Gnotobiotic Mice as a Function of Diet Treatment

DNA was extracted from the cecal contents of each mouse in each diet treatment group, quantified (Qubit) and normalized to a concentration of 0.5 ng/μL. Genomic libraries were prepared from each cecal DNA sample (n=5/treatment group) using the Illumina Nextera XT kit in a reaction volume of 2.5 μL. Paired-end 150 nucleotide datasets were generated for each library by multiplex sequencing with an Illumina NextSeq instrument. Reads were processed, assembled, annotated, and the representation of mcSEED subsystems/pathway modules was determined as described above.

Methods used for isolation of RNA from cecal contents, processing for microbial RNA-Seq [Illumina NextSeq instrument; unidirectional 75 nt reads; 6.79±3.35×10⁶ reads/sample (mean±SD); 14 samples], and analysis of the resulting datasets are described in Hibberd et al. (68). Data were analyzed at the community level (‘top-down’ view of the meta-transcriptome) and at the strain-level for F. prausnitzii JG_BgPS064 (‘bottom-up’ analysis). Differential expression was defined using DESeq2 (69). A total of 6,390 genes were found to be differentially expressed (DE) in at least one pairwise comparison of the three diets. These genes were subjected to enrichment analysis over the 58 mcSEED subsystems/pathway modules. Of the DE genes with best-scoring BLAST hits (filtered to include only those spanning at least 90% of the query amino acid sequence) within 2313 annotated mcSEED genomes representing the human gut microbiome, 1099 genes (17.7%) were attributed to the analyzed subsystems/pathway modules. mcSEED-annotated gene lists were used to generate gene sets in R and subsequently employed for pathway enrichment analysis with the GAGE R package (v2.26.1) (71). P-values were adjusted to control false discovery rate (Benjamini-Hochberg method).

### (v) Effects of Complementary Food Ingredients in Mice Harboring a Post-SAM MAM Microbiota: Histochemical and Immunohistochemical Analysis

Immediately after euthanasia, the entire length of the small intestine was removed from each animal and evenly divided into proximal (SI-1), middle (SI-2), and distal (SI-3) segments. Each of these small intestinal segments was further subdivided into thirds. The most proximal third sub-segment was placed in Carnoy's fixative. The middle third sub-segment was perfused with and embedded in Optimal Cutting Temperature (OCT) compound (Tissue-Tek) and then snap frozen in a methanol-dry ice bath. The distal third sub-segment was snap frozen in liquid nitrogen. Frozen samples were stored at −80° C.

The proximal third of each segment was transferred from Carnoy's fixative into 70% ethanol and embedded in paraffin. Five micron-thick sections were prepared and stained with hematoxylin and eosin. OCT embedded blocks of the middle third sub-segments obtained from SI-1, SI-2, and SI-3 were sectioned at 5 pm thickness onto charged, uncoated glass slides (Superfrost Plus) in a cryostat at -20° C. Following cryosectioning, slides were stained for 15 minutes at room temperature with Safranin 0 and Alcian Blue pH 2.5 (Abcam) to identify nuclei and mucosa-associated bacteria, acidic mucopolysaccharides, and glycoproteins. Slides were then dehydrated with graded alcohols (Richard-Allan Scientific), rinsed with xylenes, and stored at room temperature in an airtight container with desiccant for 12 to 16 hours. Fifty crypts in a hematoxylin and eosin-stained SI-2 segment were analyzed per mouse per treatment group (n=5 animals/group). Villus-to-crypt ratios were calculated by measuring villus and crypt lengths in the 10 best-oriented villus-crypt units per hematoxylin- and eosin-stained SI-2 section per mouse per treatment group. Goblet cell and Paneth cell numbers were scored (n=10 crypts per hematoxylin-and eosin-stained SI-2 section/animal). Submucosal lymphoid aggregates were counted and measured in sections prepared from SI-1 and SI-2. Sections were also stained with CD3 (Abcam, catalog number ab5690), CD20 (Thermo Fisher, PA5-16701), and IgA (Abcam, ab97235). A biotin-conjugated, goat anti-rabbit antibody (Jackson ImmunoResearch, 111-065-003, diluted 1:800 in PBS/0.1° A Tween 20) was applied, followed by incubation with horseradish peroxidase-conjugated streptavidin (Jackson ImmunoResearch, 016-030-084, 1:1200) and detection with betazoid 3, 3′ Diaminobenzidine (Biocare Medical). Nuclei were visualized with a hematoxylin counterstain (Leica)D

### (w) Effects of Complementary Food Ingredients in Mice Harboring a Post-SAM MAM Microbiota: Laser Capture Microdissection (LCM)

Infrared laser capture microdissection of the small intestinal epithelium in a 20× field of view of a well-oriented section, prepared from the OCT-embedded segment of SI-2, was performed using the Arcturus Pix Cell Ile system with Arcturus CapSure Macro Caps (Applied Biosystems). RNA extraction was performed immediately after LCM using the Arcturus PicoPure RNA extraction kit (Applied Biosystems) and treatment with Baseline-ZERO DNase (Epicentre). RNA quality was checked with an Agilent Bioanalyzer 2100 using RNA 6000 Pico Chips (Agilent).

All Alcian Blue-stained, mucosal-associated material present in two 20× fields of view of sections prepared from OCT-embedded SI-1, S1-2, and SI-3 sub-segments were subjected to LCM and DNA was isolated [Arcturus PicoPure DNA extraction kit (Applied Biosystems) with a 16-h incubation in proteinase K (1 pμ/μL, ThermoFischer; 65° C)]. To quantify community structure along the length of the small intestine as a function of diet, V4-16S rDNA amplicons were generated from these mucosal DNA samples as described above and sequenced. The resulting OTU table was filtered to include only OTUs with ≥0.1% relative abundance in at least two samples, and then rarefied to 2,000 reads/sample.

RNA isolated from LCM epithelium was used to characterize the effects of diet on jejunal gene expression. cDNA was synthesized from 10 ng of total RNA using the ‘SMARTer Ultra Low Input RNA for Illumina Sequencing-HV’ kit (Clontech). Successful cDNA synthesis was verified using a Bioanalyzer 2100 and High Sensitivity DNA Chips (Agilent). The products were sheared to 200-500 bp with a Covaris AFA system. A library was constructed by following the Clontech adapted Nextera (Illumina) DNA sample preparation protocol for use with ‘SMARTer ultralow DNA kit for Illumina sequencing’. A total of 21 jejunal mucosal samples were sequenced [Illumina NextSeq instrument; NextSeq Series High-Output Kit; 75 nucleotide paired-end reads; 19.8×10⁶ ±5.1×10⁶ reads/sample (mean±SD); for PS.064.S7-colonized mice, n=4 samples from animals consuming the unsupplemented Mirpur-18 diet, 4 samples from those fed Mirpur(P), and 5 samples from mice treated with Mirpur(PCSB); n=3, 2 and 3 from the corresponding groups of germ-free animals].

Reads were aligned to the Ensembl release 89 mouse primary assembly with STAR version 2.5.3a. Gene count data were derived from the number of uniquely aligned reads by featureCounts from Subread version 1.4.6-p5 (48). Sequencing performance was evaluated using RSeQC version 2.6.2. Gene counts were imported into the R/Bioconductor package edgeR and normalized (weighted trimmed mean of M-values). Transcripts from genes with less than one count per million were removed from further analyses. The TMM size factors and the matrix of counts were then imported into R/Bioconductor package limma and weighted likelihoods based on the observed mean-variance relationship of every gene/transcript and sample were calculated for all samples with the voomWithQualityWeights function. The consistency of replicates was assessed with a Spearman correlation matrix and multi-dimensional scaling plots. Gene/transcript performance was assessed with plots of residual standard deviation of every gene to their average log-count with a robustly fitted trend line of the residuals. Generalized linear models were then fitted to allow tests of gene/transcript level differential expression. Differentially expressed genes and transcripts were filtered for FDR adjusted p-values ≤0.05. GAGE and Pathview were also used for analysis of known signaling and metabolism pathways (R/Bioconductor packages GAGE and Pathview).

(x) Isolation, Sequencing, and Genome Annotation of F. prausnitzii Strain JG BgPS064

A cecal sample that had been obtained from a gnotobiotic mouse colonized with the PS.064.S7 donor community and stored at −80° C. was brought into an anaerobic Coy chamber, diluted to a concentration of 0.35 g/5 mL in Wilkins Chalgren anaerobic broth (Oxoid, Ltd.), and the slurry was vortexed 3 times at 30 second intervals. Serial dilutions to 10⁻⁸ were made in Wilkins Chalgren broth, and aliquots (100 tL each) were plated on Wilkins Chalgren agar plates or YHBHI+A plates [YHBHI (73) plus 1 mL/L acetic acid], with or without antibiotics to which F. prausnitzii is frequently resistant [sulfamethoxazole (25 mg/L) and trimethoprim (1.25 mg/L)]. Plates were incubated at 37° C. for 5 days; 32 single colonies per media type (128 colonies total) were picked and plated in duplicate. Selection for Extremely Oxygen Sensitive (EOS) bacteria was performed (74). Five single colonies were picked from plates that had remained in the anaerobic chamber but whose corresponding oxygen-exposed plate did not exhibit any growth. Each colony was added to 15 tL of lysis buffer (TE containing 0.1% Triton-X100), incubated at 95° C. for 15 min, and the solution centrifuged for 10 minutes (3,100×g at room temperature). A 1 tL aliquot of the supernatant was added to a 20 tL reaction mixture containing 10 tL High-Fidelity PCR Master Mix with HF Buffer (Phusion), 1 tL of a 10 tM solution of primer Fprau02, 1 tL of a 10 tM solution of primer Fprau07 (75) and 7 tL of nuclease-free H2O. DNA was amplified (initial denaturation for 2.5 minutes at 98° C., followed by 30 cycles of 98° C. for 10 seconds, 67° C. for 30 seconds and 72° C. for 30 seconds, followed by extension for 5 minutes at 72° C.). An isolate with a positive amplicon was confirmed to be F. prausnitzii by performing PCR with primers 8F and 1391R and sequencing of the resulting full-length 16S rDNA amplicon.

Genomic libraries were prepared from four replicate cultures of the colony-purified F. prausnitzii isolate using the DNA extraction method and the scaled-down Illumina Nextera XT kit described above. The resulting libraries were sequenced using an Illumina MiniSeq instrument (paired-end 150 nt reads). Nextera adapter sequences were trimmed (cutadapt). The isolate genome was assembled using SPAdes (60), initially annotated using Prokka (47) and then subjected to in silico metabolic reconstructions. Strain JG_BgPS064 recovered from the post SAM-MAM donor microbiota contains 2,824 predicted genes; it shares 2268 genes with the SSTS_Bg7063 isolate used for the diet oscillation screens of complementary foods in gnotobiotic mice (see, Table s6F of Gehrig et al. Science, 2019, 365(6449):eaau4732, which is incorporated by reference in its entirety). Of the 266 genes involved in the metabolic reconstructions described for SSTS_Bg7063, there are 248 orthologs in JG_BgPS064; the 19 missing genes are predicted to be non-essential for their respective metabolic pathways because for each there is an iso-functional paralog or alternative pathway. The JG_BgPS064 strain is predicted to produce all amino acids except His and Trp (although its genome contains committed His and Trp salvage ABC transporters). In contrast to the SSTS_Bg7063 strain, this isolate possesses intact LeuC-LeuD genes involved in leucine biosynthesis and thus is likely a Leu prototroph. Metabolic reconstructions suggest JG_BgPS064 can utilize galactose and beta-galactosides, glucose and beta-glucosides, maltose and maltodextrin, fructose and fructooligosaccharides, sialic acids, N-acetylgalactosamine, hexuronic acids (glucoronate, galacturonate), lacto-N-biose (only galactose moiety), and rhamnogalacturonides (only glucoronate moiety). Additionally, this isolate possesses fermentative pathways for production of butyrate, formate, and acetate.

### (v) Gnotobiotic Piglet Study

Experiments involving gnotobiotic piglets were performed under the supervision of a veterinarian using protocols approved by the Washington University Animal Studies Committee.

Preparation of diets—MDCF(PCSB) and MDCF(CS) were produced using ingredients described in Table 11. Diets were packed in vacuum-sealed plastic bags (2 kg double-bagged aliquots), sterilized by gamma-irradiation (20-50 kGy) and stored at—20° C.

Re-deriving piglets as germ-free—The protocol used for generating germ-free piglets was based on our previous publication (28) with several modifications. A pregnant domestic sow (mixture of Landrace and Yorkshire genetic backgrounds), artificially inseminated with semen from a Duroc breed domestic boar, was delivered one day prior to the date of farrow (i.e., on day 113 of gestation). The sow was sedated with ketamine (20 mg/kg, administrated intramuscularly) and anesthetized with isofluorane (2-3%, delivered by mask). The paralum bar abdominal area was disinfected with povidone-iodine. A local incisional block was achieved using 60-80 mL of 2% lidocaine (subcutaneous injection). Each horn of the bicornate uterus was opened and each piglet was removed from its amniochorionic sac while it was still located in the opened uterine horn. The umbilical cord was tied off and each piglet was passed immediately, prior to its first breath, into and through a sterile tank filled with 2% chlorhexidine (10 second procedure) to prevent contamination with residual viable microbes that might be present on the sow's skin. The tank was connected to a sterile, flexible film ‘nursery’ isolator so that the piglets could be directly passed into this temporary housing unit. After the Caesarean section, the sow was euthanized by pentobarbital overdose (150 mg/kg intravenously).

Piglets were revived in the isolator and kept on a heated pad until the remaining piglets in the litter were delivered. Within 24 h, all piglets were transferred from nursery isolators to larger gnotobiotic isolator tubs (Class Biologically Clean Ltd., Madison, Wis.). Before colonization on postnatal day 4 (see below), the germ-free status of piglets was confirmed by aerobic and anaerobic culture of rectal swabs in LYBHI medium (73) before colonization on postnatal day 4. Piglets were group-housed (4 piglets per isolator, with equivalent size range between groups, complying with USDA animal housing regulations). Isolators were maintained at 95-100° F. for the first 7-10 postnatal days, and gradually decreased to 85-90° F. as the thermoregulatory capacity of the animals improved.

Feeding protocol—Piglets were initially bottle-fed with an irradiated sow's milk replacement (Soweena Litter Life, Merrick catalog number C30287N). The powdered sow's milk replacement was prepared in 120 g vacuum-sealed sterilized packets (gamma-irradiated with >20 Gy) and was reconstituted as a liquid solution in the gnotobiotic isolator (120 g/ L autoclaved water). Piglets were fed at 3-hour intervals for the first 3 postnatal days, at 4-hour intervals from postnatal days 4 to 10, and at 6-hour intervals from postnatal day 10 to the end of the experiment. Introduction of solid foods commenced at postnatal day 4 and weaning was accomplished by day 14. Each gnotobiotic isolator was equipped with five stainless steel bowls. During the first three days after birth, all five bowls were filled with Soweena. From days 4 to 7, at each feeding, one bowl was filled with an MDCF prototype while the remaining four bowls were filled with Soweena. On day 8, one bowl of milk was replaced with a bowl of water. On day 9, another bowl of milk was replaced with water (i.e., each isolator at each feed contained 2 bowls of water, 2 bowls of Soweena and 1 bowl of MDCF). On day 10, each feed consisted of placement of one bowl of Soweena, two bowls of water, and two bowls of MDCF into the isolator. From day 11 to day 13, only one bowl was provided with Soweena, and the amount of milk added was reduced by one half each day during this period. On day 14, the last bowl of milk was replaced with a bowl of water, thereby completing the weaning process. Health status was evaluated every three to four hours throughout the day and night during weaning. After weaning, three bowls of fresh sterilized water and two bowls of fresh MDCF were introduced into each isolator every 6 hours to ensure ad libitum feeding. MDCF consumption was monitored by noting the amount of input food required to fill each bowl during a 24-hour period. Piglets were weighed daily using a sling (catalog number 887600; Premier Inc., Charlotte, N.C.). Environmental enrichment was provided within the isolators including plastic balls for ‘rooting’ activity and rubber hoses and stainless steel toys for chewing and manipulating. The behavior and health status of the piglets were monitored every day throughout the experiment to ensure their well-being.

Colonizing piglets—Bacterial strains were cultured under anaerobic conditions in pre-reduced MegaMedium (55, 57). An equivalent mixture of each age-/growth-discriminatory strain was prepared by adjusting the volumes of each culture based on optical density (600 nm) readings. An equal volume of pre-reduced PBS containing 30% glycerol was added to the mixture and aliquots were frozen and stored at −80° C. until use. Each piglet received an intragastric gavage (Kendall KangarooTM 2.7 mm diameter feeding tube; catalog number 8888260406; Covidien, Minneapolis, Minn.) of 11 mL of a solution containing a mixture of the bacterial consortium and Soweena (1:10 v/v).

Biospecimen collection—Piglets were fasted for 6 hours, removed from their gnotobiotic isolator, sedated with ketamine (20 mg/kg, administered intramuscularly) and anesthetized with isofluorane (2%, delivered by mask). Euthanasia was performed on experimental day 31 following American Veterinary Medfical Association (AVMA) guidelines. Blood was collected from the heart after the piglets were anesthetized but prior to administration of pentobarbital. Serum was recovered from clotted blood samples after centrifugation (4000×g, 10 minutes, 4° C.). Luminal contents were harvested from the distal 5% of the small intestine ('ileum'), cecum, and distal 10 cm of the colon. Samples of the biceps femoris and liver were placed in liquid nitrogen and stored at −80° C. The left femur was also obtained at the time of euthanasia; after removing soft tissue and muscle, the bone was wrapped in sterile PBS-soaked gauze and stored at −20° C.

Micro-computed tomography—Femoral bone was analyzed with a VivaCT 40 instrument [ScanCO Medical, BrUttisellen, Switzerland; 70kVp/114 pA (tube energy), with 300 ms of integration time]. The voxel dimension for the scan was set at 25 μm³. The epiphyseal plate was used as a 0% reference point. Slices obtained between 40 to 50% from the epiphyseal plate were used for cortical bone analysis (76). Images were analyzed using a custom MatLab script based on the 3-D structural measuring method (77).

LC-MS/MS-based serum proteomics—The protein concentration of each serum sample was quantified [bicinchoninic acid (BCA) assay, Pierce]. An aliquot containing 500 μg of protein was diluted to 5 μg/pL with 100 mM ammonium bicarbonate (ABC) buffer to a total volume of 100 μL. Samples were further diluted with 100 μL ABC buffer containing 8% sodium deoxycholate (SDC) plus 10 mM dithiothreitol (DTT), pH 8.0, and incubated at 90° C. for 5 minutes. Cysteines were alkylated/blocked with 15 mM iodoacetamide followed by incubation at room temperature for 20 minutes in the dark. Samples were then loaded onto a 10 kDa MWCO spin filter (Vivaspin500; Sartorius) and centrifuged at 10,000×g for 20 minutes to concentrate proteins atop the filter. Concentrated proteins were washed with 400 pL ABC buffer, the filter was centrifuged, and proteins were resuspended in 200 pL of ABC buffer containing 10 pg of sequencing-grade trypsin (Sigma Aldrich). Proteolytic digestion atop the filter membrane was allowed to proceed for 4 hours at 37° C. followed by a second application of trypsin (10 pg in 200 pL ABC buffer; overnight incubation). Sample filters were transferred to new 2 mL microfuge tubes and centrifuged at 10,000×g for 20 minutes to collect tryptic peptides in the flow-though. Peptide samples were acidified with 0.5% formic acid and the resulting sodium deoxycholate precipitate was removed by ethyl acetate extraction (78). The peptide-containing aqueous phase was concentrated in a SpeedVac and peptide concentrations were measured by BCA assay.

Peptide samples were analyzed by automated 2D LC-MS/MS using a Vanquish UHPLC with autosampler plumbed directly in-line with a Q Exactive Plus mass spectrometer (Thermo Scientific) outfitted with a triphasic back column [RP-SCX-RP; reversed-phase (5 j.tm Kinetex C18) and strong-cation exchange (5 j.tm Luna SCX) chromatographic resins, Phenomenex] coupled to an in-house pulled nanospray emitter packed with 30 cm Kinetex C18 resin. For each sample, peptides (5 j.tg) were auto-loaded, desalted, separated and analyzed across two successive salt cuts of ammonium acetate (50 and 500 mM), each followed by a 105-minute organic gradient (79). Eluting peptides were measured and sequenced by data-dependent acquisition on the Q Exactive instrument.

MS/MS spectra were searched with MyriMatch v.2.2 (80) against the Sus scrofa proteome (derived from genome assembly 11.1, GCA_000003025.6, January 2017) concatenated with common protein contaminants. Reversed-sequence entries were also provided to estimate false-discovery rates (FDR). Peptide-spectrum matches (PSM) were required to be fully tryptic with any number of missed cleavages; a static carbamidomethylation of cysteines (+57.0214 Da) and variable modifications of oxidation (+15.9949 Da) on methionine. PSMs were filtered using IDPicker v.3.0 (81) with an experiment-wide FDR controlled at <1% at the peptide-level. Peptide intensities were assessed by chromatographic area-under-the-curve (label-free quantification option in IDPicker). To remove cases of extreme sequence redundancy, the Sus scrofa proteome was clustered at 90% sequence identity (UCLUST) (82), and peptide intensities were summed to their respective protein groups/seeds to estimate overall protein abundance. Protein abundance distributions were then log-transformed, normalized across samples (LOESS and mean-centered), and missing values imputed to simulate the mass spectrometer's limit of detection.

### Example 7

### Growth Promotion by a Microbiota-Directed Complementary Food in Children with Moderate Acute Malnutrition

During the first two years of postnatal life, the human gut microbiota normally follows a process of assembly (maturation) that parallels healthy host development. To date, there is limited information about how the microbiota regulates host physiology in ways that contribute to the many facets of normal growth. Childhood undernutrition is a global health challenge manifested by impaired linear and ponderal growth (stunting and wasting), immune and metabolic dysfunctions, altered central nervous system (CNS) development as well as other abnormalities (Black et al., 2008; Black et al., 2013). Undernutrition is typically classified based on anthropometric measurements: e.g., the degree of wasting in children with moderate acute malnutrition (MAM) is defined by a weight-for-length Z score that is 2-3 standard deviations below the median of a reference multi-national cohort of children with healthy growth (WHO, 2009), while children with severe acute malnutrition (SAM) have WLZ scores more than 3 standard deviations below the healthy median. Recent work has shown that children with MAM and SAM have defects in development of their gut microbiota leaving them with communities that appear younger than those of their healthy counterparts. Current nutritional interventions designed to treat MAM and SAM have not focused on the microbiota as a therapeutic target. Coincidentally, existing therapies have limited efficacy in treating the long-term sequelae that affect undernourished children (Dewey et al., 2016; Goudet et al., 2019), or in repairing their microbiota (Examples 1-6).

We previously identified a network ('ecogroup') of 15 bacterial strains whose covarying representation describes normal gut microbial community development during the first 2 years of postnatal life in healthy members of birth cohorts from several geographically distinct low and middle-income countries (Raman et al., 2019). Changes in the abundances of ecogroup taxa provided a way of defining the severity of microbiota perturbations in children with untreated MAM and SAM as well as the incomplete repair that occurs when different commonly used therapeutic food formulations were administered to children with SAM and MAM (Examples 1-6, Raman et al., 2019). Comparisons of gnotobiotic mice colonized with fecal microbiota from chronologically age-matched healthy children or those with wasting and stunting have revealed bacterial strains discriminatory for weight gain; a number of these strains are ecogroup taxa (Blanton et al., 2016; Raman et al., 2019). Addition of a consortium of five of these strains, cultured from the gut communities of children representing a population where the burden of disease is great, to microbiota from a wasted/stunted child prevented transmission of an impaired weight gain phenotype to just-weaned germ-free mice (Blanton et al., 2016). Based on these observations, gnotobiotic mice and gnotobiotic piglets were used to screen food staples for their ability to increase the fitness and expressed beneficial functions of target ecogroup/growth-discriminatory strains. This effort led to the development of several microbiota-directed complementary food (MDCF) prototypes (Examples 1-6). Three of these MDCF formulations, and a current standard ready-to-use supplementary food (RUSF), were tested in a small, 1-month-long, randomized controlled trial of 12-18-month-old children with MAM from an urban slum located in the Mirpur district of Dhaka, Bangladesh. The results revealed a lead formulation (‘MDCF-2’) that repaired the microbiota towards a configuration present in chronologically aged-matched healthy Mirpur children (Raman et al., 2019). This microbiota repair was accompanied by changes in the abundances of a number of plasma proteins involved in regulating various facets of growth, including bone biology, metabolic regulation, neurodevelopment and immune function (Examples 1-6). Based on these observations, we have now performed a larger, longer proof-of-concept study to compare the effects of MDCF-2 and RUSF on clinical outcomes. As described below, the superior improvement in rate of weight gain achieved with MDCF-2, and the accompanying microbiota repair and changes in the plasma proteome, reveal how components of the gut community are mechanistically linked to growth, and provide evidence supporting a microbiota-directed therapeutic approach for undernutrition exemplified by MDCF-2.

### Results

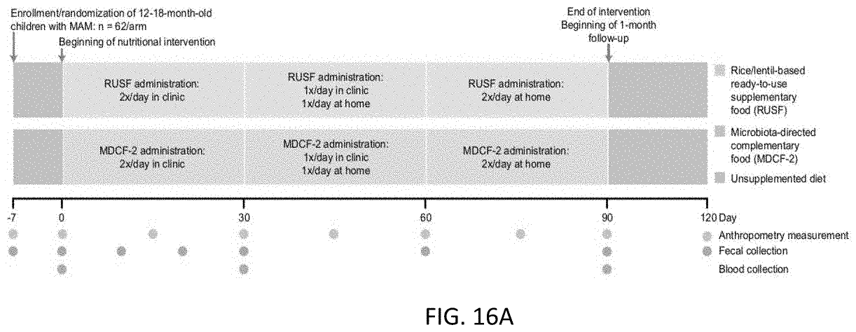

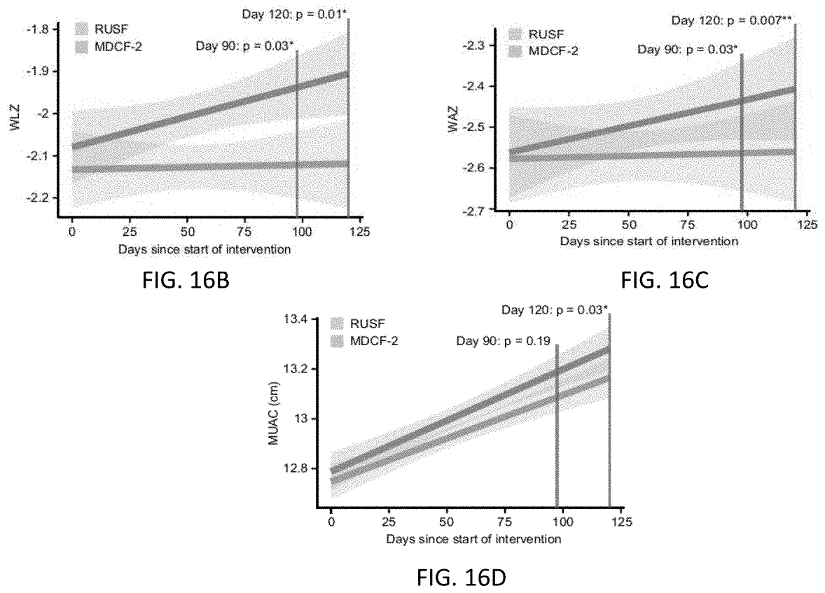

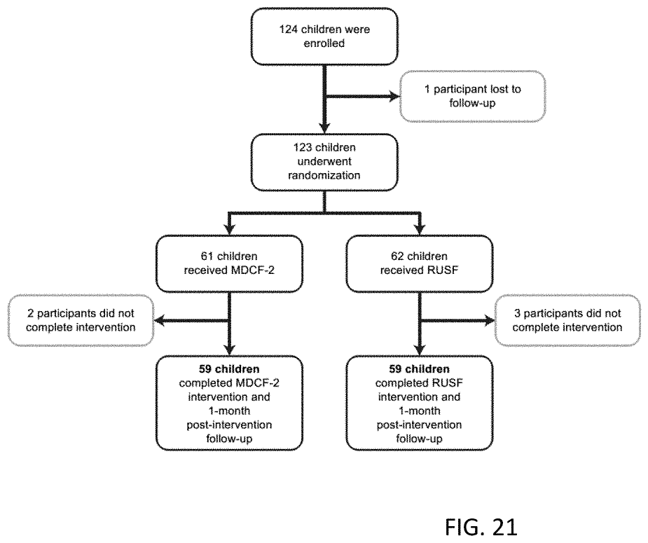

Clinical characteristics and response to nutritional intervention—Bangladeshi children between 12-18 months of age [15.4±2.0 (mean±SD)] with MAM living in Mirpur were enrolled in a randomized study that involved twice-daily dietary supplementation with either MDCF-2 (n=61) or RUSF (n=62) (Table 16 for the nutrient content of these supplementary foods). FIG. 16A summarizes the study design (see Methods for details). In brief, during the first month each child was brought to a local study center twice a day where they were given a 25 g serving of MDCF-2 or RUSF under direct supervision; the amount left unconsumed at each visit was determined by weighing. In the second month, one, and in the third month, both of the supervised feedings occurred at home, again with documentation of the amount consumed. Other than being asked to avoid feeding their children during the 2-hour period before each visit, mothers were advised to continue their usual breast feeding and complementary feeding practices throughout the study. Daily food consumption histories for each child were collected. Anthropometry was measured, and plasma and fecal samples were obtained from children at regular intervals throughout the study (FIG. 16A). Children were followed for one month after completing the intervention to measure the durability of their response to the nutritional intervention. Fifty-nine children in each arm completed the 3-month intervention and 1-month follow-up (FIG. 16A, FIG. 21).

At enrollment, socio-demographic characteristics did not significantly differ between children in the two arms (Table 14). The average time between enrollment and the first day of treatment (i.e. baseline) was 5.88±0.14 (mean±SEM) weeks. At baseline, anthropometric features did not differ significantly between the two groups (Table 15). There was no difference in the percentage of total supplement consumed between children receiving MDCF-2 [92.5±0.73% (SEM) of the amount provided] or RUSF [92.7%±1.15% (SEM), p=0.87]. Notably, the caloric density of MDCF-2 is lower than RUSF (204 versus 247 kcal/50 g daily dose). There were no significant differences between the two groups regarding the effects of treatment on breastfeeding (Chi-squared test, p=0.57; Table 14).

TABLE 14
Clinical characteristics at enrollment

| | MDCF-2 | | RUSF | | MDCF-2 vs |
|---|---|---|---|---|---|
| | (n = 61) | | (n = 62) | | RUSF p-value |
| Demographic Features | | | | | |
| Age (months) | 15.4 ± 1.9 | | 15.5 ± 2.0 | | 0.64 |
| Female - no. (%) | 35 | (57%) | 36 | (58%) | 1 |
| Anthropometry at | | | | | |
| enrollment | | | | | |
| WLZ | −2.31 ± 0.29 | | −2.40 ± 0.27 | | 0.05 |
| WAZ | −2.69 ± 0.67 | | −2.76 ± 0.62 | | 0.55 |
| LAZ | −2.08 ± 1.16 | | −2.08 ± 1.12 | | 0.96 |
| MUAC (cm) | 12.8 ± 0.53 | | 12.7 ± 0.44 | | 0.30 |
| Breastfeeding status† | | | | | |
| Not breastfed since birth - | 1 | (2%) | 0 | (0%) | 0.57 |
| no. (%) | | | | | |
| Partial breastfeeding - | 46 | (75%) | 46 | (74%) | |
| no. (%) | | | | | |
| Exclusive breastfeeding - | 14 | (23%) | 16 | (26%) | |
| no. (%) | | | | | |
| Immunization status† | | | | | |
| Complete - no. (%) | 53 | (87%) | 52 | (84%) | 0.12 |
| Partial - no. (%) | 8 | (13%) | 6 | (10%) | |
| None - no. (%) | 0 | (0%) | 4 | (6%) | |

WLZ: weight-for-length z-score. WAZ: weight-for-age z-score. LAZ: length-for-age z-score. MUAC: mid-upper arm circumference. CI: confidence interval.
Values represent: mean ± SD; number (%); median [interquartile range]. Statistically significant differences in characteristics between groups were performed using a two-tailed unpaired t-test unless otherwise noted.
†Statistical significance determined using a Chi-squared test.
*p < 0.05

TABLE 15
Clinical response to MDCF-2 or RUSF supplementation
Anthropometry at the start of intervention i.e. baseline

| | | | | | p-value |
|---|---|---|---|---|---|
| | MDCF-2 (n = 59)† | RUSF (n = 59)† | β Treatment (95% CI)‡ | | treatment |
| WLZ | −2.22 ± 0.39 | −2.29 ± 0.36 | 0.086 | (−0.056, 0.228) | 0.228 |
| WAZ | −2.66 ± 0.67 | −2.71 ± 0.64 | 0.036 | (−0.213, 0.285) | 0.771 |
| LAZ | −2.14 ± 1.14 | −2.13 ± 1.13 | −0.044 | (−0.467, 0.380) | 0.837 |
| MUAC | 12.8 ± 0.51 | 12.7 ± 0.44 | 0.077 | (−0.100, 0.254) | 0.385 |
| (cm) | | | | | |

Rate of growth during intervention (Δ anthropometry/week)

| | β growth rate for | β growth rate for | | β Interaction | | p-value |
|---|---|---|---|---|---|---|
| | MDCF-2 (95% CI)§ | RUSF (95% CI)§ | | (95% CI)¤ | | interaction |
| WLZ | 0.021 (0.014, 0.029)*** | 0.010 | (0.003, 0.017)** | 0.011 | (0.001, 0.021) | 0.027* |
| WAZ | 0.017 (0.012, 0.022)*** | 0.010 | (0.004, 0.015)*** | 0.008 | (0.001, 0.015) | 0.030* |
| LAZ | 0.004 (0.002, 0.007)*** | 0.005 | (0.003, 0.008)*** | −0.001 | (−0.005, 0.003) | 0.610 |
| MUAC | 0.031 (0.029, 0.034)*** | 0.029 | (0.025, 0.032)*** | 0.003 | (−0.001, 0.007) | 0.192 |
| (cm) | | | | | | |

Rate of growth considering the 1-month follow-up (Δ anthropometry/week)

| | β growth rate for | β growth rate for | | β Interaction | | p-value |
|---|---|---|---|---|---|---|
| | MDCF-2 (95% CI)§ | RUSF (95% CI)§ | | (95% CI)¤ | | interaction |
| WLZ | 0.010 (0.005, 0.016)*** | 0.000 | (−0.005, 0.006) | 0.001 | (0.002, 0.018) | 0.011* |
| WAZ | 0.009 (0.005, 0.013)*** | 0.001 | (−0.003, 0.005) | 0.008 | (0.002, 0.013) | 0.007** |
| LAZ | 0.004 (0.002, 0.006)*** | 0.003 | (0.001, 0.006)** | 0.000 | (−0.003, 0.003) | 0.787 |
| MUAC | 0.028 (0.026, 0.031)*** | 0.024 | (0.022, 0.027)*** | 0.004 | (0.000, 0.007) | 0.028* |
| (cm) | | | | | | |

WLZ: weight-for-length z-score. WAZ: weight-for-age z-score. LAZ: length-for-age z-score. MUAC: mid-upper arm circumference. CI: confidence interval.
†Values represent the mean ± SD.
‡Linear model predicting anthropometry at the start of the intervention as a function of treatment group, controlling for baseline age, gender, and any illness 7 days prior to starting the intervention. β indicates the mean difference in anthropometry between participants who were assigned to the MDCF-2 and RUSF arms at the start of the intervention.
§Mixed effects linear model predicting anthropometry as a function of weeks since starting nutritional supplementation, controlling for the main effects of baseline age, gender, any illness 7 days prior to starting the intervention, and a random intercept for each participant. β indicates the growth rate in unit/week.
¤Mixed effects linear model predicting anthropometry as a function of the interaction between treatment group and weeks since starting nutritional supplementation, controlling for the main effects of baseline age, gender, any illness 7 days prior to starting the intervention, weeks in the intervention, treatment group, and a random intercept for each participant. β indicates the interaction between treatment and growth rate in unit/week (positive means a faster growth rate in children receiving MDCF-2).
*p < 0.05;
**p < 0.01;
***p < 0.001

TABLE 16
Nutritional composition of MDCF-2 and RUSF

| | |
|---|---|
| MDCF-2 | RUSF |

Nutrient composition (g/100 g)

| | | |
|---|---|---|
| Micronutrient mix | 3.14 | 3.14 |
| Protein | 11.6 | 10.2 |
| Fat | 20.8 | 29.5 |
| Total Carbohydrate | 46.2 | 48.8 |
| Fiber | 4.5 | 4.7 |

Ingredient composition (g/100 g)

| | | |
|---|---|---|
| Chickpea flour | 10 | 0 |
| Peanut flour | 10 | 0 |
| Soybean flour | 8 | 0 |
| Green banana | 19 | 0 |
| Rice | 0 | 18.9 |
| Lentil | 0 | 21.5 |
| Powdered skimmed milk | 0 | 10.5 |
| Sugar | 29.8 | 17 |
| Soybean oil | 20 | 29 |

Energy content

| | | |
|---|---|---|
| Protein-energy ratio | 11.4 | 8.2 |
| Fat-energy ratio | 46 | 53.6 |
| Total calories (kcal) | 406.8 | 494.6 |

The primary outcome of the 3-month dietary intervention was the rate of change (β) in ponderal growth, as defined by every 15-day measurements of WLZ and weight-for-age z-score (WAZ). Both β-WLZ and β-WAZ improved significantly in children in both arms during the intervention period [β_{WLZ}=0.021±0.004 (SEM), β_{WAZ}=0.017±0.003 (SEM) for MDCF-2; β_{WLZ}=0.010±0.004, β_{WAZ}=0.010±0.003 for RUSF] (Table 15). Despite its lower caloric density, children who received MDCF-2 exhibited a statistically significant faster rate of weight gain (WLZ and WAZ) over the course of the 3-month intervention compared to those consuming RUSF (p=0.03); this difference in rate of improvement in WLZ and WAZ was sustained during the 1-month period following the intervention (FIG. 16B and FIG. 16C; Table 15).

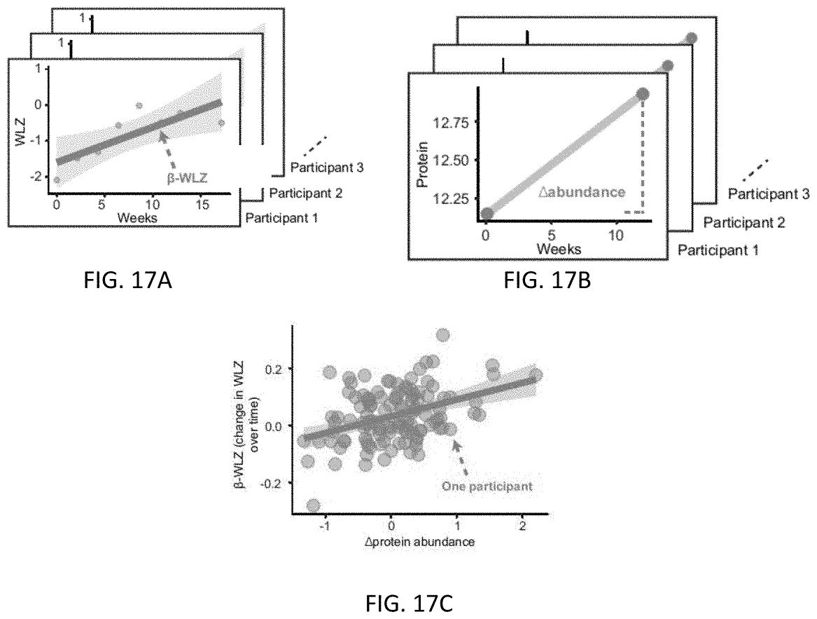

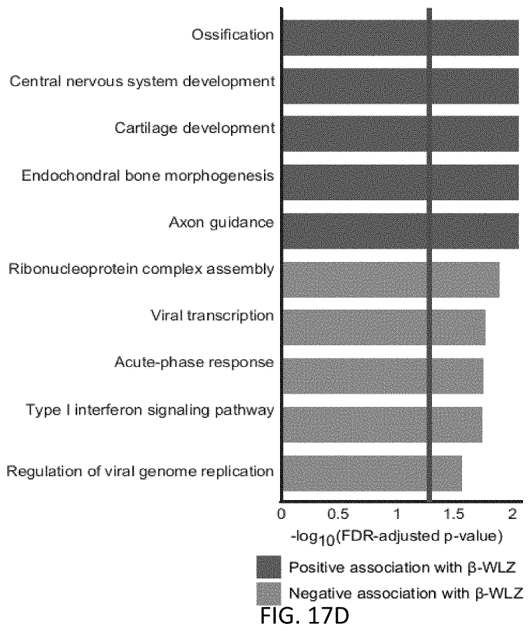

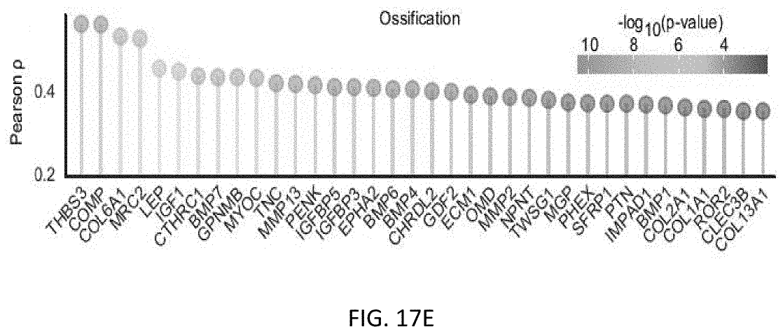

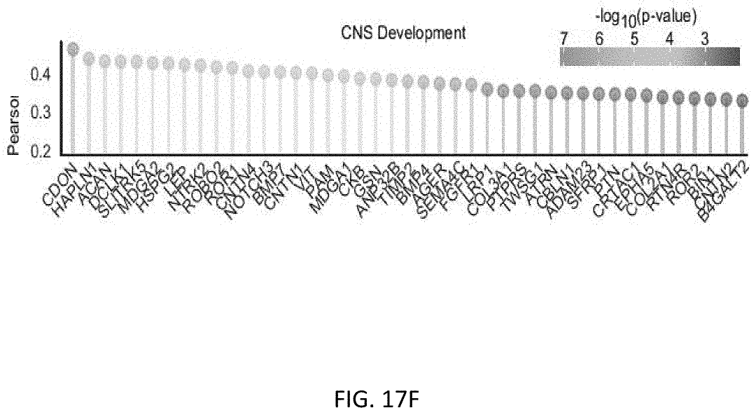

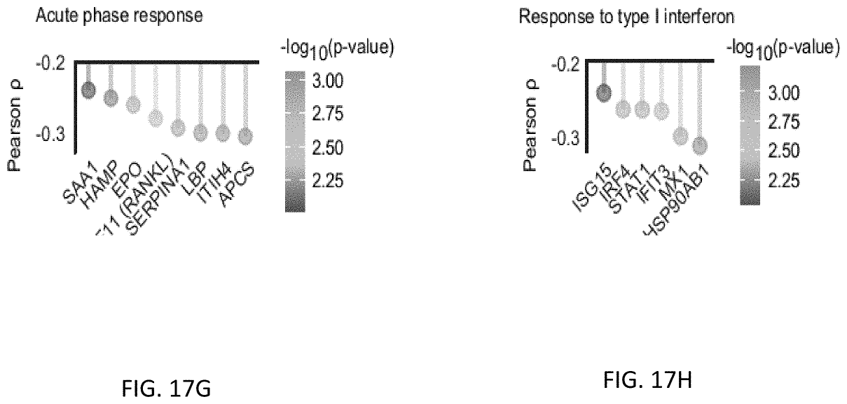

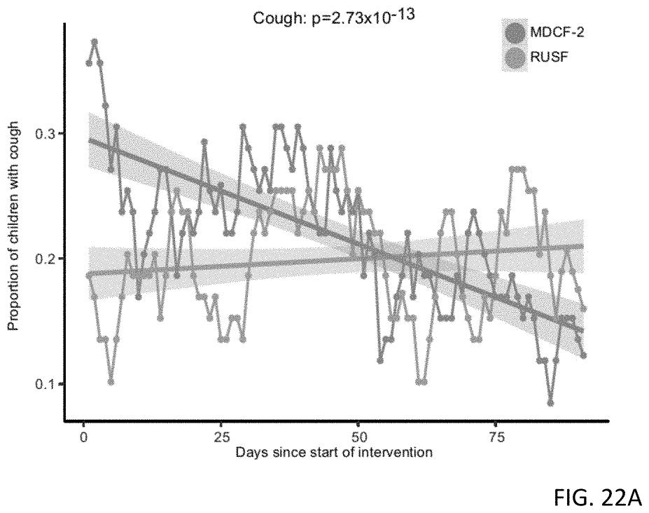

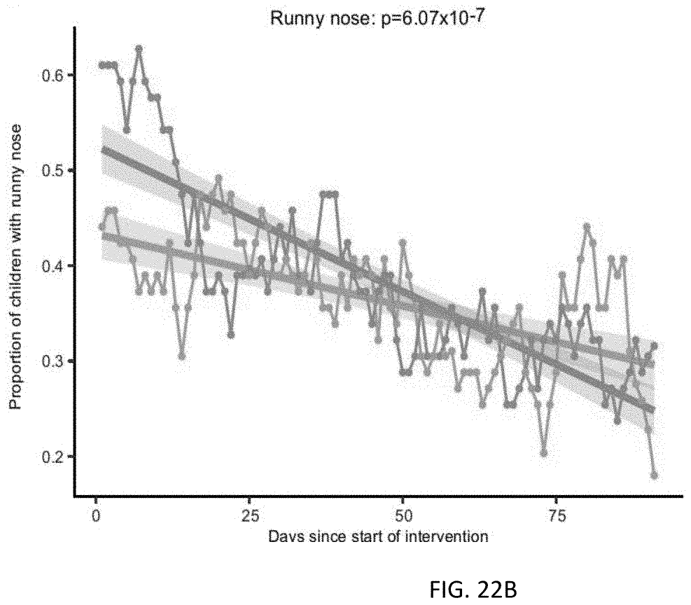

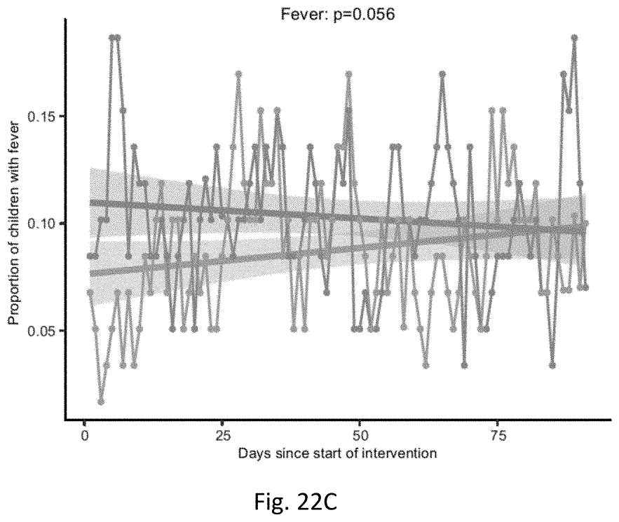

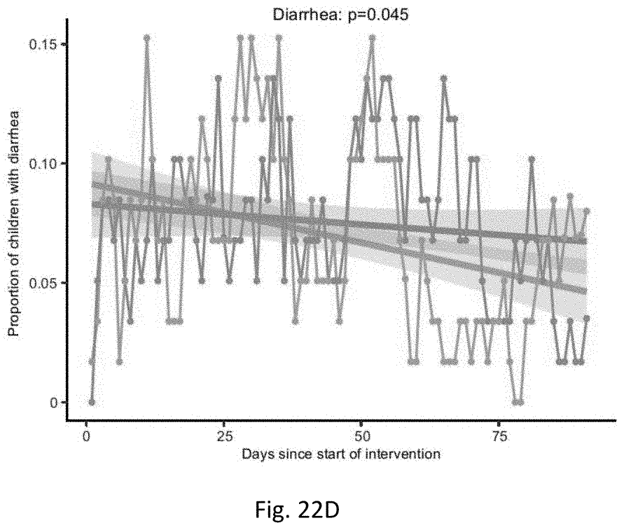

Mid-upper arm circumference (MUAC) is another measure of growth that complements WLZ (Chiabi et al., 2016, Grellety et al., 2018). MUAC improved significantly in children in both arms during the intervention period (Table 15). Considering the 4-month period between the time of initiation of the intervention and the end of the 1-month follow-up, children who received MDCF-2 had significantly faster improvement in MUAC (b-MUAC) compared to those treated with RUSF (p=0.03, FIG. 16D, Table 15). Although both MDCF-2 and RUSF supplementation produced significant improvements in linear growth as defined by length-for-age z-scores (LAZ), there were no significant differences in the rates of change of LAZ between the two arms of the study (Table 15). The effects of treatment on four symptoms cough, runny-nose, fever and diarrhea were assessed daily. The prevalence of cough and runny-nose (rhinorrhea) reported over the course of each week was significantly reduced by MDCF-2 compared to RUSF supplementation [β_{cough}=−0.096±0.014 (SEM), p<0.001; βᵣₕᵢₙₒᵣᵣₕₑₐ=−0.060±0.012 (SEM), p<0.001; FIG. 22A, FIG. 22B, Table 17]. MCDF-2 administration was also associated with a greater reduction in fever although the difference between treatments did not achieve statistical significance (p=0.056; FIG. 17C, Table 17). Taken together, these results demonstrate that a 3-month, twice-daily supplementation of MDCF-2 produces a more rapid and durable ponderal growth response in these children with MAM compared to RUSF.

TABLE 17
Effects of supplementation on co-morbidity and illness
Prevalence of co-morbidities throughout the 3-month intervention (prevalence/week)

| | β prevalence for | | β prevalence for | | β Interaction | | p-value |
|---|---|---|---|---|---|---|---|
| | MDCF-2 (95% CI)† | | RUSF (95% CI)† | | (95% CI)‡ | | interaction |
| Cough | −0.085 | (−0.105, −0.065)*** | 0.012 | (−0.007, 0.031) | −0.096 | (−0.124, −0.069) | 2.73 × 10⁻¹³*** |
| Runny-nose | −0.114 | (−0.131, 0.097)*** | −0.114 | (−0.131, 0.097)*** | −0.060 | (−0.083, −0.036) | 6.07 × 10⁻⁷*** |
| (Rhinorrhea) | | | | | | | |
| Fever | −0.012 | (−0.036, 0.012) | 0.022 | (−0.004, 0.048) | −0.034 | (−0.069, 0.002) | 0.056 |
| Diarrhea | −0.019 | (−0.047, 0.009) | −0.060 | (−0.090, −0.030)*** | 0.041 | (0.000, 0.082) | 0.045* |

CI: confidence interval.
†Generalized mixed effects linear model predicting prevalence of co-morbidity (binomial presence or absence) as a function of weeks since starting nutritional supplementation, controlling for a random intercept for each participant. β indicates the log-odds of reporting a co-morbidity per week; positive indicates increasing likelihood while negative indicates decreasing likelihood during intervention.
‡Generalized mixed effects linear model predicting prevalence of co-morbidity (binomial presence or absence) as a function of the interaction between treatment group and weeks since starting nutritional supplementation, controlling for a random intercept for each participant. β indicates the increased/decreased weekly likelihood of reporting a co-morbidity in the MDCF-2 arm over the RUSF arm; positive indicates a higher likelihood while negative indicates a lower likelihood of reporting a co-morbidity in children receiving MDCF-2 compared to those receiving RUSF.
*p < 0.05;
***p < 0.001

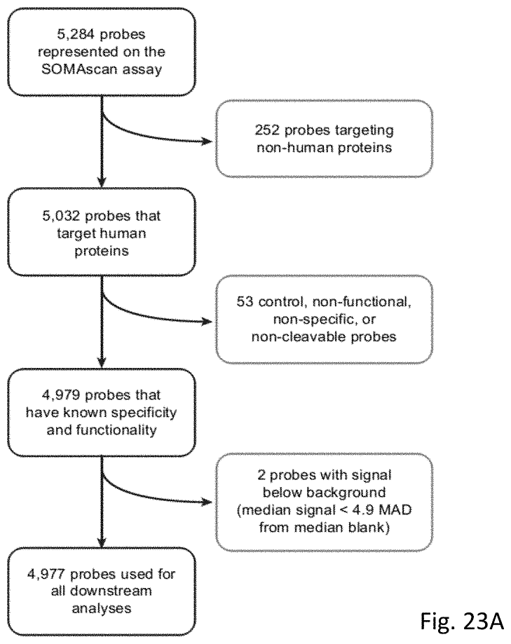

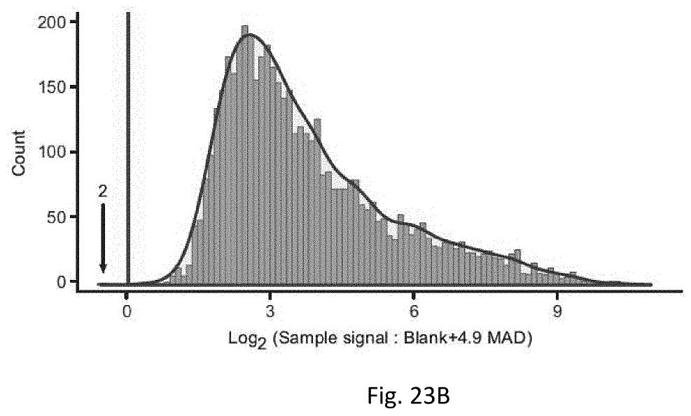

Effects of nutritional intervention on host biological state—To identify the mechanisms by which MDCF-2 improved ponderal growth, we used an aptamer-based proteomic assay (Gold et al., 2014) to quantify the abundances of 4,977 proteins in plasma samples collected from all 118 children in the study at the 0, 1 and 3 month time points (FIG. 23A, FIG. 23B). For each child, a linear model relating the changes in his/her WLZ during the 3-month intervention was constructed; β-WLZ was then correlated with changes in plasma protein abundances prior to and after completing supplementation (Δprotein abundance, FIG. 17A-C). This method allowed us to assess how changes in plasma protein abundances relate to changes in ponderal growth, despite having more timepoints of recorded anthropometry than of quantified plasma proteins for each participant.

A total of 75 plasma proteins were identified as significantly correlated (positively or negatively) with β-WLZ [false discovery rate (FDR)-adjusted q<0.1, Table 18]. Gene set enrichment analysis (GSEA) querying Gene Ontology ‘biological processes’ (GO terms) revealed that proteins positively correlated with β-WLZ were significantly enriched (GSEA q<0.1) for mediators of bone growth and ossification; they include (i) cartilage oligomeric matrix protein (COMP), an extracellular matrix protein critical for endochondral bone growth that increases in serum after growth hormone supplementation (Burger et al., 2020, Bjarnason et al., 2004), (ii) secreted frizzled-related protein 4 (SFRP4), a Wnt inhibitor that prevents excessive osteoclast erosion of bone and is an early biomarker of type-2 diabetes and metabolic syndrome in adults (Chen et al., 2019, Bix et al., 2015, Hoffman et al., 2014), (iii) leptin (LEP), a circulating hormone produced by adipocytes and enterocytes that modulates energy balance, indicates adipose reserves, and predicts survival in children with severe acute malnutrition undergoing treatment (Bartz et al., 2014, Njunge et al., 2019), (iv) insulin-like growth factor 1 (IGF1), a key effector of linear and ponderal growth, and (v) IGF acid-labile subunit, an IGF-1 stabilizing protein that increases the half-life of IGF-1 in circulation (FIG. 17D, FIG. 17E, Table 18). Proteins positively associated with ponderal growth rates were also significantly enriched for effectors of CNS development; these included the axon guidance protein SLIT and NTRK-like protein 5 (SLITRKS), BDNF/NT-3 growth factor receptor (NTRK3), and roundabout homolog 2 (ROBO2), an axon guidance receptor with reported pro-osteoblastic/anti-osteoclastic activity (Kim et al., 2018) (FIG. 17F, Table 18). The group of proteins whose changes in abundances negatively correlated with changes in ponderal growth were significantly enriched for acute phase reactants and actuators of immune activation [e.g., hepcidin (HAMP), which reduces iron absorption and induces iron sequestration during inflammatory states; the osteoclast-promoting factor RANKL; granulysin (GNLY), a proinflammatory cytokine produced by activated cytotoxic T- and NK-cells; interferon-induced protein with tetratricopeptide repeat 3 (IFIT3), which inhibits the replication of multiple viral pathogens (Diamond et al., 2013); and immunoglobulin A (IGHA1)] (FIG. 17G, FIG. 17H, Table 18).

TABLE 18
Plasma proteins were identified as significantly
correlated (positively or negatively) with β-WLZ

| Entrez | | | | | |
|---|---|---|---|---|---|
| Gene | Entrez | | Pearson | | |
| Symbol | Gene ID | Description | Rho | p-value | q-value |
| THBS4 | 7060 | Thrombospondin-4 | 0.57 | 1.20E−11 | 6.00E−08 |
| THBS3 | 7059 | Thrombospondin-3 | 0.56 | 3.71E−11 | 1.85E−07 |
| COMP | 1311 | Cartilage oligomeric matrix protein | 0.56 | 4.01E−11 | 1.99E−07 |
| | | (Thrombospondin-5) | | | |
| DLK1 | 8788 | Protein delta homolog 1 | 0.55 | 8.72E−11 | 4.34E−07 |
| RET | 5979 | Proto-oncogene tyrosine-protein | 0.55 | 1.42E−10 | 7.06E−07 |
| | | kinase receptor Ret | | | |
| SFRP4 | 6424 | Secreted frizzled-related protein 4 | 0.55 | 1.52E−10 | 7.55E−07 |
| FAP | 2191 | Prolyl endopeptidase FAP | 0.54 | 2.85E−10 | 1.42E−06 |
| COL6A1 | 1291 | Collagen alpha-1(VI) chain | 0.51 | 2.63E−09 | 1.31E−05 |
| DPT | 1805 | Dermatopontin | 0.51 | 4.62E−09 | 2.30E−05 |
| MRC2 | 9902 | C-type mannose receptor 2 | 0.51 | 4.89E−09 | 2.43E−05 |
| EMILIN3 | 90187 | EMILIN-3 | 0.50 | 5.88E−09 | 2.92E−05 |
| SCARF2 | 91179 | Scavenger receptor class F member | 0.50 | 6.82E−09 | 3.39E−05 |
| | | 2 | | | |
| CRABP2 | 1382 | Cellular retinoic acid-binding protein 2 | 0.50 | 9.00E−09 | 4.47E−05 |
| DLK1 | 8788 | Protein delta homolog 1 | 0.49 | 1.56E−08 | 7.75E−05 |
| CD248 | 57124 | Endosialin | 0.49 | 1.84E−08 | 9.11E−05 |
| IGDCC4 | 57722 | Immunoglobulin superfamily DCC | 0.49 | 1.99E−08 | 9.88E−05 |
| | | subclass member 4 | | | |
| COL15A1 | 1306 | Collagen alpha-1(XV) chain | 0.49 | 2.52E−08 | 0.00012 |
| CILP2 | 148113 | Cartilage intermediate layer protein 2 | 0.48 | 2.81E−08 | 0.00014 |
| COL6A3 | 1293 | Collagen alpha-3(VI) chain | 0.48 | 2.87E−08 | 0.00014 |
| C1QTNF3 | 114899 | Complement C1q tumor necrosis | 0.48 | 2.95E−08 | 0.00015 |
| | | factor-related protein 3 | | | |
| COLEC12 | 81035 | Collectin-12 | 0.48 | 2.96E−08 | 0.00015 |
| COL28A1 | 340267 | Collagen alpha-1(XXVIII) chain | 0.48 | 3.48E−08 | 0.00017 |
| PXDN | 7837 | Peroxidasin homolog | 0.48 | 4.55E−08 | 0.00023 |
| MATN4 | 8785 | Matrilin-4 | 0.48 | 4.85E−08 | 0.00024 |
| LRRC15 | 131578 | Leucine-rich repeat-containing protein | 0.47 | 5.71E−08 | 0.00028 |
| | | 15 | | | |
| CD93 | 22918 | Complement component C1q | 0.47 | 5.81E−08 | 0.00029 |
| | | receptor | | | |
| CDON | 50937 | Cell adhesion molecule-related/down- | 0.47 | 8.41E−08 | 0.00042 |
| | | regulated by oncogenes | | | |
| CA6 | 765 | Carbonic anhydrase 6 | 0.46 | 1.15E−07 | 0.00057 |
| CBLN4 | 140689 | Cerebellin-4 | 0.46 | 1.23E−07 | 0.00061 |
| HSPB6 | 126393 | Heat shock protein beta-6 | 0.46 | 2.05E−07 | 0.00101 |
| MXRA8 | 54587 | Matrix-remodeling-associated protein | 0.45 | 2.63E−07 | 0.0013 |
| | | 8 | | | |
| KAL1 | 3730 | Anosmin-1 | 0.45 | 2.72E−07 | 0.00135 |
| MLN | 4295 | Promotilin | −0.45 | 3.63E−07 | 0.00179 |
| GNLY | 10578 | Granulysin | −0.44 | 5.94E−07 | 0.00293 |
| PTK7 | 5754 | Inactive tyrosine-protein kinase 7 | 0.44 | 6.09E−07 | 0.00301 |
| MFAP2 | 4237 | Microfibrillar-associated protein 2 | 0.44 | 6.16E−07 | 0.00305 |
| ANTXR2 | 118429 | Anthrax toxin receptor 2 | 0.44 | 8.03E−07 | 0.00397 |
| NELL2 | 4753 | Protein kinase C-binding protein | 0.43 | 1.03E−06 | 0.0051 |
| | | NELL2 | | | |
| PPIC | 5480 | Peptidyl-prolyl cis-trans isomerase C | 0.43 | 1.39E−06 | 0.00688 |
| SMOC2 | 64094 | SPARC-related modular calcium- | 0.42 | 1.64E−06 | 0.0081 |
| | | binding protein 2 | | | |
| HAPLN1 | 1404 | Hyaluronan and proteoglycan link | 0.42 | 1.67E−06 | 0.00823 |
| | | protein 1 | | | |
| LRRC4C | 57689 | Leucine-rich repeat-containing protein | 0.42 | 2.55E−06 | 0.01259 |
| | | 4C | | | |
| CCDC80 | 151887 | Coiled-coil domain-containing protein | 0.42 | 2.59E−06 | 0.0128 |
| | | 80 | | | |
| EHMT2 | 10919 | Histone-lysine N-methyltransferase | 0.42 | 2.91E−06 | 0.01437 |
| | | EHMT2 | | | |
| ACAN | 176 | Aggrecan core protein | 0.41 | 3.26E−06 | 0.0161 |
| DCLK1 | 9201 | Serine/threonine-protein kinase | 0.41 | 3.51E−06 | 0.01731 |
| | | DCLK1 | | | |
| BPIFA2 | 140683 | BPI fold-containing family A member | 0.41 | 3.54E−06 | 0.01745 |
| | | 2 | | | |
| SLITRK5 | 26050 | SLIT and NTRK-like protein 5 | 0.41 | 3.71E−06 | 0.01829 |
| PMEL | 6490 | Melanocyte protein PMEL | 0.41 | 3.72E−06 | 0.01833 |
| MAP2K4 | 6416 | Dual specificity mitogen-activated | 0.41 | 4.25E−06 | 0.02093 |
| | | protein kinase kinase 4 | | | |
| CDH5 | 1003 | Cadherin-5 | 0.41 | 4.43E−06 | 0.02183 |
| TNXB | 7148 | Tenascin-X | 0.41 | 4.87E−06 | 0.02398 |
| SCIN | 85477 | Adseverin | 0.41 | 5.23E−06 | 0.02577 |
| MDGA2 | 161357 | MAM domain-containing | 0.41 | 5.24E−06 | 0.02579 |
| | | glycosylphosphatidylinositol anchor | | | |
| | | protein 2 | | | |
| HSPG2 | 3339 | Basement membrane-specific | 0.40 | 5.96E−06 | 0.02933 |
| | | heparan sulfate proteoglycan core | | | |
| | | protein | | | |
| IGFALS | 3483 | Insulin-like growth factor-binding | 0.40 | 6.53E−06 | 0.03214 |
| | | protein complex acid labile subunit | | | |
| IL1RL1 | 9173 | Interleukin-1 receptor-like 1 | −0.40 | 6.90E−06 | 0.03396 |
| ANTXR1 | 84168 | Anthrax toxin receptor 1 | 0.40 | 7.33E−06 | 0.03604 |
| IL11RA | 3590 | Interleukin-11 receptor subunit alpha | 0.40 | 7.66E−06 | 0.03769 |
| LEP | 3952 | Leptin | 0.40 | 8.37E−06 | 0.04115 |
| LYVE1 | 10894 | Lymphatic vessel endothelial | 0.40 | 8.54E−06 | 0.04201 |
| | | hyaluronic acid receptor 1 | | | |
| OLFM2 | 93145 | Noelin-2 | 0.39 | 9.58E−06 | 0.04709 |
| LUM | 4060 | Lumican | 0.39 | 9.75E−06 | 0.04794 |
| NTRK2 | 4915 | BDNF/NT-3 growth factors receptor | 0.39 | 9.92E−06 | 0.04875 |
| ANGPTL1 | 9068 | Angiopoietin-related protein 1 | 0.39 | 1.01E−05 | 0.04941 |
| ITIH3 | 3699 | Inter-alpha-trypsin inhibitor heavy | −0.39 | 1.04E−05 | 0.05127 |
| | | chain H3 | | | |
| CNTN5 | 53942 | Contactin-5 | 0.39 | 1.24E−05 | 0.06088 |
| FLRT2 | 23768 | Leucine-rich repeat transmembrane | 0.39 | 1.34E−05 | 0.06561 |
| | | protein FLRT2 | | | |
| COL9A1 | 1297 | Collagen alpha-1(IX) chain | 0.39 | 1.38E−05 | 0.06781 |
| ROBO2 | 6092 | Roundabout homolog 2 | 0.39 | 1.54E−05 | 0.07564 |
| IGF1 | 3479 | Insulin-like growth factor I | 0.39 | 1.57E−05 | 0.07687 |
| IGHA1 | 3493 | Immunoglobulin A | −0.39 | 1.58E−05 | 0.07758 |
| ROR1 | 4919 | Inactive tyrosine-protein kinase | 0.38 | 1.81E−05 | 0.08885 |
| | | transmembrane receptor ROR1 | | | |
| POSTN | 10631 | Periostin | 0.38 | 1.97E−05 | 0.09685 |
| DEPP | 11067 | Protein DEPP | 0.38 | 2.04E−05 | 0.09991 |

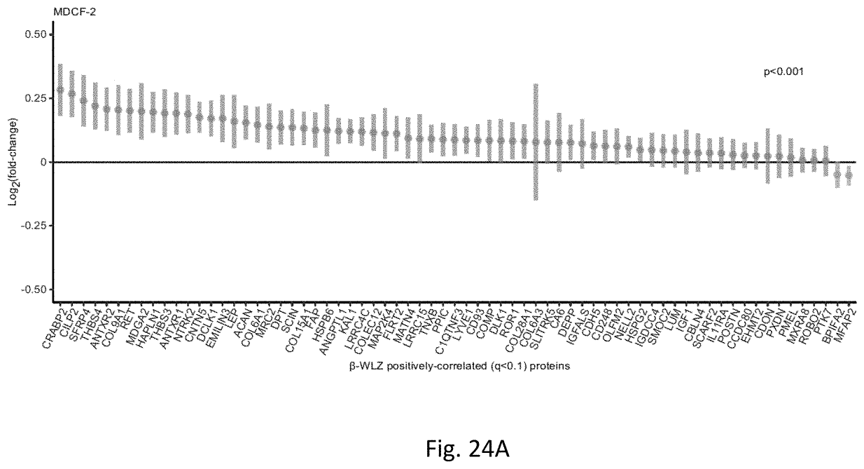

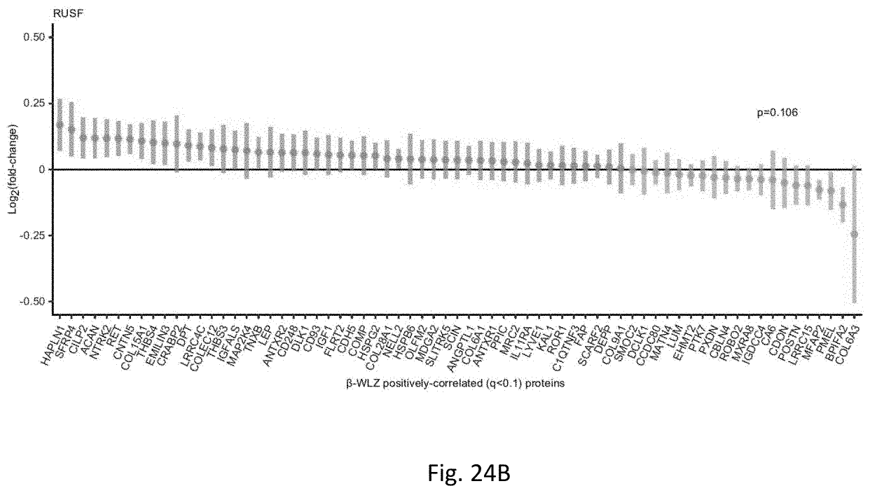

The 70 plasma proteins whose changes in abundances were significantly positively correlated with ponderal growth rates (‘WLZ-associated’ proteins) served as a starting point to compare the effects of MDCF-2 and RUSF on host physiologic state. A total of 714 proteins exhibited significantly higher or lower levels after the 3-month period of supplementation with MDCF-2 (296 more abundant, 418 less abundant). In contrast, 82 proteins showed significant alterations in their abundances after RUSF intervention (46 more abundant, 36 less abundant) (limma q<0.1). Proteins whose abundances increased after 3-month supplementation with MDCF-2 were significantly enriched for the 70 ‘WLZ-associated’ proteins (GSEA p<0.001), while those that were increased after RUSF intervention were not (GSEA p=0.11, FIG. 24A, FIG. 24B). Comparing the two treatments revealed that proteins whose abundances were increased more by MDCF-2 were significantly enriched for WLZ-associated proteins (GSEA p<0.001, FIG. 171). Cartilage intermediate layer protein 2 (CILP2) was the WLZ-associated protein whose abundance was most significantly increased after MDCF-2 supplementation, but not by RUSF. CILP2 is a glycoprotein that forms complexes with collagen VI to promote articular cartilage formation (Bernardo et al., 2011, Rzehak et al., 2016, Willer et al., 2008, Saxena et al., 2014). Other proteins significantly increased after MDCF-2 but not RUSF supplementation include Thrombospondin-4 (THBS4), a multifunctional protein involved in bone, skeletal muscle, vascular, and nervous system development (Stenina-Agonravi et al., 2017), and SFRP4. Together, these results provide evidence that mediators of bone growth, neurodevelopment, and inflammation distinguish the host response to the microbiota-directed nutritional intervention from that of RUSF.

Effects of MDCF-2 and RUSF on the gut microbiota—Fecal samples were obtained every 10 days during the first month of the intervention, every 15 days thereafter (in concert with anthropometric measures) and at the end of the 1-month follow-up period. Bacterial strains were identified by sequencing PCR amplicons generated from variable region 4 of 16S rDNA genes present in the fecal biospecimens. A linear mixed-effects model was used to determine the relationship between the abundances of strains (defined by the representation of amplicon sequence variants, ASVs) and WLZ in each participant (FIG. 18A).

We identified 23 ASVs that were significantly associated with WLZ (‘WLZ-associated’ taxa), 21 of which were positively associated (FIG. 18B, FIG. 18C); the two whose abundances were significantly negatively correlated with WLZ were assigned to Bifidobacterium (ASV_1, likely B. longum) and Escherichia coli (ASV_3) (FIG. 18B). Six of the WLZ-associated taxa (bolded ASVs in FIG. 18C) are members of the ecogroup network of 15 bacterial strains whose representation describes normal gut microbial community development (Raman et al., 2019). In the 1-month pre-POC study described in the Introduction, the ability of MDCF-2 to repair the gut microbiota of 12-18-month-old Mirpur children with MAM towards a configuration present in chronologically aged-matched healthy Mirpur children was exemplified by changes in two of these six WLZ-correlated ecogroup taxa: increases in the relative abundance of Prevotella copri and reductions in Bifidobacterium longum (Raman et al., 2019). Five of the WLZ-associated ASVs (denoted by an asterisk in FIG. 18) share taxonomic similarity with strains identified as discriminatory for weight gain in gnotobiotic mice colonized with fecal microbiota from children with healthy growth phenotypes versus those with wasting and stunting; these taxa include Faecalibacterium prausnitzii, Dorea formicigenerans, Ruminococcus gnavus, and a member of Clostridium. The complementary food ingredients incorporated into MDCF-2 were based on their ability to increase the fitness and expressed beneficial functions of these age- and growth-discriminatory strains (Gehrig et al., 2019).

ASVs whose abundances were significantly increased by MDCF-2 were enriched for WLZ-associated taxa (p<0.001, Fisher's Exact Test) while those whose abundances were increased by RUSF were not (p=0.246). FIG. 18D rank WLZ-associated taxa based on the magnitude and statistical significance of their changes in relative abundances during the 3-month intervention with MDCF-2; the greatest increases occur with P. copri and Faecalibacterium prausnitzii while Bifidobacterium (likely B. longum) exhibits the greatest decrease. Quantitative PCR assays of 23 enteropathogens in fecal samples revealed no statistically significant differences in the effects of MDCF-2 and RUSF on their representation (data not shown).

Based on these results, we concluded the WLZ-associated taxa identified in our 3-month long POC study provided evidence of pre-clinical to clinical translation; i.e., MDCF-2 exhibits its intended target profile in the microbiota of children with MAM, and the microbiota is causally linked to ponderal growth.

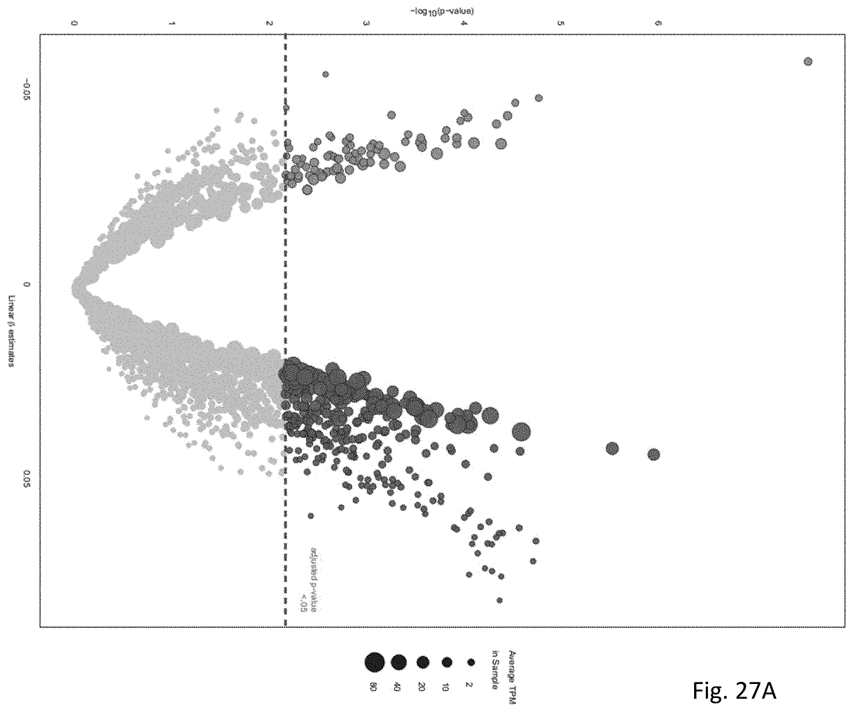

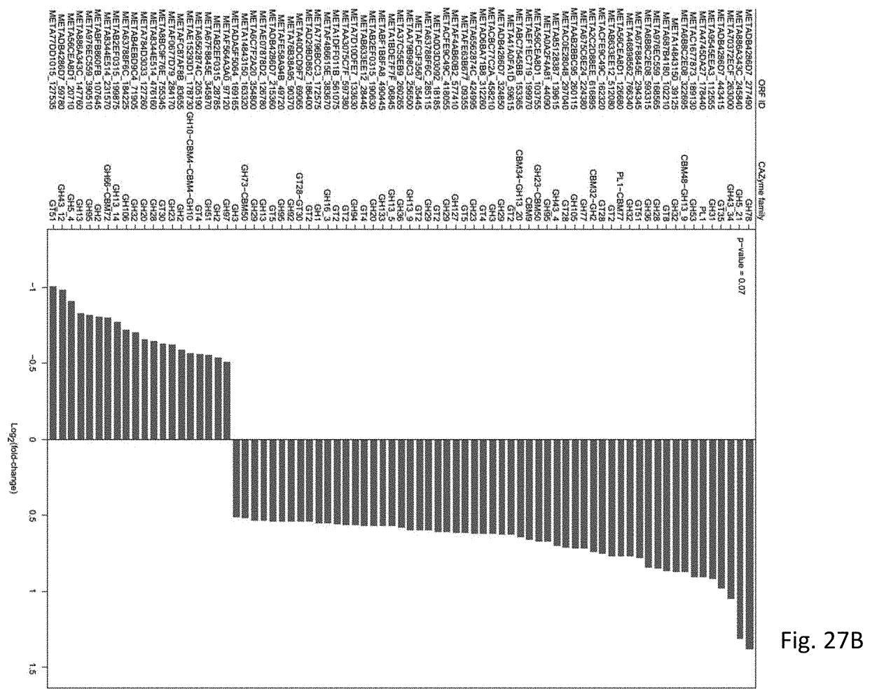

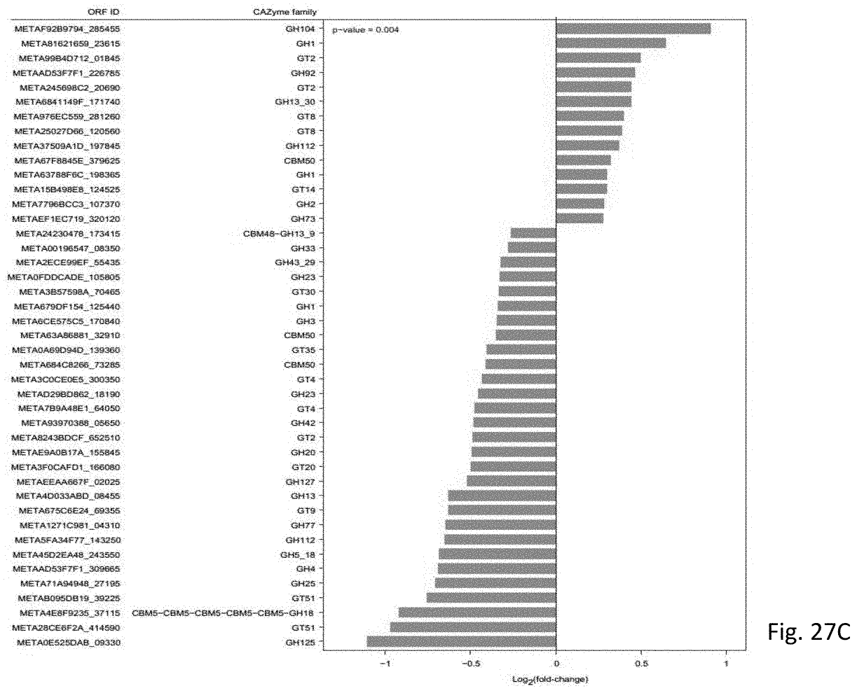

To further investigate the link between the microbiota and ponderal growth, a total of 29,401 gene clusters were annotated as encoding carbohydrate-active enzymes (CAZymes) with 2,653 represented in more than 20% of fecal samples. Of these 2,653 CAZyme genes, the abundances of 294 were significantly positively correlated with WLZ while 84 were significantly negatively correlated (mixed-effects linear model q<0.05). Comparison of the CAZyme responses in the MDCF-2 versus RUSF arm revealed that negative WLZ-correlated CAZymes were significantly suppressed by MDCF-2 supplementation compared to RUSF (p=0.004). Positive WLZ-correlated CAZymes were enriched by MDCF-2 supplementation compared to RUSF, although this enrichment did not achieve statistical significance (p=0.07, FIG. 27A, FIG. 27B; Table A and Table B).

Comparing the functional repertoire of upper- versus lower-quartile b-WLZ responders revealed that CAZymes whose abundances were increased more in MDCF-2 upper-quartile responders compared to lower-quartile responders were significantly enriched for WLZ-associated CAZymes (p=0.002). Among the CAZymes that were most enriched in the upper quartile b-WLZ responders were proteins involved in the breakdown of human milk oligosaccharides, including GH29 and GH95 a-L-fucosidases and GT2 β-galactosidase, proteins involved in the breakdown of glucose polymers, including GH13 amylase and GH133 amylo-α-1,6-glucosidase, and proteins involved in the breakdown of mannose, including GH92 mannosidase and GH26 β-mannanase.

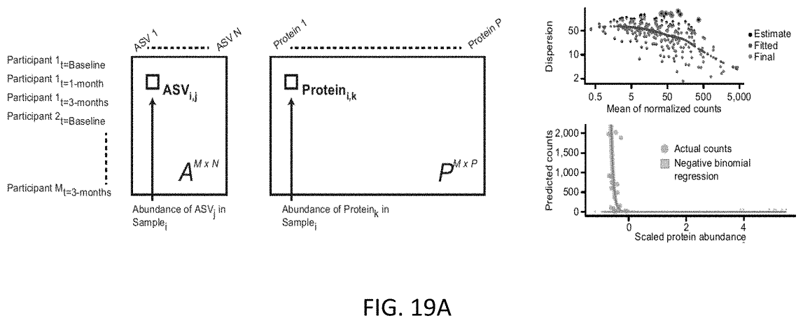

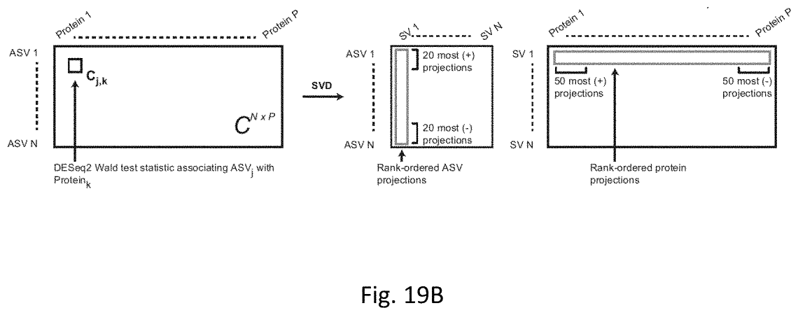

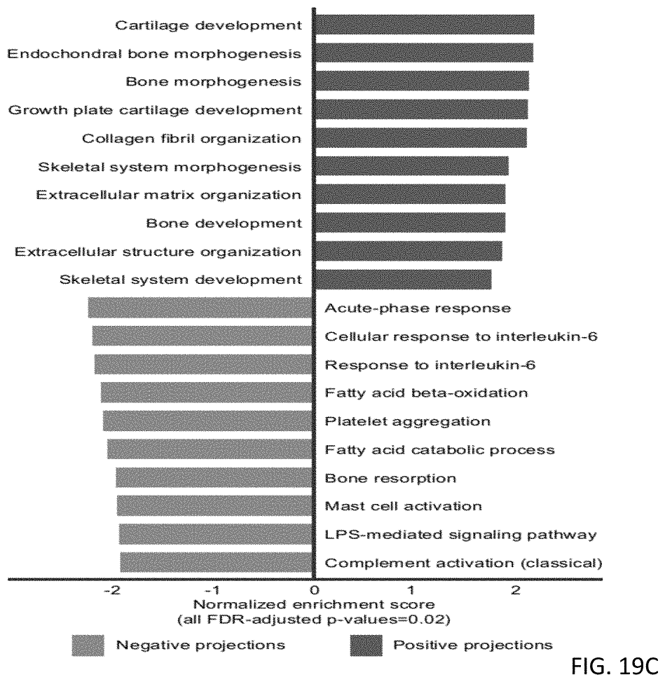

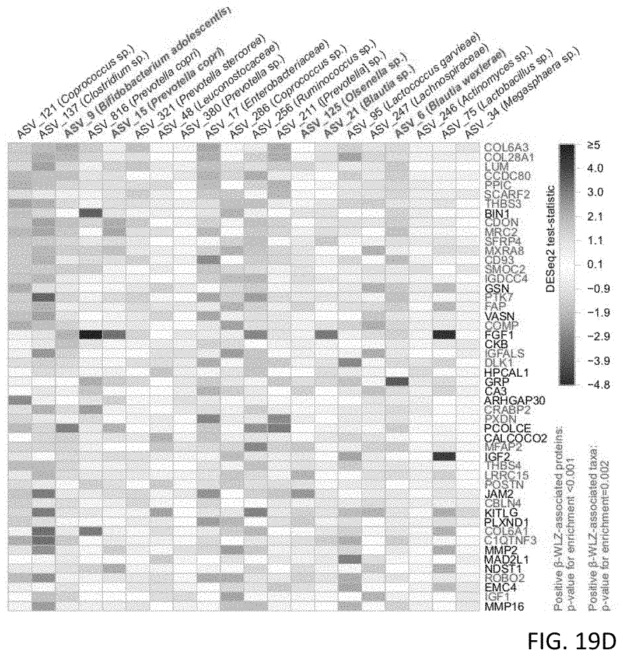

Relating features of the plasma proteome to members of the gut microbiota—We next turned to the question of whether and how features of the plasma proteome co-vary with members of the gut microbiota, especially those associated with ponderal growth. We previously described cross-correlation singular value decomposition (CC-SVD), an unbiased method for relating disparate feature types measured from the same individual. However, the distribution of ASV abundances measured in fecal samples from children in this study followed a negative binomial distribution, invalidating the statistical assumptions of CC-SVD. Therefore, we generalized CC-SVD to account for this distributional difference and developed negative binomial SVD (NB-SVD). We performed NB-SVD by first creating an association matrix where each row represents a bacterial taxon, each column represents a plasma protein, and each element of the matrix represents the test-statistic describing how strongly plasma protein k predicts the abundance of taxon j under an Empirical Bayes negative binomial regression model—a ‘correlation’ equivalent for count-based data (FIG. 19A). SVD was then performed on this association matrix to identify groups of plasma proteins that were ‘correlated’ to similar sets of bacterial taxa. Each singular vector (SV) represents a unique ‘association profile’ between proteins and ASVs that is distinct from other SVs. ASVs with positive projections onto an SV (SV+taxa) show coordinated positive associations with plasma proteins with positive projections (SV⁺ proteins) and negative associations with proteins with negative projections (SV⁻ proteins) onto that SV. Concordantly, ASVs with negative projections (SV⁻ taxa) show positive associations with SV⁻ proteins and negative associations with SV⁺ proteins (FIG. 19B). The resulting analysis provided a way to relate host biological responses with changes in the configuration of the gut microbiota during nutritional supplementation (see Supplementary Methods).

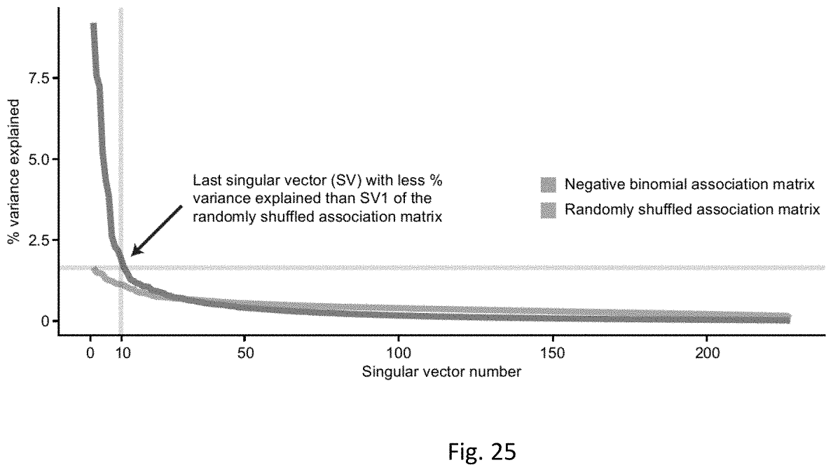

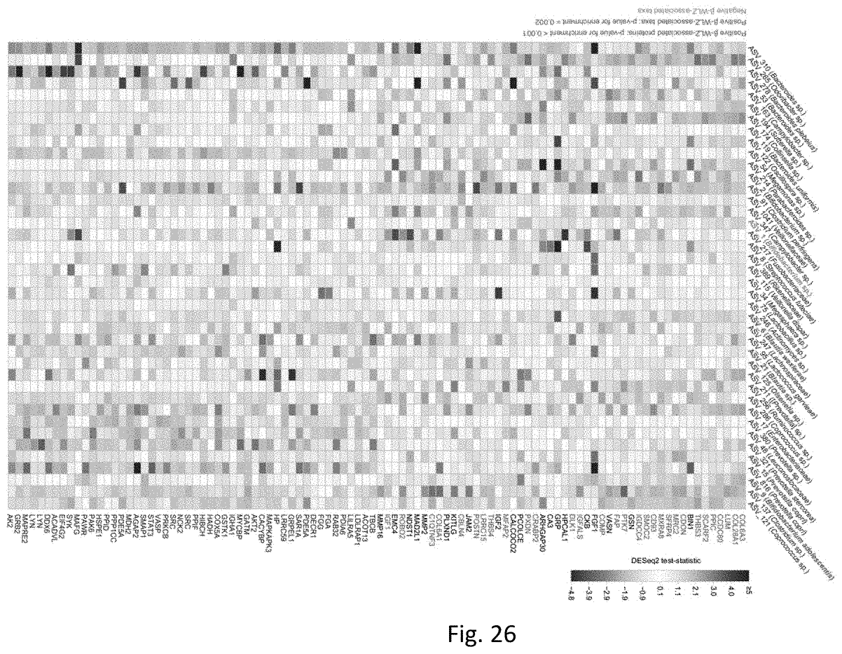

NB-SVD analysis revealed that of the ten singular vectors that carried cross-association information above noise SV8 was the only one that was significantly enriched for ‘WLZ-associated’ taxa (GSEA p=0.002, FIG. 25). Therefore, we focused on the plasma protein-ASV cross-association profile represented by SV8. The association strength of the top 20 positively projecting ASVs and the top 50 most positively projecting proteins are shown in FIG. 19C (see FIG. 26 for negatively projecting ASVs and proteins).

The top 20 taxa with positive projections on SV8 included several that were identified as significantly ‘WLZ-associated’ [e.g., Bifidobacterium adolescentis, Prevotella copri, an Olsenella sp., and two Blautia sp.] (FIG. 19C). Remarkably, SV8⁺ proteins (i.e., those that are positively associated with SV8⁺ taxa) were significantly enriched for mediators of cartilage development and bone growth; they include SFRP4, COMP, THBS4, ROBO2, and IGF1 (discussed above), as well as collagen type VI α-3 chain (COL6A3), a key regulator of skeletal muscle development and bone density (Okada et al., 2007, Mullin et al., 2018) (FIG. 194C, FIG. 19D). Additionally, the SV8⁺ proteins were significantly enriched for members of the set of 70 WLZ-associated proteins (GSEA p<0.001).

In contrast, SV8⁻ proteins (i.e., those that are negatively associated with SV8+ taxa) were significantly enriched for mediators of acute phase response, interleukin-6 (IL-6) activation, fatty acid oxidation, and bone resorption (FIG. 19C, FIG. 19D, FIG. 26). The top 20 SV8⁻ taxa included several Bacteroides sp., Campylobacter sp., and the Bifidobacterium sp. that was significantly negatively associated with β-WLZ; these bacteria were negatively associated with SV8⁺ proteins enriched for bone growth and positively associated with SV8⁻ proteins related to inflammation, beta-oxidation, and bone resorption (FIG. 22). The results provided by NB-SVD analysis reveal that the abundances of protein mediators of bone growth and inflammation are coupled to the representation of WLZ-associated taxa, providing further evidence of potential mechanisms by which components of the gut microbiota can operate to regulate ponderal growth.

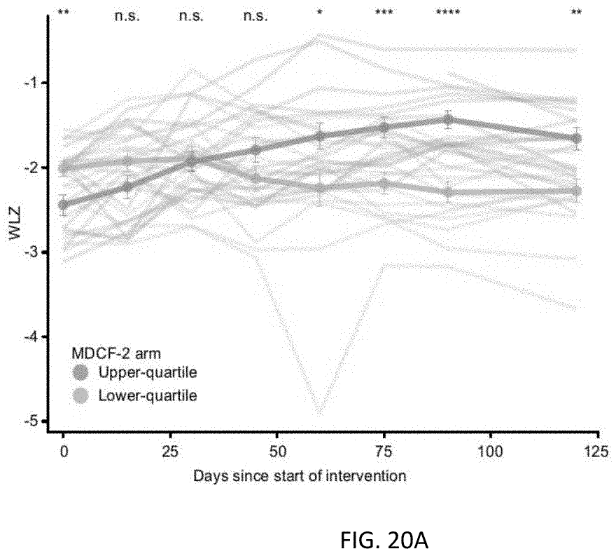

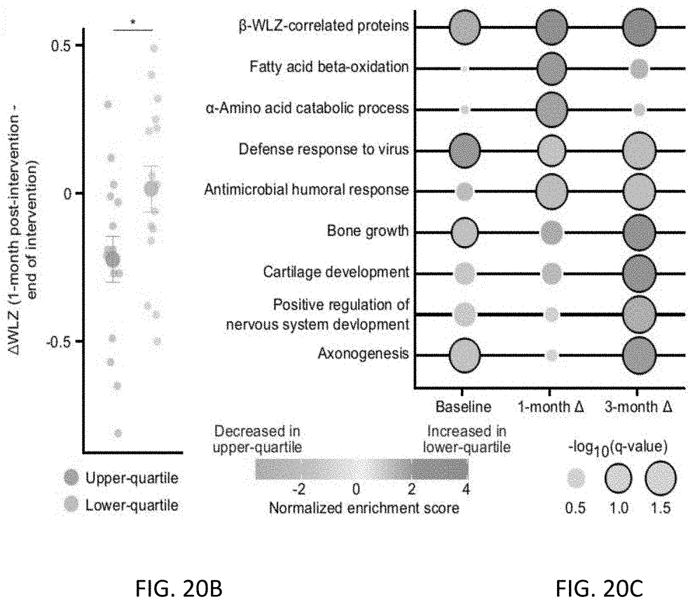

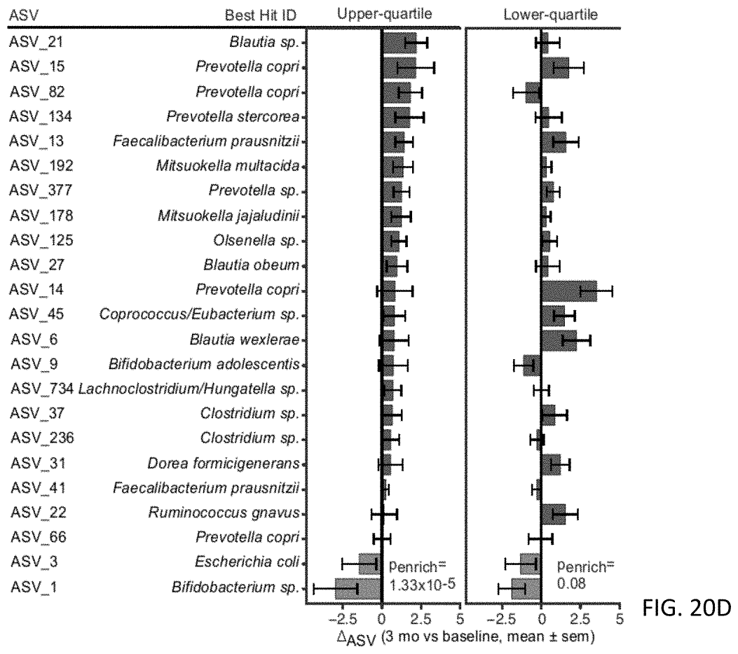

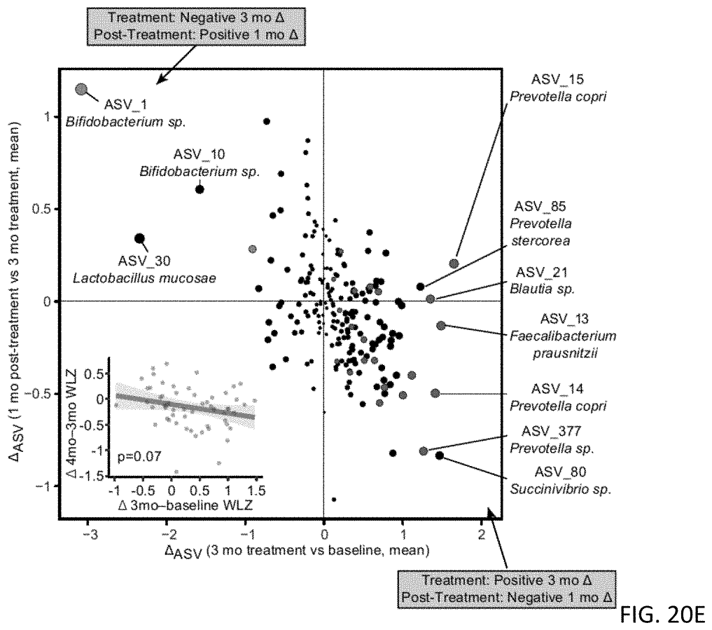

Determinants of MDCF-2 responsiveness and durability of response—To further characterize mechanisms underlying the ponderal growth response to MDCF-2, we divided the cohort of children given MDCF-2 into upper and lower quartiles based on their ponderal growth rates (β-WLZ; n=15 children/group). Those in the upper-quartile started off significantly more wasted at baseline (p=0.008, t-test), but within the first month of intervention showed complete catch-up growth to the lower-quartile responders (p=0.82; FIG. 20A). By the end of the 3-month intervention, these children had significantly higher WLZ than those in the lower-quartile (p<0.0001), suggesting that the differences in growth rates were not simply due to regression toward the mean (FIG. 20B). During the 1-month follow-up period after completion of MDCF-2 supplementation, children in the upper-quartile exhibited a greater drop in WLZ compared to those in the lower-quartile (p=0.04), but still maintained a significantly higher WLZ at this timepoint (p<0.003; FIG. 20B).

Comparison of the plasma proteomes of the two groups revealed that at baseline, those in the upper-quartile had higher levels of proteins associated with anti-viral immune activation including interferon α-1 (IFNA1), interferon λ-2 (IFNL2), IL-1β, IL-6, and CXCL9, and to a lesser degree, protein mediators of antimicrobial humoral immune responses (FIG. 20C). Conversely, at baseline, upper-quartile responders had lower levels of mediators of bone growth and axonogenesis, as well as WLZ-associated proteins (e.g., COMP, COL6A3, THBS3, SLITRK3, SLITRK5, and LEP) compared to children in the lower-quartile of ponderal growth response (FIG. 20C). These results indicate that children with a pro-inflammatory, growth mediator-depleted state at baseline exhibit more rapid increases in β-WLZ during MDCF-2 treatment.

After one month of supplementation, WLZ-associated proteins, and to a lesser extent, bone growth-related proteins, increased while anti-viral defense and antimicrobial immune activation-related proteins decreased more in children in the upper- compared to lower-quartile (FIG. 20C). Proteins related to axonogenesis did not show significantly different changes in abundances between the two responder groups after one month of treatment (FIG. 20C). Interestingly, proteins associated with amino acid catabolism and fatty-acid oxidation increased significantly more after one month of intervention in those in the upper-quartile compared to the lower-quartile, but normalized after completion of the intervention (FIG. 20C). The transient metabolic shift toward amino acid utilization may reflect more successful adaptation to the additional dietary protein provided by MDCF-2 in upper-quartile individuals and is consistent with previous work on nutritional supplementation in children with severe acute malnutrition (Gehrig et al., 2019).

After three months of supplementation, bone growth and cartilage development-related proteins were significantly more increased by MDCF-2 in the children manifesting the upper-quartile β-WLZ responses compared to those in the lower-quartile (FIG. 20C). Matrilin-4 (MATN4), a cartilage extracellular matrix protein required for normal joint development and maintenance (Li et al., 2020), THBS3, COMP, COL6A3, and LEP were the bone growth-associated proteins that were most significantly increased after MDCF-2 supplementation in the upper-quartile compared to lower-quartile responders. Interestingly, the inhibitory IGF binding protein IGFBP-2 and growth factor differentiation factor 15 (GDF15), which is associated with anorexia and lipolytic biomarkers in children with severe acute malnutrition (Gehrig et al., 2019), were significantly decreased by MDCF-2 treatment in upper-quartile responders compared to lower-quartile β-WLZ responders. Additionally, proteins related to axonogenesis and the positive regulation of nervous system development were also significantly increased more in the upper-quartile versus lower-quartile responders; they include cellular retinoic acid-binding protein 2 (CRABP2), a facilitator of the conversion of dietary carotenoids to Vitamin A (Napoli et al., 2020), SLITRKS, NTRK2, and the axon guidance receptor UNCSB.

In contrast, proteins involved in antimicrobial humoral immune response were significantly decreased more after 3 months of MDCF-2 supplementation in upper-compared to lower-quartile β-WLZ responders (FIG. 20C). The three most significantly reduced pro-inflammatory proteins were GNLY, CXCL11, which is a T-cell chemoattractant and ligand for the Th1 T-cell receptor CXCR3, and IGHA1; other inflammatory proteins that were decreased more (although not significantly so after adjustment for multiple hypotheses) after MDCF-2 supplementation in upper-quartile compared to lower-quartile responders included the neutrophil gelatinase lipocalin-2 (LCN2) which is elevated in a litany of inflammatory disorders (Moschen et al., 2017), Lithostathine-1-α and -β (REG1A, REG1B), and the C-type lectin regenerating islet-derived protein 3-α (REG3A). While expression of REG-family proteins is normally confined to the intestine, REG1A and REG1B are elevated in the serum of children with Celiac Disease and in the feces of undernourished children at risk for stunting, respectively (Planas et al., 2011, Peterson et al., 2013). REG3A is elevated in the plasma of patients with inflammatory bowel disease, and highly correlated with the abundances of inflammatory proteins in the proximal intestine of stunted children with environmental enteropathy (Marafini et al., 2017, Chen et al., 2020).

A comparison of the microbiota response to MDCF-2 between the baseline and 3-month time points revealed that ASVs whose abundances were increased in those with upper-quartile β-WLZ responses were significantly enriched for WLZ-associated taxa (p<0.001, Fisher's Exact Test); this enrichment was not observed in lower-quartile responders (p=0.08, Fisher's Exact Test; FIG. 20D). FIG. 20E compares the microbiota configurations of children at the end of the 3-month intervention versus at the end of the 1-month follow-up period. The results show that the magnitude of change in representation of the majority of MDCF-2 responsive ASVs (16 of the 21 WLZ-associated taxa) had begun to diminish during this period, just as WLZ scores trended lower, albeit not to a statistically significant degree (FIG. 20A). This latter observation provides a rationale for assessing the effects of longer duration interventions on the microbiota, the plasma proteome and host physiologic phenotypes, including linear growth and neurodevelopment.

Prospectus: We describe the results of a randomized study testing the effects of a microbiota-directed complementary food (MDCF-2) against an existing supplemental food (RUSF) on ponderal growth in 12-18-month-old Bangladeshi children with MAM. Despite its lower caloric density, MDCF-2 elicited a significantly greater rate of weight gain, changes in plasma protein mediators of bone growth, neurodevelopment and immune function and more complete repair of the gut microbiota compared to RUSF. The results provide an example of the ability to harness preclinical gnotobiotic animal models to identify microbiota-targeted therapies that translate to improved health outcomes.

The clinical outcome reported here, combined with mechanistic insights about how components of the gut community are linked to ponderal growth responses, prompt several additional questions about how WLZ improvement translates to other outcomes, as well as the timing and duration of interventions of this type. First, our study did not define the effects of MDCF-2 and RUSF on body composition (changes in fat versus lean mass). Chronic undernutrition in early life induces metabolic reprogramming that may enable a child to more efficiently capture and store energy as fat during times of nutrient scarcity (Sawaya et al., 2003). While adaptive in the short-term, this metabolic shift predisposes children to developing diabetes, hypertension, and cardiovascular disease later in life, creating a ‘double burden of malnutrition’ in areas where childhood undernutrition is endemic (Popkin et al., 2020). MDCF-2 elicits a concerted change in WLZ-associated proteins, a number of which are effectors of bone growth and skeletal muscle development. However, some of these proteins have also been implicated in metabolic disorders (e.g., cartilage intermediate layer protein 2; Wu et al., 2019). Augmenting growth of bone and skeletal muscle may promote a rebalancing of the rapid ‘catch-up’ fat accretion, observed when undernourished children are given standard nutritional interventions, towards a more appropriate lean-to-fat mass ratio, simultaneously improving growth and protecting from later obesity (Conlisk et al., 2004; Kinra et al., 2008). Given that the MDCF formulation described in this report influences host biology in ways that are distinct from conventional supplementary foods, it will be important to conduct long-term follow-up studies to ascertain its effects on body composition and metabolic health. Second, studies conducted in children with MAM in Malawi and Ethiopia indicated that increases in WLZ, MUAC, and especially ‘fat-free’ mass accretion in the first two years of life were associated with better cognitive and motor development (Abera et al., 2018; Olsen et al., 2019). Children who received MDCF-2 had increased abundances of plasma proteins associated with axonal growth and CNS development. While the source of these proteins is not known, it will be important to follow this and other cohorts of children with MAM treated with MDCF-2 for sufficiently long periods to assess its effects on cognitive development and its relationship to changes in body composition. Third, many of the ‘WLZ-associated’ taxa identified were members of a network of co-varying bacteria strains (‘ecogroup’) that define the normal postnatal ‘maturation’ of the gut microbiota (Raman et al., 2019). A hallmark of a successfully executed program of gut community development is the transition from a Bifidobacterium longum dominant to a Prevotella copri dominant microbiota. While B. longum has been associated with numerous beneficial outcomes in breast-feeding infants, its abundance was negatively associated with ponderal growth rate in the 12-18-month-old children enrolled in the present study. This observation emphasizes that the design and delivery of this and/or other MDCFs should consider how the timing of nutritional intervention aligns with the state of microbiota development. Fourth, evaluation of the microbiota of subjects in the current study one month after cessation of treatment revealed that improvements in the representation of a majority of MDCF-2 responsive WLZ-associated ASVs had begun to diminish, just as β-WLZ was diminishing, further underscoring the need for trials where children are treated for significantly longer periods. Finally, the WLZ-associated plasma and microbiota biomarkers identified in this study should enable better characterization/stratification of participant populations and adaptive study designs.

### Methods

Human study design: The human study entitled ‘Community-based Clinical Trial With Microbiota-Directed Complementary Foods (MDCFs) Made of Locally Available Food Ingredients for the Management of Children With Primary Moderate Acute Malnutrition (MAM)’ was approved by the Ethical Review Committee at the icddr,b. (ClinicalTrials.gov identifier: NCT04015999). The study was conducted in Mirpur, an urban slum in Dhaka, Bangladesh between November 2018 and December 2019. The parents/guardians of all study participants provided written informed consent. The objective of the study was to determine whether twice daily, controlled administration of a locally-produced microbiota-directed complementary food (MDCF-2) for 3 months to children with MAM provided superior improvements in weight gain, microbiota repair, and improvements in the levels of key plasma biomarkers/mediators of healthy growth compared to a standard rice/lentil-based ready-to-use supplementary food (RUSF) formulation used in Bangladesh that was not designed to repair the gut microbiota (see Table 16 for compositions and nutritional analysis of the two formulations).

A total of 124 male and female children with MAM (WLZ between -2 and -3) aged between 12- and 18-months-old who satisfied the inclusion/exclusion criteria were enrolled, with 62 children randomly assigned to each treatment arm using the permuted block randomization method. Participants/care providers and outcomes assessors were blinded to the intervention assignments (see Mostafa et al., 2020 for detailed descriptions of the study design, sample size calculation, preparation of the MDCF-2 and RUSF formulations and data collection methods).

At enrollment, anthropometric data and a fecal sample was collected from each child. On the first day of starting nutritional supplementation, anthropometric measurements were obtained together with a fecal and plasma biospecimen; these data and biospecimens were subsequently collected at regular intervals throughout the 3-month intervention period (see below and FIG. 16). Socio-demographic characteristics of participants' families were collected at enrollment. Data related to morbidity (cough, runny-nose, fever and diarrhea) were documented daily throughout the intervention period.

During the first month of the study, each child was brought to a study center twice daily (morning and afternoon). On each visit, mothers were provided 25 g of their assigned food supplement (MDCF-2 or RUSF) and asked to spoon feed their child, under the supervision of trained study personnel, until she/he refused to eat further. The amount of food consumed at each visit was recorded by subtracting that left over from the offered amount; pre-weighed napkins were used to collect any food regurgitated or spilled, which was deducted from the amount provided. Other than being requested not to feed their child for 2 hours prior to visiting the study center, mothers were advised to continue their usual breastfeeding/ complementary feeding practices throughout the study. Children were monitored daily by Field Research Assistants for any side effects/adverse events and treated according to standard of care if needed. In the second month, each child was provided 25 g of their assigned food supplement at the feeding center, and an additional 25 g was provided in a clean container to feed at home. In the third month, two separate containers containing 25 g of study diet were delivered each day to each enrolled child at their home. Any unconsumed diet from each feeding was retained in the container. Each day, food consumption histories for each child were collected and the weight of study diet consumed was determined by weighing the food remaining in the container. After completing 3 months of intervention, children returned to their normal feeding routine, but continued to be monitored, with fecal sampling and anthropometry, for a period of 1 month (and subsequently every 6 months for 2 years).

Fecal samples were collected at participants' homes within 20 minutes of production by study personnel, transferred in 2 mL cryovials to Cryo Exchange vapor shippers (Taylor-Wharton/Worthington Industries, CX-100) and transported to the study center where they were recorded and stored at −80° C. EDTA-plasma was prepared from blood collected during scheduled visits to the study center as previously described (Gehrig et al., 2019) and stored at −80° C. Coded biospecimens were shipped to Washington University on dry ice where they were stored at −80 ° C., along with associated metadata, in a dedicated repository with approval from the Washington University Human Research Protection Office.

Analysis of clinical characteristics: Enrollment and baseline characteristics—Comparisons of demographic, anthropometric, and environmental features at enrollment between children receiving MDCF-2 or RUSF were performed using two-sided unpaired t-tests for normally distributed features, Wilcoxon rank-sum tests for measurements with skewed distributions, or Chi-squared tests for categorical variables. Immunization status was classified as complete, partial or none. Breastfeeding status at enrollment was categorized as exclusively breast fed, partially breast fed or never breast fed since birth (see ref. X for details of this classification scheme). Notably, the first day of intervention began on average of 5.88±0.14 (SEM) days after enrollment; for all analyses of anthropometric measurements (including for the primary analysis described below), the first day of intervention was used as the baseline measurement. Comparisons of baseline anthropometric measures between children receiving RUSF and those receiving MDCF-2 were performed using a linear model controlling for baseline age, gender, and any history of illness 7 days prior to enrollment.

Analysis of clinical characteristics: Primary analysis of anthropometric response to MDCF-2 or RUSF—For each intervention, the primary outcome of ponderal growth rate was calculated using a mixed-effects linear model that predicted WLZ from weeks in the intervention, controlling for baseline age, gender, any history of illness 7 days preceding enrollment, and a random intercept for each participant. The model took the form:

WLZ˜β₁(weeks in intervention)+β₂(baseline age)+β₃(gender)+β₄(history of illness)+(1|PID) (1)

The rates of growth for children receiving MDCF-2 or RUSF reported in Table 15 are β₁ in Equation (1) and represent how much WLZ increased per week in a given treatment arm. The same equation was used to calculate WAZ, LAZ, and MUAC growth rates, substituting WLZ in Equation (1) for the appropriate anthropometric feature of interest.

A comparison of the effects between MDCF-2 and RUSF on growth rates was performed using a mixed effects linear model predicting WLZ from the interaction between weeks in the intervention and treatment, controlling for baseline age, gender, any history of illness 7 days preceding enrollment, weeks in the intervention, treatment, and a random intercept for each participant. The model took the form of (2):

WLZ˜β₁(treatment: weeks in intervention)+β₂(baseline age)+β₃(gender)+β₄(history of illness)+β₅(treatment)+β₆(weeks in intervention)+(1|PID) (2)

The differential rate of ponderal growth as a function of treatment arm (MDCF-2 vs RUSF) reported in Table 15 is β₁ in Equation (2) and represents how much more WLZ improved in children receiving MDCF-2 compared to those receiving RUSF per week. Equation (2) was also used to compare the effects of MDCF-2 and RUSF on rates of change of WAZ, LAZ, and MUAC by substituting WLZ for the anthropometric measure of interest.

Analysis of clinical characteristics: Analysis of illness and co-morbidities during supplementation—For each intervention, the change in prevalence of fever, diarrhea, cough, or runny-nose was quantified using a generalized mixed-effects linear model with a logit link function that predicted a given co-morbidity from weeks in the intervention, controlling for a random intercept for each participant. The model took the form of (3):

morbidity˜β₁(weeks in intervention)+(1|PID) (3)

The within-treatment log-odds ratio detailed in Table 17is β₁ in Equation (3) and represents how much more/less likely it would be to have a co-morbidity each week during the intervention period.

A comparison of the effects between MDCF-2 and RUSF on the prevalence of fever, diarrhea, cough, or runny-nose was performed using a generalized mixed effects linear model with a logit link function predicting a particular co-morbidity from the interaction between weeks in the intervention and treatment, controlling for the main effects of treatment, weeks in the intervention, and a random intercept for each participant. The model took the form of (4):

morbidity˜β₁(treatment: weeks in intervention)+β₂(treatment)+β₃(weeks in intervention)+(1PID) (4)

The differential prevalence of co-morbidity as a function of treatment arm (MDCF-2 vs RUSF) reported in Table 17 is β₁ in Equation (4) and represents how much more likely a given co-morbidity is to be reported each week in the MDCF-2 compared to the RUSF arm.

Analysis of the plasma proteome: Processing of plasma samples—The aptamer based SomaScan 5K Proteomic Assay plasma/serum kit (SomaLogic) was used to quantify the abundances of 5,284 proteins in plasma samples collected from children prior to, undergoing, and immediately after nutritional supplementation with MDCF-2 or RUSF. Plasma samples were processed according to manufacturer's instructions as previously described (Chen et al., 2020). Briefly, 50 pL of plasma were incubated with NHS-biotin-tagged, protein-specific aptamer probes ('SOMAmers') to form protein-SOMAmer complexes that were immobilized on streptavidin beads. The complexes were subsequently cleaved, denatured, eluted, and hybridized to a custom Agilent DNA microarray. The arrays were scanned with an Agilent SureScan instrument at 5 pm resolution and the Cy3 fluorescence signal was quantified and processed using SomaLogic's SomaScan standardization procedures (Chen et al., 2020).

Additional quality control (QC) steps were performed in-house. SOMAmers that were not specific to human proteins or that were marked by SOMAlogic as deprecated were removed. Additionally, SOMAmers were removed whose median fluorescence signal across all samples were within 4.9 median average deviances (MAD) from blanks, resulting in a total of 4,977 SOMAmers that passed quality control (FIG. 23). Protein abundances were loge-transformed and quantile-normalized prior to all downstream plasma proteomic analyses (Chen et al., 2020).

Analysis of the plasma proteome: Identification of ‘WLZ-associated’ proteins—Pearson correlations between changes in protein abundances and ponderal growth rates were used to nominate ‘WLZ-associated’ proteins. Because WLZ was measured every 15 days (a total of seven measurements throughout the course of the intervention) while plasma protein abundances were only quantified at three time points (baseline, one and three months following the start of intervention), we developed the following strategy to maximize information used to quantify protein-anthropometry relationships. First, a linear model predicting WLZ from time in the intervention was created for each participant, yielding 118 β-coefficients that describe the ponderal growth rate (β-WLZ) of each child from whom matched anthropometric and proteomic data were available. Next, for each participant, the change in protein abundances between the start-of-intervention and the end-of-intervention timepoint was calculated, producing 118 A-abundances for each protein. Finally, the 118 β-WLZs were correlated against the 118 A-abundances for each of the 4,977 proteins that passed QC, resulting in 4,977 Pearson correlation coefficients that captured the associations between changes in protein abundances and changes in ponderal growth. ‘WLZ-associated’ proteins were defined as proteins whose changes in abundances were significantly positively correlated with β-WLZ [FDR-adjusted p-value (q-value) less than 0.1)]. Enrichment for GO ‘biological processes’ was performed by rank-ordering proteins by their Pearson correlation coefficient, then performing gene set enrichment analysis (GSEA) using the fgsea package in R (Sergushichev, 2016) to calculate enrichment p-values (10,000 permutations).

Analysis of the plasma proteome: Differential abundance analysis—Differential abundance analyses between timepoints, intervention, or the interaction between timepoint and intervention were performed using limma (Ritchie et al., 2015). The ‘duplicateCorrelation’ function, which corrects for correlations within a blocked design, was used to account for the repeated measurements taken from each participant, resulting in the equivalent of a mixed effects linear model with a random intercept for each child. The term ‘significant’ was reserved solely for statistical inferences that had a q-value<0.1; differences that did not reach this threshold were not described as ‘significant’. Enrichment for GO ‘biological processes’ was performed by rank-ordering proteins by their limma test-statistic, then employing the fgsea package in R to calculate enrichment as described above.

Analysis of fecal microbial communities: V4-16S gene sequencing and analysis—Fecal samples were pulverized in liquid nitrogen. DNA was extracted, purified, and indexed IIlumina libraries of the V4 region of the bacterial rRNA gene were prepared from ˜50 mg of pulverized material as previously described (Gehrig et al, 2019). Libraries were quantified, pooled, and sequenced using an Illumina MiSeq instrument to generate paired-end, 250 nt reads (3.29×10⁴±9.93×10³ reads/sample; mean±SD). Amplicon sequences were processed to trim adapter and primer sequences using bbtools (v37.02). DADA2 (Callahan et al., 2016) was used to analyze preprocessed, paired-end sequence data to obtain and quantify error-corrected amplicon sequence variants (ASVs) in R (v3.6.1). Taxonomic assignments were performed using the DADA2 implementation of the Ribosomal Database Project Naïve Bayesian Classifier (database v16) at a minimum bootstrap confidence of 80% (option ‘minboot=80’). Tables of ASV abundances (counts) for each sample were combined with sample metadata and taxonomic assignment into a phyloseq (v1.3.0) object in R. Samples with fewer than 2000 reads were excluded from further analysis. Contaminating mitochondrial or chloroplast ASV sequences were removed, along with any bacterial-origin ASVs lacking Phylum-level taxonomic classification. A count filter was applied to remove any ASVs present below five counts in fewer than 5% of samples, yielding a filtered table containing 209 ASVs across 939 samples. This filtered ASV table was adjusted for library size and normalized (variance stabilizing transformation) using DESeq2 (Love et al., 2014). Mixed-effects linear models (R packages Ime4 v1.1.23 and ImerTest v3.1.1) were used to relate the abundance of ASVs in each trial participant to the same participant's anthropometric characteristics using model formulas of form of (5):

WLZ˜β₁(ASV abundance)+β₂(week sinces baseline)+(1|PID) (5)

ANOVA was used to determine the significance of relationships between model terms and WLZ. WLZ-associated ASVs were identified as those exhibiting false-discovery-rate adjusted p-values 0.05. Differences in ASV abundance were calculated for each taxon in each trial participant between the beginning and end of the respective therapeutic food intervention and between the end of intervention and the one-month follow-up timepoint. These ASV responses were averaged within and compared between the (i) MDCF2 and RUSF trial arms and (ii) upper-quartile and lower-quartile b-WLZ response participants, and the enrichment of WLZ-associated ASV responses for these comparisons was calculated using Fisher's Exact Test. The durability of ASV responses was determined by comparing the beginning to end of treatment response of each taxon to the end of treatment to one-month follow-up response in each trial participant for the comparisons described above.

Negative-binomial singular value decomposition (NB-SVD) analysis—We previously described cross-correlation singular value decomposition (CC-SVD), an analytical technique that can be used to reveal associations between disparate feature types measured in the same individuals (Chen et al., 2020). However, because bacterial abundances measured in fecal samples from this study followed a negative binomial distribution, the statistical assumptions of CC-SVD were violated. Thus, we developed negative binomial SVD (NB-SVD), a statistical method that can be used to identify associations between disparate feature types measured from the same individuals when one feature type follows a negative-binomial distribution. NB-SVD analysis begins with two abundance matrices—one for the abundances of ASVs, the other for the abundances of proteins. Each element of the ASV abundance matrix A^{M×N} contains A_{i,j}—the abundance of ASV j in fecal sample i—while each element of the protein abundance matrix P^{M×P} contains element P_{i,k}, which is the abundance of protein k in plasma sample i. Each row i represents abundances quantified in matched plasma and fecal samples taken from the same individual at the same timepoint during intervention (baseline, one month, or three months after starting intervention). All 118 participants who had available fecal and plasma samples at baseline, one month, and three months were included as rows in A^{M×N} and P^{M×P}. Additionally, A^{M×N} was filtered to remove any ASV that was present in less than 5% of samples.

Next, a cross-association matrix between proteins and ASVs is created. For a given plasma protein k, negative binomial regression with Empirical Bayes shrinkage was used to predict the expected counts of each ASV from the abundance of protein k (Love et al., 2014). This procedure was implemented using the R package DESeq2 with the model formula ‘˜proteinₖ’, a local fit for the Empirical Bayes shrinkage, and default settings for all other parameters. The output for DESeq2 is the estimated log₂(fold-change) in the expected counts for ASVs_{1:j=N} for a one-unit change in the abundance of protein k, as well as the test-statistic (z-score) for the estimated coefficient. The reported DESeq2 z-score for each ASV-protein relationship represents a standardized metric that quantifies the likelihood and direction of association between the abundance of bacterial taxa j and protein k. Repeating this procedure for all 4,977 proteins yields a taxa-by-protein association matrix C^{N×P} where each element C_{j,k} of the matrix is the test-statistic reported by DESeq2 for that taxa-protein pair.

Singular value decomposition (SVD) is then performed on the association matrix C^{N×P} to identify distinct cross-association profiles between groups of proteins and groups of bacterial taxa. SVD is a technique that separates modes of variation into statistically uncorrelated components, called singular vectors (SVs). SVs are ordered by the amount of variation they explain about the rows and columns of C^{N×P}; SV1 explains the most variation, SV2 explains second most, etc. SVD generates both row and column SVs, which contain the projections of the rows (ASVs) and columns (proteins) of C^{N×P} respectively. A projection onto an SV represents how much a given feature correlates with that SV. Because C^{N×P} contains the association (i.e. the negative binomial regression test-statistic calculated by DESeq2) between the abundances of bacterial taxa and proteins, an SV represents a cross-association profile between these two feature types. Therefore, ASVs or proteins with the largest magnitude projections will have a cross-association profile most similar to that of the SV they most strongly project on. The most positively projecting ASVs will be strongly associated with the most positively projecting proteins and negatively associated with the most negatively projecting proteins. Similarly, the most negatively projecting ASVs will be strongly associated with the most negatively projecting proteins and negatively associated with the most positively projecting proteins. Rank-ordering features by their projections onto each SV and choosing the top most positively and negatively projecting features—20 in each direction for ASVs, 50 in each direction for proteins—provides a rational way for identifying coordinated groups of bacteria and proteins whose abundances are tightly coupled.

Because SVD identifies uncorrelated components, each SV represents a unique cross-association profile distinct from that of other SVs. To determine the number of SVs that contain cross-association information above noise, a random-matrix approximation was employed (Plerou et al., 2002). Briefly, CNIxP was shuffled along each column to produce a randomized association matrix without any information about the relationship between taxa and proteins. SVD was performed on the randomized matrix, and the percent variance explained by SV1 was used as the noise threshold; any SV calculated from the SVD of C^{N×P} that explained less variation than SV1 of the shuffled matrix was deemed noise (FIG. 25). Using this method, the first 10 SVs were retained for downstream enrichment analyses.

To identify whether any of the first 10 bacterial SVs were enriched for WLZ-associated taxa, GSEA was performed on the rank-ordered ASV projections along each SV, using the list of ‘WLZ-associated’ taxa (described above) as the reference set. The same procedure was performed for protein projections to determine whether any protein SVs were enriched for WLZ-associated proteins.

Preparation of MDCF-2 and RUSF: A food processing laboratory was established in the Mirpur area, in close proximity to the nutrition centers where the intervention was provided. All raw ingredients were purchased from a single local market in Dhaka. Each step of food preparation, including cleaning, roasting, particle size reduction, homogeneous blending, and supply to the nutrition centers was performed and monitored by icddr,b study investigators and field supervisors. Upon receiving the raw dry food ingredients (rice, lentils, chickpeas, soybeans, peanuts), any foreign material, grains or seeds were removed manually and by using a sieve. The ingredients were roasted in an open pan at 120-130 ° C. for 8-10 minutes, then allowed to cool and were subsequently ground. At this stage, peanut was ready for mixing. The other food ingredients were converted into fine particles by blending for 4 to 5 minutes and sieving. Sugar was ground and the resulting fine powder was mixed with the other ingredients. Unpeeled whole green bananas were placed in a deep pan and boiled in water for 17-20 minutes until they were tender. The peel was removed and the fruit was grated into small pieces, which after cooling, were mashed with a potato masher. The weights of all the ingredients required for preparing MDCF-2 and RUSF were recorded, pre-weighed micronutrient premix powder was added and the supplementary foods were produced in small batches by mixing all ingredients in an electric blender.

The MDCF-2 and RUSF formulations were prepared fresh daily and dispensed and fed to participants on the same day. Samples of the food were routinely cultured at the icddr,b Food Safety Laboratory; tests included scoring total aerobes on plates, total coliforms, Escherichia coli, Enterobacteriaceae, Bacillus cereus, Salmonella spp, Shigella spp, Campylobacter spp, coagulase positive and other Staphylococci, as well as yeasts and molds. The nutritional composition (energy content, moisture, protein, total fat, total carbohydrate, dietary fiber, ash) of the ingredients was assessed at the Institute of Nutrition, Mahidol University, Thailand following standard procedures.

### REFERENCES

M. Abera, M. Tesfaye, B. Admassu, C. Hanlon, C. Ritz, R. Wibaek, K. F. Michaelsen, H. Friis, J. C. Wells, G. S. Andersen, R. Girma, P. Kaestel. Body composition during early infancy and developmental progression from 1 to 5 years of age: The Infant Anthropometry and Body Composition (iABC) cohort study among Ethiopian children. Br. J. Nutr.119,1263-1273 (2018).

S. Bartz, A. Mody, C. Hornik, J. Bain, M. Muehlbauer, T. Kiyimba, E. Kiboneka, R. Stevens, J. Bartlett, J. V. St Peter, C. B. Newgard, M. Freemark. Response to treatment, and predictors of mortality. J. Clin. Endocr. Metab.99, 2128-2137 (2013).

B. C. Bernardo, D. Belluoccio, L. Rowley, C. B. Little, U Hansen, J. F. Bateman. Cartilage intermediate layer protein 2 (CILP-2) is expressed in articular and meniscal cartilage and down-regulated in experimental osteoarthritis. J. Biol. Chem. 286, 37758-37767 (2011).

R. Bjarnason, B. Andersson, H. S. Kim, B. Olsson, D. Swolin-Eide, R. Wickelgren, B. Kriström, B., Carlsson, K. Albertsson-Wikland, L. M. S. Carlsson. Cartilage oligomeric matrix protein increases in serum after the start of growth hormone treatment in prepubertal children. J. Clin. Endocr. Metab. 89, 5156-5160 (2004).

R. E. Black, L. H., Allen, Z. A. Bhutta, L. E. Caufield, M. de Onis, M. Ezzati, C. Mathers, J. Rivera Maternal and child undernutrition: global and regional exposures and health consequences. Lancet. 371, 243-260 (2008).

R. E. Black, C. G. Victora , S. P. Walker, Z. A. Bhutta, P. Christian, M. de Onis, M. Ezzati, S. Grantham-McGregor, J. Katz, R. Martorell, R. Uauy. Maternal and child undernutrition and overweight in low-income and middle-income countries. Lancet. 382, 427-451 (2013).

J. M. Brix, E. C. Krzizek, C. Hoebaus, B. Ludvik, G. Schernthaner, G. H. Schernthaner. Secreted Frizzled-Related Protein 4 (SFRP4) is Elevated in Patients with Diabetes Mellitus. Horm. Metab. Res. 48, 345-348 (2016).

A. Burger, J. Roosenboom, M. Hossain, S. M. Weinberg, J. T. Hecht, K. L. Posey. Mutant COMP shapes growth and development of skull and facial structures in mice and humans. Mol. Genet. Genomic Med. 1-8 (2020).

R. K. Campbell, K. J. Schulze, S. Shaikh, R. Raqib, L. S. F. Wu, H. Ali, S. Mehra, K. P. West, P. Christian. Environmental enteric dysfunction and systemic inflammation predict reduced weight but not length gain in rural Bangladeshi children. Br. J. Nutr. 119, 407-414 (2018).

K. Chen, P. Y. Ng, R. Chen, D. Hu, S. Berry, R. Baron, F. Gori, Sfrp4 repression of the Ror2/Jnk cascade in osteoclasts protects cortical bone from excessive endosteal resorption. Proc. Natl. Acad.Sci. U.S.A. 116, 14138-14143 (2019).

R. Y. Chen, V. L. Kung, D. Subhashish, S. Hossain, M. C. Hibberd, J. Guruge, M. Mahfuz, K. N. Begum, M. M. Rahman, S. M. Fahim, Md.A.Gazi, M. R. Hague, S. A. Sarker, R. N. Mazunder, B. Di Luccia, K. Ahsan, E. Kennedy, J. Santiago-Borges, D. A. Rodionov, S. A. Leyn, A. L. Osterman, M. J. Barratt, T. Ahmed, J. I. Gordon. Duodenal microbiota in stunted undernourished children with enteropathy. New Engl. J. Med. (2020).

A. Chiabi, C. Mbanga, E. Mah, F. N. Dongmo, S. Nguefack, F. Fru, V. Takou, V., A. Fru. Weight-for-height Z score and mid-upper arm circumference as predictors of mortality in children with severe acute malnutrition. J. Trop. Pediatrics 63, 260-266 (2017).

N. S. Children, S. E. Lee, C. P. Stewart, K. J. Schulze, R. N. Cole, L.S. Wu, J. D. Yager, J. D. Groopman, S. K. Khatry, R. K. Adhikari, P. Christian, Jr, K. P. W. The Plasma Proteome Is Associated with Anthropometric Status of Undernourished. J. Nutr. 3, 10 (2017).

A. J. Conlisk, H. X. Barnhart, R. Martorell, R. Grajeda, A. D. Stein. Maternal and Child Nutritional Supplementation Are Inversely Associated with Fasting Plasma Glucose Concentration in Young Guatemalan Adults. J. Nutr. 134,890-897 (2004).

K. G. Dewey, M. G. Hawck, K. H. Brown, A. Lartey, R. J. Cohen, J. M. Peerson. Infant weight-for-length is positively associated with subsequent linear growth across four different populations. Matern. Child Nutr.1, 11-20 (2005).

K. G. Dewey. Reducing stunting by improving maternal, infant and young child nutrition in regions such as South Asia: evidence, challenges and opportunities. Matern Child Nutr. 12, (Suppl 1) 27-38 (2016).

M.S. Diamond, M. Farzan. The broad-spectrum antiviral functions of IFIT and IFITM proteins. Nature Rev. Immunol. 13,46-57 (2013).

M. L. Estes, A. K. McAllister. Maternal immune activation: Implications for neuropsychiatric disorders. Science 353,772-777 (2016).

A. J. Etheredge, K. Manji, M. Kellogg, H. Tran, E. Liu, C. M. McDonald, R. Kisenge, S. Aboud, W. Fawzi, D. Bellinger, A. T. Gewirtz, C. P. Duggan. Markers of Environmental Enteric Dysfunction Are Associated with Neurodevelopmental Outcomes in Tanzanian Children. J.Pediatr.Gastr. Nutr. 66,953-959 (2018).

J. L. Gehrig, S. Venkatesh, H. W. Chang, M. C. Hibberd, V. L. Kung, J. Cheng, R. Y. Chen, S. Subramanian, C. A. Cowardin, M.Meier, D. O′Donnell, M. Talcott, L. D. Spears, C. C. Semenkovich, B. Henrissat, R. J. Giannone, R. L. Hettich, O. Ilkayeva, M. Muehlbauer, C. B. Newgard, C. Sawyer, R. D. Head, D. A. Rodionov, A. A. Arzamasov, S. A. Leyn, A. L. Osterman, Md I. Hossain, M. Islam, N. Choudhury, S. A. Sarker, S. Huq, I. Mahnud, I. Mostafa, M. Mahfuz, M. J. Barratt, T. Ahmed, J. I. Gordon. Effects of microbiota-directed foods in gnotobiotic animals and undernourished children. Science 365, eaau4732 (2019).

L. Gold, L., D. Ayers, J. Bertino, C. Bock, A. Bock, E. N. Brody, J. Carter, A. B. Dalby, B. E. Eaton, T. Fitzwater, D. Flather, A. Forbes, T. Foreman, C. Fowler, B. Gawande, M. Goss, M. Gunn, S. Gupta, D. Halladay, J. Heil, J. Heilig, B. Hicke, G. Husar, N. Janjic, T. Harvis, S. Jennings, E. Katilius, T. R. Kenney, N. Kim. T. H. Koch, S. Kraemer, L. Kroiss, N. Le, D. Levine, W. Lindsey, B. Lollo, W. Mayfield, M. Mehan, R. Mehler, S. K. Nelson, M. Nelson, D. Nieuwlandt, M. Nikrad, U. Ochsner, R. M. Ostroff, M. Otis, T. Parker, S. Pietrasiewicz, D. L. Resnicow, J. Rohloff, G. Sanders, S. Sattin, D. Schneider, B. Singer, M. Stanton, A. Sterkel, A. Stewart, S. Stratford, J. D. Vaught, M. Vrkljan, J. J. Walker, M. Watrobka, S. Waugh, A. Weiss, S. Wilcox, A. Wolfson, S. Wolk, C. Zhang, D. Zichi. Aptamer-based multiplexed proteomic technology for biomarker discovery. PLoS ONE 5 (2010).

S. M. Goudet, B. A. Bogin, N. J. Madise, P. L. Griffiths. Nutritional interventions for preventing stunting in children (birth to 59 months) living in urban slums in low- and middle-income countries (LMIC). Cochrane Database of Systematic Reviews, 6, Art. No. CD011695 (2019).

E. Grellety, M. H. Golden. Severely malnourished children with a low weight-for-height have a higher mortality than those with a low mid-upper-arm-circumference: III. Effect of case-load on malnutrition related mortality-policy implications. Nutr. J. 17, 1-10 (2018).

M. M. Hoffmann, C. Werner, M. BOhm, U. Laufs, K. Winkler. Association of secreted frizzled-related protein 4 (SFRP4) with type 2 diabetes in patients with stable coronary artery disease. Cardio. Diabetol. 13, 1-8 (2014).

H. Kim, Y. J. Choi, Y. S. Lee, S. Y. Park, J. E. Baek, H. K. Kim, B. J. Kim, S. H. Lee, J. M. Koh. SLIT3 regulates endochondral ossification by β-catenin suppression in chondrocytes. Biochem. Biophys. Res. Comm. 506, 847-853 (2018).

S. Kinra, K. V. Rameshwar Sarma, Ghafoorunissa, V. V. R. Mendu, R. Ravikumar, V. Mohan, I. B. Wilkinson, J. R. Cockcroft, J. R., G. D. Smith. Y. Ben-Shlomo, Effect of integration of supplemental nutrition with public health programmes in pregnancy and early childhood on cardiovascular risk in rural Indian adolescents: Long term follow-up of Hyderabad nutrition trial. BMJ. 337, 445-449 (2008).

M. N. Kosek, T. Ahmed, Z. A. Bhutta, L. Caulfield, R. L. Guerrant, E. Houpt, G. Kang, G. Lee, A. A. M. Lima, B. J. J. McCormick, B. J. J. Platts-Mills, J. C. Seidman, R. R. Blank, M. Gottlieb, S. L. Knobler, D. R. Lang, M. A. Miller, W. Checkley, D. R. Trigoso. Causal Pathways from Enteropathogens to Environmental Enteropathy: Findings from the MAL-ED Birth Cohort Study. EBioMed.18, 109-117 (2017).

P. Li, L. Fleischhauer, C. Nicolae, C. Prein, Z. Farkas, M.M. Saller, W.C. Prall, R. Wagener, J. Heilig, A. Niehoff, H. Clausen-Schaumann, P. Alberton, A. Aszodi. Mice lacking the matrilin family of extracellular matrix proteins develop mild skeletal abnormalities and are susceptible to age-associated osteoarthritis. Intnl. J. Mol. Sciences 21(2) (2020).

M. I. Love, W. Huber, S. Anders Moderated estimation of fold change and dispersion for RNA-seq data with DESeq2. Genome Biology 15(550) (2014). I. Marafini, A. Di Sabatino, F. Zorzi, I. Monteleone, S. Sedda, M. L. Cupi, C. Antenucci, P. Biancheri, P. Giuffrida, M. Di Stefano, G. R. Corazza, F. Pallone, G. Monteleone, Serum regenerating islet-derived 3-alpha is a biomarker of mucosal enteropathies. Aliment. Pharm. Ther. 40,974-981 (2014).

A. R. Moschen, T. E. Adolph, R. R. Gerner, V. Wieser, H. Tilg. Lipocalin-2: A Master Mediator of Intestinal and Metabolic Inflammation. Trends Endocrin. Met. 28, 388-397 (2017).

I. Mostafa, N. N. Nahar, Md. M. Islam, S. Huq, M. Mustafa, M. J. Barratt, J. I. Gordon, T. Ahmed. Proof-of-concept study of the efficacy of a microbiota-directed complementary food formulation (MDCF) for treating moderate acute malnutrition. BMC Public Health 20,242 (2020)

J. L. Napoli, H. S. Yoo. Retinoid metabolism and functions mediated by retinoid binding-proteins. In Methods in Enzymology (1st ed., Vol. 637). Elsevier Inc (2020).

M. M. Ngari, P. O. Iversen, J. Thitiri, L. Mwalekwa, M. Timbwa, G. W. Fegan, J. A. Berkley. Linear growth following complicated severe malnutrition: 1-year follow-up cohort of Kenyan children. Arch. Dis. Child. 104,229-235 (2019).

J. M. Njunge, A. Gwela, N. K. Kibinge, M. Ngari, L. Nyamako, E. Nyatichi, J. Thitiri, G. B. Gonzales, R. H. J. Bandsma, J. L. Walson, E. N. Gitau, J. A. Berkley. Biomarkers of post-discharge mortality among children with complicated severe acute malnutrition. Scientific Rep. 9,1-12 (2019).

M. F. Olsen, A. S. Iuel-Brockdorff, C. W. Yaméogo, B. Cichon, C. Fabiansen, S. Filteau, K. Phelan, A. Ouedraogo, J. C. Wells, A. Briend, K. F. Michaelsen, L. Lauritzen, C. Ritz, P. Ashorn, V. B. Christensen, M. Gladstone, H. Friss. Early development in children with moderate acute malnutrition: A cross-sectional study in Burkina Faso. Mat. Child Nutr.16, 1-14 (2020).

M. de Onis, A. W. Onyango, E. Borghi, C. Garza. Comparison of the World Health Organization (WHO) Child Growth Standards and the National Center for Health Statistics/WHO international growth reference : implications for child health programmes. Public Health Nutr. 9,942-947 (2006).

K. M. Peterson, J. Buss, R. Easley, Z. Yang, P. S. Korpe, F. Niu, J. Z. Ma, M. P. Olortegui, R. Hague, M. N. Kosek, W. A. Petri. REG1B as a predictor of childhood stunting in Bangladesh and Peru1-3. Am. J. Clin. Nutr. 97,1129-1133 (2013).

R. Planas, I. Pujol-Autonell, E. Ruiz, M. Montraveta, E. Cabre, A. Lucas-Martin, R. Pujol-Borrell, E. Martinez-Caceres, M. Vives-Pi. Regenerating gene la is a biomarker for diagnosis and monitoring of celiac disease: A preliminary study. Trans. Res.158, 140-145 (2011).

V. Plerou, P. Gopikrishnan, B. Rosenow, L. A. N Amaral, H. E. Stanley. A random matrix theory approach to financial cross-correlations. Phys Rev E Stat Nonlin Soft Matter Phys. 65(6) (2002)

B. M. Popkin, C. Corvalan, L. M. Grummer-Strawn, Dynamics of the double burden of malnutrition and the changing nutrition reality. Lancet 395,65-74 (2020).

A. S. Raman, J. L. Gehrig, S Venkatesh, H. W. Chang, M. C. Hibberd, S. Subramanian, G. Kang, P. O. Bessong, A. A. M. Lima, M. N. Kosek,W. A. Petri, D. A. Rodionov, A. A. Arzamasov, S. A. Leyn, A. L. Osterman, S. Huq, I Mostafa, M. Islam, M. Mahfuz, R. Hague, T. Ahmed, M.J. Barratt, J.I. Gordon. A sparse covarying unit that describes healthy and impaired human gut microbiota development. Science 365, eaau4735 (2019).

M. E. Ritchie, B. Phipson, D. Wu, Y. Hu, C. W. Law, W. Shi, G. K. Smyth. limma powers differential expression analyses for RNA-sequencing and microarray studies. Nucleic Acids Research 43(7) e47 (2015)

P. Rzehak, M. Covic, R. Saffery, E. Reischl, S. Wahl, V. Grote, M. Weber, A. Xhonneux, J. Langhendries, N. Ferre, J. Escribano, E. Verduci, E. Riva, P. Socha, DNA-Methylation and Body Composition in Preschool Children : Epigenome-Wide-Analysis in the European Childhood Obesity Project (CHOP) Study. Sci. Rep. 1-13 (2017).

A. L. Sawaya, P. Martins, D. Hoffman, S. B. Roberts. The Link Between Childhood Undernutrition and Risk of Chronic Diseases in Adulthood: A Case Study of Brazil. Nutr. Rev. 61, 168-175 (2003).

R. Saxena, C. C. Elbers, Y. Guo, I. Peter,T. R. Gaunt, J. L. Mega, M. B. Lanktree, A. Tare, B. A. Castillo, Y. R. Li, T. Johnson, M. Bruinenberg, D. Gilbert-Diamond, R. Rajagopalan, B. F. Voight, A. Balasubramanyan, J. Barnard, P. S. Braund, F. Bauer, Vliet-ostaptchouk, J. V. Van. Large-Scale Gene-Centric Meta-Analysis across 39 Studies Identifies Type 2 Diabetes Loci. Am. J. Hum. Genet. 410-425 (2012).

A. Sergushichev. An algorithm for fast preranked gene set enrichment analysis using cumulative statistic calculation. bioRxiv (2016).

O. Stenina-adognravi, E.F. Plow. Thrombospondin-4 in tissue remodeling. Matrix Biol. 75-76, 300-313 (2019).

S. Subramanian, S. Huq, T.Yatsunenko, R. Hague, M. Mahfuz, M. A. Alam, A. Benezra, J. Destefano, M. F. Meier, B. D. Muegge, M. J. Barratt, L. G. VanArendonk, Q. Zhang, M. A. Province, W. A. Petri, T. Ahmed, J. I. Gordon. Persistent gut microbiota immaturity in malnourished Bangladeshi children. Nature 510, 417-421 (2014).

WHO Multicentre Growth Reference Study Group. WHO Child Growth Standards: Growth velocity based on weight, length and head circumference: Methods and development. Geneva: World Health Organization (2009).

C. J. Willer, S. Sanna, A. U. Jackson, A. Scuteri, L. L. Bonnycastle, R. Clarke, S. C. Heath, N. J. Timpson, S. S. Najjar, H. M. Stringham, J. Strait, W. L. Duren, A. Maschio, F. Busonero, A. Mulas, G. Albai, A. J. Swift, M. A. Morken, N. Narisu, K. L. Mohlke. Newly identified loci that influence lipid concentrations and risk of coronary artery disease. Nature Genet. 40, 161-169 (2008).

T. Wu, Q. Zhang, S. Wu, W. Hu, T. Zhou, K. Li, D. Liu. CILP- 2 is a novel secreted protein and associated with insulin resistance. J. Mol. Cell. Biol. 11, 1083-1094 (2019).

What is claimed is:

1. A composition comprising chickpea flour, peanut flour, soy flour, green banana, and a micronutrient premix, wherien the micronutrient premix provides at least 60% of the recommended daily allowance of vitamin A, vitamin C, vitamin D, vitamin E, vitamin B, calcium, copper, iron, magnesium, manganese, phosphorus, potassium, and zinc for a child aged 12-18 months;
wherein the composition contains no milk, powdered milk or milk product;
wherein the composition has about 300 to about 560 kcal per 100 g of the composition, a protein energy ratio (PER) of about 8% to about 20%, and a fat energy ratio (FER) of about 30% to about 60%, and wherein the amount of protein is at least 11 g per 100 g of the composition and the amount of fat is not more than 36 g per 100 g of the composition; and
wherein the chickpea flour, the peanut flour, the soy flour, and the green banana, in total, provide at least 9 g of protein per 100 g of the composition.

2. The composition of claim 1, wherein the composition has about 350 kcal to about 560 kcal per 100 g of the composition.

3. The composition of claim 1, wherein the composition has about 400 kcal to about 560 kcal per 100 g of the composition.

4. A composition comprising chickpea flour, peanut flour, soy flour, green banana, and a micronutrient premix, wherien the micronutrient premix provides at least 60% of the recommended daily allowance of vitamin A, vitamin C, vitamin D, vitamin E, vitamin B, calcium, copper, iron, magnesium, manganese, phosphorus, potassium, and zinc for a child aged 12-18 months;
wherein the composition contains no milk, powdered milk or milk product;
wherein the composition has about 400 to about 560 kcal per 100 g of the composition, about 20 g to about 36 g of fat per 100 g of the composition, about 11 g to about 16 g of protein per 100 g of the composition, a protein energy ratio (PER) of about 8% to about 12%, and a fat energy ratio (FER) of about 45% to about 60%; and
wherein the chickpea flour, the peanut flour, the soy flour, and the green banana, in total, provide at least 9 g of protein per 100 g of the composition.

5. The composition of any one of the preceding claims, wherein the chickpea flour, the peanut flour, the soy flour, and the green banana provide at least 10 g of protein per 100 g of the composition.

6. The composition of any one of the preceding claims, wherein the chickpea flour, the peanut flour, the soy flour, and the green banana provide at least 11 g of protein per 100 g of the composition.

7. The composition of any one of the preceding claims, wherein the weight ratio of the chickpea flour, the peanut flour, the soy flour and the green banana is about 1:
about 1: about 0.8: about 1.9 respectively (chickpea flour: peanut flour: soy flour: green banana).

8. A composition comprising chickpea flour, peanut flour, soy flour, green banana, and a micronutrient premix, wherein the micronutrient premix provides at least 60% of the recommended daily allowance of vitamin A, vitamin C, vitamin D, vitamin E, vitamin B, calcium, copper, iron, magnesium, manganese, phosphorus, potassium, and zinc for a child aged 12-18 months;
wherein the composition contains no milk, powdered milk or milk product;
wherein the composition has about 400 to about 560 kcal per 100 g of the composition, about 20 g to about 36 g of fat per 100 g of the composition, about 11 g to about 16 g of protein per 100 g of the composition, a protein energy ratio (PER) of about 8% to about 12%, and a fat energy ratio (FER) of about 45% to about 60%;
wherein some or all the chickpea flour is replaced with a glycan equivalent of chickpea flour, some or all the peanut flour is replaced with a glycan equivalent of peanut flour, some or all the soy flour is replaced with a glycan equivalent of soy flour, or some or all the green banana is replaced with a glycan equivalent of green banana; and
wherein the chickpea flour or equivalent, the peanut flour or equivalent, the soy flour or equivalent, and the green banana or equivalent, in total, provide at least 9 g of protein per 100 g of the composition.

9. A composition of claim 8, wherein some or all the chickpea flour is replaced with a glycan equivalent of chickpea flour, and some or all the peanut flour, the soy flour and/or the green banana is replaced with a glycan equivalent thereof.

10. A composition of claim 8, wherein some or all the peanut flour is replaced with a glycan equivalent of peanut flour, and some or all the chickpea flour, the soy flour and/or the green banana is replaced with a glycan equivalent thereof.

11. A composition of claim 8, wherein some or all the soy flour is replaced with a glycan equivalent of soy flour, and some or all the chickpea flour, the peanut flour, and/or the green banana is replaced with a glycan equivalent thereof.

12. A composition of claim 8, wherein some or all the green banana is replaced with a glycan equivalent of green banana, and some or all the chickpea flour, the peanut flour, and/or the soy flour is replaced with a glycan equivalent thereof.

13. The composition of any one of the preceding claims, wherein the composition further comprises a fat chosen from animal fat or vegetable oil.

14. The composition of any one of claims 1 to 13, wherein the composition further comprises a fat chosen from avocado oil, canola oil, coconut oil, corn oil, cottonseed oil, flaxseed oil, grape seed oil, hemp seed oil, olive oil, palm oil, peanut oil, rice bran oil, safflower oil, soybean oil, or sunflower oil.

15. The composition of any one of claims 1 to 13, wherein the composition further comprises soybean oil, and the soybean oil provides at least 50 wt % of the total fat in the composition.

16. The composition of claim 15, wherein the soybean oil provides at least 75 wt % of the total fat in the composition.

17. The composition of claim 15, wherein the soybean oil provides at least 90 wt % of the total fat in the composition.

18. The composition of claim 15, wherein the soybean oil provides at least 95 wt % of the fat in the composition.

19. The composition of any one of claims 15 to 18, wherein, in addition to soybean oil, the composition further comprises a fat chosen from animal fat or vegetable oil.

20. The composition of any one of the preceding claims, wherein the composition further comprises additional glycans.

21. The composition of claim 20, wherein the glycan is sucrose.

22. The composition of any one of the preceding claims, wherein the composition contains no (a) seeds, nuts or nut butters, (b) cocoa nibs, cocoa powder or chocolate, (c) rice flour or lentil flour, (d) dried fruit, or any combination of (a) to (d).

23. The composition of any one of the preceding claims, wherein 50 g of the composition, when administered daily as one or more servings for at least 4 weeks to a child that is 6 months of age or older with moderate acute malnutrition, repairs the gut microbiota of the malnourished child.

24. The composition of any one of the preceding claims, wherein 50 g of the composition, when administered daily as one or more servings for at least 4 weeks to a child that is 6 months of age or older with with moderate acute malnutrition and an immature gut microbiota, aids in improving the child's growth, as defined by a statistically significant change in one or more anthropometric measurement in a manner towards healthy children of a similar chronological age.

25. The composition of claim 24, wherein an anthropometric measurement is chosen from LAZ, WLZ, WAZ, or MUAC.

26. The composition of claim 24, wherein an anthropometric measurement is chosen from WLZ, WAZ, or MUAC.

27. The composition of claim 24, wherein improvement in the child's growth is defined by a statistically significant change, in a manner towards healthy children of a similar chronological age, in (a) WLZ, WAZ, and MUAC; (b) WLZ and WAZ; (c) WAZ and MUAC; or (d) WLZ and MUAC.

28. The composition of any one of the preceding claims, wherein 50 g of the composition, when administered daily as one or more servings for at least 4 weeks to a child that is 6 months of age or older with moderate acute malnutrition and an immature gut microbiota, aids in improving the child's growth, as defined by a statistically significant change, in a manner towards chronologically-age matched reference healthy children, in the relative abundance of one or more protein in Table F, one or more protein in Table G, one or more protein in Table H, or one or more protein of Table 18.

29. The composition of any one of the preceding claims, wherein the compositon is a therapeutic food, a ready-to-eat composition, or a combination thereof.

30. A method for repairing a malnourished subject's gut microbiota, improving a malnourished subject's growth, or improving the health of a malnourished subject, the method comprising administering to the subject an effective amount of acomposition of any one of claims 1 to 26.

31. The method of claim 30, wherein the subject has severe acute malnutrition.

32. The method of claim 30, wherein the subject has moderate acute malnutrition.

33. The method of claim 30, 31, or 32, wherein the subject is a child that is at least 6 months of age.

34. The method of claim 30, 31, or 32, wherein the subject is about 6 months of age to about 18 years of age, about 6 months of age to about 15 years of age, about 6 months of age to about 10 years of age, about 6 months of age to about 5 years of age, about 6 months of age to about 2 years of age.

35. A method of analyzing the efficacy of a therapeutic intervention on the nutritional status of a subject in need thereof, the method comprising (a) determining the concentration of a plurality of healthy-discriminatory protein in a biological sample obtained from the subject, (b) administering the therapeutic intervention, (c) determining the post-therapeutic intervention concentration of each healthy-discriminatory protein from step (a), (d) determining if the concentration of each healthy-discriminatory protein was modified by the therapeutic intervention, and (e) categorizing the therapeutic intervention as efficacious in improving the nutritional status of the subject when the concentrations of more than 50% of the healthy-discriminatory proteins statistically change in a manner towards those encountered in healthy individuals after administration of the therapeutic intervention.

36. The method of claim 35, wherein the biological sample is a blood sample and the concentration of a plurality of proteins from Table 18 is measured.

37. A method of analyzing the efficacy of a therapeutic intervention on the nutritional status of a subject in need thereof, the method comprising (a) determining the concentration of a plurality of SAM-discriminatory protein in a biological sample obtained from the subject, (b) administering the therapeutic intervention, (c) determining the post-therapeutic intervention concentration of each SAM-discriminatory protein measured in step (a), (d) determining if the concentration of each of the SAM-discriminatory proteins was modified by the therapeutic intervention, and (e) categorizing the therapeutic intervention as efficacious in improving the nutritional status of the subject when more than 50% of the SAM-discriminatory protein concentrations statistically change in a manner towards those encountered in healthy individuals.

38. The method of claim 37, wherein the biological sample is a blood sample and the concentration of one or more health-discriminatory proteins from Table 18 is measured.

39. A method of analyzing the efficacy of a therapeutic intervention on the physical characteristics of a subject in need thereof, the method comprising (a) determining the concentration of a plurality of LAZ-discriminatory proteins or WHZ-discriminatory proteins in a biological sample from the subject, (b) administering the therapeutic intervention, (c) determining the post-therapeutic intervention concentration of each LAZ-discriminatory proteins or WLZ-discriminatory protein measured in step (a), (d) determining if the concentration of each of the LAZ or WLZ-discriminatory proteins was modified by the therapeutic intervention, and (e) categorizing the therapeutic intervention as efficacious in improving the physical characteristics of the subject when more than 50% of the positively correlated LAZ or WLZ-discriminatory protein concentrations rose after administration of the therapeutic intervention, or when more than 50% of the negatively correlated LAZ-discriminatory protein concentrations fell after administration of the therapeutic intervention.

40. A method of analyzing the efficacy of a therapeutic intervention on the maturity of a subject's gut microbiota, the method comprising (a) measuring the subject's gut microbiota health; (b) administering the therapeutic intervention; (c) re-measuring the subject's gut microbiota health by the method used in step (a); and (d) categorizing the therapeutic intervention as efficacious when the subject's gut microbiota health is repaired.

41. The method of any one of claims 35 to 40, wherein the therapeutic intervention is a food, a prebiotic, a probiotic, or a nutritional supplement.

42. A method of treating malnutrition in a subject in need thereof, the method comprising administering an effective amount of a composition of any one of claims 1 to 29 to a subject.

43. The method of claim 42, wherein the subject has severe acute malnutrition.

44. The method of claim 42, wherein the subject has moderate acute malnutrition.

45. The method of claim 42, 43, or 44, wherein the subject is about 6 months of age to about 18 years of age, about 6 months of age to about 15 years of age, about 6 months of age to about 10 years of age, about 6 months of age to about 5 years of age, or about 6 months of age to about 2 years of age.

46. The method of any one of claims 42 to 45, wherein about 10 g to about 1000 g of the composition is administered per day as one or more servings.

47. The method of claim 46, wherein the subject is about 6 months to about 24 months in age and about 30 g to about 150 g of the composition is administered per day in one or more servings.

48. The method of any one of claims 42 to 47, wherein the composition is administered for at least 2 weeks.

49. The method of claim 48, wherein the composition is administered for at least 4 weeks.

50. The method of claim 48, wherein the composition is administered for at least 2 months.

51. The method of claim 48, wherein the composition is administered for at least 3 months.

52. The method of claim 48, wherein the composition is administered for at least 6 months.

53. The method of claim 48, wherein the composition is administered for at least 12 months.

54. A method for increasing the abundance of mediators of bone growth, mediators of neurodevelopment, mediators of inflammation, or any combination thereof, the method comprising administering an effective amount of a composition of any one of claims 1 to 29 to the subject.
